(19) United States
(12) Patent Application Publication
(10) Pub. No.: US 2025/0034148 A1
(43) Pub. Date: Jan. 30, 2025

(54) METHODS OF USING ALK2 INHIBITORS

(71) Applicant: Keros Therapeutics, Inc., Lexington, MA (US)

(72) Inventors: Jasbir S. SEEHRA, Lexington, MA (US); Jennifer LACHEY, Lexington, MA (US); Christopher R. ROVALDI, Swampscott, MA (US); Elissa FURUTANI, Belmont, MA (US)

(21) Appl. No.: 18/706,647

(22) PCT Filed: Nov. 2, 2022
(86) PCT No.: PCT/US2022/048703

§ 371 Date: May 1, 2024

(51) Int. Cl. C07D 487/04; A61K 31/496; A61K 31/5025; A61K 31/517; A61K 31/519; A61K 31/5377; A61K 31/541; A61K 31/55; A61K 31/551; A61K 31/675; A61P 7/06; C07D 213/56; C07D 405/14; C07D 471/04; C07D 519/00

(52) U.S. Cl. CPC C07D 487/04; A61K 31/496; A61K 31/5025; A61K 31/517; A61K 31/519; A61K 31/5377; A61K 31/541; A61K 31/55; A61K 31/551; A61K 31/675; A61P 7/06; C07D 213/56; C07D 405/14; C07D 471/04; C07D 519/00

(57) ABSTRACT

The invention relates to methods of treating functional iron deficiency (FID) associated with low, very low, or intermediate risk myelodysplastic syndromes. FID associated with primary myelofibrosis (PMF), post-polycythemia vera myelofibrosis (post-PV MF), or post-essential thrombocythemia myelofibrosis (post-ET MF), and FID associated with myelodysplastic syndromes/myeloproliferative neoplasm (MPN) overlap syndromes using small molecule ALK2 inhibitors.

### BACKGROUND

Anemia is common in patients with hematologic malignancies, occurring in up to 70% of patients. For example, approximately 80% to 90% of patients with myelodysplastic syndromes (MDS) develop anemia, of whom approximately 40% to 50% become transfusion-dependent, and within a year of diagnosis, 51% of patients with myelofibrosis (MF) will develop anemia. Anemia results in fatigue, weakness, impaired concentration, and diminished quality of life. In cancer-related anemia, inhibition of erythropoiesis can result from functional iron deficiency (FID)-decreased iron availability in the bone marrow for synthesis of hemoglobin, despite adequate body iron stores. FID is linked to elevated hepcidin, the master iron-regulatory hormone, which negatively regulates serum iron by inducing internalization and degradation of the iron exporter protein, ferroportin.

Currently, there are no therapies approved for the treatment of cancer-associated FID anemia. Treatments generally used to treat cancer-associated anemia do not target the underlying pathophysiology and include oral and parenteral iron and red blood cell (RBC) transfusions, which are associated with significant side effects and can increase hepcidin levels further. High-dose oral iron is difficult to tolerate and intravenous (IV) iron has restrictive and time-consuming administration requirements which results in underutilization. RBC transfusions provide temporary benefits to patients but are associated with both acute and chronic health risks, including risk of bacterial infection and allergic reactions to the donor blood, and place a significant burden on both the patient and the healthcare system. RBC transfusions are also associated with iron overload, which can lead to organ dysfunction over time and requires iron chelation therapy with deferoxamine. Therefore, there is an unmet need for more effective, well tolerated, and more convenient treatments for cancer-associated FID anemia.

### SUMMARY OF THE INVENTION

The present invention features methods of treating subjects having functional iron deficiency (FID), including FID anemia, using a small molecule ALK2 inhibitor. The FID (e.g., FID anemia) may be associated with myelodysplastic syndromes (MDS), myelofibrosis (MF), or myelodysplastic syndrome/myeloproliferative neoplasm overlap syndromes (MDS/MPN overlap syndromes). The small molecule ALK2 inhibitors may increase hemoglobin levels, increase reticulocyte hemoglobin, increase reticulocytes, increase mean corpuscular hemoglobin, increase serum iron, reduce plasma hepcidin, reduce transfusion burden, and/or promote transfusion independence.

In a first aspect, the invention provides a method of treating a subject having or at risk of developing anemia associated with a very low, low, or intermediate risk MDS (e.g., as determined by the Revised International Prognostic Scoring System (IPSS—R)) by administering to the subject a therapeutically effective amount of a small molecule ALK2 inhibitor or a pharmaceutically acceptable salt thereof.

In another aspect, the invention provides a method of treating a subject having or at risk of developing functional iron deficiency (FID) associated with a very low, low, or intermediate risk MDS (e.g., as determined by the IPSS—R) by administering to the subject a therapeutically effective amount of a small molecule ALK2 inhibitor or a pharmaceutically acceptable salt thereof.

In some embodiments of any of the foregoing aspects, the MDS is very low risk MDS. In some embodiments of any of the foregoing aspects, the MDS is low risk MDS. In some embodiments of any of the foregoing aspects, the MDS is intermediate risk MDS. In some embodiments of any of the foregoing aspects, the MDS is myelodysplastic syndrome with unilineage dysplasia (MDS-SLD). In some embodiments of any of the foregoing aspects, the MDS is myelodysplastic syndrome with multilineage dysplasia (MDS-MLD). In some embodiments of any of the foregoing aspects, the MDS is myelodysplastic syndrome with ring sideroblasts (MDS-RS, which includes single lineage dysplasia (MDS—RS-SLD) and multilineage dysplasia (MDS-RS-MLD)). In some embodiments of any of the foregoing aspects, the MDS is myelodysplastic syndrome associated with isolated del chromosome abnormality (MDS with isolated del (5q)). In some embodiments of any of the foregoing aspects, the MDS is myelodysplastic syndrome with excess blasts (MDS-EB; which includes myelodysplastic syndrome with excess blasts-type 1 (MDS-EB-1) and myelodysplastic syndrome with excess blasts-type 2 (MDS-EB-2)). In some embodiments of any of the foregoing aspects, the MDS is myelodysplastic syndrome, unclassifiable (MDS-U). In some embodiments of any of the foregoing aspects, the myelodysplastic syndrome is a ring sideroblast positive myelodysplastic syndrome. In some embodiments of any of the foregoing aspects, myelodysplastic syndrome is a non-ring sideroblast myelodysplastic syndrome. In some embodiments of any of the foregoing aspects, the subject has a bone marrow blast percentage of less than five percent prior to treatment with the small molecule ALK2 inhibitor.

In another aspect, the invention provides a method of treating a subject having or at risk of developing anemia associated with primary myelofibrosis (PMF), post-polycythemia vera myelofibrosis (post-PV MF), or post-essential thrombocythemia myelofibrosis (post-ET MF) by administering to the subject a therapeutically effective amount of a small molecule ALK2 inhibitor or a pharmaceutically acceptable salt thereof.

In another aspect, the invention provides a method of treating a subject having or at risk of developing FID associated with primary myelofibrosis (PMF), post-polycythemia vera myelofibrosis (post-PV MF), or post-essential thrombocythemia myelofibrosis (post-ET MF) by administering to the subject a therapeutically effective amount of a small molecule ALK2 inhibitor or a pharmaceutically acceptable salt thereof.

In some embodiments of any of the foregoing aspects, the anemia or FID is associated with PMF. In some embodiments of any of the foregoing aspects, the anemia or FID is associated with post-PV MF. In some embodiments of any of the foregoing aspects, the anemia or FID is associated with post-ET MF. In some embodiments of any of the foregoing aspects, the subject has a bone marrow and peripheral blood blast percentage that is less than two percent, or that is stable between two to five percent prior to treatment with the small molecule ALK2 inhibitor.

In another aspect, the invention provides a method of treating a subject having or at risk of developing anemia associated with an MDS/MPN overlap syndrome by administering to the subject a therapeutically effective amount of a small molecule ALK2 inhibitor or a pharmaceutically acceptable salt thereof.

In another aspect, the invention provides a method of treating a subject having or at risk of developing FID associated with an MDS/MPN overlap syndrome by administering to the subject a therapeutically effective amount of a small molecule ALK2 inhibitor or a pharmaceutically acceptable salt thereof.

In some embodiments of any of the foregoing aspects, the MDS/MPN overlap syndrome is chronic myelomonocytic leukemia. In some embodiments of any of the foregoing aspects, the MDS/MPN overlap syndrome is juvenile myelomonocytic leukemia. In some embodiments of any of the foregoing aspects, the MDS/MPN overlap syndrome is atypical chronic myeloid leukemia. In some embodiments of any of the foregoing aspects, the MDS/MPN overlap syndrome is myelodysplastic/myeloproliferative neoplasm, unclassifiable (MDS/MPN-U). In some embodiments of any of the foregoing aspects, the MDS/MPN overlap syndrome is myelodysplastic/myeloproliferative neoplasm with ring sideroblasts and thrombocytosis (MDS/MPN-RS-T). In some embodiments of any of the foregoing aspects, the subject has a bone marrow blast percentage of less than five percent prior to treatment with the small molecule ALK2 inhibitor.

In some embodiments of any of the foregoing aspects, the anemia is functional iron deficiency (FID) anemia.

In some embodiments of any of the foregoing aspects, the subject has or is at risk of developing anemia.

In some embodiments of any of the foregoing aspects, the subject does not receive concurrent treatment with a moderate to strong Cytochrome P450 3A4 (CYP3A4) inhibitor or inducer, a drug that is a substrate of Cytochrome P450 2B6 (CYP2B6) or multidrug and toxin extrusion protein 1 (MATE1), an inhibitor of P-glycoprotein, or an inhibitor of breast cancer resistance protein (BCRP).

In some embodiments of any of the foregoing aspects, the method reduces the subject's transfusion burden (e.g., in a subject who received 1-3 units of RBCs in the eight weeks prior to treatment initiation with the small molecule ALK2 inhibitor).

In some embodiments of any of the foregoing aspects, the subject achieves transfusion independence (e.g., a subject who received 1-3 units of RBCs in the eight weeks prior to treatment initiation with the small molecule ALK2 inhibitor) for at least four consecutive weeks during treatment (e.g., achieves transfusion independence for 4, 5, 6, 7, 8, or more weeks during treatment).

In some embodiments of any of the foregoing aspects, the subject achieves transfusion independence (e.g., a subject who received 1-3 units of RBCs in the eight weeks prior to treatment initiation with the small molecule ALK2 inhibitor) for at least eight consecutive weeks during treatment (e.g., achieves transfusion independence for 8, 10, 12, 14, 16, 18, 20, 22, 24, or more weeks during treatment).

In some embodiments of any of the foregoing aspects, the method increases the subject's mean hemoglobin by greater than or equal to 1.0 g/dL (e.g., greater than or equal to 1.0 g/dL, 1.5 g/dL, 2.0 g/dL or more during treatment with the small molecule ALK2 inhibitor). In some embodiments, the subject is a non-transfused subject (e.g., a subject who did not receive an RBC transfusion during the eight weeks immediately preceding treatment with the small molecule ALK2 inhibitor).

In some embodiments of any of the foregoing aspects, the method increases the subject's hemoglobin levels to be in the non-anemic range (e.g., >12.0 g/dL for women and >13.0 g/dL for men). In some embodiments, the subject is a non-transfused subject (e.g., a subject who did not receive an RBC transfusion during the eight weeks immediately preceding treatment with the small molecule ALK2 inhibitor).

In some embodiments of any of the foregoing aspects, the method increases reticulocyte hemoglobin by greater than 2.0 picogram (pg) from baseline or increases the subject's reticulocyte hemoglobin to greater than 32 pg.

In some embodiments of any of the foregoing aspects, the small molecule ALK2 inhibitor or a pharmaceutically acceptable salt thereof is administered in an amount sufficient to increase serum iron, increase transferrin saturation, increase iron bioavailability, increase reticulocyte hemoglobin, promote formation of hemoglobin-containing red blood cells, reestablish iron homeostasis, reduce hepcidin concentration in plasma, increase mean corpuscular hemoglobin, increase absolute reticulocyte count, or treat anemia.

In some embodiments of any of the above aspects, the small molecule ALK2 inhibitor is a compound of any one of Formulas I-III, Compounds 1-7, BCX9250, INCB00928, dorsomorphin, LDN-212854, LDN-193189, and LDN-21411or a pharmaceutically acceptable salt thereof.

In some embodiments of any of the foregoing aspects, the subject is a human.

In some embodiments of any of the foregoing aspects, the small molecule ALK2 inhibitor or a pharmaceutically acceptable salt thereof is a compound of Formula I:

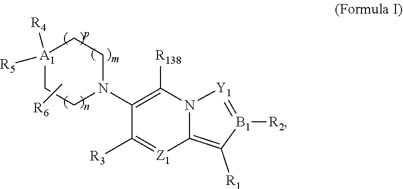

or a pharmaceutically acceptable salt thereof, wherein:
R¹ is hydrogen or an optionally substituted substituent selected from cycloalkyl, aryl, heteroaryl, and heterocyclyl;
R₂ is optionally absent, hydrogen, CN, NO₂, or an optionally substituted substituent selected from alkyl and amino;
R₃ is hydrogen, CN, NO₂, or an optionally substituted substituent selected from alkyl, alkoxy, heterocyclyloxy, heteroaryloxy, aryloxy, cycloalkyloxy, carbonyl, amino, amido, sulfonyl, sulfonamido, cycloalkyl, aryl, heterocyclyl, and heteroaryl;
R₄ is optionally absent, hydrogen, O—, halo, CN, NO₂, hydroxy, or an optionally substituted substituent selected from alkyl, alkenyl, alkynyl, carbonyl, cycloalkyl, aryl, alkoxy, aryloxy, cycloalkyloxy, amino, amido, alkoxycarbonyl, carboxy, sulfonyl, sulfonamido, thio, heterocyclyl, heterocyclyloxy, heteroaryl, and heteroaryloxy;
R₅ is optionally absent, hydrogen, halo, hydroxy, or optionally substituted alkyl;
R₁₃₈ is hydrogen, halo, hydroxy, or an optionally substituted substituent selected from alkyl, carbonyl, alkoxy, thio, amino, amido, heterocyclyl, aryl, and heteroaryl;
R₅ is independently one or more of hydrogen, halo, CN, NO₂, hydroxy, or an optionally substituted substituent selected from alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl and oxo; B1 is C or N; Y₁ is N or CR₁₃₉, wherein R₁₃₉ is hydrogen, halo, hydroxy, or an optionally substituted substituent selected from alkyl, carbonyl, alkoxy, thio, amino, amido, heterocyclyl, aryl, and heteroaryl; Z₁ is N or CR₁₄₀, wherein R₁₄₀ is hydrogen, halo, hydroxy, or an optionally substituted substituent selected from alkyl, carbonyl, alkoxy, thio, amino, amido, heterocyclyl, aryl, or heteroaryl; A1 is C, N, O, C(O), S, SO, or SO₂; m is 0, 1, 2, or 3; n is 0, 1, 2, or 3; and p is 0 or 1; wherein optionally any two or more of R₄, R₅, or R₆ may be joined together to form one or more rings.

In some embodiments, the compound of Formula I has a structure of Formula I-a:

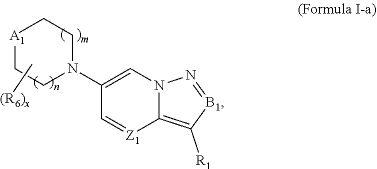

or a pharmaceutically acceptable salt thereof, wherein:
A₁ is NR^{4a} or CR^{4b}R⁵;
B₁ is N or CR²;
Z₁ is N or CR³;
R¹ is selected from cycloalkyl, aryl, heteroaryl, and heterocyclyl;
R² is H, CN, NO₂, alkyl, or amino;
R³ is selected from H, CN, NO₂, alkyl, alkoxy, heterocyclyloxy, heteroaryloxy, aryloxy, cycloalkyloxy, carbonyl, amino, amido, sulfonyl, sulfonamido, cycloalkyl, aryl, heterocyclyl, and heteroaryl;
R^{4a} is selected from alkyl, alkenyl, alkynyl, carbonyl, O—, alkoxycarbonyl, cycloalkyl, aryl, heterocyclyl, and heteroaryl;
R^{4b} is selected from halo, CN, NO₂, hydroxy, alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl;
R⁵ is selected from H, halo, hydroxy and alkyl, or
R^{4b} and R⁵ together with A₁ form a ring selected from cycloalkyl and heterocyclyl;
each R⁵ is independently selected from H, halo, CN, NO₂, hydroxy, alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl and oxo;
n is 0 or 1;
m is 0 or 1; and
x is 0, 1, 2, 3, or 4.

In some embodiments of the compound of Formula I-a,
A₁ is NR^{4a} or CR^{4b}R⁵;
B₁ is N or CR²;
Z₁ is N or CR³;
R¹ is selected from aryl, heteroaryl, and heterocyclyl;
R² is H or amino;
R³ is H or heterocyclyloxy;
R^{4a} is selected from alkyl, O, aryl, heterocyclyl, and heteroaryl;
R^{4b} is selected from alkyl, alkoxy, amino, aryl, heterocyclyl, and heteroaryl;
R⁵ is selected from H and alkyl, or
R^{4b} and R³ together with A₁ form a ring selected from cycloalkyl and heterocyclyl;
each R⁶ is independently selected from H, halo, alkyl and oxo;
n is 0 or 1;
m is 0 or 1; and
x is 0, 1, 2, 3, or 4.

In some embodiments of the compound of Formula I-a,
R^{4a} is selected from alkyl, O—, heterocyclyl, and heteroaryl;
R^{4b} is selected from alkyl, alkoxy, amino, amido, heterocyclyl, and heteroaryl;
R⁵ is selected from H and alkyl, or
R^{4b} and R³ together with A₁ form a heterocyclyl; and
each R⁵ is independently selected from H, halo, and alkyl; and x is 0 or 1.

In some embodiments of the compound of Formula I-a, R¹ is selected from H, aryl, 5-6 membered heteroaryl,

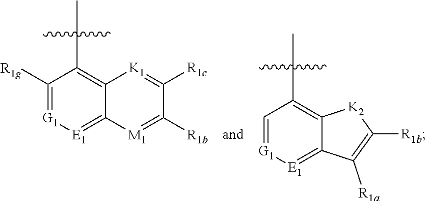

wherein:
each E₁ is independently selected from N and CR^{1d};
each G₁ is independently selected from N and CR^{1e};
K₁ is N or CH;
K₂ is NH or S;
M₁ is N or CR^{1a};
R^{1a} is selected from H, halo, alkyl, haloalkyl, and amido;
R^{1b} is selected from H, halo, CN, alkyl, haloalkyl, hydroxy, alkoxy, and haloalkoxy;
R^{1c} is selected from H, halo, CN, alkyl, haloalkyl, hydroxy, alkoxy, haloalkoxy, amino and amido, or
R^{1b} and R^{1c} together with the carbon atoms to which they are attached form a heterocyclyl;
R^{1d} is selected from H, CN, alkyl, haloalkyl, hydroxy, amido and sulfonamido;
R^{1e} is selected from H, alkyl and amino; and
R^{1g} is H or halo.

In some embodiments of the compound of Formula I-a,
R^{4a} is selected from alkyl, O, heterocyclyl, and heteroaryl;
R^{4b} is selected from alkyl, alkoxy, amino, amido, heterocyclyl, and heteroaryl;
R³ is selected from H and alkyl, or
R^{4b} and R³ together with A₁ form a heterocyclyl; and
each R⁶ is independently selected from H, halo, and alkyl; and
x is 0 or 1.

In some embodiments, R¹ is selected from H, aryl, 5-6 membered heteroaryl,

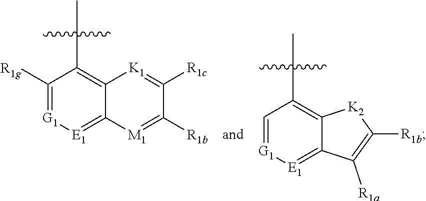

wherein:
each E₁ is independently selected from N and CR^{1d};
each G₁ is independently selected from N and CR^{1e};
K₁ is N or CH;
K₂ is NH or S;
M₁ is CR^{1a};
R^{1a} is selected from H and amido;
R^{1b} is selected from H, halo, alkyl, and alkoxy;
R^{1c} is selected from H, alkyl, and alkoxy, or
R^{1b} and R^{1c} together with the carbon atoms to which they are attached form a heterocyclyl;
R^{1a} is selected from H, alkyl, hydroxy, amido and sulfonamido;
R^{1e} is selected from H, alkyl and amino;
R^{1f} is H; and
R^{1g} is H.

In some embodiments, the compound of Formula I has a structure of any one of Formulas I-1 to I-200 or a pharmaceutically acceptable salt thereof. In some embodiments, the compound of Formula I has the structure of Formula II-1. In some embodiments, the compound of Formula I has the structure of Formula II-2. In some embodiments, the compound of Formula I has the structure of Formula I-3. In some embodiments, the compound of Formula I has the structure of Formula II-4. In some embodiments, the compound of Formula I has the structure of Formula II-5. In some embodiments, the compound of Formula I has the structure of Formula II-6. In some embodiments, the compound of Formula I has the structure of Formula II-7. In some embodiments, the compound of Formula I has the structure of Formula 1-8. In some embodiments, the compound of Formula I has the structure of Formula II-9. In some embodiments, the compound of Formula I has the structure of Formula II-10. In some embodiments, the compound of Formula I has the structure of Formula II-11. In some embodiments, the compound of Formula I has the structure of Formula I-12. In some embodiments, the compound of Formula I has the structure of Formula II-13. In some embodiments, the compound of Formula I has the structure of Formula II-14. In some embodiments, the compound of Formula I has the structure of Formula II-15. In some embodiments, the compound of Formula I has the structure of Formula II-16. In some embodiments, the compound of Formula I has the structure of Formula II-17. In some embodiments, the compound of Formula I has the structure of Formula II-18. In some embodiments, the compound of Formula I has the structure of Formula I-19. In some embodiments, the compound of Formula I has the structure of Formula II-20. In some embodiments, the compound of Formula I has the structure of Formula I-21. In some embodiments, the compound of Formula I has the structure of Formula II-22. In some embodiments, the compound of Formula I has the structure of Formula II-23. In some embodiments, the compound of Formula I has the structure of Formula II-24. In some embodiments, the compound of Formula I has the structure of Formula II-25. In some embodiments, the compound of Formula I has the structure of Formula II-26. In some embodiments, the compound of Formula I has the structure of Formula II-27. In some embodiments, the compound of Formula I has the structure of Formula II-28. In some embodiments, the compound of Formula I has the structure of Formula II-29. In some embodiments, the compound of Formula I has the structure of Formula II-30. In some embodiments, the compound of Formula I has the structure of Formula II-31. In some embodiments, the compound of Formula I has the structure of Formula II-32. In some embodiments, the compound of Formula I has the structure of Formula II-33. In some embodiments, the compound of Formula I has the structure of Formula II-34. In some embodiments, the compound of Formula I has the structure of Formula II-35. In some embodiments, the compound of Formula I has the structure of Formula II-36. In some embodiments, the compound of Formula I has the structure of Formula II-37. In some embodiments, the compound of Formula I has the structure of Formula II-38. In some embodiments, the compound of Formula I has the structure of Formula II-39. In some embodiments, the compound of Formula I has the structure of Formula II-40. In some embodiments, the compound of Formula I has the structure of Formula II-41. In some embodiments, the compound of Formula I has the structure of Formula II-42. In some embodiments, the compound of Formula I has the structure of Formula II-43. In some embodiments, the compound of Formula I has the structure of Formula II-44. In some embodiments, the compound of Formula I has the structure of Formula II-45. In some embodiments, the compound of Formula I has the structure of Formula II-46. In some embodiments, the compound of Formula I has the structure of Formula II-47. In some embodiments, the compound of Formula I has the structure of Formula II-48. In some embodiments, the compound of Formula I has the structure of Formula II-49. In some embodiments, the compound of Formula I has the structure of Formula II-50. In some embodiments, the compound of Formula I has the structure of Formula II-51. In some embodiments, the compound of Formula I has the structure of Formula II-52. In some embodiments, the compound of Formula I has the structure of Formula II-53. In some embodiments, the compound of Formula I has the structure of Formula II-54. In some embodiments, the compound of Formula I has the structure of Formula II-55. In some embodiments, the compound of Formula I has the structure of Formula II-56. In some embodiments, the compound of Formula I has the structure of Formula II-57. In some embodiments, the compound of Formula I has the structure of Formula II-58. In some embodiments, the compound of Formula I has the structure of Formula II-59. In some embodiments, the compound of Formula I has the structure of Formula II-60. In some embodiments, the compound of Formula I has the structure of Formula II-61. In some embodiments, the compound of Formula I has the structure of Formula II-62. In some embodiments, the compound of Formula I has the structure of Formula II-63. In some embodiments, the compound of Formula I has the structure of Formula II-64. In some embodiments, the compound of Formula I has the structure of Formula II-65. In some embodiments, the compound of Formula I has the structure of Formula II-66. In some embodiments, the compound of Formula I has the structure of Formula II-67. In some embodiments, the compound of Formula I has the structure of Formula II-68. In some embodiments, the compound of Formula I has the structure of Formula II-69. In some embodiments, the compound of Formula I has the structure of Formula II-70. In some embodiments, the compound of Formula I has the structure of Formula II-71. In some embodiments, the compound of Formula I has the structure of Formula II-72. In some embodiments, the compound of Formula I has the structure of Formula II-73. In some embodiments, the compound of Formula I has the structure of Formula II-74. In some embodiments, the compound of Formula I has the structure of Formula II-75. In some embodiments, the compound of Formula I has the structure of Formula II-76. In some embodiments, the compound of Formula I has the structure of Formula II-77. In some embodiments, the compound of Formula I has the structure of Formula II-78. In some embodiments, the compound of Formula I has the structure of Formula II-79. In some embodiments, the compound of Formula I has the structure of Formula II-80. In some embodiments, the compound of Formula I has the structure of Formula II-81. In some embodiments, the compound of Formula I has the structure of Formula II-82. In some embodiments, the compound of Formula I has the structure of Formula II-83. In some embodiments, the compound of Formula I has the structure of Formula II-84. In some embodiments, the compound of Formula I has the structure of Formula II-85. In some embodiments, the compound of Formula I has the structure of Formula II-86. In some embodiments, the compound of Formula I has the structure of Formula II-87. In some embodiments, the compound of Formula I has the structure of Formula II-88. In some embodiments, the compound of Formula I has the structure of Formula II-89. In some embodiments, the compound of Formula I has the structure of Formula II-90. In some embodiments, the compound of Formula I has the structure of Formula II-91. In some embodiments, the compound of Formula I has the structure of Formula II-92. In some embodiments, the compound of Formula I has the structure of Formula II-93. In some embodiments, the compound of Formula I has the structure of Formula II-94. In some embodiments, the compound of Formula I has the structure of Formula II-95. In some embodiments, the compound of Formula I has the structure of Formula II-96. In some embodiments, the compound of Formula I has the structure of Formula II-97. In some embodiments, the compound of Formula I has the structure of Formula II-98. In some embodiments, the compound of Formula I has the structure of Formula II-99. In some embodiments, the compound of Formula I has the structure of Formula II-100. In some embodiments, the compound of Formula I has the structure of Formula II-101. In some embodiments, the compound of Formula I has the structure of Formula II-102. In some embodiments, the compound of Formula I has the structure of Formula II-103. In some embodiments, the compound of Formula I has the structure of Formula II-104. In some embodiments, the compound of Formula I has the structure of Formula II-105. In some embodiments, the compound of Formula I has the structure of Formula II-106. In some embodiments, the compound of Formula I has the structure of Formula II-107. In some embodiments, the compound of Formula I has the structure of Formula II-108. In some embodiments, the compound of Formula I has the structure of Formula II-109. In some embodiments, the compound of Formula I has the structure of Formula II-110. In some embodiments, the compound of Formula I has the structure of Formula II-111. In some embodiments, the compound of Formula I has the structure of Formula II-112. In some embodiments, the compound of Formula I has the structure of Formula II-113. In some embodiments, the compound of Formula I has the structure of Formula II-114. In some embodiments, the compound of Formula I has the structure of Formula II-115. In some embodiments, the compound of Formula I has the structure of Formula II-116. In some embodiments, the compound of Formula I has the structure of Formula II-117. In some embodiments, the compound of Formula I has the structure of Formula II-118. In some embodiments, the compound of Formula I has the structure of Formula II-119. In some embodiments, the compound of Formula I has the structure of Formula I-120. In some embodiments, the compound of Formula I has the structure of Formula II-121. In some embodiments, the compound of Formula I has the structure of Formula II-122. In some embodiments, the compound of Formula I has the structure of Formula II-123. In some embodiments, the compound of Formula I has the structure of Formula II-124. In some embodiments, the compound of Formula I has the structure of Formula II-125. In some embodiments, the compound of Formula I has the structure of Formula II-126. In some embodiments, the compound of Formula I has the structure of Formula II-127. In some embodiments, the compound of Formula I has the structure of Formula II-128. In some embodiments, the compound of Formula I has the structure of Formula II-129. In some embodiments, the compound of Formula I has the structure of Formula II-130. In some embodiments, the compound of Formula I has the structure of Formula II-131. In some embodiments, the compound of Formula I has the structure of Formula II-132. In some embodiments, the compound of Formula I has the structure of Formula II-133. In some embodiments, the compound of Formula I has the structure of Formula II-134. In some embodiments, the compound of Formula I has the structure of Formula II-135. In some embodiments, the compound of Formula I has the structure of Formula I-136. In some embodiments, the compound of Formula I has the structure of Formula II-137. In some embodiments, the compound of Formula I has the structure of Formula II-138. In some embodiments, the compound of Formula I has the structure of Formula II-139. In some embodiments, the compound of Formula I has the structure of Formula II-140. In some embodiments, the compound of Formula I has the structure of Formula II-141. In some embodiments, the compound of Formula I has the structure of Formula II-142. In some embodiments, the compound of Formula I has the structure of Formula II-143. In some embodiments, the compound of Formula I has the structure of Formula II-144. In some embodiments, the compound of Formula I has the structure of Formula II-145. In some embodiments, the compound of Formula I has the structure of Formula II-146. In some embodiments, the compound of Formula I has the structure of Formula II-147. In some embodiments, the compound of Formula I has the structure of Formula II-148. In some embodiments, the compound of Formula I has the structure of Formula II-149. In some embodiments, the compound of Formula I has the structure of Formula II-150. In some embodiments, the compound of Formula I has the structure of Formula II-151. In some embodiments, the compound of Formula I has the structure of Formula II-152. In some embodiments, the compound of Formula I has the structure of Formula II-153. In some embodiments, the compound of Formula I has the structure of Formula II-154. In some embodiments, the compound of Formula I has the structure of Formula II-155. In some embodiments, the compound of Formula I has the structure of Formula II-156. In some embodiments, the compound of Formula I has the structure of Formula II-157. In some embodiments, the compound of Formula I has the structure of Formula II-158. In some embodiments, the compound of Formula I has the structure of Formula II-159. In some embodiments, the compound of Formula I has the structure of Formula II-160. In some embodiments, the compound of Formula I has the structure of Formula II-161. In some embodiments, the compound of Formula I has the structure of Formula II-162. In some embodiments, the compound of Formula I has the structure of Formula II-163. In some embodiments, the compound of Formula I has the structure of Formula II-164. In some embodiments, the compound of Formula I has the structure of Formula II-165. In some embodiments, the compound of Formula I has the structure of Formula II-166. In some embodiments, the compound of Formula I has the structure of Formula II-167. In some embodiments, the compound of Formula I has the structure of Formula II-168. In some embodiments, the compound of Formula I has the structure of Formula II-169. In some embodiments, the compound of Formula I has the structure of Formula II-170. In some embodiments, the compound of Formula I has the structure of Formula II-171. In some embodiments, the compound of Formula I has the structure of Formula II-172. In some embodiments, the compound of Formula I has the structure of Formula II-173. In some embodiments, the compound of Formula I has the structure of Formula II-174. In some embodiments, the compound of Formula I has the structure of Formula II-175. In some embodiments, the compound of Formula I has the structure of Formula I-176. In some embodiments, the compound of Formula I has the structure of Formula II-177. In some embodiments, the compound of Formula I has the structure of Formula II-178. In some embodiments, the compound of Formula I has the structure of Formula I-179. In some embodiments, the compound of Formula I has the structure of Formula II-180. In some embodiments, the compound of Formula I has the structure of Formula I-181. In some embodiments, the compound of Formula I has the structure of Formula II-182. In some embodiments, the compound of Formula I has the structure of Formula II-183. In some embodiments, the compound of Formula I has the structure of Formula II-184. In some embodiments, the compound of Formula I has the structure of Formula II-185. In some embodiments, the compound of Formula I has the structure of Formula II-186. In some embodiments, the compound of Formula I has the structure of Formula II-187. In some embodiments, the compound of Formula I has the structure of Formula II-188. In some embodiments, the compound of Formula I has the structure of Formula II-189. In some embodiments, the compound of Formula I has the structure of Formula I-190. In some embodiments, the compound of Formula I has the structure of Formula I-191. In some embodiments, the compound of Formula I has the structure of Formula II-192. In some embodiments, the compound of Formula I has the structure of Formula II-193. In some embodiments, the compound of Formula I has the structure of Formula II-194. In some embodiments, the compound of Formula I has the structure of Formula II-195. In some embodiments, the compound of Formula I has the structure of Formula II-196. In some embodiments, the compound of Formula I has the structure of Formula I-197. In some embodiments, the compound of Formula I has the structure of Formula II-198. In some embodiments, the compound of Formula I has the structure of Formula II-199. In some embodiments, the compound of Formula I has the structure of Formula II-200.

In some embodiments of any of the foregoing aspects, the small molecule ALK2 inhibitor or a pharmaceutically acceptable salt thereof is a compound of Formula II:

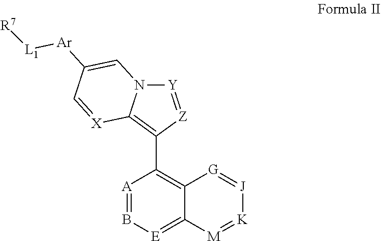

or a pharmaceutically acceptable salt thereof, wherein
X and Y are independently selected from CR¹⁵ and N, preferably both N;
Z is selected from CR³′ and N, preferably CR³′, most preferably CH;
Ar is a substituted or unsubstituted aryl ring or a substituted or unsubstituted heteroaryl ring;
L₁ is absent or selected from substituted or unsubstituted alkyl, substituted or unsubstituted cycloalkyl, substituted or unsubstituted cycloalkylalkyl, cycloalkyl-heteroalkyl, substituted or unsubstituted heterocyclyl, substituted or unsubstituted heterocyclylalkyl, substituted or unsubstituted heterocyclyl-heteroalkyl, and substituted or unsubstituted heteroalkyl; and
J and K are both absent or, independently for each occurrence, are each CR¹⁶;
A is CR¹⁶;
B and E are each independently CR¹⁷;
if J and K are absent, then G is R¹⁶ and M is R¹⁷; if J and K are not absent, then G is CR¹⁶ and M is CR¹⁷;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from

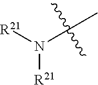

and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido, preferably H;
R¹⁶, independently for each occurrence, is selected from H, OH, halogen, cyano, carboxyl, and substituted or unsubstituted acyl, alkanol, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkoxy, alkylthio, acyloxy, amino, acylamino, carbamate, amido, amidino, sulfonyl, sulfoxido, sulfamoyl, or sulfonamide;
R¹⁷, independently for each occurrence, is selected from R¹⁶ and —R²², —NH₂, —NHR²², —N(R²²)₂, halogen, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)═NR²², —C(N(R²²)₂)—NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²,

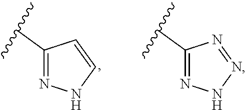 [SMILES: *c1cc[nH]n1 ; *c1nn[nH]n1]

—CH(OH)R²², —C(OH)(R²²)₂, —CH(NH₂)(R²²), —CH(NHR²²)(R²²), —CH(N(R²²)₂)(R²²), pyrazol-3-yl, pyrazol-4-yl, and —OR²², provided that at least one R¹⁷ is —R²², —NH₂, —NHR²², —N(R²²)₂, halogen, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)═NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²

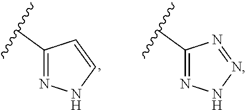 [SMILES: *c1cc[nH]n1 ; *c1nn[nH]n1]

—CH(OH)R²²_C(OH)(R²²)₂, —CH(NH₂)(R²²), —CH(NHR²²)(R²²), —CH(N(R²²)₂)(R²²), pyrazol-3-yl, pyrazol-4-yl, or —OR²²;
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide, preferably from H and substituted or unsubstituted alkyl, aralkyl, cycloalkyl, heterocyclyl, aryl, heteroaryl, heteroaralkyl, cycloalkylalkyl, heterocyclylalkyl, more preferably from H and substituted or unsubstituted alkyl, and most preferably from H and lower alkyl, such as methyl or ethyl; and
R²², independently for each occurrence, is selected from lower alkyl (e.g., CH₃ or CF₃) and cycloalkyl (preferably cyclopropyl or cyclobutyl).

In some embodiments of any of the foregoing aspects, the small molecule ALK2 inhibitor or a pharmaceutically acceptable salt thereof is a compound of Formula II, wherein
X and Y are each N;
Z is CR³;
Ar is a substituted or unsubstituted aryl ring or a substituted or unsubstituted heteroaryl ring;
L₁ is absent or selected from substituted or unsubstituted cycloalkyl, substituted or unsubstituted cycloalkylalkyl, cycloalkyl-heteroalkyl, substituted or unsubstituted heterocyclyl, substituted or unsubstituted heterocyclylalkyl, substituted or unsubstituted heterocyclylheteroalkyl, and

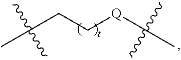

wherein Q is selected from CR¹⁰′R¹¹, NR¹², O, S, S(O), and SO₂; R¹⁰ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido; R¹² is selected from H and substituted or unsubstituted alkyl, cycloalkyl, heterocyclyl, heterocyclylalkyl, amino, acylamino, carbamate, amido, amidino, sulfonyl, sulfamoyl, or sulfonamide; and t is an integer selected from 0, 2, 3, and 4, wherein any CH₂ subunit of L₁ is optionally substituted with one or two lower alkyl groups, or represents a carbon atom in a 3-5-membered cycloalkyl or heterocyclyl ring; and
J and K are both absent or, independently for each occurrence, are each CR¹⁶;
A is CR¹⁵;
B and E are each independently CR¹⁷;
if J and K are absent, then G is R¹⁶ and M is R¹⁷; if J and K are not absent, then G is CR¹⁶ and M is CR¹⁷;
R³ is H;
R⁷ is selected from

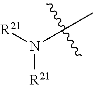

and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, OH, cyano, carboxyl, and substituted or unsubstituted acyl, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkylthio, acyloxy, amino, acylamino, carbamate, amido, amidino, sulfonyl, sulfoxido, sulfamoyl, or sulfonamide; R¹⁷, independently for each occurrence, is selected from R¹⁶ and —R²², —NH₂, —NHR²², —N(R²²)₂, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)—NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²,

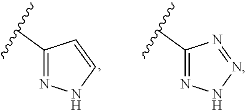 [SMILES: *c1cc[nH]n1 ; *c1nn[nH]n1]

—CH(OH)R²², —C(OH)(R²²)₂, —CH(NH₂)(R²²), —CH(NHR²²)(R²²), —CH(N(R²²)₂)(R²²), pyrazol-3-yl, pyrazol-4-yl, and —OR²², provided that at least one R¹⁷ is —R²², —NH₂, —NHR²², —N(R²²)₂, —CO₂H, —CO₂R²², —CONH₂, —CONHR²², —CON(R²²)₂, —C(NH₂)═N(OH), —C(NHR²²)═N(OH), —C(N(R²²)₂)—N(OH), —C(NH₂)═NH, —C(NHR²²)═NH, —C(NHR²²)—NR²², —C(N(R²²)₂)═NH, —C(N(R²²)₂)—NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²,

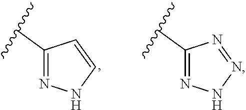 [SMILES: *c1cc[nH]n1 ; *c1nn[nH]n1]

—CH(OH)R²², —C(OH)(R²²)₂, —CH(NH₂)(R²²), —CH(NHR²²)(R²²), —CH(N(R²²)₂)(R²²), pyrazol-3-yl, or pyrazol-4-yl,
where at least one R¹⁷ represents a moiety selected from—CO₂H, —CONH₂, —CH₂OH, —CN, —C(O)CH₃, —CH(OH) CH₃, —C(OH)(CH₃)₂, —C(O)CF₃, —CH(NH₂) CF₃, —SO₂CH₃, —SO₂NH₂ and

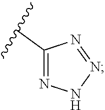 [SMILES: *c1nn[nH]n1]

R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide; and
R²², independently for each occurrence, is selected from lower alkyl and cycloalkyl;
wherein at least one R¹⁶ or one R¹⁷ is not H.

In some embodiments of any of the foregoing aspects, the small molecule ALK2 inhibitor or a pharmaceutically acceptable salt thereof is a compound of Formula II, wherein
X and Y are each N;
Z is CR³;
Ar is a substituted or unsubstituted aryl ring or a substituted or unsubstituted heteroaryl ring;
L₁ is absent or

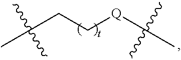

wherein Q is selected from CR¹⁰′R¹¹, NR¹², O, S, S(O), and SO₂; R¹⁰′ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido; R¹² is selected from H and substituted or unsubstituted alkyl, cycloalkyl, heterocyclyl, heterocyclylalkyl, amino, acylamino, carbamate, amido, amidino, sulfonyl, sulfamoyl, or sulfonamide; and t is selected from 0, 2, 3, and 4, wherein any CH₂ subunit of L₁ is optionally substituted with one or two lower alkyl groups, or represents a carbon atom in a 3-5-membered cycloalkyl or heterocyclyl ring; and
J and K are both absent or, independently for each occurrence, are each CR¹⁶;
A and B, independently for each occurrence, are CR¹⁶;
E is CR¹⁷;
if J and K are absent, then G and M are each independently R¹⁶; if J and K are not absent, then G and M are each independently CR¹⁷;
R³ is H;
R⁷ is

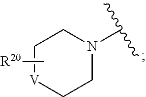

V is NR³⁰;
R²⁰ is absent or represents from 1-6 substituents on the ring to which it is attached, independently selected from substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfoxido, sulfamoyl, and sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, OH, cyano, carboxyl, and substituted or unsubstituted acyl, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkylthio, acyloxy, amino, acylamino, carbamate, amido, amidino, sulfonyl, sulfoxido, sulfamoyl, sulfonamide, tetrazolyl, or trifluoromethylacyl;
R¹⁷, independently for each occurrence, is selected from R¹⁶ and H, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃,

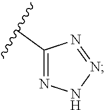 [SMILES: *c1nn[nH]n1]

—CH(OH) CH₃, —C(O)CF₃, and —OCH₃, provided that at least one R¹⁷ is H, —CO₂H, —CONH₂, —CONHCH₃, —CON(CH₃)₂, —C(NH₂)—N(OH), —C(NH₂)═NH, —CN, —CH₂OH, —SO₂NH₂, —CH₂NH₂, —C(O)CH₃,

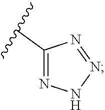 [SMILES: *c1nn[nH]n1]

—CH(OH) CH₃, or —C(O)CF₃; and
R³⁰, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide;
wherein at least one R¹⁸ or one R¹⁷ is not H.

In some embodiments of any of the foregoing aspects, the small molecule ALK2 inhibitor or a pharmaceutically acceptable salt thereof is a compound of Formula II or a pharmaceutically acceptable salt thereof, wherein
X and Y are independently selected from CR¹⁵ and N, preferably both N;
Z is selected from CR³ and N, preferably CR³′, most preferably CH;
Ar is a substituted or unsubstituted aryl ring (e.g., a substituted or unsubstituted phenyl ring) or a substituted or unsubstituted heteroaryl ring (e.g., a pyridyl or pyrimidyl ring);
L₁ is absent or selected from substituted or unsubstituted alkyl and heteroalkyl; and
J and K are both absent or, independently for each occurrence, are each CR¹⁶;
A and B, independently for each occurrence, are CR¹⁸;
E is CR¹⁷;
if J and K are absent, then G and M are each independently R¹⁶; if J and K are not absent, then G and M are each independently CR¹⁷;
R³′ is selected from H, halogen, cyano, and substituted or unsubstituted alkyl, cycloalkyl, acylamino, carbamate, sulfonyl, sulfoxido, sulfamoyl, or sulfonamido;
R⁷ is selected from

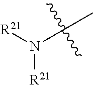

and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, D, OH, halogen, cyano, carboxyl, and substituted or unsubstituted acyl, alkanol, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkoxy, alkylthio, acyloxy, amino, acylamino, carbamate, amido, amidino, sulfonyl, sulfoxido, sulfamoyl, sulfonamide, tetrazolyl, or trifluoromethylacyl;
R¹⁷, independently for each occurrence, is selected from R¹⁶ and H, D, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃,

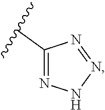 [SMILES: *c1nn[nH]n1]

—CH(OH) CH₃, —C(O)CF₃, and —OCH₃, provided that at least one R¹⁷ is H, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃,

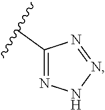 [SMILES: *c1nn[nH]n1]

—CH(OH) CH₃, —C(O)CF₃, or —OCH₃;
and
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide.

In some embodiments of any of the foregoing aspects, the small molecule ALK2 inhibitor or a pharmaceutically acceptable salt thereof is a compound of Formula II or a pharmaceutically acceptable salt thereof, wherein
X and Y are independently selected from CR¹⁵ and N, preferably both N;
Z is selected from CR³ and N, preferably CR³′, most preferably CH;
Ar is a phenyl ring substituted with at least one non-protium (1H) substituent or a substituted or unsubstituted heteroaryl ring;
L₁ is absent or selected from substituted or unsubstituted alkyl and heteroalkyl; and
G, J, K, and M are all absent or, independently for each occurrence, are selected from CR¹⁶ and N;
A, B, and E, independently for each occurrence, are selected from CR¹⁶ and N; provided that no more than three (and preferably no more than two) of A, B, E, G, J, K, and M are N, and at least one of E and M is N, and that if G, J, K, and M are absent then the carbon atom adjacent to E and M is optionally substituted with R¹⁶;
R³′ is selected from H, halogen, cyano, and substituted or unsubstituted alkyl, cycloalkyl, acylamino, carbamate, sulfonyl, sulfoxido, sulfamoyl, or sulfonamido;
R⁷ is selected from H, hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido; and
R¹⁶, independently for each occurrence, is absent or is selected from H (including, and in certain embodiments preferably, D), OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, or sulfonamide.

In some embodiments, the ALK2 inhibitor is a compound of Formula II or a pharmaceutically acceptable salt thereof, wherein
X and Y are independently selected from CR¹⁵ and N, preferably both N;
Z is selected from CR³′ and N, preferably CR³′, most preferably CH;
Ar is selected from substituted or unsubstituted aryl and heteroaryl;
L₁ is absent or selected from substituted or unsubstituted alkyl and heteroalkyl; and
G, J, K, and M are all absent or, independently for each occurrence, are selected from CR 16 and N;
A, B, and E, independently for each occurrence, are selected from CR¹⁶ and N;
provided that no more than three (and preferably no more than two) of A, B, E, G, J, K, and M are N, and at least one of E and M is N, and that if G, J, K, and M are absent then the carbon atom adjacent to E and M is optionally substituted with R¹⁶;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from H, hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido; and
R¹⁶, independently for each occurrence, is absent or is selected from H (including, and in certain embodiments preferably, D), OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, and sulfonamide;
wherein B is C—R²⁵ when E is N or K is C—R²⁵ when M is N or both such that at least one of B and K is C—R²⁵, where R²⁵ is selected from deuterium, halogen (preferably fluorine or chlorine), hydroxyl, lower alkyl (preferably methyl), and lower alkoxy (preferably methoxy), such as deuterium, fluorine, chlorine, methyl, ethyl, hydroxy, or methoxy.

In some embodiments of the compound of Formula II,
X and Y are independently selected from CR¹⁵ and N;
Z is selected from CR³ and N;
Ar is selected from substituted or unsubstituted aryl and heteroaryl;
L₁ is absent or selected from substituted or unsubstituted alkyl and heteroalkyl;
G, J, K, and M are all absent or, independently for each occurrence, are selected from CR¹⁶ and N;
A, B, and E, independently for each occurrence, are selected from CR 16 and N;
provided that:
no more than three of A, B, E, G, J, K, and M are N, at least one of E and M is N, and
that if G, J, K, and M are absent, then the carbon atom drawn as connected to variable M is optionally substituted with R¹⁶;
R³ is selected from H, halogen, cyano, and substituted or unsubstituted alkyl, cycloalkyl, acylamino, carbamate, sulfonyl, sulfoxido, sulfamoyl, and sulfonamido;
R⁷ is selected from hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, and sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, and sulfonamido; and
R¹⁶, independently for each occurrence, is absent or is selected from H, OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, and sulfonamide;
provided that:
i) if Ar is a phenyl ring, it is substituted with at least one non-protium (1H) substituent;
ii) B is C—R²⁵ when E is N, or K is C—R²⁵ when M is N, or both, such that at least one of B and K is C—R²⁵, wherein
R²⁵ is selected from deuterium, halogen, hydroxyl, lower alkyl, and lower alkoxy; and/or
iii) R⁷ is

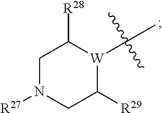

W is N, CH, or CCH₃;
R²⁷ is selected from H and substituted or unsubstituted alkyl, acyl, and ester; and
R²⁸ and R²⁹ are each independently H or alkyl, or
R²⁸ forms a one- or two-carbon bridge to the carbon atom adjacent to R²⁹ and NR²⁷;
wherein either W is CH or CCH₃, or R²⁸ and R²⁹ are not both H.

In some embodiments, the compound of Formula II has a structure of any one of Formulas II-1 to II-275 or is a pharmaceutically acceptable salt thereof. In some embodiments, the compound of Formula II has the structure of Formula II-1. In some embodiments, the compound of Formula II has the structure of Formula II-2. In some embodiments, the compound of Formula II has the structure of Formula II-3. In some embodiments, the compound of Formula II has the structure of Formula II-4. In some embodiments, the compound of Formula II has the structure of Formula II-5. In some embodiments, the compound of Formula II has the structure of Formula II-6. In some embodiments, the compound of Formula II has the structure of Formula II-7. In some embodiments, the compound of Formula II has the structure of Formula II-8. In some embodiments, the compound of Formula II has the structure of Formula II-9. In some embodiments, the compound of Formula II has the structure of Formula II-10. In some embodiments, the compound of Formula II has the structure of Formula II-11. In some embodiments, the compound of Formula II has the structure of Formula II-12. In some embodiments, the compound of Formula II has the structure of Formula II-13. In some embodiments, the compound of Formula II has the structure of Formula II-14. In some embodiments, the compound of Formula II has the structure of Formula II-15. In some embodiments, the compound of Formula II has the structure of Formula II-16. In some embodiments, the compound of Formula II has the structure of Formula II-17. In some embodiments, the compound of Formula II has the structure of Formula II-18. In some embodiments, the compound of Formula II has the structure of Formula II-19. In some embodiments, the compound of Formula II has the structure of Formula II-20. In some embodiments, the compound of Formula II has the structure of Formula II-21. In some embodiments, the compound of Formula II has the structure of Formula II-22. In some embodiments, the compound of Formula II has the structure of Formula II-23. In some embodiments, the compound of Formula II has the structure of Formula II-24. In some embodiments, the compound of Formula II has the structure of Formula II-25. In some embodiments, the compound of Formula II has the structure of Formula II-26. In some embodiments, the compound of Formula II has the structure of Formula II-27. In some embodiments, the compound of Formula II has the structure of Formula II-28. In some embodiments, the compound of Formula II has the structure of Formula II-29. In some embodiments, the compound of Formula II has the structure of Formula II-30. In some embodiments, the compound of Formula II has the structure of Formula II-31. In some embodiments, the compound of Formula II has the structure of Formula II-32. In some embodiments, the compound of Formula II has the structure of Formula II-33. In some embodiments, the compound of Formula II has the structure of Formula II-34. In some embodiments, the compound of Formula II has the structure of Formula II-35. In some embodiments, the compound of Formula II has the structure of Formula II-36. In some embodiments, the compound of Formula II has the structure of Formula II-37. In some embodiments, the compound of Formula II has the structure of Formula II-38. In some embodiments, the compound of Formula II has the structure of Formula II-39. In some embodiments, the compound of Formula II has the structure of Formula II-40. In some embodiments, the compound of Formula II has the structure of Formula II-41. In some embodiments, the compound of Formula II has the structure of Formula II-42. In some embodiments, the compound of Formula II has the structure of Formula II-43. In some embodiments, the compound of Formula II has the structure of Formula II-44. In some embodiments, the compound of Formula II has the structure of Formula II-45. In some embodiments, the compound of Formula II has the structure of Formula II-46. In some embodiments, the compound of Formula II has the structure of Formula II-47. In some embodiments, the compound of Formula II has the structure of Formula II-48. In some embodiments, the compound of Formula II has the structure of Formula II-49. In some embodiments, the compound of Formula II has the structure of Formula II-50. In some embodiments, the compound of Formula II has the structure of Formula II-51a. In some embodiments, the compound of Formula II has the structure of Formula II-51b. In some embodiments, the compound of Formula II has the structure of Formula II-52. In some embodiments, the compound of Formula II has the structure of Formula II-53. In some embodiments, the compound of Formula II has the structure of Formula II-54. In some embodiments, the compound of Formula II has the structure of Formula II-55. In some embodiments, the compound of Formula II has the structure of Formula II-56. In some embodiments, the compound of Formula II has the structure of Formula II-57. In some embodiments, the compound of Formula II has the structure of Formula II-58. In some embodiments, the compound of Formula II has the structure of Formula II-59. In some embodiments, the compound of Formula II has the structure of Formula II-60. In some embodiments, the compound of Formula II has the structure of Formula II-61. In some embodiments, the compound of Formula II has the structure of Formula II-62. In some embodiments, the compound of Formula II has the structure of Formula II-63. In some embodiments, the compound of Formula II has the structure of Formula II-64. In some embodiments, the compound of Formula II has the structure of Formula II-65. In some embodiments, the compound of Formula II has the structure of Formula II-66. In some embodiments, the compound of Formula II has the structure of Formula II-67. In some embodiments, the compound of Formula II has the structure of Formula II-68. In some embodiments, the compound of Formula II has the structure of Formula II-69. In some embodiments, the compound of Formula II has the structure of Formula II-70. In some embodiments, the compound of Formula II has the structure of Formula II-71. In some embodiments, the compound of Formula II has the structure of Formula II-72. In some embodiments, the compound of Formula II has the structure of Formula II-73. In some embodiments, the compound of Formula II has the structure of Formula II-74. In some embodiments, the compound of Formula II has the structure of Formula II-75. In some embodiments, the compound of Formula II has the structure of Formula II-76. In some embodiments, the compound of Formula II has the structure of Formula II-77. In some embodiments, the compound of Formula II has the structure of Formula II-78. In some embodiments, the compound of Formula II has the structure of Formula II-79. In some embodiments, the compound of Formula II has the structure of Formula II-80. In some embodiments, the compound of Formula II has the structure of Formula II-81. In some embodiments, the compound of Formula II has the structure of Formula II-82. In some embodiments, the compound of Formula II has the structure of Formula II-83. In some embodiments, the compound of Formula II has the structure of Formula II-84. In some embodiments, the compound of Formula II has the structure of Formula II-85. In some embodiments, the compound of Formula II has the structure of Formula II-86. In some embodiments, the compound of Formula II has the structure of Formula II-87. In some embodiments, the compound of Formula II has the structure of Formula II-88. In some embodiments, the compound of Formula II has the structure of Formula II-89. In some embodiments, the compound of Formula II has the structure of Formula II-90. In some embodiments, the compound of Formula II has the structure of Formula II-91. In some embodiments, the compound of Formula II has the structure of Formula II-92. In some embodiments, the compound of Formula II has the structure of Formula II-93. In some embodiments, the compound of Formula II has the structure of Formula II-94. In some embodiments, the compound of Formula II has the structure of Formula II-95. In some embodiments, the compound of Formula II has the structure of Formula II-96. In some embodiments, the compound of Formula II has the structure of Formula II-97. In some embodiments, the compound of Formula II has the structure of Formula II-98. In some embodiments, the compound of Formula II has the structure of Formula II-99. In some embodiments, the compound of Formula II has the structure of Formula II-100. In some embodiments, the compound of Formula II has the structure of Formula II-101. In some embodiments, the compound of Formula II has the structure of Formula II-102. In some embodiments, the compound of Formula II has the structure of Formula II-103. In some embodiments, the compound of Formula II has the structure of Formula II-104. In some embodiments, the compound of Formula II has the structure of Formula II-105. In some embodiments, the compound of Formula II has the structure of Formula II-106. In some embodiments, the compound of Formula II has the structure of Formula II-107. In some embodiments, the compound of Formula II has the structure of Formula II-108. In some embodiments, the compound of Formula II has the structure of Formula II-109. In some embodiments, the compound of Formula II has the structure of Formula II-110. In some embodiments, the compound of Formula II has the structure of Formula II-111. In some embodiments, the compound of Formula II has the structure of Formula II-112. In some embodiments, the compound of Formula II has the structure of Formula II-113. In some embodiments, the compound of Formula II has the structure of Formula II-114. In some embodiments, the compound of Formula II has the structure of Formula II-115. In some embodiments, the compound of Formula II has the structure of Formula II-116. In some embodiments, the compound of Formula II has the structure of Formula II-117. In some embodiments, the compound of Formula II has the structure of Formula II-118. In some embodiments, the compound of Formula II has the structure of Formula II-119. In some embodiments, the compound of Formula II has the structure of Formula II-120. In some embodiments, the compound of Formula II has the structure of Formula II-121. In some embodiments, the compound of Formula II has the structure of Formula II-122. In some embodiments, the compound of Formula II has the structure of Formula II-123. In some embodiments, the compound of Formula II has the structure of Formula II-124. In some embodiments, the compound of Formula II has the structure of Formula II-125. In some embodiments, the compound of Formula II has the structure of Formula II-126. In some embodiments, the compound of Formula II has the structure of Formula II-127. In some embodiments, the compound of Formula II has the structure of Formula II-128. In some embodiments, the compound of Formula II has the structure of Formula II-129. In some embodiments, the compound of Formula II has the structure of Formula II-130. In some embodiments, the compound of Formula II has the structure of Formula II-131. In some embodiments, the compound of Formula II has the structure of Formula II-132. In some embodiments, the compound of Formula II has the structure of Formula II-133. In some embodiments, the compound of Formula II has the structure of Formula II-134. In some embodiments, the compound of Formula II has the structure of Formula II-135. In some embodiments, the compound of Formula II has the structure of Formula II-136. In some embodiments, the compound of Formula II has the structure of Formula II-137. In some embodiments, the compound of Formula II has the structure of Formula II-138. In some embodiments, the compound of Formula II has the structure of Formula II-139. In some embodiments, the compound of Formula II has the structure of Formula II-140. In some embodiments, the compound of Formula II has the structure of Formula II-141. In some embodiments, the compound of Formula II has the structure of Formula II-142. In some embodiments, the compound of Formula II has the structure of Formula II-143. In some embodiments, the compound of Formula II has the structure of Formula II-144. In some embodiments, the compound of Formula II has the structure of Formula II-145. In some embodiments, the compound of Formula II has the structure of Formula II-146. In some embodiments, the compound of Formula II has the structure of Formula II-147. In some embodiments, the compound of Formula II has the structure of Formula II-148. In some embodiments, the compound of Formula II has the structure of Formula II-149. In some embodiments, the compound of Formula II has the structure of Formula II-150. In some embodiments, the compound of Formula II has the structure of Formula II-151. In some embodiments, the compound of Formula II has the structure of Formula II-152. In some embodiments, the compound of Formula II has the structure of Formula II-153. In some embodiments, the compound of Formula II has the structure of Formula II-154. In some embodiments, the compound of Formula II has the structure of Formula II-155. In some embodiments, the compound of Formula II has the structure of Formula II-156. In some embodiments, the compound of Formula II has the structure of Formula II-157. In some embodiments, the compound of Formula II has the structure of Formula II-158. In some embodiments, the compound of Formula II has the structure of Formula II-159. In some embodiments, the compound of Formula II has the structure of Formula II-160. In some embodiments, the compound of Formula II has the structure of Formula II-161. In some embodiments, the compound of Formula II has the structure of Formula II-162. In some embodiments, the compound of Formula II has the structure of Formula II-163. In some embodiments, the compound of Formula II has the structure of Formula II-164. In some embodiments, the compound of Formula II has the structure of Formula II-165. In some embodiments, the compound of Formula II has the structure of Formula II-166. In some embodiments, the compound of Formula II has the structure of Formula II-167. In some embodiments, the compound of Formula II has the structure of Formula II-168. In some embodiments, the compound of Formula II has the structure of Formula II-169. In some embodiments, the compound of Formula II has the structure of Formula II-170. In some embodiments, the compound of Formula II has the structure of Formula II-171. In some embodiments, the compound of Formula II has the structure of Formula II-172. In some embodiments, the compound of Formula II has the structure of Formula II-173. In some embodiments, the compound of Formula II has the structure of Formula II-174. In some embodiments, the compound of Formula II has the structure of Formula II-175. In some embodiments, the compound of Formula II has the structure of Formula II-176. In some embodiments, the compound of Formula II has the structure of Formula II-177. In some embodiments, the compound of Formula II has the structure of Formula II-178. In some embodiments, the compound of Formula II has the structure of Formula II-179. In some embodiments, the compound of Formula II has the structure of Formula II-180. In some embodiments, the compound of Formula II has the structure of Formula II-181. In some embodiments, the compound of Formula II has the structure of Formula II-182. In some embodiments, the compound of Formula II has the structure of Formula II-183. In some embodiments, the compound of Formula II has the structure of Formula II-184. In some embodiments, the compound of Formula II has the structure of Formula II-185. In some embodiments, the compound of Formula II has the structure of Formula II-186. In some embodiments, the compound of Formula II has the structure of Formula II-187. In some embodiments, the compound of Formula II has the structure of Formula II-188. In some embodiments, the compound of Formula II has the structure of Formula II-189. In some embodiments, the compound of Formula II has the structure of Formula II-190. In some embodiments, the compound of Formula II has the structure of Formula II-191. In some embodiments, the compound of Formula II has the structure of Formula II-192. In some embodiments, the compound of Formula II has the structure of Formula II-193. In some embodiments, the compound of Formula II has the structure of Formula II-194. In some embodiments, the compound of Formula II has the structure of Formula II-195. In some embodiments, the compound of Formula II has the structure of Formula II-196. In some embodiments, the compound of Formula II has the structure of Formula II-197. In some embodiments, the compound of Formula II has the structure of Formula II-198. In some embodiments, the compound of Formula II has the structure of Formula II-199. In some embodiments, the compound of Formula II has the structure of Formula II-200. In some embodiments, the compound of Formula II has the structure of Formula II-201. In some embodiments, the compound of Formula II has the structure of Formula II-202. In some embodiments, the compound of Formula II has the structure of Formula II-203. In some embodiments, the compound of Formula II has the structure of Formula II-204. In some embodiments, the compound of Formula II has the structure of Formula II-205. In some embodiments, the compound of Formula II has the structure of Formula II-206. In some embodiments, the compound of Formula II has the structure of Formula II-207. In some embodiments, the compound of Formula II has the structure of Formula II-208. In some embodiments, the compound of Formula II has the structure of Formula II-209. In some embodiments, the compound of Formula II has the structure of Formula II-210. In some embodiments, the compound of Formula II has the structure of Formula II-211. In some embodiments, the compound of Formula II has the structure of Formula II-212. In some embodiments, the compound of Formula II has the structure of Formula II-213. In some embodiments, the compound of Formula II has the structure of Formula II-214. In some embodiments, the compound of Formula II has the structure of Formula II-215. In some embodiments, the compound of Formula II has the structure of Formula II-216. In some embodiments, the compound of Formula II has the structure of Formula II-217. In some embodiments, the compound of Formula II has the structure of Formula II-218. In some embodiments, the compound of Formula II has the structure of Formula II-219. In some embodiments, the compound of Formula II has the structure of Formula II-220. In some embodiments, the compound of Formula II has the structure of Formula II-221. In some embodiments, the compound of Formula II has the structure of Formula II-222. In some embodiments, the compound of Formula II has the structure of Formula II-223. In some embodiments, the compound of Formula II has the structure of Formula II-224. In some embodiments, the compound of Formula II has the structure of Formula II-225. In some embodiments, the compound of Formula II has the structure of Formula II-226. In some embodiments, the compound of Formula II has the structure of Formula II-227. In some embodiments, the compound of Formula II has the structure of Formula II-228. In some embodiments, the compound of Formula II has the structure of Formula II-229. In some embodiments, the compound of Formula II has the structure of Formula II-230. In some embodiments, the compound of Formula II has the structure of Formula II-231. In some embodiments, the compound of Formula II has the structure of Formula II-232. In some embodiments, the compound of Formula II has the structure of Formula II-233. In some embodiments, the compound of Formula II has the structure of Formula II-234. In some embodiments, the compound of Formula II has the structure of Formula II-235. In some embodiments, the compound of Formula II has the structure of Formula II-236. In some embodiments, the compound of Formula II has the structure of Formula II-237. In some embodiments, the compound of Formula II has the structure of Formula II-238. In some embodiments, the compound of Formula II has the structure of Formula II-239. In some embodiments, the compound of Formula II has the structure of Formula II-240. In some embodiments, the compound of Formula II has the structure of Formula II-241. In some embodiments, the compound of Formula II has the structure of Formula II-242. In some embodiments, the compound of Formula II has the structure of Formula II-243. In some embodiments, the compound of Formula II has the structure of Formula II-244. In some embodiments, the compound of Formula II has the structure of Formula II-245. In some embodiments, the compound of Formula II has the structure of Formula II-246. In some embodiments, the compound of Formula II has the structure of Formula II-247. In some embodiments, the compound of Formula II has the structure of Formula II-248. In some embodiments, the compound of Formula II has the structure of Formula II-249. In some embodiments, the compound of Formula II has the structure of Formula II-250. In some embodiments, the compound of Formula II has the structure of Formula II-251. In some embodiments, the compound of Formula II has the structure of Formula II-252. In some embodiments, the compound of Formula II has the structure of Formula II-253. In some embodiments, the compound of Formula II has the structure of Formula II-254. In some embodiments, the compound of Formula II has the structure of Formula II-255. In some embodiments, the compound of Formula II has the structure of Formula II-256. In some embodiments, the compound of Formula II has the structure of Formula II-257. In some embodiments, the compound of Formula II has the structure of Formula II-258. In some embodiments, the compound of Formula II has the structure of Formula II-259. In some embodiments, the compound of Formula II has the structure of Formula II-260. In some embodiments, the compound of Formula II has the structure of Formula II-261. In some embodiments, the compound of Formula II has the structure of Formula II-262. In some embodiments, the compound of Formula II has the structure of Formula II-263. In some embodiments, the compound of Formula II has the structure of Formula II-264. In some embodiments, the compound of Formula II has the structure of Formula II-265. In some embodiments, the compound of Formula II has the structure of Formula II-266. In some embodiments, the compound of Formula II has the structure of Formula II-267. In some embodiments, the compound of Formula II has the structure of Formula II-268. In some embodiments, the compound of Formula II has the structure of Formula II-269. In some embodiments, the compound of Formula II has the structure of Formula II-270. In some embodiments, the compound of Formula II has the structure of Formula II-271. In some embodiments, the compound of Formula II has the structure of Formula II-272. In some embodiments, the compound of Formula II has the structure of Formula II-273. In some embodiments, the compound of Formula II has the structure of Formula II-274. In some embodiments, the compound of Formula II has the structure of Formula II-275.

In some embodiments of any of the foregoing aspects, the small molecule ALK2 inhibitor or a pharmaceutically acceptable salt thereof is a compound of Formula III:

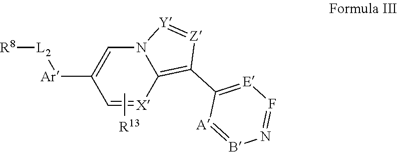

or a pharmaeutically acceptable salt thereof, wherein
X′ is selected from CR¹⁵ and N;
Y′ is selected from CR¹⁵ and N;
Z′ is selected from CR²⁸ and N;
Ar′ is selected from substituted or unsubstituted aryl and heteroaryl, e.g., a six-membered ring, such as phenyl;
L₂ is absent or selected from substituted or unsubstituted alkyl and heteroalkyl;
A and B, independently for each occurrence, are selected from CR¹⁶ and N, preferably CR¹⁶, e.g., CH;
E and F, independently for each occurrence, are selected from CR⁵′ and N, preferably CR⁵;
preferably chosen such that no more than two of A, B, E, and F are N;
R²⁶ represents a substituent, e.g., selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido, e.g., lower alkyl;
R⁸ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido, e.g., substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, or sulfonamido, preferably substituted or unsubstituted heterocyclyl or heteroaryl;
R⁵, independently for each occurrence, represents a substituent, e.g., selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano), or two occurrences of R⁵′taken together with the atoms to which they are attached form a substituted or unsubstituted 5- or 6-membered cycloalkyl, heterocycloalkyl, aryl, or heteroaryl ring, preferably an aryl or heteroaryl ring, e.g., a substituted or unsubstituted benzo ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably substituted or unsubstituted alkyl, heteroalkyl, halogen, hydroxyl, alkoxyl, alkylthio, acyloxy, acylamino, carbamate, or cyano;
R¹⁵′, independently for each occurrence, represents a substituent, e.g., selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably H or substituted or unsubstituted alkyl, heteroalkyl, halogen, hydroxyl, alkoxyl, alkylthio, acyloxy, acylamino, carbamate, or cyano;
R¹⁶′, independently for each occurrence, represents a substituent, e.g., selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, or cyano. In some embodiments, the compound of Formula III has a structure of Formula III-a:

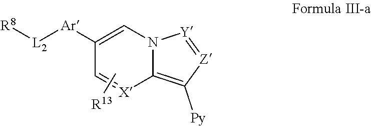

or a pharmaceutically acceptable salt thereof, wherein
X′ is selected from CR¹⁵ and N;
Y′ is selected from CR¹⁵ and N;
Z′ is selected from CR²⁶ and N;
Ar′ is selected from substituted or unsubstituted aryl and heteroaryl, e.g., a six-membered ring, such as phenyl;
L₂ is absent or selected from substituted or unsubstituted alkyl and heteroalkyl;
Py is substituted or unsubstituted 4-pyridinyl or 4-quinolinyl, e.g., optionally substituted with substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, amino, acylamino, carbamate, amido, amidino, cyano, sulfonyl, sulfoxido, sulfamoyl, or sulfonamido; and
R²⁶ represents a substituent, e.g., selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido, e.g., lower alkyl;
R⁸ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido, e.g., substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, or sulfonamido, preferably substituted or unsubstituted heterocyclyl or heteroaryl;
R⁵′, independently for each occurrence, represents a substituent, e.g., selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano), or two occurrences of R²⁶ taken together with the atoms to which they are attached form a substituted or unsubstituted 5- or 6-membered cycloalkyl, heterocycloalkyl, aryl, or heteroaryl ring, preferably an aryl or heteroaryl ring, e.g., a substituted or unsubstituted benzo ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably substituted or unsubstituted alkyl, heteroalkyl, halogen, hydroxyl, alkoxyl, alkylthio, acyloxy, acylamino, carbamate, or cyano;
R¹⁵′, independently for each occurrence, represents a substituent, e.g., selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably H or substituted or unsubstituted alkyl, heteroalkyl, halogen, hydroxyl, alkoxyl, alkylthio, acyloxy, acylamino, carbamate, or cyano;
R¹⁶′, independently for each occurrence, represents a substituent, e.g., selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, or cyano.

In some embodiments, the compound of Formula III has a structure of Formula III-b:

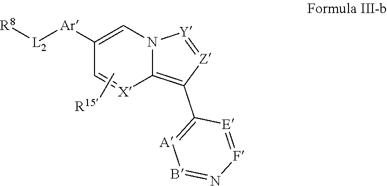

or a pharmaceutically acceptable salt thereof, wherein
X′ and Y′ are each N;
Z′ is CR²⁶;
Ar′ is substituted or unsubstituted phenyl;
L₂ is absent or selected from substituted or unsubstituted alkyl and heteroalkyl;
A′ and B′ are both CR¹⁶;
E′ and F′ are both CR⁵′ and both occurrences of R⁵′ taken together with E′ and F′ form a substituted or unsubstituted 5- or 6-membered cycloalkyl, heterocycloalkyl, aryl, or heteroaryl ring;
R²⁶ is selected from H and substituted or unsubstituted alkyl;
R⁸ is selected from H and substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R¹⁵′, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido; and
R¹⁶′, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In some embodiments, the compound of Formula III has a structure of Formula III-b, or a pharmaceutically acceptable salt thereof, wherein
X′ and Y′ are each N;
Z′ is CR²⁶;
Ar′ is selected from substituted or unsubstituted aryl and heteroaryl;
L₂ is absent or selected from substituted or unsubstituted alkyl and heteroalkyl;
A′ and B′ are both CR^{16′;}
E′ and F′ are both CR⁵ and both occurrences of R⁵ taken together with E′ and F′ form a substituted or unsubstituted 5- or 6-membered cycloalkyl, heterocycloalkyl, aryl, or heteroaryl ring;
R²⁶ is selected from H and substituted or unsubstituted alkyl;
R⁸ is selected from H and substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R¹⁵′, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido;
R¹⁶′, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In some embodiments, the compound of Formula III has a structure of any one of Formulas III-1 to III-35 or is a pharmaceutically acceptable salt thereof. In some embodiments, the compound of Formula III has the structure of Formula III-1. In some embodiments, the compound of Formula III has the structure of Formula III-2. In some embodiments, the compound of Formula III has the structure of Formula III-3. In some embodiments, the compound of Formula III has the structure of Formula III-4. In some embodiments, the compound of Formula III has the structure of Formula III-5. In some embodiments, the compound of Formula III has the structure of Formula III-6. In some embodiments, the compound of Formula III has the structure of Formula III-7. In some embodiments, the compound of Formula III has the structure of Formula III-8. In some embodiments, the compound of Formula III has the structure of Formula III-9. In some embodiments, the compound of Formula III has the structure of Formula III-10. In some embodiments, the compound of Formula III has the structure of Formula III-11. In some embodiments, the compound of Formula III has the structure of Formula III-12. In some embodiments, the compound of Formula III has the structure of Formula III-13. In some embodiments, the compound of Formula III has the structure of Formula III-14. In some embodiments, the compound of Formula III has the structure of Formula III-15. In some embodiments, the compound of Formula III has the structure of Formula III-16. In some embodiments, the compound of Formula III has the structure of Formula III-17. In some embodiments, the compound of Formula III has the structure of Formula III-18. In some embodiments, the compound of Formula III has the structure of Formula III-19. In some embodiments, the compound of Formula III has the structure of Formula III-20. In some embodiments, the compound of Formula III has the structure of Formula III-21. In some embodiments, the compound of Formula III has the structure of Formula III-22. In some embodiments, the compound of Formula III has the structure of Formula III-23. In some embodiments, the compound of Formula III has the structure of Formula III-24. In some embodiments, the compound of Formula III has the structure of Formula III-25. In some embodiments, the compound of Formula III has the structure of Formula III-26. In some embodiments, the compound of Formula III has the structure of Formula III-27. In some embodiments, the compound of Formula III has the structure of Formula III-28. In some embodiments, the compound of Formula III has the structure of Formula III-29. In some embodiments, the compound of Formula III has the structure of Formula III-30. In some embodiments, the compound of Formula III has the structure of Formula III-31. In some embodiments, the compound of Formula III has the structure of Formula III-32. In some embodiments, the compound of Formula III has the structure of Formula III-33. In some embodiments, the compound of Formula III has the structure of Formula III-34. In some embodiments, the compound of Formula III has the structure of Formula III-35.

In some embodiments of any of the foregoing aspects, the small molecule ALK2 inhibitor or a pharmaceutically acceptable salt thereof is Compound 1:

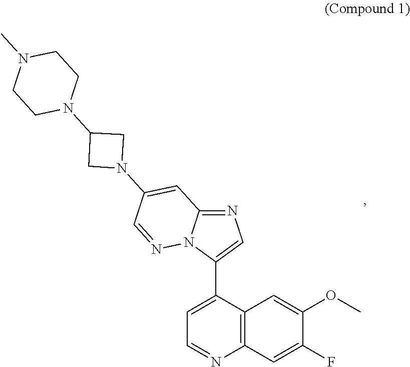

or a pharmaceutically acceptable salt thereof.

In some embodiments of any of the foregoing aspects, the small molecule ALK2 inhibitor or a pharmaceutically acceptable salt thereof is Compound 2:

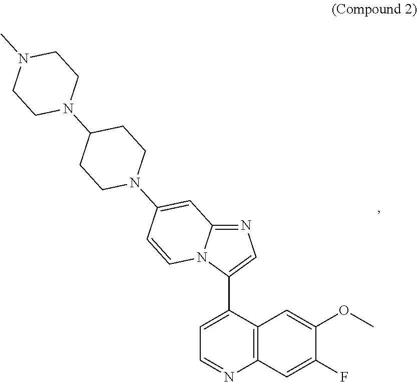

or a pharmaceutically acceptable salt thereof.

In some embodiments of any of the foregoing aspects, the small molecule ALK2 inhibitor or a pharmaceutically acceptable salt thereof is Compound 3:

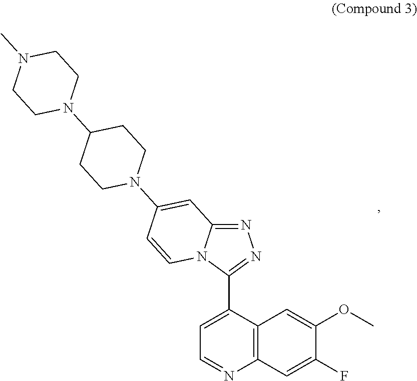

or a pharmaceutically acceptable salt thereof.

In some embodiments of any of the foregoing aspects, the small molecule ALK2 inhibitor or a pharmaceutically acceptable salt thereof is Compound 4:

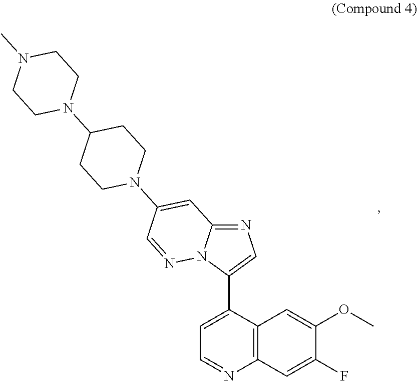

or a pharmaceutically acceptable salt thereof.

In some embodiments of any of the foregoing aspects, the small molecule ALK2 inhibitor or a pharmaceutically acceptable salt thereof is Compound 5:

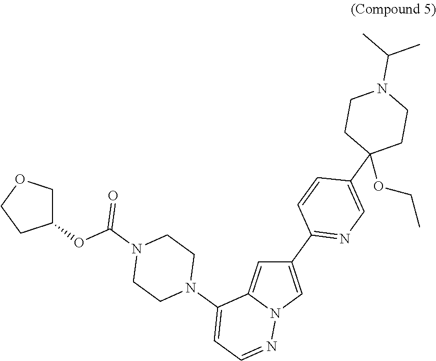 [SMILES: CCOC1(c2ccc(-c3cc4c(N5CCN(C(=O)O[C@@H]6CCOC6)CC5)ccnn4c3)nc2)CCN(C(C)C)CC1]

or a pharmaceutically acceptable salt thereof.

In some embodiments of any of the foregoing aspects, the small molecule ALK2 inhibitor or a pharmaceutically acceptable salt thereof is Compound 6:

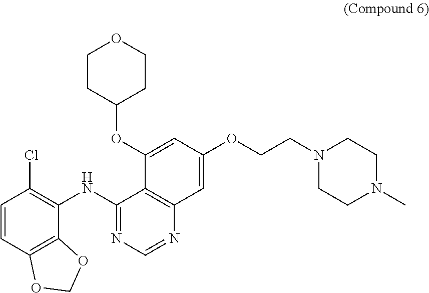 [SMILES: CN1CCN(CCOc2cc(OC3CCOCC3)c3c(Nc4c(Cl)ccc5c4OCO5)ncnc3c2)CC1]

or a pharmaceutically acceptable salt thereof.

In some embodiments of any of the foregoing aspects, the small molecule ALK2 inhibitor or a pharmaceutically acceptable salt thereof is Compound 7:

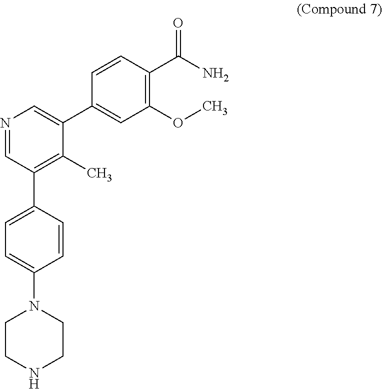 [SMILES: COc1cc(-c2cncc(-c3ccc(N4CCNCC4)cc3)c2C)ccc1C(N)=O]

or a pharmaceutically acceptable salt thereof.

In some embodiments of any of the foregoing aspects, the small molecule ALK2 inhibitor or a pharmaceutically acceptable salt thereof is BCX9250 or a pharmaceutically acceptable salt thereof.

In some embodiments of any of the foregoing aspects, the small molecule ALK2 inhibitor or a pharmaceutically acceptable salt thereof is INCB00928 or a pharmaceutically acceptable salt thereof.

In some embodiments of any of the foregoing aspects, the small molecule ALK2 inhibitor or a pharmaceutically acceptable salt thereof is dorsomorphin or a pharmaceutically acceptable salt thereof.

In some embodiments of any of the foregoing aspects, the small molecule ALK2 inhibitor or a pharmaceutically acceptable salt thereof is LDN-212854 or a pharmaceutically acceptable salt thereof.

In some embodiments of any of the foregoing aspects, the small molecule ALK2 inhibitor or a pharmaceutically acceptable salt thereof is LDN-193189 or a pharmaceutically acceptable salt thereof.

In some embodiments of any of the foregoing aspects, the small molecule ALK2 inhibitor or a pharmaceutically acceptable salt thereof is LDN-21411 or a pharmaceutically acceptable salt thereof.

In some embodiments of any of the foregoing aspects, the small molecule ALK2 inhibitor is a compound selected from the group consisting of: (R)-3-(6-(3-Methylpiperazin-1-yl)pyridin-3-yl)-5-(piperidin-1-yl)-1H-pyrazolo[4,3-b]pyridine; (R)-3-(6-(3-Methylpiperazin-1-yl)pyridin-3-yl)-5-(piperidin-1-yl)-1H-pyrazolo[3,4-c]pyridine; (R)-3-(6-(3-methylpiperazin-1-yl)pyridin-3-yl)-5-(piperidin-1-yl)-1H-pyrazolo[4,3-d]pyrimidine; 3-(6-((R)-3-methylpiperazin-1-yl)pyridin-3-yl)-5-(2-methylpiperidin-1-yl)-1H-pyrazolo[4,3-d]pyrimidine; 5-(cis-2,6-Dimethylpiperazin-1-yl)-3-(6-((R)-3-methylpiperazin-1-yl)pyridin-3-yl)-1H-pyrazolo[4,3-d]pyrimidine; (3R,5S)—N,N,3,5-tetramethyl-4-(3-(6-((R)-3-methylpiperazin-1-yl)pyridin-3-yl)-1H-pyrazolo[4,3-d]pyrimidin-5-yl)piperazine-1-carboxamide; 5-((2R,6S)-2,6-dimethylpiperidin-1-yl)-3-(6-((R)-3-methylpiperazin-1-yl)pyridin-3-yl)-1H-pyrazolo[4,3-d]pyrimidine; (3R,5S)-3,5-dimethyl-4-(3-(6-((R)-3-methylpiperazin-1-yl)pyridin-3-yl)-1H-pyrazolo[4,3-d]pyrimidin-5-yl)morpholine; ((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)(pyrrolidin-1-yl)methanone; 5-(7-azabicyclo[2.2.1]heptan-7-yl)-3-(6-((R)-3-methylpiperazin-1-yl)pyridin-3-yl)-1H-pyrazolo[4,3-d]pyrimidine; (1R,5S,6S)-8-(3-(6-((R)-3-methylpiperazin-1-yl)pyridin-3-yl)-1H-pyrazolo[4,3-d]pyrimidin-5-yl)-8-azabicyclo[3.2.1]octan-6-ol; 1-((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)-2-methylpropan-1-one; 1-((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)-2-methoxyethan-1-one; (3R,5S)—N-isopropyl-3,5-dimethyl-4-(3-(6-((R)-3-methylpiperazin-1-yl)pyridin-3-yl)-1H-pyrazolo[4,3-d]pyrimidin-5-yl)piperazine-1-carboxamide; (3R,5S)—N,N,3,5-tetramethyl-4-(3-(6-((R)-3-methylpiperazin-1-yl)pyridin-3-yl)-1H-pyrazolo[4,3-d]pyrimidin-5-yl)piperazine-I-carboxamide; ((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)(pyrrolidin-1-yl)methanone: ((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)(3-fluoropyrrolidin-1-yl)methanone; Ethyl (3R,5S)-3,5-dimethyl-4-(3-(6-((R)-3-methylpiperazin-1-yl)pyridin-3-yl)-1H-pyrazolo[4,3-d]pyrimidin-5-yl)piperazine-1-carboxylate; 2-Fluoroethyl (3R,5S)-3,5-dimethyl-4-(3-(6-((R)-3-methylpiperazin-1-yl)pyridin-3-yl)-1H-pyrazolo[4,3-d]pyrimidin-5-yl)piperazine-1-carboxylate; Cyclopropyl((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)methanone; ((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)(pyrrolidin-I-yl)methanone; 4-((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)benzonitrile; 5-((2R,6S)-2,6-dimethylpiperidin-1-yl)-3-(4-(4-methylpiperazin-1-yl)phenyl)-1H-pyrazolo[4,3-d]pyrimidine; Methyl (3R,5S)-3,5-dimethyl-4-(3-(6-((R)-3-methylpiperazin-1-yl)pyridin-3-yl)-1H-pyrazolo[4,3-d]pyrimidin-5-yl)piperazine-1-carboxylate; (R)-5-(1-Methyl-1,4,6,7-tetrahydro-5H-imidazo[4,5-c]pyridin-5-yl)-3-(6-(3-methylpiperazin-1-yl)pyridin-3-yl)-1H-pyrazolo[4,3-d]pyrimidine; (R)-5-(3-Methyl-5,6-dihydroimidazo[1,2-a]pyrazin-7 (8H)-yl)-3-(6-(3-methylpiperazin-1-yl)pyridin-3-yl)-1H-pyrazolo[4,3-d]pyrimidine; (5-(3-(6-((R)-3-Methylpiperazin-1-yl)pyridin-3-yl)-1H-pyrazolo[4,3-d]pyrimidin-5-yl)-2,5-diazabicyclo[2.2.2]octan-2-yl)(pyrrolidin-1-yl)methanone; ((1S,4S)-5-(3-(6-((R)-3-Methylpiperazin-1-yl)pyridin-3-yl)-1H-pyrazolo[4,3-d]pyrimidin-5-yl)-2,5-diazabicyclo[2.2.1]heptan-2-yl)(pyrrolidin-1-yl)methanone; Methyl (3R,5S)-3,5-dimethyl-4-(3-(4-(4-methylpiperazin-1-yl)phenyl)-1H-pyrazolo[4,3-d]pyrimidin-5-yl)piperazine-1-carboxylate; Methyl (3R,5S)-4-(3-(3-fluoro-4-(4-methylpiperazin-1-yl)phenyl)-1H-pyrazolo[4,3-d]pyrimidin-5-yl)-3,5-dimethylpiperazine-1-carboxylate; Methyl (3R,5S)-3,5-dimethyl-4-(3-(6-(4-methylpiperazin-1-yl)pyridin-3-yl)-1H-pyrazolo[4,3-d]pyrimidin-5-yl)piperazine-1-carboxylate; Methyl (3R,5S)-4-(3-(4-(4-ethylpiperazin-1-yl)-3-methylphenyl)-1H-pyrazolo[4,3-d]pyrimidin-5-yl)-3,5-dimethylpiperazine-1-carboxylate; Methyl (3R,5S)-3,5-dimethyl-4-(3-(4-(methylcarbamoyl)phenyl)-1H-pyrazolo[4,3-d]pyrimidin-5-yl)piperazine-1-carboxylate: Methyl (3R,5S)-3,5-dimethyl-4-(3-(4-(1-methylpyrrolidin-3-yl)phenyl)-1H-pyrazolo[4,3-d]pyrimidin-5-yl)piperazine-1-carboxylate; Methyl (3R,5S)-4-(3-(4-(1-(2-hydroxyethyl)pyrrolidin-3-yl)phenyl)-1H-pyrazolo[4,3-d]pyrimidin-5-yl)-3,5-dimethylpiperazine-1-carboxylate; Methyl (3R,5S)-3,5-dimethyl-4-(3-(4-(1-methylpyrrolidin-3-yl)phenyl)-1H-pyrazolo[4,3-d]pyrimidin-5-yl)piperazine-1-carboxylate; Methyl (3R,5S)-4-(3-(4-(4-(1-hydroxypropan-2-yl)piperazin-1-yl)phenyl)-1H-pyrazolo[4,3-d]pyrimidin-5-yl)-3,5-dimethylpiperazine-1-carboxylate; Methyl (3R,5S)-3,5-dimethyl-4-(3-(4-(4-(tetrahydro-2H-pyran-4-yl)piperazin-1-yl)phenyl)-1H-pyrazolo[4,3-d]pyrimidin-5-yl)piperazine-1-carboxylate; 2-(4-(4-(5-((2R,6S)-4-(Methoxycarbonyl)-2,6-dimethylpiperazin-1-yl)-1H-pyrazolo[4,3-d]pyrimidin-3-yl)phenyl)piperazin-1-yl)-2-methylpropanoic acid; Methyl (3R,5S)-3,5-dimethyl-4-(3-(6-((R)-2-methylmorpholino)pyridin-3-yl)-1H-pyrazolo[4,3-d]pyrimidin-5-yl)piperazine-1-carboxylate; Methyl (3R,5S)-4-(3-(6-((R)-3,4-dimethylpiperazin-1-yl)pyridin-3-yl)-1H-pyrazolo[4,3-d]pyrimidin-5-yl)-3,5-dimethylpiperazine-1-carboxylate; Methyl (3R,5S)-4-(3-(4-(4-hydroxypiperidin-1-yl)phenyl)-1H-pyrazolo[4,3-d]pyrimidin-5-yl)-3,5-dimethylpiperazine-1-carboxylate; Methyl (3R,5S)-4-(3-(4-((7S,8aR)-7-hydroxyhexahydropyrrolo[1,2-a]pyrazin-2 (1H)-yl)phenyl)-1H-pyrazolo[4,3-d]pyrimidin-5-yl)-3,5-dimethylpiperazine-1-carboxylate; 1-Methyl-4-(3-(1-methyl-1H-pyrazol-4-yl)-1H-pyrazolo[4,3-d]pyrimidin-5-yl)piperazin-2-one; 1-Cyclopropyl-4-(3-(1-methyl-1H-pyrazol-4-yl)-1H-pyrazolo[4,3-d]pyrimidin-5-yl)piperazin-2-one; 1,3-Dimethyl-4-(3-(1-methyl-1H-pyrazol-4-yl)-1H-pyrazolo[4,3-d]pyrimidin-5-yl)piperazin-2-one; 3-Ethyl-1-methyl-4-(3-(1-methyl-1H-pyrazol-4-yl)-1H-pyrazolo[4,3-d]pyrimidin-5-yl)piperazin-2-one; 6-(3-(1-Methyl-1H-pyrazol-4-yl)-1H-pyrazolo[4,3-d]pyrimidin-5-yl)-5,6,7,8-tetrahydro-1,6-naphthyridine; 3-(1-Methyl-1H-pyrazol-4-yl)-5-(1-(trifluoromethyl)-5,6-dihydroimidazo[1,5-a]pyrazin-7 (8H)-yl)-1H-pyrazolo[4,3-d]pyrimidine; 5-(3-(1-Methyl-1H-pyrazol-4-yl)-1H-pyrazolo[4,3-d]pyrimidin-5-yl)-4,5,6,7-tetrahydrothiazolo[5,4-c]pyridine; 3-(1-Methyl-1H-pyrazol-4-yl)-5-(8-methyl-5,6-dihydroimidazo[1,2-a]pyrazin-7 (8H)-yl)-1H-pyrazolo[4,3-d]pyrimidine; 3-(1-Methyl-1H-pyrazol-4-yl)-5-(1,4,5,7-tetrahydro-6H-pyrazolo[3,4-c]pyridin-6-yl)-1H-pyrazolo[4,3-d]pyrimidine; 9-(3-(1-Methyl-1H-pyrazol-4-yl)-1H-pyrazolo[4,3-d]pyrimidin-5-yl)-1,4,5,6,7,8-hexahydro-4,7-epiminocyclohepta[c]pyrazole; 1-(2,2-Difluoroethyl)-3-methyl-4-(3-(1-methyl-1H-pyrazol-4-yl)-1H-pyrazolo[4,3-d]pyrimidin-5-yl)piperazin-2-one; 1-Isopropyl-3-methyl-4-(3-(1-methyl-1H-pyrazol-4-yl)-1H-pyrazolo[4,3-d]pyrimidin-5-yl)piperazin-2-one; 1-(2-Methoxyethyl)-3-methyl-4-(3-(1-methyl-1H-pyrazol-4-yl)-1H-pyrazolo[4,3-d]pyrimidin-5-yl)piperazin-2-one; 3-Methyl-8-(3-(1-methyl-1H-pyrazol-4-yl)-1H-pyrazolo[4,3-d]pyrimidin-5-yl)-3,8-diazabicyclo[3.2.1]octan-2-one: 3-Ethyl-8-(3-(1-methyl-1H-pyrazol-4-yl)-1H-pyrazolo[4,3-d]pyrimidin-5-yl)-3,8-diazabicyclo[3.2.1]octan-2-one; 3-(2-Fluoroethyl)-8-(3-(1-methyl-1H-pyrazol-4-yl)-1H-pyrazolo[4,3-d]pyrimidin-5-yl)-3,8-diazabicyclo[3.2.1]octan-2-one; 3-Isopropyl-8-(3-(1-methyl-1H-pyrazol-4-yl)-1H-pyrazolo[4,3-d]pyrimidin-5-yl)-3,8-diazabicyclo[3.2.1]octan-2-one; 8-(3-(1-(2-Methoxyethyl)-1H-pyrazol-4-yl)-1H-pyrazolo[4,3-d]pyrimidin-5-yl)-3-methyl-3,8-diazabicyclo[3.2.1]octan-2-one; 3-Methyl-8-(3-(1-(tetrahydro-2H-pyran-4-yl)-1H-pyrazol-4-yl)-1H-pyrazolo[4,3-d]pyrimidin-5-yl)-3,8-diazabicyclo[3.2.1]octan-2-one; 8-(3-(1-((1R,4S)-4-Hydroxycyclohexyl)-1H-pyrazol-4-yl)-1H-pyrazolo[4,3-d]pyrimidin-5-yl)-3-methyl-3,8-diazabicyclo[3.2.1]octan-2-one; N, N-Dimethyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)piperidine-1-carboxamide; N,N-Dimethyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzamide; 8-(3-(1-(1-Isobutyrylpiperidin-4-yl)-1H-pyrazol-4-yl)-1H-pyrazolo[4,3-d]pyrimidin-5-yl)-3-methyl-3,8-diazabicyclo[3.2.1]octan-2-one; 3-Methyl-8-(3-(1-(2-morpholinoethyl)-1H-pyrazol-4-yl)-1H-pyrazolo[4,3-d]pyrimidin-5-yl)-3,8-diazabicyclo[3.2.1]octan-2-one; 3-Methyl-8-(3-(1-(pyridin-4-ylmethyl)-1H-pyrazol-4-yl)-1H-pyrazolo[4,3-d]pyrimidin-5-yl)-3,8-diazabicyclo[3.2.1]octan-2-one; 3-Methyl-8-(3-(6-(4-methylpiperazin-1-yl)pyridin-3-yl)-1H-pyrazolo[4,3-d]pyrimidin-5-yl)-3,8-diazabicyclo[3.2.1]octan-2-one; 8-(3-(1-Ethyl-1H-pyrazol-4-yl)-1H-pyrazolo[4,3-d]pyrimidin-5-yl)-3-methyl-3,8-diazabicyclo[3.2.1]octan-2-one; 8-(3-(1-Isopropyl-1H-pyrazol-4-yl)-1H-pyrazolo[4,3-d]pyrimidin-5-yl)-3-methyl-3,8-diazabicyclo[3.2.1]octan-2-one; 8-(3-(1-Cyclobutyl-1H-pyrazol-4-yl)-1H-pyrazolo[4,3-d]pyrimidin-5-yl)-3-methyl-3,8-diazabicyclo[3.2.1]octan-2-one; 8-(3-(1-Cyclopropyl-1H-pyrazol-4-yl)-1H-pyrazolo[4,3-d]pyrimidin-5-yl)-3-methyl-3,8-diazabicyclo[3.2.1]octan-2-one; 3-Methyl-8-(3-(1-(2,2,2-trifluoroethyl)-1H-pyrazol-4-yl)-1H-pyrazolo[4,3-d]pyrimidin-5-yl)-3,8-diazabicyclo[3.2.1]octan-2-one; 3-Methyl-8-(3-(1-(pyridin-4-yl)-1H-pyrazol-4-yl)-1H-pyrazolo[4,3-d]pyrimidin-5-yl)-3,8-diazabicyclo[3.2.1]octan-2-one; 3-Methyl-8-(3-(1-(pyridin-3-yl)-1H-pyrazol-4-yl)-1H-pyrazolo[4,3-d]pyrimidin-5-yl)-3,8-diazabicyclo[3.2.1]octan-2-one: 4-(4-(5-(3-Methyl-2-oxo-3,8-diazabicyclo[3.2.1]octan-8-yl)-1H-pyrazolo[4,3-d]pyrimidin-3-yl)-1H-pyrazol-1-yl)benzonitrile; 3-Methyl-8-(3-(1-(2-methylpyridin-4-yl)-1H-pyrazol-4-yl)-1H-pyrazolo[4,3-d]pyrimidin-5-yl)-3,8-diazabicyclo[3.2.1]octan-2-one; 8-(3-(1-(6-(Dimethylamino)pyridin-3-yl)-1H-pyrazol-4-yl)-1H-pyrazolo[4,3-d]pyrimidin-5-yl)-3-methyl-3,8-diazabicyclo[3.2.1]octan-2-one; 5-(4-(5-(3-Methyl-2-oxo-3,8-diazabicyclo[3.2.1]octan-8-yl)-1H-pyrazolo[4,3-d]pyrimidin-3-yl)-1H-pyrazol-1-yl) nicotinonitrile; 5-(4-(5-(3-Methyl-2-oxo-3,8-diazabicyclo[3.2.1]octan-8-yl)-1H-pyrazolo[4,3-d]pyrimidin-3-yl)-1H-pyrazol-1-yl)picolinonitrile; 8-(3-(1-(Imidazo[1,2-a]pyridin-6-yl)-1H-pyrazol-4-yl)-1H-pyrazolo[4,3-d]pyrimidin-5-yl)-3-methyl-3,8-diazabicyclo[3.2.1]octan-2-one; 2-Methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzonitrile; 3-Methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzonitrile; 2-Fluoro-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzonitrile; N,N-Dimethyl-4-(4-(5-(3-methyl-2-oxo-3,8-diazabicyclo[3.2.1]octan-8-yl)-1H-pyrazolo[4,3-d]pyrimidin-3-yl)-1H-pyrazol-I-yl)benzamide; N-Methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzamide; 2-Fluoro-N-methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzamide; N-Methyl-5-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)picolinamide; 4-(6-Methyl-7-(4-(piperazin-1-yl)phenyl)imidazo[1,2-b]pyridazin-3-yl)quinoline; Methyl 7-(4-(piperazin-1-yl)phenyl)-3-(quinolin-4-yl)imidazo[1,2-b]pyridazine-6-carboxylate; N-((1R,4R)-4-Hydroxy-4-methylcyclohexyl)-7-(4-(piperazin-1-yl)phenyl)-3-(quinolin-4-yl)imidazo[1,2-b]pyridazine-6-carboxamide; Ethyl 4-(7-(4-(piperazin-1-yl)phenyl)-3-(quinolin-4-yl)imidazo[1,2-b]pyridazine-6-carbonyl)piperazine-1-carboxylate; 7-(4-(Piperazin-1-yl)phenyl)-3-(quinolin-4-yl)imidazo[1,2-b]pyridazine-6-carboxamide; 7-(4-(piperazin-1-yl)phenyl)-3-(quinolin-4-yl)imidazo[1,2-b]pyridazine-6-carbonitrile; (7-(4-(Piperazin-1-yl)phenyl)-3-(quinolin-4-yl)imidazo[1,2-b]pyridazin-6-yl)methanol; 4-(6-(Methoxymethyl)-7-(4-(piperazin-1-yl)phenyl)imidazo[1,2-b]pyridazin-3-yl)quinoline; 4-(6-Methyl-7-(1-(piperidin-4-ylmethyl)-1H-pyrazol-4-yl)imidazo[1,2-b]pyridazin-3-yl)-7-(pyridin-4-yl)quinolone; (trans)-4-(4-(6-Methyl-3-(7-(pyridin-4-yl)quinolin-4-yl)imidazo[1,2-b]pyridazin-7-yl)-1H-pyrazol-1-yl)cyclohexan-1-ol; 2-(4-(4-(6-Methyl-3-(7-(pyridin-4-yl)quinolin-4-yl)imidazo[1,2-b]pyridazin-7-yl)phenyl)piperazin-1-yl)ethan-1-ol; 4-Methyl-1-(4-(6-methyl-3-(7-(pyridin-4-yl)quinolin-4-yl)imidazo[1,2-b]pyridazin-7-yl)phenyl)piperazin-2-one; 4-(6-Methyl-7-(3-(4-methylpiperazin-1-yl)phenyl)imidazo[1,2-b]pyridazin-3-yl)-7-(pyridin-4-yl)quinolone; (4-(6-Methyl-3-(7-(pyridin-4-yl)quinolin-4-yl)imidazo[1,2-b]pyridazin-7-yl)phenyl)(4-methylpiperazin-1-yl)methanone: (R)-4-(7-(3-fluoro-4-((2-methylpyrrolidin-1-yl)methyl)phenyl)-6-methylimidazo[1,2-b]pyridazin-3-yl)-7-(pyridin-4-yl)quinoline; N-methyl-4-(6-methyl-7-(4-(piperazin-1-yl)phenyl)imidazo[1,2-b]pyridazin-3-yl)quinoline-7-carboxamide; (4-(6-methyl-7-(4-(piperazin-1-yl)phenyl)imidazo[1,2-b]pyridazin-3-yl)quinolin-7-yl)(4-methylpiperazin-1-yl)methanone; 7-(1-methyl-1H-pyrazol-3-yl)-4-(6-methyl-7-(4-(piperazin-1-yl)phenyl)imidazo[1,2-b]pyridazin-3-yl)quinoline; (4-(4-(6-methyl-7-(4-(piperazin-1-yl)phenyl)imidazo[1,2-b]pyridazin-3-yl)quinolin-7-yl)pyridin-2-yl)methanol; 4-(4-(6-methyl-7-(4-(piperazin-1-yl)phenyl)imidazo[1,2-b]pyridazin-3-yl)quinolin-7-yl)morpholine; 2-(4-(6-methyl-7-(4-(piperazin-1-yl)phenyl)imidazo[1,2-b]pyridazin-3-yl)quinolin-7-yl)-6-oxa-2-azaspiro[3.4]octane; 4-(6-methyl-7-(4-(piperazin-1-yl)phenyl)imidazo[1,2-b]pyridazin-3-yl)-N-(pyridin-3-yl)quinolin-7-amine; 1-(4-(4-(6-methyl-7-(4-(piperazin-1-yl)phenyl)imidazo[1,2-b]pyridazin-3-yl)quinolin-7-yl)piperazin-1-yl)ethan-1-one; 4-((4-(6-methyl-7-(4-(piperazin-1-yl)phenyl)imidazo[1,2-b]pyridazin-3-yl)quinolin-7-yl)methyl)morpholine; (3,5-difluorophenyl)(4-(6-methyl-7-(4-(piperazin-1-yl)phenyl)imidazo[1,2-b]pyridazin-3-yl)quinolin-7-yl)methanol; (4-(6-methyl-7-(4-(piperazin-1-yl)phenyl)imidazo[1,2-b]pyridazin-3-yl)quinolin-7-yl)methanol; 4-(6-methyl-7-(4-(piperazin-1-yl)phenyl)imidazo[1,2-b]pyridazin-3-yl)-1,8-naphthyridine; 5-(6-methyl-7-(4-(piperazin-1-yl)phenyl)imidazo[1,2-b]pyridazin-3-yl)quinoline-8-carbonitrile; 8-(6-methyl-7-(4-(piperazin-1-yl)phenyl)imidazo[1,2-b]pyridazin-3-yl)-2,3-dihydro-[1,4]dioxino[2,3-b]pyridine; 4-(6-methyl-7-(4-(piperazin-1-yl)phenyl)imidazo[1,2-b]pyridazin-3-yl)quinoline-7-carbonitrile; N-(2-hydroxyethyl)-4-(6-methyl-7-(4-(piperazin-1-yl)phenyl)imidazo[1,2-b]pyridazin-3-yl)quinoline-8-carboxamide; (4-(6-methyl-7-(1-(tetrahydro-2H-pyran-4-yl)-1H-pyrazol-4-yl)imidazo[1,2-b]pyridazin-3-yl)-2,3-dihydro-1H-pyrrolo[2,3-b]pyridin-1-yl)(pyridin-4-yl)methanone: 1-((1S,4S)-5-(4-(7-(1-((1R,4R)-4-hydroxycyclohexyl)-1H-pyrazol-4-yl)-6-methylimidazo[1,2-b]pyridazin-3-yl)quinolin-7-yl)-2,5-diazabicyclo[2.2.1]heptan-2-yl)ethan-1-one; 2-(4-(7-(1-((1R,4R)-4-hydroxycyclohexyl)-1H-pyrazol-4-yl)-6-methylimidazo[1,2-b]pyridazin-3-yl)quinolin-7-yl) hexahydropyrrolo[1,2-a]pyrazin-6 (2H)-one; 4-(6-methyl-7-(1-(piperidin-4-yl)-1H-pyrazol-4-yl)imidazo[1,2-a]pyridin-3-yl)quinoline; 2-fluoro-4-(6-methyl-7-(1-(piperidin-4-yl)-1H-pyrazol-4-yl)imidazo[1,2-a]pyridin-3-yl)benzamide; (1R,4R)-4-(4-(6-Methyl-3-(7-(pyridin-2-yl)-1,8-naphthyridin-4-yl)imidazo[1,2-b]pyridazin-7-yl)-1H-pyrazol-1-yl)cyclohexan-1-ol; 5-(7-(4-(Azetidin-1-ylmethyl)phenyl)-6-methylimidazo[1,2-b]pyridazin-3-yl)-2-(pyridin-2-yl)-1,8-naphthyridine; 5-(7-(1-Ethyl-1H-pyrazol-4-yl)-6-methylimidazo[1,2-b]pyridazin-3-yl)-2-(pyridin-2-yl)-1,8-naphthyridine; 5-(7-(1-Isopropyl-1H-pyrazol-4-yl)-6-methylimidazo[1,2-b]pyridazin-3-yl)-2-(pyrimidin-2-yl)-1,8-naphthyridine; (1R,4R)-4-(4-(6-Methyl-3-(7-(pyrazin-2-yl)-1,8-naphthyridin-4-yl)imidazo[1,2-b]pyridazin-7-yl)-1H-pyrazol-1-yl)cyclohexan-1-ol; 5-(7-(4-(Azetidin-1-ylmethyl)phenyl)-6-methylimidazo[1,2-b]pyridazin-3-yl)-2-(pyrazin-2-yl)-1,8-naphthyridine; 5-(7-(1-Isopropyl-1H-pyrazol-4-yl)-6-methylimidazo[1,2-b]pyridazin-3-yl)-2-(pyrazin-2-yl)-1,8-naphthyridine; 5-(6-Methyl-7-(1-(tetrahydro-2H-pyran-4-yl)-1H-pyrazol-4-yl)imidazo[1,2-b]pyridazin-3-yl)-2-(pyrazin-2-yl)-1,8-naphthyridine; 5-(7-(1-Isopropyl-1H-pyrazol-4-yl)-6-methylimidazo[1,2-b]pyridazin-3-yl)-2-(1-methyl-1H-pyrazol-3-yl)-1,8-naphthyridine; 5-(7-(4-(Azetidin-1-ylmethyl)phenyl)-6-methylimidazo[1,2-b]pyridazin-3-yl)-2-(1-methyl-1H-pyrazol-3-yl)-1,8-naphthyridine; N, N-dimethyl-4-(6-methyl-3-(7-(1-methyl-1H-pyrazol-3-yl)-1,8-naphthyridin-4-yl)imidazo[1,2-b]pyridazin-7-yl)benzamide; 5-(7-(4-(Azetidin-1-ylmethyl)phenyl)-6-methylimidazo[1,2-b]pyridazin-3-yl)-2-(5-methyl-1H-pyrazol-3-yl)-1,8-naphthyridine; 4-(4-(6-methyl-3-(7-(5-methyl-1H-pyrazol-3-yl)-1,8-naphthyridin-4-yl)imidazo[1,2-b]pyridazin-7-yl)benzyl)morpholine; 5-(7-(1-Isopropyl-1H-pyrazol-4-yl)-6-methylimidazo[1,2-b]pyridazin-3-yl)-2-(5-methyl-1H-pyrazol-3-yl)-1,8-naphthyridine; 5-(7-(1-Isopropyl-1H-pyrazol-4-yl)-6-methylimidazo[1,2-b]pyridazin-3-yl)-2-(1-methyl-1H-pyrazol-4-yl)-1,8-naphthyridine; 2-(1-Ethyl-1H-imidazol-4-yl)-5-(6-methyl-7-(4-(pyrrolidin-1-ylmethyl)phenyl)imidazo[1,2-b]pyridazin-3-yl)-1,8-naphthyridine; 4-(4-(3-(7-(1-Ethyl-1H-imidazol-4-yl)-1,8-naphthyridin-4-yl)-6-methylimidazo[1,2-b]pyridazin-7-yl)benzyl)morpholine; 1-(4-(3-(7-(1-Ethyl-1H-imidazol-4-yl)-1,8-naphthyridin-4-yl)-6-methylimidazo[1,2-b]pyridazin-7-yl)phenyl)-N, N-dimethylmethanamine; 2-(1-Ethyl-1H-imidazol-4-yl)-5-(7-(1-isopropyl-1H-pyrazol-4-yl)-6-methylimidazo[1,2-b]pyridazin-3-yl)-1,8-naphthyridine; 5-(7-(4-(Azetidin-1-ylmethyl)phenyl)-6-methylimidazo[1,2-b]pyridazin-3-yl)-2-(1-ethyl-1H-imidazol-4-yl)-1,8-naphthyridine; 5-(7-(4-((3,3-Difluoroazetidin-1-yl) yl)methyl)phenyl)-6-methylimidazo[1,2-b]pyridazin-3-yl)-2-(1-ethyl-1H-imidazol-4-yl)-1,8-naphthyridine; 5-(7-(4-((3,3-Difluoroazetidin-1-yl)methyl)phenyl)-6-methylimidazo[1,2-b]pyridazin-3-yl)-2-(1-ethyl-1H-imidazol-4-yl)-1,8-naphthyridine; (S)-4-(4-(3-(7-(1-ethyl-1H-imidazol-4-yl)-1,8-naphthyridin-4-yl)-6-methylimidazo[1,2-b]pyridazin-7-yl)benzyl)-2-methylmorpholine; (S)-(4-(4-(3-(7-(1-ethyl-1H-imidazol-4-yl)-1,8-naphthyridin-4-yl)-6-methylimidazo[1,2-b]pyridazin-7-yl)benzyl)morpholin-2-yl)methanol; 4-(6-methyl-7-(1-(piperidin-4-yl)-1H-pyrazol-4-yl)imidazo[1,2-a]pyridin-3-yl)quinolone; (R)-4-(7-(3-fluoro-4-((2-methylpyrrolidin-1-yl)methyl)phenyl)-6-methylimidazo[1,2-b]pyridazin-3-yl)-7-(pyridin-4-yl)quinolone; 1-((1S,4S)-5-(4-(7-(1-((1R,4R)-4-hydroxycyclohexyl)-1H-pyrazol-4-yl)-6-methylimidazo[1,2-b]pyridazin-3-yl)quinolin-7-yl)-2,5-diazabicyclo[2.2.1]heptan-2-yl)ethan-1-one; 7-(1-methyl-1H-pyrazol-3-yl)-4-(6-methyl-7-(4-(piperazin-1-yl)phenyl)imidazo[1,2-b]pyridazin-3-yl)quinolone; 4-(6-Methyl-7-(4-(piperazin-1-yl)phenyl)imidazo[1,2-b]pyridazin-3-yl)quinolone; 4-(6-(Methoxymethyl)-7-(4-(piperazin-1-yl)phenyl)imidazo[1,2-b]pyridazin-3-yl)quinolone; 2-(4-Methyl-2H-1,2,3-triazol-2-yl)-5-(6-methyl-7-(4-(pyrrolidin-1-ylmethyl)phenyl)imidazo[1,2-b]pyridazin-3-yl)-1,8-naphthyridine; 5-(7-(1-Isopropyl-1H-pyrazol-4-yl)-6-methylimidazo[1,2-b]pyridazin-3-yl)-2-(4-methyl-2H-1,2,3-triazol-2-yl)-1,8-naphthyridine; 2-(4-Methyl-2H-1,2,3-triazol-2-yl)-5-(6-methyl-7-(1-(tetrahydro-2H-pyran-4-yl)-1H-pyrazol-4-yl)imidazo[1,2-b]pyridazin-3-yl)-1,8-naphthyridine; N, N-dimethyl-1-(4-(6-methyl-3-(7-(4-methyl-2H-1,2,3-triazol-2-yl)-1,8-naphthyridin-4-yl)imidazo[1,2-b]pyridazin-7-yl)phenyl)methanamine; 4-(4-(6-Methyl-3-(7-(4-methyl-2H-1,2,3-triazol-2-yl)-1,8-naphthyridin-4-yl)imidazo[1,2-b]pyridazin-7-yl)benzyl)morpholine; N,N-dimethyl-1-(4-(6-methyl-3-(7-(4-methyl-2H-1,2,3-triazol-2-yl)-1,8-naphthyridin-4-yl)imidazo[1,2-b]pyridazin-7-yl)phenyl)methanamine; (S)-3-methyl-1-(4-(6-methyl-3-(7-(4-methyl-2H-1,2,3-triazol-2-yl)-1,8-naphthyridin-4-yl)imidazo[1,2-b]pyridazin-7-yl)benzyl)pyrrolidin-3-ol; 1-(4-(6-methyl-3-(7-(4-methyl-2H-1,2,3-triazol-2-yl)-1,8-naphthyridin-4-yl)imidazo[1,2-b]pyridazin-7-yl)benzyl)piperidin-4-ol; 4-methyl-1-(4-(6-methyl-3-(7-(4-methyl-2H-1,2,3-triazol-2-yl)-1,8-naphthyridin-4-yl)imidazo[1,2-b]pyridazin-7-yl)benzyl)piperidin-4-ol; 3-methyl-1-(4-(6-methyl-3-(7-(4-methyl-2H-1,2,3-triazol-2-yl)-1,8-naphthyridin-4-yl)imidazo[1,2-b]pyridazin-7-yl)benzyl) azetidin-3-ol; 2-(4-methyl-2H-1,2,3-triazol-2-yl)-5-(6-methyl-7-(4-((4-methylpiperazin-1-yl)methyl)phenyl)imidazo[1,2-b]pyridazin-3-yl)-1,8-naphthyridine; 1-(4-(3-(7-(1-Ethyl-1H-imidazol-4-yl)-1,8-naphthyridin-4-yl)-6-methylimidazo[1,2-b]pyridazin-7-yl)benzyl)piperidin-4-ol; 1-(4-(3-(7-(1-Ethyl-1H-imidazol-4-yl)-1,8-naphthyridin-4-yl)-6-methylimidazo[1,2-b]pyridazin-7-yl)benzyl)-4-methylpiperidin-4-ol; and pharmaceutically acceptable salts thereof.

In some embodiments of any of the foregoing aspects, the compound of Formula II-11 is a crystalline compound having the structure of Formula I-11 or a salt thereof. In some embodiments, the crystalline compound has the structure of Formula II-11

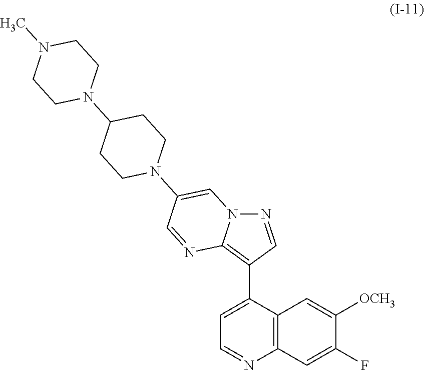 [SMILES: COc1cc2c(-c3cnn4cc(N5CCC(N6CCN(C)CC6)CC5)cnc34)ccnc2cc1F]

mono-succinate salt. In some embodiments, the compound is anhydrous. In some embodiments, the compound has 20 values of about 7.05±0.2, 15.16 ±0.2, 21.05±0.2, 21.26±0.2, and 24.47±0.2. In some embodiments, the compound has 20 values of about 3.58±0.2, 7.05±0.2, 13.8±0.2, 14.16±0.2, 15.16±0.2, 16.18±0.2, 16.80±0.2, 17.15±0.2, 17.69±0.2, 18.29±0.2, 18.84±0.2, 20.29±0.2, 21.05±0.2, 21.26±0.2, 22.68±0.2, 23.84±0.2, 24.47±0.2, 24.84±0.2, and 28.47±0.2. In some embodiments, the compound has 20 values of about 3.58±0.2, 7.05±0.2, 10.59±0.2, 10.75±0.2, 13.80±0.2, 14.16±0.2, 15.16±0.2, 15.68±0.2, 16.18±0.2, 16.80±0.2, 17.15±0.2, 17.69±0.2, 17.97±0.2, 18.29±0.2, 18.59±0.2, 18.84±0.2, 19.27±0.2, 20.29±0.2, 21.05±0.2, 21.26±0.2, 21.56±0.2, 21.78±0.2, 22.68±0.2, 23.84±0.2, 24.47±0.2, 24.84±0.2, 25.15±0.2, 26.10±0.2, 27.12±0.2, 27.78±0.2, 28.47±0.2, and 29.06±0.2.

In some embodiments, the compound has 20 values of about 9.79±0.2, 13.05±0.2, 22.91±0.2, 23.60±0.2, and 26.25±0.2. In some embodiments, the compound has 20 values of about 3.25±0.2, 9.79±0.2, 13.05±0.2, 16.75±0.2, 19.50±0.2, 22.91±0.2, 23.60±0.2, and 26.25±0.2. In some embodiments, the compound has 20 values of about 3.25±0.2, 9.79±0.2, 13.05±0.2, 13.61±0.2, 14.39±0.2, 16.75±0.2, 18.50±0.2, 19.50±0.2, 22.91±0.2, 23.60±0.2, and 26.25±0.2.

In some embodiments of any of the foregoing aspects, the compound of Formula II-11 is a crystalline compound having the structure of Formula II-11

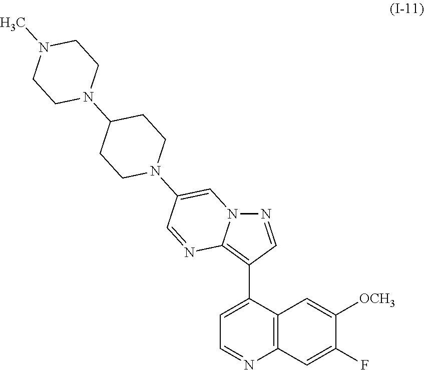 [SMILES: COc1cc2c(-c3cnn4cc(N5CCC(N6CCN(C)CC6)CC5)cnc34)ccnc2cc1F]

free base. In some embodiments, the compound has 20 values of about 6.00±0.2, 12.00±0.2, 16.14±0.2, 17.72±0.2, 18.00±0.2, 18.64±0.2, and 23.50±0.2.

In some embodiments of any of the foregoing aspects, the compound of any one of Formulas I-III is administered in a pharmaceutical composition further including one or more pharmaceutically acceptable excipients.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. The definitions and terminology used herein are provided to aid in describing particular embodiments, and are not intended to limit the claimed invention.

Unless stated otherwise, the terms “a,” “an,” and “the,” and similar references used in the context of describing one or more embodiments of the application (especially in the context of claims), can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia and is used herein to indicate a non-limiting example. Thus, the abbreviation “e.g.” is synonymous with the term “for example.” No language in the specification should be construed as indicating any non-claimed element essential to the practice of the application.

Unless defined otherwise, technical and scientific terms used herein have the same meaning as commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, NY 2006); Smith, March's Advanced Organic Chemistry Reactions, Mechanisms and Structure 7th ed., J. Wiley & Sons (New York, NY 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, NY 2012), provide one skilled in the art with a general guide to many of the terms used in the present application.

As used herein, the term “about” refers to a value that is within 10% above or below the value being described.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “aliphatic,” as used herein, includes straight, chained, branched or cyclic hydrocarbons which are completely saturated or contain one or more units of unsaturation. Aliphatic groups may be substituted or unsubstituted.

The term “alkoxy” refers to an oxygen having an alkyl group attached thereto. Representative alkoxy groups include methoxy, ethoxy, propoxy, tert-butoxy and the like.

The term “alkenyl,” as used herein, refers to an aliphatic group containing at least one double bond and is intended to include both “unsubstituted alkenyls” and “substituted alkenyls,” the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. In preferred embodiments, a straight chain or branched chain alkenyl has 1-12 carbons in its backbone, preferably 1-8 carbons in its backbone, and more preferably 1-6 carbons in its backbone. Exemplary alkenyl groups include allyl, propenyl, butenyl, 2-methyl-2-butenyl, and the like.

The term “alkyl” refers to the radical of saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g., methyl, ethyl, n-propyl, i-propyl, n-butyl and n-pentyl. Moreover, the term “alkyl” (or “lower alkyl”) as used throughout the specification, examples, and claims is intended to include both “unsubstituted alkyls” and “substituted alkyls,” the latter of which refers to alkyl moieties having substituents replacing a hydrogen on one or more carbons of the hydrocarbon backbone. In certain embodiments, a straight chain or branched chain alkyl has 30 or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In other embodiments, the chain has six or fewer carbon (C₁-C₆) atoms in its backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino,” as used herein, refers to an amino group substituted with at least one alkyl group.

The term “alkylthio,” as used herein, refers to a thiol group substituted with an alkyl group and may be represented by the general formula alkylS-.

The term “alkynyl,” as used herein, refers to an aliphatic group containing at least one triple bond and is intended to include both “unsubstituted alkynyls” and “substituted alkynyls,” the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated. In preferred embodiments, an alkynyl has 1-12 carbons in its backbone, preferably 1-8 carbons in its backbone, and more preferably 1-6 carbons in its backbone. Alkynyl groups include propynyl, butynyl, 3-methylpent-1-ynyl, and the like.

The term “amide,” as used herein, refers to a group

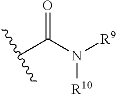

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

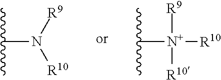

wherein R⁹, R¹⁰, and R¹⁰ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl,” as used herein, refers to an alkyl group substituted with an amino group.

The term “aralkyl,” as used herein, refers to an alkyl group substituted with one or more aryl groups.

The term “aryl,” as used herein, include substituted or unsubstituted single-ring aromatic groups in which each atom of the ring is carbon. Preferably the ring is a 5- to 7-membered ring, more preferably a 6-membered ring. Aryl groups include phenyl, phenol, aniline, and the like.

The term “carbamate” is art-recognized and refers to a group

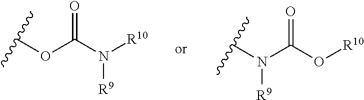

wherein R⁹ and R¹⁰ independently represent hydrogen or a hydrocarbyl group, such as an alkyl group.

The terms “carbocycle,” “carbocyclyl,” and “carbocyclic,” as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl,” as used herein, refers to an alkyl group substituted with a carbocycle group.

The term “carbonate” is art-recognized and refers to a group —OCO₂—R⁹, wherein R⁹ represents a hydrocarbyl group, such as an alkyl group.

The term “carboxy,” as used herein, refers to a group represented by the formula-CO₂H. The term “cycloalkyl,” as used herein, refers to the radical of a saturated aliphatic ring. In preferred embodiments, cycloalkyls have from 3-10 carbon atoms in their ring structure, and more preferably from 5-7 carbon atoms in the ring structure. Suitable cycloalkyls include cycloheptyl, cyclohexyl, cyclopentyl, cyclobutyl and cyclopropyl.

The term “ester,” as used herein, refers to a group —C(O) OR⁹ wherein R⁹ represents a hydrocarbyl group, such as an alkyl group or an aralkyl group.

The term “ether,” as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen,” as used herein, means halogen and includes chloro, fluoro, bromo, and iodo.

The term “heteroalkyl,” as used herein, refers to a saturated or unsaturated chain of carbon atoms including at least one heteroatom (e.g., O, S, or NR⁵⁰, such as where R⁵⁰ is H or lower alkyl), wherein no two heteroatoms are adjacent.

The terms “hetaralkyl” and “heteroaralkyl,” as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom (e.g., O, N, or S), preferably one to four or one to 3 heteroatoms, more preferably one or two heteroatoms. When two or more heteroatoms are present in a heteroaryl ring, they may be the same or different. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Preferred polycyclic ring systems have two cyclic rings in which both of the rings are aromatic. Heteroaryl groups include, for example, pyrrole, furan, thiophene, imidazole, oxazole, thiazole, pyrazole, pyridine, pyrazine, pyridazine, quinoline, and pyrimidine, and the like.

The term “heteroatom,” as used herein, means an atom of any element other than carbon or hydrogen. Preferred heteroatoms are nitrogen, oxygen, and sulfur.

The terms “heterocyclyl,” “heterocycle,” and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. Heterocyclyl groups include, for example, piperidine, piperazine, pyrrolidine, morpholine, lactones, lactams, and the like.

The term “heterocyclylalkyl,” as used herein, refers to an alkyl group substituted with a heterocycle group.

The term “hydrocarbyl,” as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl,” for example, refers to an alkyl group that contains ten or fewer carbon atoms, preferably six or fewer. Examples of straight chain or branched chain lower alkyl include methyl, ethyl, isopropyl, propyl, butyl, tertiary-butyl, and the like. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitation aralkyl (in which case, for example, the atoms within the aryl group are not counted when counting the carbon atoms in the alkyl substituent).

As used herein, the term “pharmaceutically acceptable salt” means any pharmaceutically acceptable salt of a compound described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The terms “polycyclyl,” “polycycle,” and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Preferred polycycles have 2-3 rings. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt or ester thereof.

The term “sulfonamide” is art-recognized and refers to the group represented by the general formulae

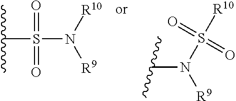

wherein R⁹ and R¹⁰ independently represents hydrogen or hydrocarbyl, such as alkyl.

The term “sulfoxide” is art-recognized and refers to the group —S(O)—R⁹, wherein R⁹ represents a hydrocarbyl, such as alkyl, aryl, or heteroaryl. The term “sulfonate” is art-recognized and refers to the group —SO₃H, or a pharmaceutically acceptable salt or ester thereof.

The term “sulfone” is art-recognized and refers to the group —S(O)₂—R⁹, wherein R⁹ represents a hydrocarbyl, such as alkyl, aryl, or heteroaryl.

The term “thioester,” as used herein, refers to a group —C(O)SR⁹ or —SC(O)R⁹ wherein R⁵ represents a hydrocarbyl, such as alkyl.

The term “thioether,” as used herein, is equivalent to an ether, wherein the oxygen is replaced with a sulfur.

The term “urea” is art-recognized and may be represented by the general formula

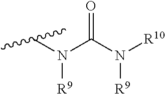

wherein R⁹ and R¹⁰ independently represent hydrogen or a hydrocarbyl, such as alkyl. At various places in the present specification substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, propyl, isopropyl, n-butyl, sec-butyl, isobutyl, etc.

As used herein, “administration” refers to providing or giving a subject a therapeutic agent (e.g., an ALK2 inhibitor described herein), by any effective route. Exemplary routes of administration are described herein below.

As used herein, the term “anemia” refers to any abnormality in hemoglobin or red blood cells that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels. For example, a subject having a hemoglobin level <11 g/dL or receiving red blood cell (RBC) transfusions can be identified as having anemia.

As used herein, the terms “erythropoiesis stimulating agent” and “ESA” refer to a class of drugs that act on the proliferation stage of red blood cell development by expanding the pool of early-stage progenitor cells. Examples of erythropoiesis-stimulating agents are epoetin alfa and darbepoetin alfa.

As used herein, the terms “functional iron deficiency” and “FID” refer to decreased iron availability in the bone marrow for synthesis of hemoglobin despite adequate body iron stores. FID can lead to inhibition of erythropoiesis, for example, in cancer-related anemia.

As used herein, the terms “subject” and “patient” refer to a mammal, e.g., a human. Mammals include, but are not limited to, humans and domestic and farm animals, such as monkeys (e.g., a cynomolgus monkey), mice, dogs, cats, horses, and cows, etc. A subject to be treated according to the methods described herein may be one who has been diagnosed with anemia associated with very low, low, or intermediate risk MDS, anemia associated with primary MF, post-polycythemia vera MF, or post-essential thrombocythemia MF, or anemia associated with an MDS/MPN overlap syndrome. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

The term “small molecule” refers to an organic molecule having a molecular weight less than about 2500 amu, less than about 2000 amu, less than about 1500 amu, less than about 1000 amu, or less than about 750 amu. In some embodiments a small molecule contains one or more heteroatoms.

As used herein, the term “small molecule ALK2 inhibitor” refers to a small molecule that inhibits the activity of ALK2 (e.g., human ALK2) with an IC50 of 10 μM or lower (e.g., 1 μM, 500 nm, 100 nM, 50 nM, or lower, such as between 1 μM and 1 nM, 1 μM and 10 nM, 1 μM and 50 nM, 1 μM and 100 nM, 500 nM and 1 nM, 250 nM and 1 nM, 100 nM and 1 nM, and 50 nM and 1 nM). The small molecule ALK2 inhibitor may be selective for ALK2 (e.g., inhibits the activity of ALK2 with an IC50 that is lower by a factor of 5 or more (e.g., 5, 10, 25, 50, 100, 200, 300, 400, 500, 600, 800, 1000 or more) than its IC50 for inhibiting the activity of ALK1, ALK3, ALK4, ALK5, or ALK6), or the ALK2 small molecule inhibitor may exhibit similar inhibitory effects on multiple BMP receptors (e.g., ALK2 and ALK1, ALK3, ALK4, ALK5, or ALK6).

The phrase “activity of ALK2” means ALK2 enzymatic activity (e.g., such as kinase activity; the ability of ALK2 to phosphorylate BMP-responsive SMAD proteins) and/or ALK2-mediated signaling (e.g., such as the ability of ALK2 to mediate downstream signal transduction and transcriptional activity following activation of ALK2 by binding of BMP ligands). In some embodiments, “activity of ALK2” means ALK2-mediated BMP signaling. In some embodiments, “activity of ALK2” means ALK2-mediated BMP-responsive gene transcription (e.g., transcriptional activity mediated by BMP/ALK2 signal transduction).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the terms “increasing” and “decreasing” refer to modulating resulting in, respectively, greater or lesser amounts, of function, expression, level, or activity of a metric relative to a reference. For example, subsequent to administration of an ALK2 inhibitor of the invention in a method described herein, the amount of a marker of a metric (e.g., reticulocyte hemoglobin) as described herein may be increased in a subject relative to the amount of the marker prior to administration or relative to an untreated subject, or the amount of a marker of a metric (e.g., serum hepcidin levels) as described herein may be decreased in a subject relative to the amount of the marker prior to administration or relative to an untreated subject. Generally, the metric is measured subsequent to administration at a time that the administration has had the recited effect, e.g., at least one week, one month, 3 months, or 6 months, after a treatment regimen has begun. The increase or decrease may be an increase or decrease of at least 10% as compared to a reference level, for example an increase or decrease of at least about 20%, or at least about 30%, or at least about 40%, or at least about 50%, or at least about 60%, or at least about 70%, or at least about 80%, or at least about 90% or up to and including a 100% increase or decrease or any increase or decrease between 10-100% as compared to a reference level, or at least about a 2-fold, or at least about a 3-fold, or at least about a 4-fold, or at least about a 5-fold, or at least about a 10-fold, or at least about a 20-fold, or at least about a 50-fold, or at least about a 100-fold, or at least about a 1000-fold increase or decrease or more as compared to a reference level.

The term “pharmaceutically acceptable” can refer to compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier or excipient” refers to an excipient or diluent in a pharmaceutical composition. The pharmaceutically acceptable carrier must be compatible with the other ingredients of the formulation and suitable for contact with the tissues of a subject without excessive toxicity, irritation, allergic response, and other problem complications commensurate with a reasonable benefit/risk ratio. In the present invention, the pharmaceutically acceptable carrier or excipient must provide adequate pharmaceutical stability to the ALK2 inhibitor. The nature of the carrier or excipient differs with the mode of administration. For example, for intravenous administration, an aqueous solution carrier is generally used; for oral administration, a solid carrier is preferred. Examples include any of the standard pharmaceutical carriers such as a phosphate buffered saline solution, water, emulsions such as an oil/water emulsion, and various types of wetting agents.

As used herein, the term “diagnosis” refers to the process of identifying a disease by its signs, symptoms, and/or results of various tests. The conclusion reached through that process is also called “a diagnosis.” Forms of testing commonly performed include physical examination, blood tests, medical imaging, genetic analysis, urinalysis, and biopsy.

As used herein, the term “inhibitor” refers to any chemical compound, nucleic acid molecule, small molecule, peptide, or polypeptide (such as an antibody) that can reduce activity of a gene product or interfere with expression of a gene. In some examples, an inhibitor can reduce or inhibit the activity of a protein that is encoded by a gene either directly or indirectly. Direct inhibition can be accomplished, for example, by binding to a protein and thereby preventing the protein from binding an intended target, such as a receptor. Indirect inhibition can be accomplished, for example, by binding to a protein's intended target, such as a receptor or binding partner, thereby blocking or reducing activity of the protein. In some examples, an inhibitor of the disclosure can inhibit a gene by reducing or inhibiting expression of the gene, inter alia by interfering with gene expression (transcription, processing, translation, post-translational modification), for example, by interfering with the gene's mRNA and blocking translation of the gene product or by post-translational modification of a gene product, or by causing changes in intracellular localization. In various embodiments of the present invention, an inhibitor is one or more compounds of Formula I, Formula II, and Formula III, Compounds 1-7, BCX9250, and INCB00928 or a pharmaceutically acceptable salt thereof.

As used herein, the terms “effective amount,” “therapeutically effective amount,” and “sufficient amount” of a composition or ALK2 inhibitor described herein refer to a quantity sufficient to, when administered to the subject effect beneficial or desired results, including clinical results, and, as such, an “effective amount” or synonym thereto depends upon the context in which it is being applied. For example, in the context of treating patient having anemia associated with very low, low, or intermediate risk MDS, anemia associated with primary MF, post-polycythemia vera MF, or post-essential thrombocythemia MF, or anemia associated with an MDS/MPN overlap syndrome, it is an amount of the composition or ALK2 inhibitor sufficient to achieve a treatment response as compared to the response obtained without administration of the composition or ALK2 inhibitor. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art by routine methods known in the art. Dosage regimen may be adjusted to provide the optimum therapeutic response.

The term “substantially pure,” as used herein, refers to a crystalline polymorph that is greater than 90% pure, meaning that contains less than 10% of any other compound, including the corresponding amorphous compound or an alternative polymorph of the crystalline salt. Preferably, the crystalline polymorph is greater than 95% pure, or even greater than 98% pure.

### DETAILED DESCRIPTION OF THE INVENTION

The invention features methods of treating functional iron deficiency (FID) and/or anemia, such as FID anemia, associated with myelodysplastic syndromes (MDS), myelofibrosis (MF), and myelodysplastic syndromes/myeloproliferative neoplasm (MDS/MPN) overlap syndromes using small molecule ALK2 inhibitors. The ALK2 inhibitors described herein can treat FID and/or anemia by increasing serum iron, increasing transferrin saturation, increasing iron bioavailability (e.g., by mobilizing iron from storage tissue), increasing reticulocyte hemoglobin, promoting the formation of hemoglobin-containing (e.g., hemoglobin-rich) red blood cells, reestablishing iron homeostasis, and/or reducing serum hepcidin. Treatment with an ALK2 inhibitor described herein may also reduce transfusion burden or promote transfusion independence in subjects previously treated with red blood cell transfusions.

### ALK2 Inhibitors

In some embodiments, the ALK2 inhibitor for use in the methods and compositions described herein is a small molecule inhibitor of the BMP type I receptor ALK2, encoded by gene ACVR1.

In some embodiments, the ALK2 inhibitor is a compound of Formula I:

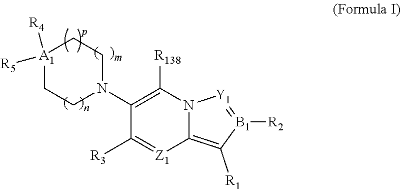

or a pharmaceutically acceptable salt thereof, wherein:
R₁ is hydrogen or an optionally substituted substituent selected from cycloalkyl, aryl, heteroaryl, and heterocyclyl;
R₂ is optionally absent, hydrogen, CN, NO₂, or an optionally substituted substituent selected from alkyl and amino;
R₃ is hydrogen, CN, NO₂, or an optionally substituted substituent selected from alkyl, alkoxy, heterocyclyloxy, heteroaryloxy, aryloxy, cycloalkyloxy, carbonyl, amino, amido, sulfonyl, sulfonamido, cycloalkyl, aryl, heterocyclyl, and heteroaryl;
R₄ is optionally absent, hydrogen, O—, halo, CN, NO₂, hydroxy, or an optionally substituted substituent selected from alkyl, alkenyl, alkynyl, carbonyl, cycloalkyl, aryl, alkoxy, aryloxy, cycloalkyloxy, amino, amido, alkoxycarbonyl, carboxy, sulfonyl, sulfonamido, thio, heterocyclyl, heterocyclyloxy, heteroaryl, and heteroaryloxy;
R₅ is optionally absent, hydrogen, halo, hydroxy, or optionally substituted alkyl;
R₁₃₈ is hydrogen, halo, hydroxy, or an optionally substituted substituent selected from alkyl, carbonyl, alkoxy, thio, amino, amido, heterocyclyl, aryl, and heteroaryl;
R₆ is independently one or more of hydrogen, halo, CN, NO₂, hydroxy, or an optionally substituted substituent selected from alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl and oxo; B₁, is C or N; Y₁ is N or CR¹³⁹, wherein R₁₃₉ is hydrogen, halo, hydroxy, or an optionally substituted substituent selected from alkyl, carbonyl, alkoxy, thio, amino, amido, heterocyclyl, aryl, and heteroaryl; Z₁ is N or CR₁₄₀, wherein R₁₄₀ is hydrogen, halo, hydroxy, or an optionally substituted substituent selected from alkyl, carbonyl, alkoxy, thio, amino, amido, heterocyclyl, aryl, or heteroaryl; A₁ is C, N, O, C(O), S, SO, or SO₂; m is 0, 1, 2, or 3; n is 0, 1, 2, or 3; and p is 0 or 1; wherein optionally any two or more of R₄, R₅, or R₆ may be joined together to form one or more rings.

Compounds of Formula I may be synthesized by methods known in the art, e.g., those described in US Patent Application Publication No. 2020/0179389, which is incorporated herein by reference.

In some embodiments, the compound of Formula I has a structure of Formula I-a:

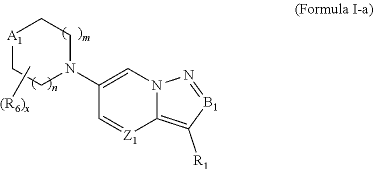

or a pharmaceutically acceptable salt thereof, wherein:
A₁ is NR^{4a} or CR_{4b}R₅;
B₁ is N or CR₂;
Z₁ is N or CR₃;
R₁ is selected from cycloalkyl, aryl, heteroaryl, and heterocyclyl;
R₂ is H, CN, NO₂, alkyl, or amino;
R₃ is selected from H, CN, NO₂, alkyl, alkoxy, heterocyclyloxy, heteroaryloxy, aryloxy, cycloalkyloxy, carbonyl, amino, amido, sulfonyl, sulfonamido, cycloalkyl, aryl, heterocyclyl, and heteroaryl;
R₄ₐ is selected from alkyl, alkenyl, alkynyl, carbonyl, O—, alkoxycarbonyl, cycloalkyl, aryl, heterocyclyl, and heteroaryl;
R_{4b} is selected from halo, CN, NO₂, hydroxy, alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl;
R₃ is selected from H, halo, hydroxy and alkyl, or
R_{4b} and R₃ together with A₁ form a ring selected from cycloalkyl and heterocyclyl;
each R₆ is independently selected from H, halo, CN, NO₂, hydroxy, alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl and oxo;
n is 0 or 1;
m is 0 or 1; and
x is 0, 1, 2, 3, or 4.

In some embodiments of the compound of Formula I-a,
A₁ is NR^{4a} or CR_{4b}R₅;
B₁ is N or CR₂;
Z₁ is N or CR₃;
R₁ is selected from aryl, heteroaryl, and heterocyclyl;
R₂ is H or amino;
R₃ is H or heterocyclyloxy;
R₄ₐ is selected from alkyl, O, aryl, heterocyclyl, and heteroaryl;
R_{4b} is selected from alkyl, alkoxy, amino, aryl, heterocyclyl, and heteroaryl;
R₅ is selected from H and alkyl, or
R_{4b} and R₃ together with A₁ form a ring selected from cycloalkyl and heterocyclyl;
each R₆ is independently selected from H, halo, alkyl and oxo;
n is 0 or 1;
m is 0 or 1; and
x is 0, 1, 2, 3, or 4.

In some embodiments of the compound of Formula I-a,
R₄ₐ is selected from alkyl, O—, heterocyclyl, and heteroaryl;
R_{4b} is selected from alkyl, alkoxy, amino, amido, heterocyclyl, and heteroaryl;
R₃ is selected from H and alkyl, or
R_{4b} and R₃ together with A₁ form a heterocyclyl; and
each R₆ is independently selected from H, halo, and alkyl; and x is 0 or 1.

In some embodiments of the compound of Formula I-a, R₁ is selected from H, aryl, 5-6 membered heteroaryl,

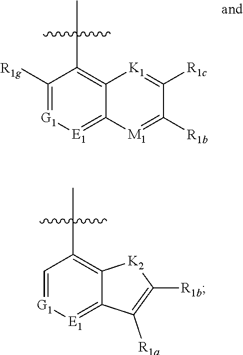

wherein:
each E₁ is independently selected from N and CR_{1d};
each G₁ is independently selected from N and CR₁ₑ;
K₁ is N or CH;
K₂ is NH or S;
M₁ is N or CR₁ₐ;
R₁ₐ is selected from H, halo, alkyl, haloalkyl, and amido;
R_{1b} is selected from H, halo, CN, alkyl, haloalkyl, hydroxy, alkoxy, and haloalkoxy;
R_{1c} is selected from H, halo, CN, alkyl, haloalkyl, hydroxy, alkoxy, haloalkoxy, amino and amido, or Rib and R_{1c} together with the carbon atoms to which they are attached form a heterocyclyl;
R_{1d} is selected from H, CN, alkyl, haloalkyl, hydroxy, amido and sulfonamido;
R₁ₑ is selected from H, alkyl and amino; and
R_{1g} is H or halo.

In some embodiments of the compound of Formula I-a,
R₄ₐ is selected from alkyl, O—, heterocyclyl, and heteroaryl;
R_{4b} is selected from alkyl, alkoxy, amino, amido, heterocyclyl, and heteroaryl;
R₃ is selected from H and alkyl, or
R_{4b} and R₃ together with A₁ form a heterocyclyl; and
each R₆ is independently selected from H, halo, and alkyl; and
x is 0 or 1.

In some embodiments, R₁ is selected from H, aryl, 5-6 membered heteroaryl,

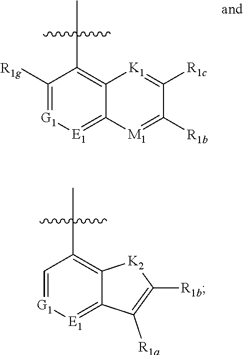

wherein:
each E₁ is independently selected from N and CR_{1d};
each G₁ is independently selected from N and CR₁ₑ;
K₁ is N or CH;
K₂ is NH or S;
M₁ is CR₁ₐ;
R₁ₐ is selected from H and amido;
R_{1b} is selected from H, halo, alkyl, and alkoxy;
R_{1c} is selected from H, alkyl, and alkoxy, or
R_{1b} and R_{1c} together with the carbon atoms to which they are attached form a heterocyclyl;
R_{1d} is selected from H, alkyl, hydroxy, amido and sulfonamido;
R₁ₑ is selected from H, alkyl and amino;
R_{1f} is H; and
R_{1g} is H.

In some embodiments, the compound of Formula I has a structure of Formula I-1:

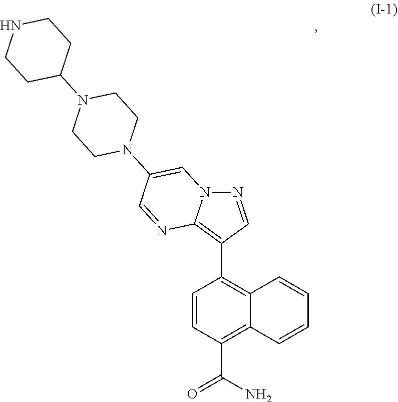 [SMILES: NC(=O)c1ccc(-c2cnn3cc(N4CCN(C5CCNCC5)CC4)cnc23)c2ccccc12]

or a pharmaceutically acceptable salt thereof.

In some embodiments, the compound of Formula I has a structure of Formula II-2:

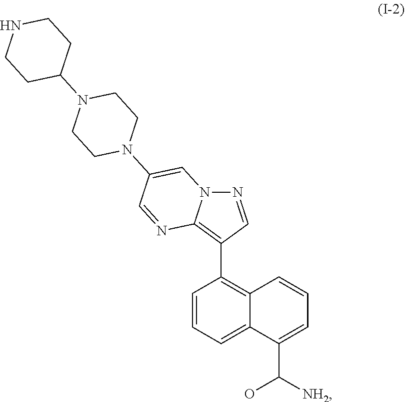 [SMILES: NC(O)c1cccc2c(-c3cnn4cc(N5CCN(C6CCNCC6)CC5)cnc34)cccc12]

or a pharmaceutically acceptable salt thereof.

In some embodiments, the compound of Formula I has a structure of Formula I-3:

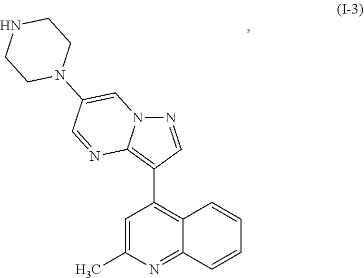 [SMILES: Cc1cc(-c2cnn3cc(N4CCNCC4)cnc23)c2ccccc2n1]

or a pharmaceutically acceptable salt thereof.

In some embodiments, the compound of Formula I has a structure of Formula II-4:

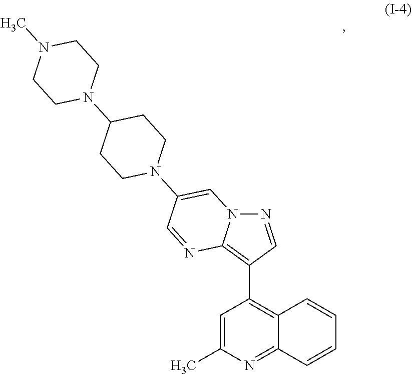 [SMILES: Cc1cc(-c2cnn3cc(N4CCC(N5CCN(C)CC5)CC4)cnc23)c2ccccc2n1]

or a pharmaceutically acceptable salt thereof.

In some embodiments, the compound of Formula I has a structure of Formula II-5:

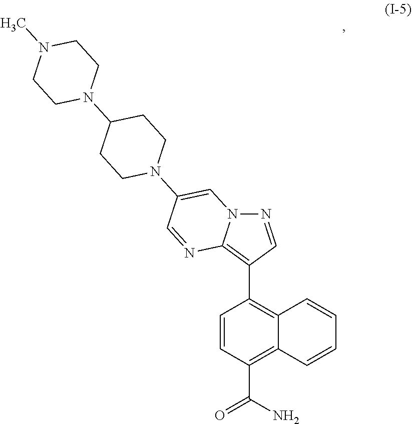 [SMILES: CN1CCN(C2CCN(c3cnc4c(-c5ccc(C(N)=O)c6ccccc56)cnn4c3)CC2)CC1]

or a pharmaceutically acceptable salt thereof.

In some embodiments, the compound of Formula I has a structure of Formula II-6:

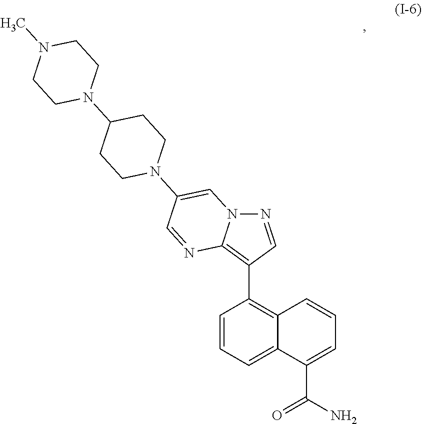 [SMILES: CN1CCN(C2CCN(c3cnc4c(-c5cccc6c(C(N)=O)cccc56)cnn4c3)CC2)CC1]

In some embodiments, the compound of Formula I has a structure of Formula II-7:

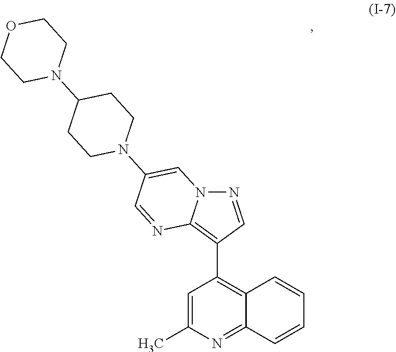 [SMILES: Cc1cc(-c2cnn3cc(N4CCC(N5CCOCC5)CC4)cnc23)c2ccccc2n1]

or a pharmaceutically acceptable sail thereof.

In some embodiments, the compound of Formula I has a structure of Formula II-8:

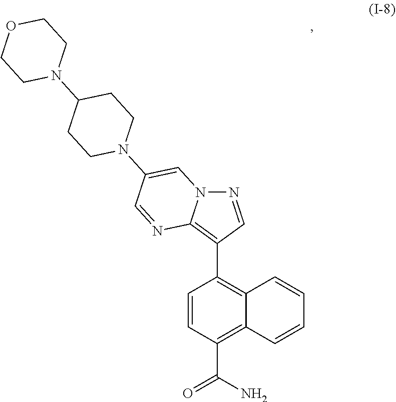 [SMILES: NC(=O)c1ccc(-c2cnn3cc(N4CCC(N5CCOCC5)CC4)cnc23)c2ccccc12]

or a pharmaceutically acceptable salt thereof.

In some embodiments, the compound of Formula I has a structure of Formula II-9:

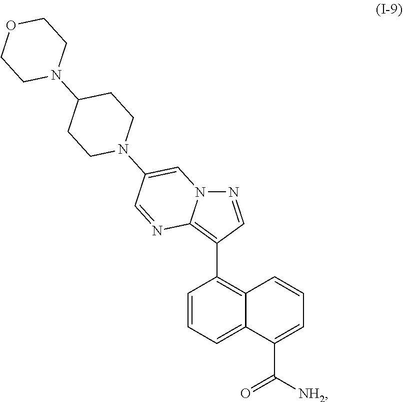 [SMILES: NC(=O)c1cccc2c(-c3cnn4cc(N5CCC(N6CCOCC6)CC5)cnc34)cccc12]

or a pharmaceutically acceptable salt thereof.

In some embodiments, the compound of Formula I has a structure of Formula II-10:

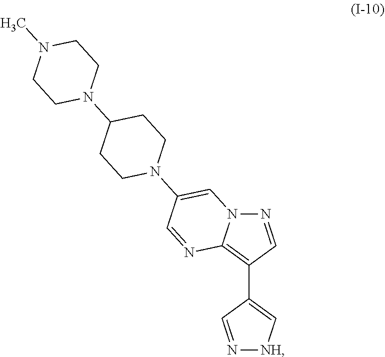 [SMILES: CN1CCN(C2CCN(c3cnc4c(-c5cn[nH]c5)cnn4c3)CC2)CC1]

or a pharmaceutically acceptable salt thereof.

In some embodiments, the compound of Formula I has a structure of Formula I-11:

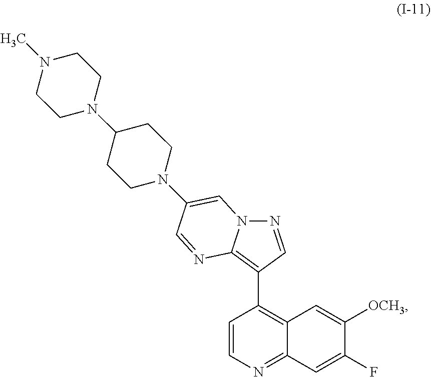 [SMILES: COc1cc2c(-c3cnn4cc(N5CCC(N6CCN(C)CC6)CC5)cnc34)ccnc2cc1F]

or a pharmaceutically acceptable salt thereof.

In some embodiments, the compound of Formula I has a structure of Formula II-12:

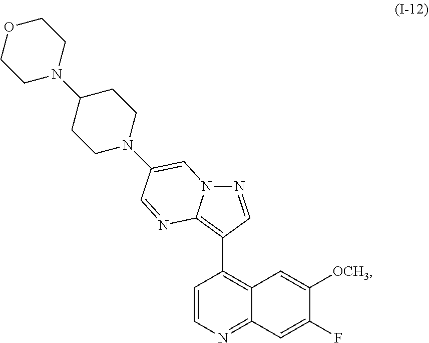 [SMILES: COc1cc2c(-c3cnn4cc(N5CCC(N6CCOCC6)CC5)cnc34)ccnc2cc1F]

In some embodiments, the compound of Formula I has a structure of Formula I-13:

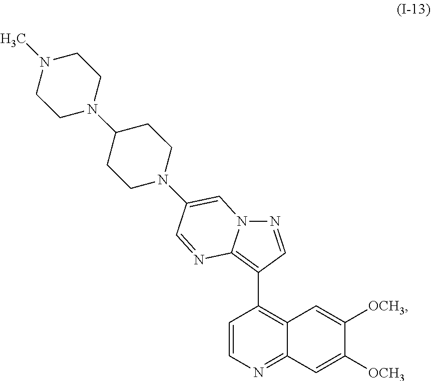 [SMILES: COc1cc2nccc(-c3cnn4cc(N5CCC(N6CCN(C)CC6)CC5)cnc34)c2cc1OC]

or a pharmaceutically acceptable salt thereof.

In some embodiments, the compound of Formula I has a structure of Formula II-14:

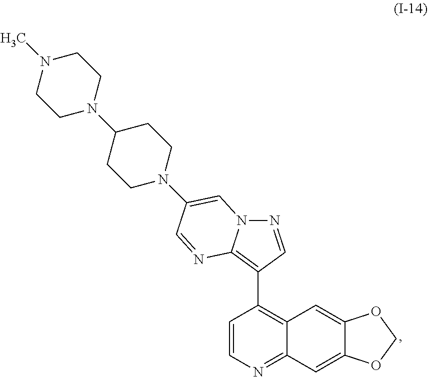 [SMILES: CN1CCN(C2CCN(c3cnc4c(-c5ccnc6cc7c(cc56)OCO7)cnn4c3)CC2)CC1]

or a pharmaceutically acceptable salt thereof.

In some embodiments, the compound of Formula I has a structure of Formula I-15:

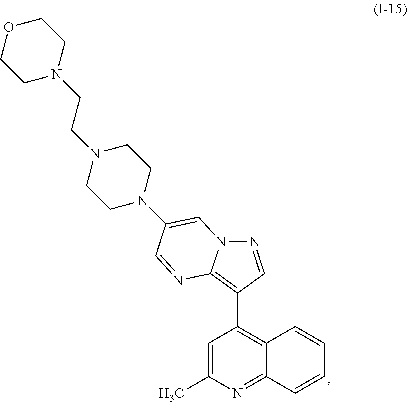 [SMILES: Cc1cc(-c2cnn3cc(N4CCN(CCN5CCOCC5)CC4)cnc23)c2ccccc2n1]

or a pharmaceutically acceptable salt thereof.

In some embodiments, the compound of Formula I has a structure of Formula II-16:

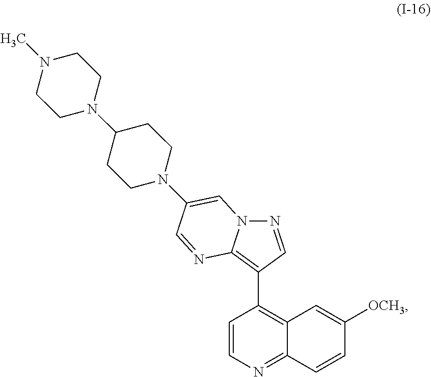 [SMILES: COc1ccc2nccc(-c3cnn4cc(N5CCC(N6CCN(C)CC6)CC5)cnc34)c2c1]

or a pharmaceutically acceptable salt thereof.

In some embodiments, the compound of Formula I has a structure of Formula I-17:

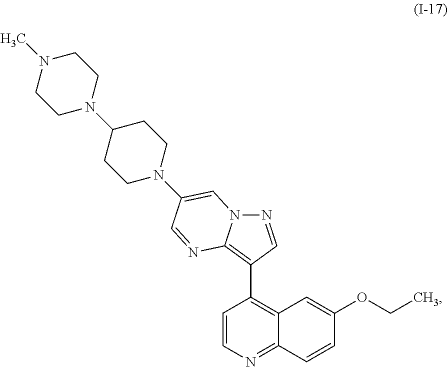 [SMILES: CCOc1ccc2nccc(-c3cnn4cc(N5CCC(N6CCN(C)CC6)CC5)cnc34)c2c1]

or a pharmaceutically acceptable salt thereof.

In some embodiments, the compound of Formula I has a structure of Formula II-18:

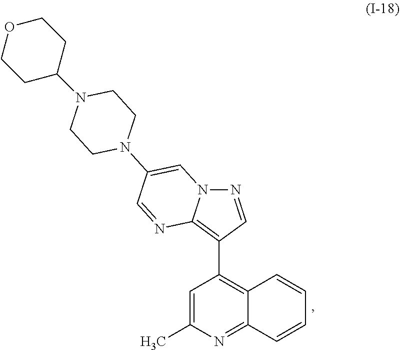 [SMILES: Cc1cc(-c2cnn3cc(N4CCN(C5CCOCC5)CC4)cnc23)c2ccccc2n1]

or a pharmaceutically acceptable salt thereof.

In some embodiments, the compound of Formula I has a structure of Formula I-19:

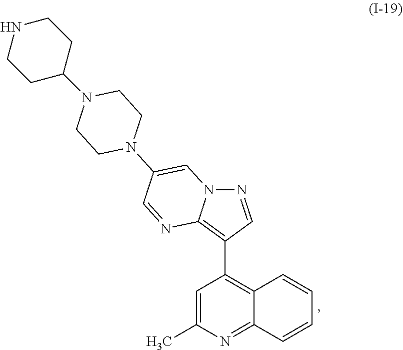 [SMILES: Cc1cc(-c2cnn3cc(N4CCN(C5CCNCC5)CC4)cnc23)c2ccccc2n1]

or a pharmaceutically acceptable salt thereof.

In some embodiments, the compound of Formula I has a structure of Formula II-20:

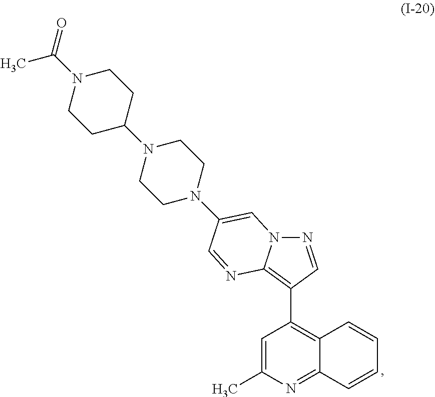 [SMILES: CC(=O)N1CCC(N2CCN(c3cnc4c(-c5cc(C)nc6ccccc56)cnn4c3)CC2)CC1]

or a pharmaceutically acceptable salt thereof.

In some embodiments, the compound of Formula I has a structure of Formula II-21:

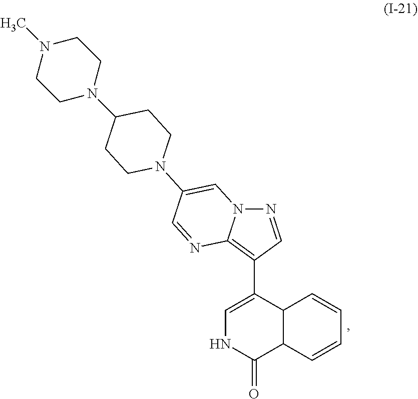 [SMILES: CN1CCN(C2CCN(c3cnc4c(C5=CNC(=O)C6C=CC=CC56)cnn4c3)CC2)CC1]

or a pharmaceutically acceptable sell thereof.

In some embodiments, the compound of Formula I has a structure of Formula II-22:

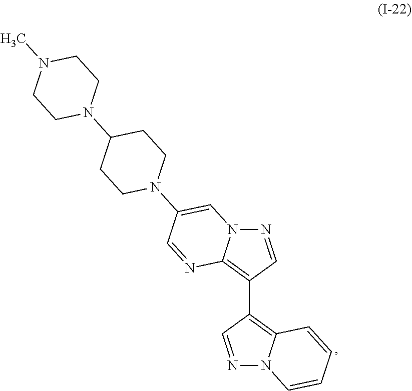 [SMILES: CN1CCN(C2CCN(c3cnc4c(-c5cnn6ccccc56)cnn4c3)CC2)CC1]

or a pharmaceutically acceptable salt thereof.

In some embodiments, the compound of Formula I has a structure of Formula II-23:

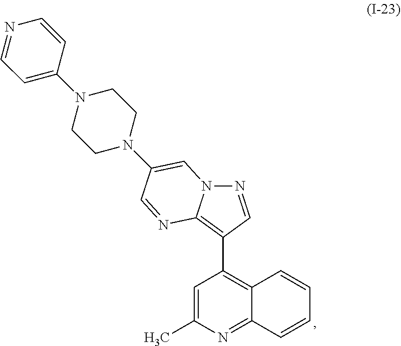 [SMILES: Cc1cc(-c2cnn3cc(N4CCN(c5ccncc5)CC4)cnc23)c2ccccc2n1]

or a pharmaceutically acceptable salt thereof.

In some embodiments, the compound of Formula I has a structure of Formula II-24:

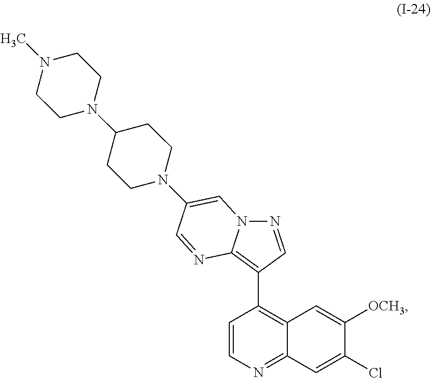 [SMILES: COc1cc2c(-c3cnn4cc(N5CCC(N6CCN(C)CC6)CC5)cnc34)ccnc2cc1Cl]

or a pharmaceutically acceptable salt thereof.

In some embodiments, the compound of Formula I has a structure of Formula II-25:

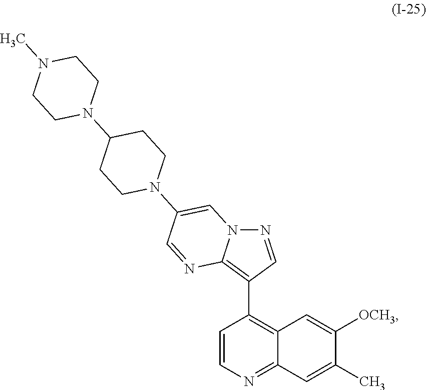 [SMILES: COc1cc2c(-c3cnn4cc(N5CCC(N6CCN(C)CC6)CC5)cnc34)ccnc2cc1C]

or a pharmaceutically acceptable salt thereof.

In some embodiments, the compound of Formula I has a structure of Formula II-26:

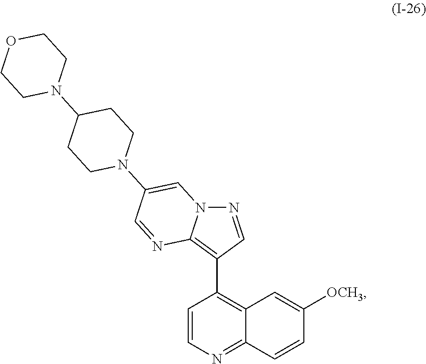 [SMILES: COc1ccc2nccc(-c3cnn4cc(N5CCC(N6CCOCC6)CC5)cnc34)c2c1]

or a pharmaceutically acceptable salt thereof.

In some embodiments, the compound of Formula I has a structure of Formula II-27:

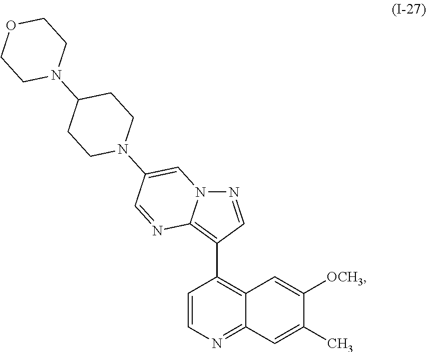 [SMILES: COc1cc2c(-c3cnn4cc(N5CCC(N6CCOCC6)CC5)cnc34)ccnc2cc1C]

In some embodiments, the compound of Formula I has a structure of Formula II-28:

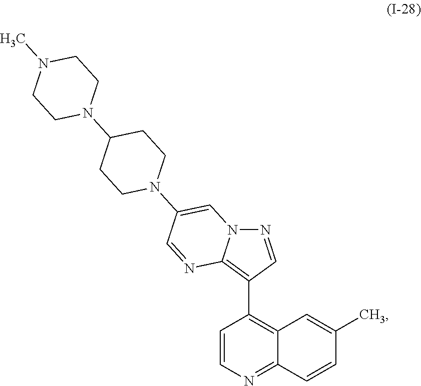 [SMILES: Cc1ccc2nccc(-c3cnn4cc(N5CCC(N6CCN(C)CC6)CC5)cnc34)c2c1]

or a pharmaceutically acceptable salt thereof.

In some embodiments, the compound of Formula I has a structure of Formula II-29:

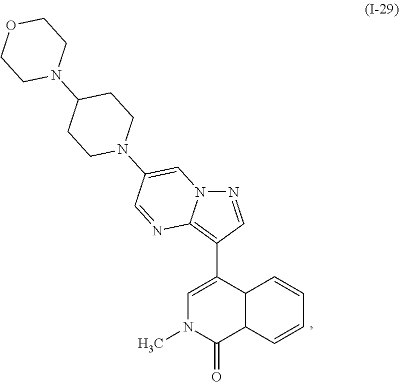 [SMILES: CN1C=C(c2cnn3cc(N4CCC(N5CCOCC5)CC4)cnc23)C2C=CC=CC2C1=O]

or a pharmaceutically acceptable salt thereof.

In some embodiments, the compound of Formula I has a structure of Formula II-30:

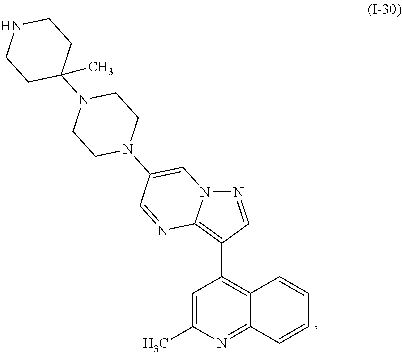 [SMILES: Cc1cc(-c2cnn3cc(N4CCN(C5(C)CCNCC5)CC4)cnc23)c2ccccc2n1]

or a pharmaceutically acceptable salt thereof.

In some embodiments, the compound of Formula I has a structure of Formula I-31:

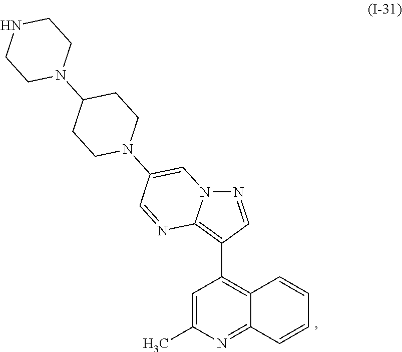 [SMILES: Cc1cc(-c2cnn3cc(N4CCC(N5CCNCC5)CC4)cnc23)c2ccccc2n1]

or a pharmaceutically acceptable salt thereof.

In some embodiments, the compound of Formula I has a structure of Formula II-32:

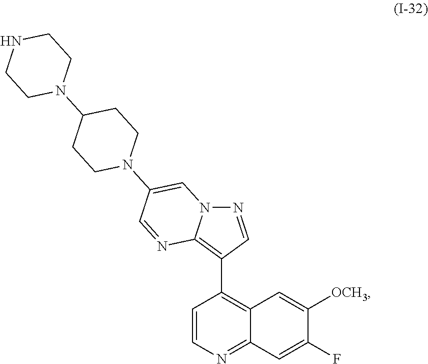 [SMILES: COc1cc2c(-c3cnn4cc(N5CCC(N6CCNCC6)CC5)cnc34)ccnc2cc1F]

or a pharmaceutically acceptable salt thereof.

In some embodiments, the compound of Formula I has a structure of Formula II-33:

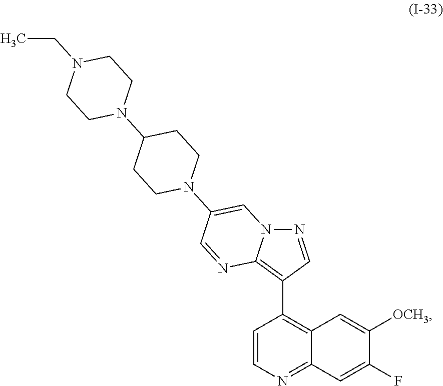 [SMILES: CCN1CCN(C2CCN(c3cnc4c(-c5ccnc6cc(F)c(OC)cc56)cnn4c3)CC2)CC1]

or a pharmaceutically acceptable salt thereof.

In some embodiments, the compound of Formula I has a structure of Formula II-34:

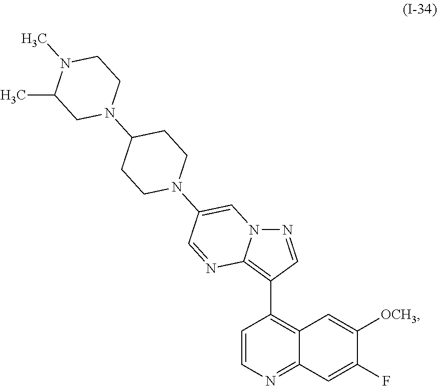 [SMILES: COc1cc2c(-c3cnn4cc(N5CCC(N6CCN(C)C(C)C6)CC5)cnc34)ccnc2cc1F]

or a pharmaceutically acceptable salt thereof.

In some embodiments, the compound of Formula I has a structure of Formula II-35:

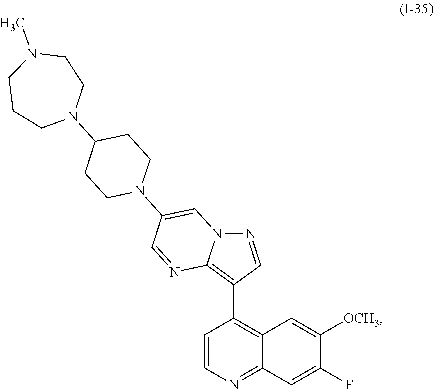 [SMILES: COc1cc2c(-c3cnn4cc(N5CCC(N6CCCN(C)CC6)CC5)cnc34)ccnc2cc1F]

or a pharmaceutically acceptable salt thereof.

In some embodiments, the compound of Formula I has a structure of Formula II-36:

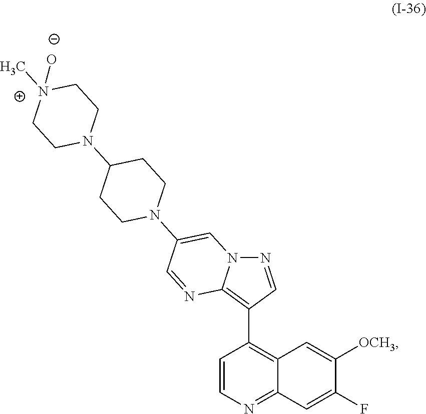

or a pharmaceutically acceptable salt thereof.

In some embodiments, the compound of Formula I has a structure of Formula II-37:

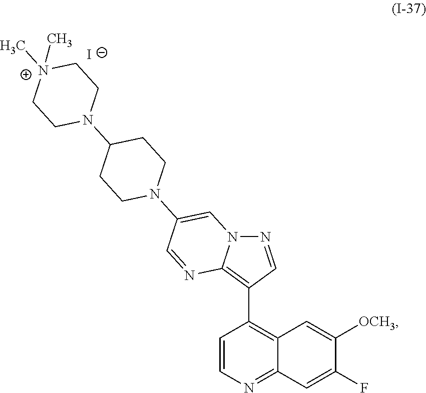 [SMILES: COc1cc2c(-c3cnn4cc(N5CCC(N6CC[N+](C)(C)CC6)CC5)cnc34)ccnc2cc1F ; [I-]]

or a pharmaceutically acceptable salt thereof.

In some embodiments, the compound of Formula I has a structure of Formula II-38:

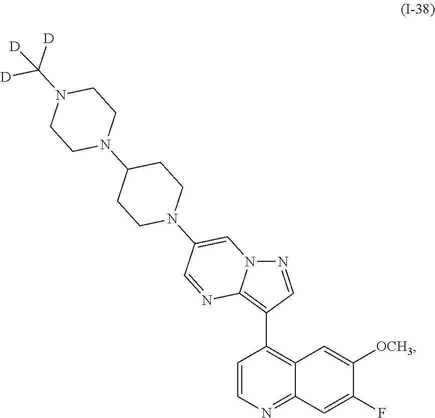 [SMILES: [2H]C([2H])([2H])N1CCN(C2CCN(c3cnc4c(-c5ccnc6cc(F)c(OC)cc56)cnn4c3)CC2)CC1]

or a pharmaceutically acceptable salt thereof.

In some embodiments, the compound of Formula I has a structure of Formula II-39:

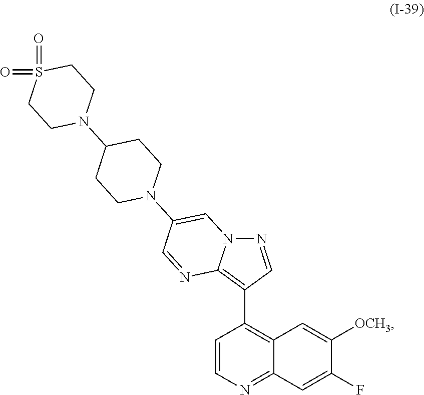 [SMILES: COc1cc2c(-c3cnn4cc(N5CCC(N6CCS(=O)(=O)CC6)CC5)cnc34)ccnc2cc1F]

In some embodiments, the compound of Formula I has a structure of Formula II-40:

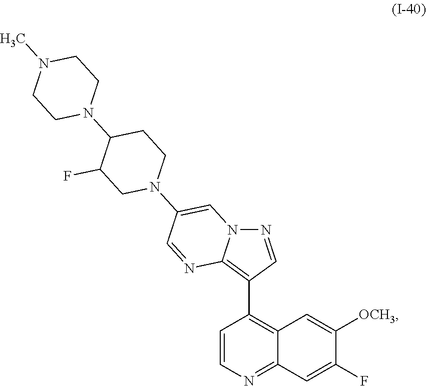 [SMILES: COc1cc2c(-c3cnn4cc(N5CCC(N6CCN(C)CC6)C(F)C5)cnc34)ccnc2cc1F]

or a pharmaceutically acceptable salt thereof.

In some embodiments, the compound of Formula I has a structure of Formula II-41:

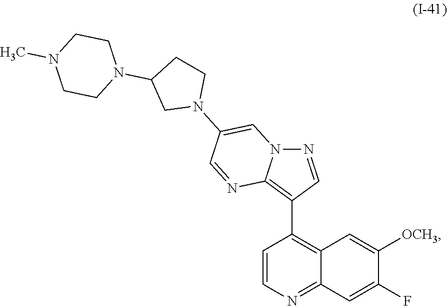 [SMILES: COc1cc2c(-c3cnn4cc(N5CCC(N6CCN(C)CC6)C5)cnc34)ccnc2cc1F]

or a pharmaceutically acceptable salt thereof.

In some embodiments, the compound of Formula I has a structure of Formula II-42:

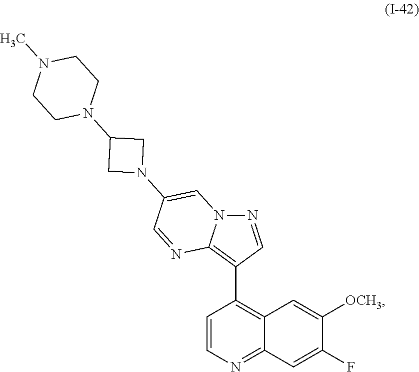 [SMILES: COc1cc2c(-c3cnn4cc(N5CC(N6CCN(C)CC6)C5)cnc34)ccnc2cc1F]

In some embodiments, the compound of Formula I has a structure of Formula II-43:

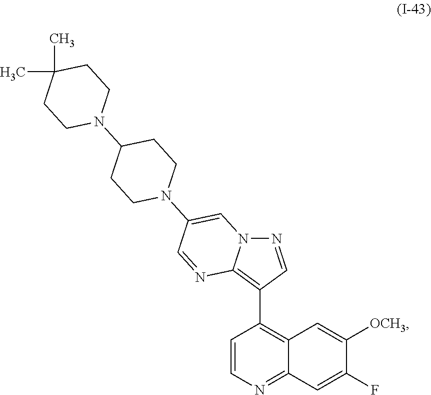 [SMILES: COc1cc2c(-c3cnn4cc(N5CCC(N6CCC(C)(C)CC6)CC5)cnc34)ccnc2cc1F]

or a pharmaceutically acceptable salt thereof.

In some embodiments, the compound of Formula I has a structure of Formula II-44:

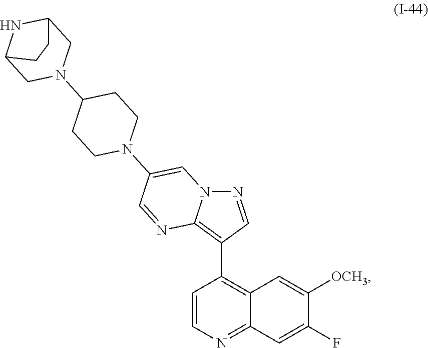 [SMILES: COc1cc2c(-c3cnn4cc(N5CCC(N6CC7CCC(C6)N7)CC5)cnc34)ccnc2cc1F]

or a pharmaceutically acceptable salt thereof.

In some embodiments, the compound of Formula I has a structure of Formula II-45:

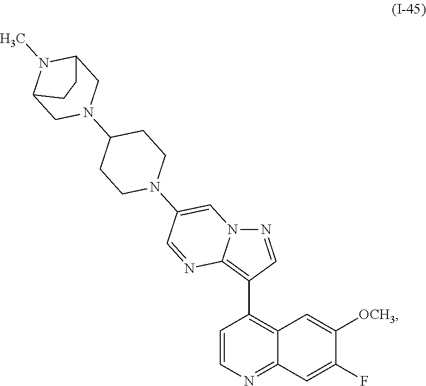 [SMILES: COc1cc2c(-c3cnn4cc(N5CCC(N6CC7CCC(C6)N7C)CC5)cnc34)ccnc2cc1F]

or a pharmaceutically acceptable salt thereof.

In some embodiments, the compound of Formula I has a structure of Formula II-46:

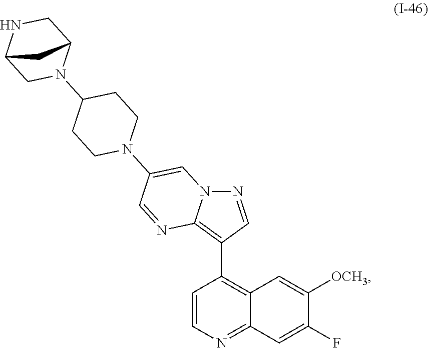 [SMILES: COc1cc2c(-c3cnn4cc(N5CCC(N6C[C@H]7C[C@@H]6CN7)CC5)cnc34)ccnc2cc1F]

or a pharmaceutically acceptable salt thereof.

In some embodiments, the compound of Formula I has a structure of Formula II-47:

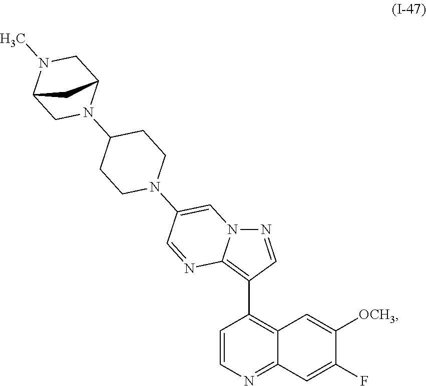 [SMILES: COc1cc2c(-c3cnn4cc(N5CCC(N6C[C@H]7C[C@@H]6CN7C)CC5)cnc34)ccnc2cc1F]

or a pharmaceutically acceptable salt thereof.

In some embodiments, the compound of Formula I has a structure of Formula II-48:

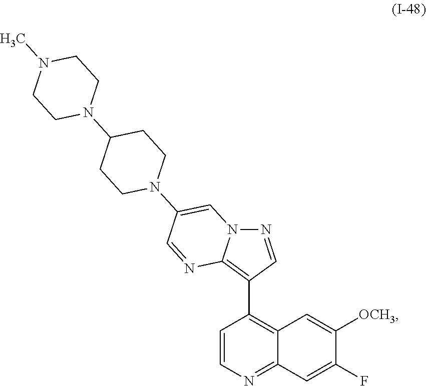 [SMILES: COc1cc2c(-c3cnn4cc(N5CCC(N6CCN(C)CC6)CC5)cnc34)ccnc2cc1F]

In some embodiments, the compound of Formula I has a structure of Formula II-49:

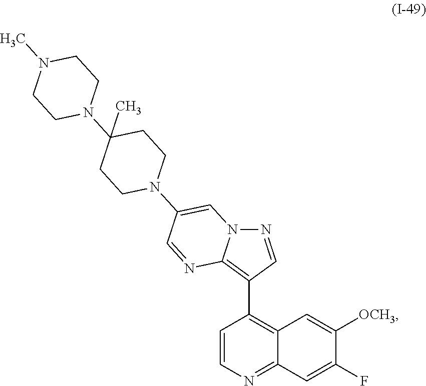 [SMILES: COc1cc2c(-c3cnn4cc(N5CCC(C)(N6CCN(C)CC6)CC5)cnc34)ccnc2cc1F]

or a pharmaceutically acceptable salt thereof.

In some embodiments, the compound of Formula I has a structure of Formula II-50:

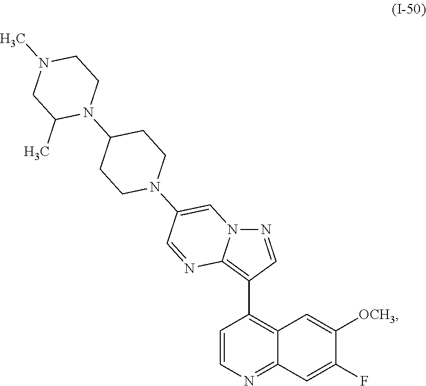 [SMILES: COc1cc2c(-c3cnn4cc(N5CCC(N6CCN(C)CC6C)CC5)cnc34)ccnc2cc1F]

or a pharmaceutically acceptable salt thereof.

In some embodiments, the compound of Formula I has a structure of Formula II-51:

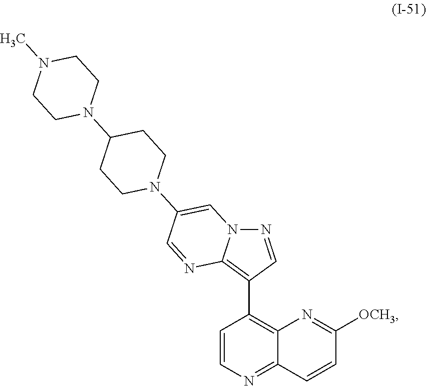 [SMILES: COc1ccc2nccc(-c3cnn4cc(N5CCC(N6CCN(C)CC6)CC5)cnc34)c2n1]

In some embodiments, the compound of Formula I has a structure of Formula II-52:

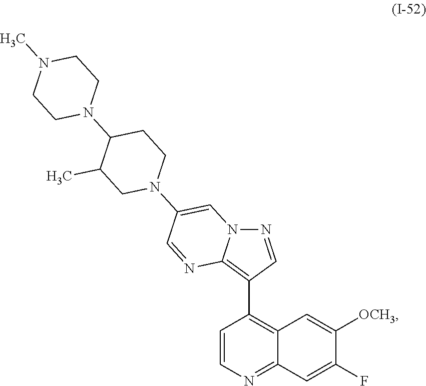 [SMILES: COc1cc2c(-c3cnn4cc(N5CCC(N6CCN(C)CC6)C(C)C5)cnc34)ccnc2cc1F]

or a pharmaceutically acceptable salt thereof.

In some embodiments, the compound of Formula I has a structure of Formula II-53:

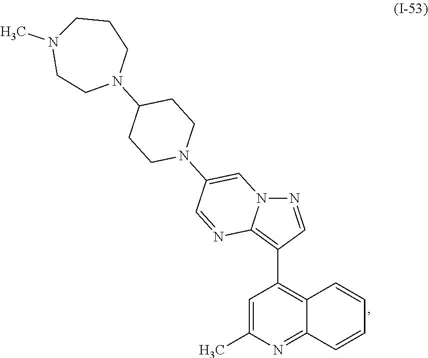 [SMILES: Cc1cc(-c2cnn3cc(N4CCC(N5CCCN(C)CC5)CC4)cnc23)c2ccccc2n1]

or a pharmaceutically acceptable salt thereof.

In some embodiments, the compound of Formula I has a structure of Formula II-54:

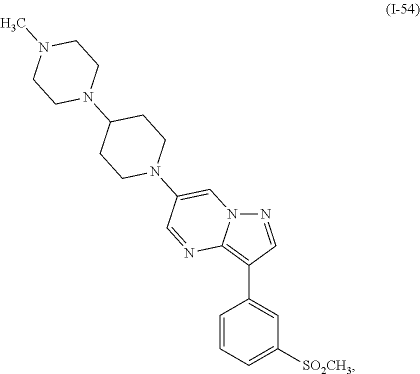 [SMILES: CN1CCN(C2CCN(c3cnc4c(-c5cccc(S(C)(=O)=O)c5)cnn4c3)CC2)CC1]

or a pharmaceutically acceptable salt thereof.

In some embodiments, the compound of Formula I has a structure of Formula II-55:

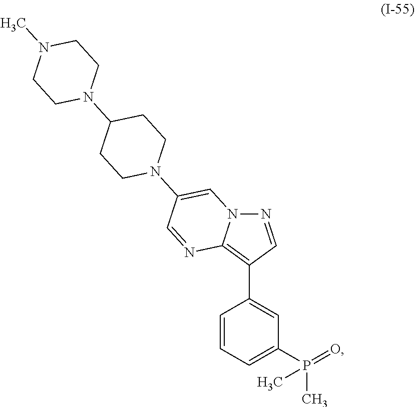 [SMILES: CN1CCN(C2CCN(c3cnc4c(-c5cccc(P(C)(C)=O)c5)cnn4c3)CC2)CC1]

or a pharmaceutically acceptable salt thereof.

In some embodiments, the compound of Formula I has a structure of Formula II-56:

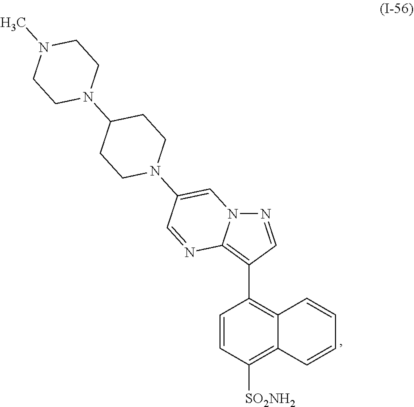 [SMILES: CN1CCN(C2CCN(c3cnc4c(-c5ccc(S(N)(=O)=O)c6ccccc56)cnn4c3)CC2)CC1]

or a pharmaceutically acceptable salt thereof.

In some embodiments, the compound of Formula I has a structure of Formula II-57:

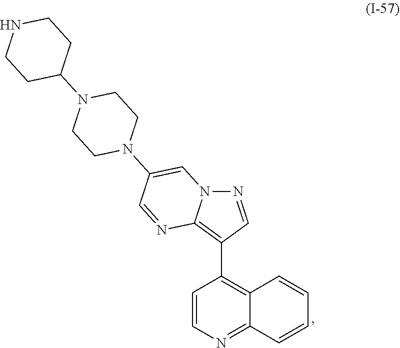 [SMILES: c1ccc2c(-c3cnn4cc(N5CCN(C6CCNCC6)CC5)cnc34)ccnc2c1]

or a pharmaceutically acceptable salt thereof.

In some embodiments, the compound of Formula I has a structure of Formula II-58:

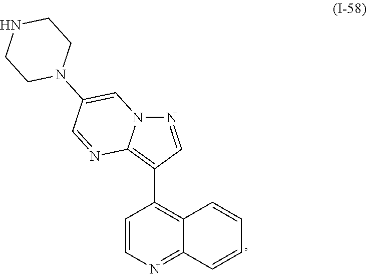 [SMILES: c1ccc2c(-c3cnn4cc(N5CCNCC5)cnc34)ccnc2c1]

or a pharmaceutically acceptable salt thereof.

In some embodiments, the compound of Formula I has a structure of Formula II-59:

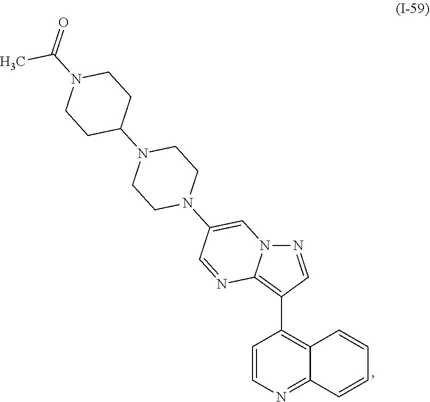 [SMILES: CC(=O)N1CCC(N2CCN(c3cnc4c(-c5ccnc6ccccc56)cnn4c3)CC2)CC1]

or a pharmaceutically acceptable salt thereof.

In some embodiments, the compound of Formula I has a structure of Formula II-60:

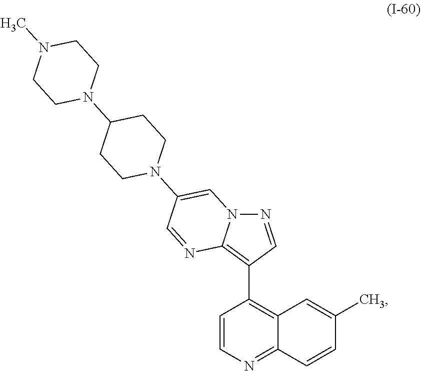 [SMILES: Cc1ccc2nccc(-c3cnn4cc(N5CCC(N6CCN(C)CC6)CC5)cnc34)c2c1]

or a pharmaceutically acceptable salt thereof.

In some embodiments, the compound of Formula I has a structure of Formula II-61:

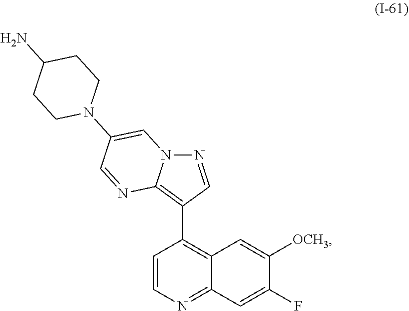 [SMILES: COc1cc2c(-c3cnn4cc(N5CCC(N)CC5)cnc34)ccnc2cc1F]

or a pharmaceutically acceptable salt thereof.

In some embodiments, the compound of Formula I has a structure of Formula II-62:

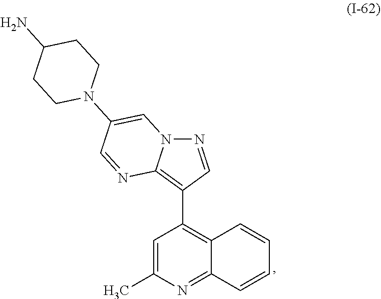 [SMILES: Cc1cc(-c2cnn3cc(N4CCC(N)CC4)cnc23)c2ccccc2n1]

or a pharmaceutically acceptable salt thereof.

In some embodiments, the compound of Formula I has a structure of Formula II-63:

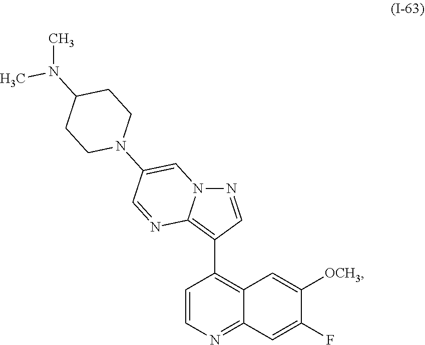 [SMILES: COc1cc2c(-c3cnn4cc(N5CCC(N(C)C)CC5)cnc34)ccnc2cc1F]

or a pharmaceutically acceptable salt thereof.

In some embodiments, the compound of Formula I has a structure of Formula II-64:

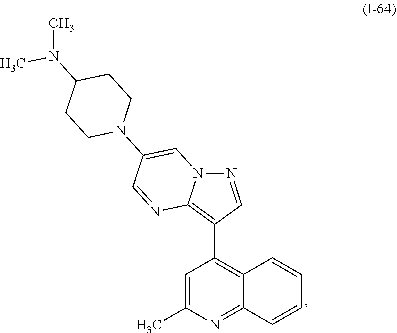 [SMILES: Cc1cc(-c2cnn3cc(N4CCC(N(C)C)CC4)cnc23)c2ccccc2n1]

or a pharmaceutically acceptable salt thereof.

In some embodiments, the compound of Formula I has a structure of Formula II-65:

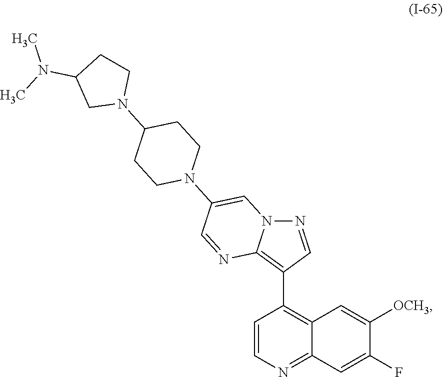 [SMILES: COc1cc2c(-c3cnn4cc(N5CCC(N6CCC(N(C)C)C6)CC5)cnc34)ccnc2cc1F]

or a pharmaceutically acceptable salt thereof.

In some embodiments, the compound of Formula I has a structure of Formula II-66:

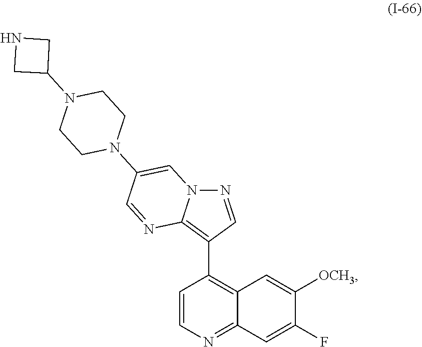 [SMILES: COc1cc2c(-c3cnn4cc(N5CCN(C6CNC6)CC5)cnc34)ccnc2cc1F]

or a pharmaceutically acceptable salt thereof.

In some embodiments, the compound of Formula I has a structure of Formula II-67:

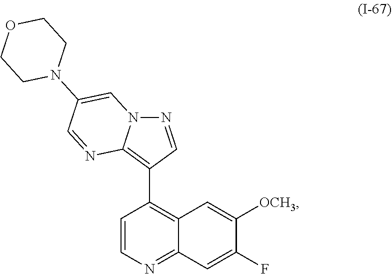 [SMILES: COc1cc2c(-c3cnn4cc(N5CCOCC5)cnc34)ccnc2cc1F]

or a pharmaceutically acceptable salt thereof.

In some embodiments, the compound of Formula I has a structure of Formula II-68:

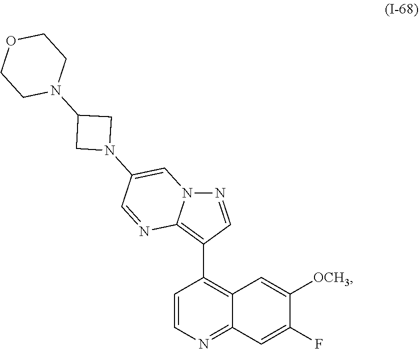 [SMILES: COc1cc2c(-c3cnn4cc(N5CC(N6CCOCC6)C5)cnc34)ccnc2cc1F]

or a pharmaceutically acceptable salt thereof.

In some embodiments, the compound of Formula I has a structure of Formula II-69:

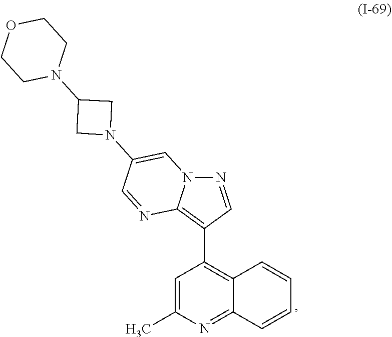 [SMILES: Cc1cc(-c2cnn3cc(N4CC(N5CCOCC5)C4)cnc23)c2ccccc2n1]

or a pharmaceutically acceptable salt thereof.

In some embodiments, the compound of Formula I has a structure of Formula II-70:

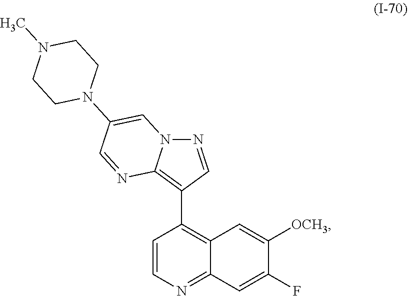 [SMILES: COc1cc2c(-c3cnn4cc(N5CCN(C)CC5)cnc34)ccnc2cc1F]

or a pharmaceutically acceptable salt thereof.

In some embodiments, the compound of Formula I has a structure of Formula II-71:

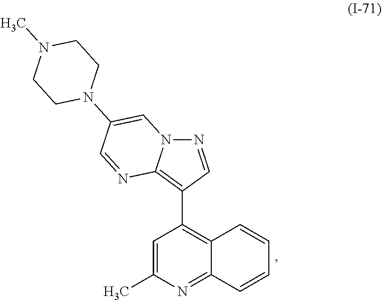 [SMILES: Cc1cc(-c2cnn3cc(N4CCN(C)CC4)cnc23)c2ccccc2n1]

or a pharmaceutically acceptable salt thereof.

In some embodiments, the compound of Formula I has a structure of Formula II-72:

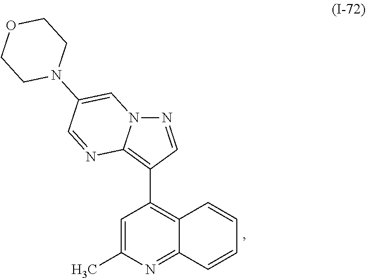 [SMILES: Cc1cc(-c2cnn3cc(N4CCOCC4)cnc23)c2ccccc2n1]

or a pharmaceutically acceptable salt thereof.

In some embodiments, the compound of Formula I has a structure of Formula II-73:

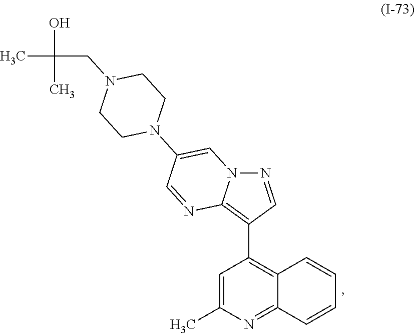 [SMILES: Cc1cc(-c2cnn3cc(N4CCN(CC(C)(C)O)CC4)cnc23)c2ccccc2n1]

or a pharmaceutically acceptable salt thereof.

In some embodiments, the compound of Formula I has a structure of Formula II-74:

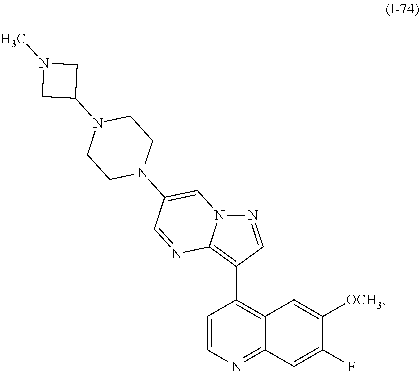 [SMILES: COc1cc2c(-c3cnn4cc(N5CCN(C6CN(C)C6)CC5)cnc34)ccnc2cc1F]

or a pharmaceutically acceptable salt thereof.

In some embodiments, the compound of Formula I has a structure of Formula II-75:

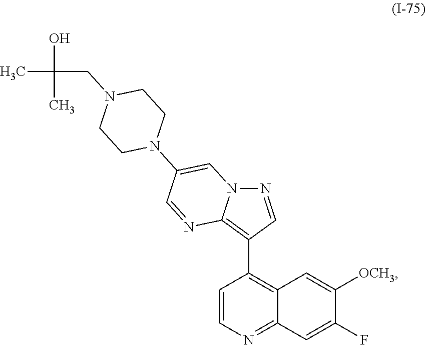 [SMILES: COc1cc2c(-c3cnn4cc(N5CCN(CC(C)(C)O)CC5)cnc34)ccnc2cc1F]

or a pharmaceutically acceptable salt thereof.

In some embodiments, the compound of Formula I has a structure of Formula II-78:

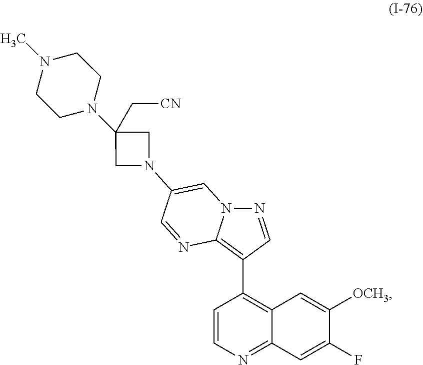 [SMILES: COc1cc2c(-c3cnn4cc(N5CC(CC#N)(N6CCN(C)CC6)C5)cnc34)ccnc2cc1F]

or a pharmaceutically acceptable salt thereof.

In some embodiments, the compound of Formula I has a structure of Formula II-77:

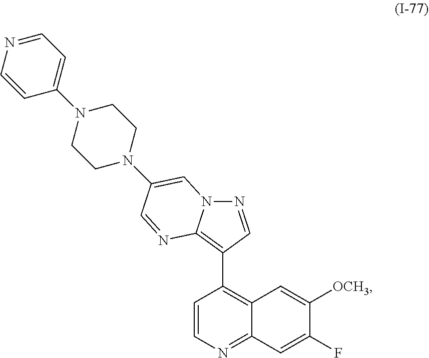 [SMILES: COc1cc2c(-c3cnn4cc(N5CCN(c6ccncc6)CC5)cnc34)ccnc2cc1F]

or a pharmaceutically acceptable salt thereof.

In some embodiments, the compound of Formula I has a structure of Formula II-78:

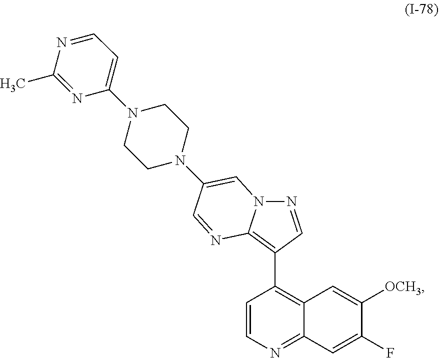 [SMILES: COc1cc2c(-c3cnn4cc(N5CCN(c6ccnc(C)n6)CC5)cnc34)ccnc2cc1F]

or a pharmaceutically acceptable salt thereof.

In some embodiments, the compound of Formula I has a structure of Formula II-79:

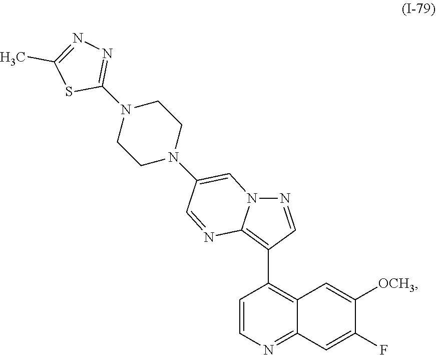 [SMILES: COc1cc2c(-c3cnn4cc(N5CCN(c6nnc(C)s6)CC5)cnc34)ccnc2cc1F]

or a pharmaceutically acceptable salt thereof.

In some embodiments, the compound of Formula I has a structure of Formula II-80:

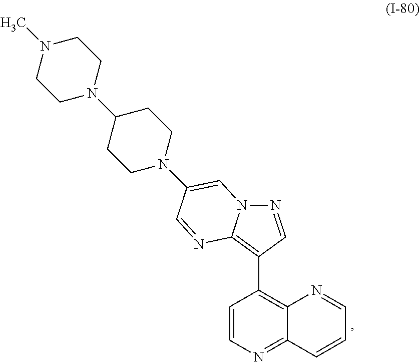 [SMILES: CN1CCN(C2CCN(c3cnc4c(-c5ccnc6cccnc56)cnn4c3)CC2)CC1]

In some embodiments, the compound of Formula I has a structure of Formula II-81:

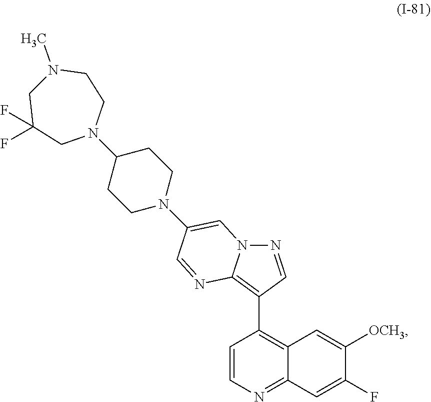 [SMILES: COc1cc2c(-c3cnn4cc(N5CCC(N6CCN(C)CC(F)(F)C6)CC5)cnc34)ccnc2cc1F]

or a pharmaceutically acceptable salt thereof.

In some embodiments, the compound of Formula I has a structure of Formula II-82:

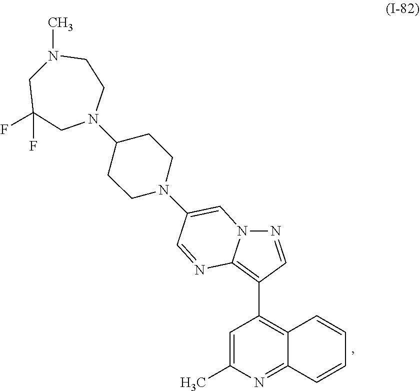 [SMILES: Cc1cc(-c2cnn3cc(N4CCC(N5CCN(C)CC(F)(F)C5)CC4)cnc23)c2ccccc2n1]

or a pharmaceutically acceptable salt thereof.

In some embodiments, the compound of Formula I has a structure of Formula II-83:

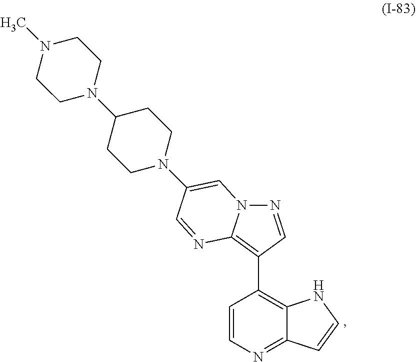 [SMILES: CN1CCN(C2CCN(c3cnc4c(-c5ccnc6cc[nH]c56)cnn4c3)CC2)CC1]

or a pharmaceutically acceptable salt thereof.

In some embodiments, the compound of Formula I has a structure of Formula II-84:

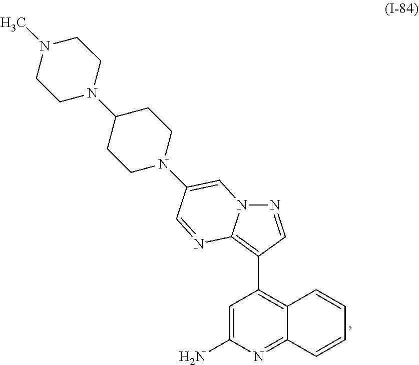 [SMILES: CN1CCN(C2CCN(c3cnc4c(-c5cc(N)nc6ccccc56)cnn4c3)CC2)CC1]

or a pharmaceutically acceptable salt thereof.

In some embodiments, the compound of Formula I has a structure of Formula II-85:

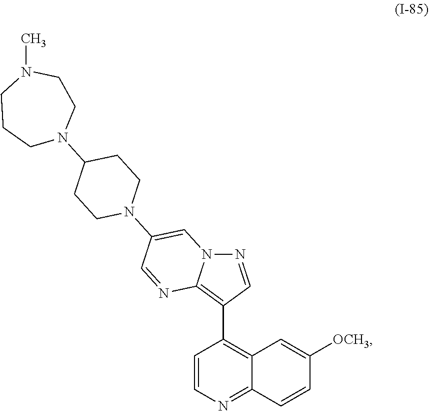 [SMILES: COc1ccc2nccc(-c3cnn4cc(N5CCC(N6CCCN(C)CC6)CC5)cnc34)c2c1]

or a pharmaceutically acceptable salt thereof.

In some embodiments, the compound of Formula I has a structure of Formula II-86:

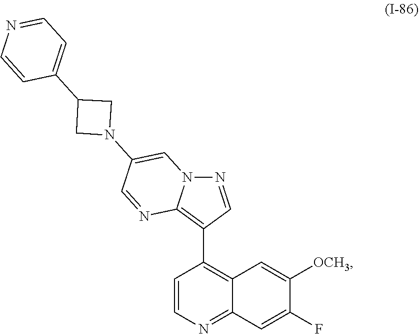 [SMILES: COc1cc2c(-c3cnn4cc(N5CC(c6ccncc6)C5)cnc34)ccnc2cc1F]

or a pharmaceutically acceptable salt thereof.

In some embodiments, the compound of Formula I has a structure of Formula II-87:

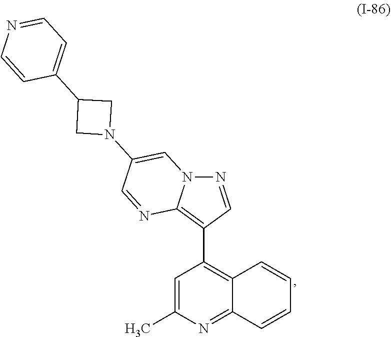 [SMILES: Cc1cc(-c2cnn3cc(N4CC(c5ccncc5)C4)cnc23)c2ccccc2n1]

or a pharmaceutically acceptable salt thereof.

In some embodiments, the compound of Formula I has a structure of Formula II-88:

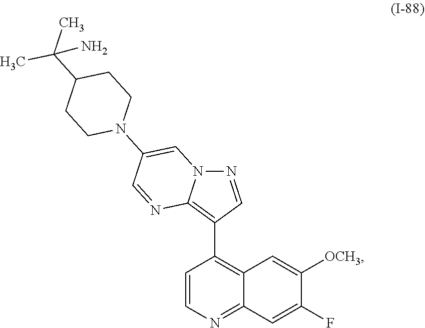 [SMILES: COc1cc2c(-c3cnn4cc(N5CCC(C(C)(C)N)CC5)cnc34)ccnc2cc1F]

or a pharmaceutically acceptable salt thereof.

In some embodiments, the compound of Formula I has a structure of Formula II-89:

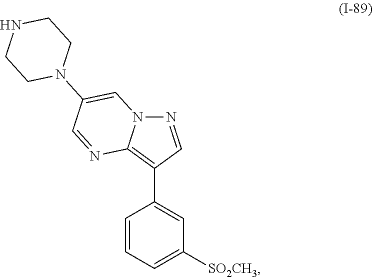 [SMILES: CS(=O)(=O)c1cccc(-c2cnn3cc(N4CCNCC4)cnc23)c1]

or a pharmaceutically acceptable salt thereof.

In some embodiments, the compound of Formula I has a structure of Formula II-00:

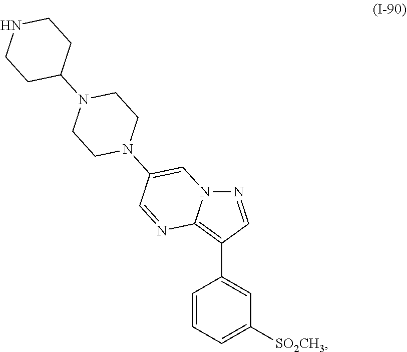 [SMILES: CS(=O)(=O)c1cccc(-c2cnn3cc(N4CCN(C5CCNCC5)CC4)cnc23)c1]

or a pharmaceutically acceptable salt thereof.

In some embodiments, the compound of Formula I has a structure of Formula II-91:

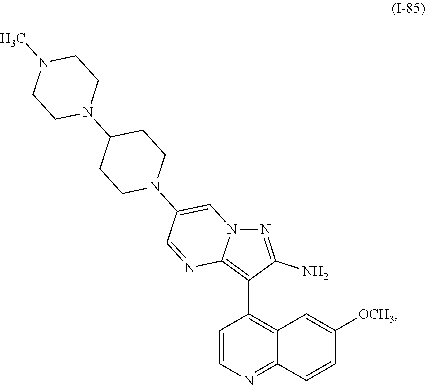 [SMILES: COc1ccc2nccc(-c3c(N)nn4cc(N5CCC(N6CCN(C)CC6)CC5)cnc34)c2c1]

or a pharmaceutically acceptable salt thereof.

In some embodiments, the compound of Formula I has a structure of Formula II-92:

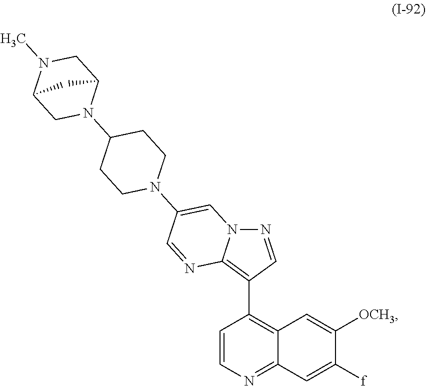

or a pharmaceutically acceptable salt thereof.

In some embodiments, the compound of Formula I has a structure of Formula II-03:

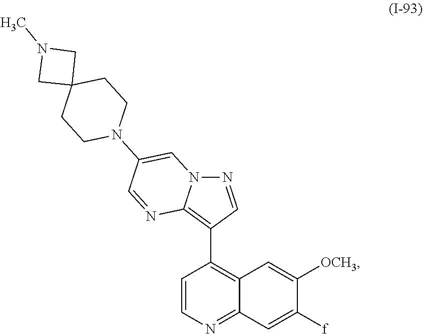

or a pharmaceutically acceptable salt thereof.

In some embodiments, the compound of Formula I has a structure of Formula II-94:

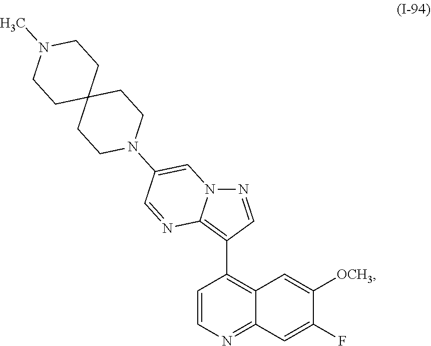 [SMILES: COc1cc2c(-c3cnn4cc(N5CCC6(CCN(C)CC6)CC5)cnc34)ccnc2cc1F]

In some embodiments, the compound of Formula I has a structure of Formula II-95:

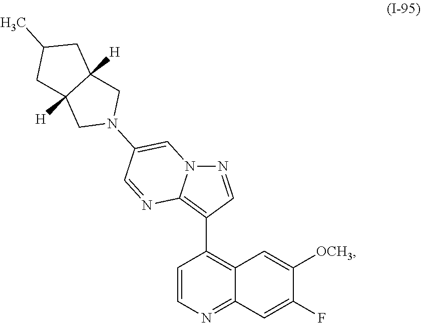 [SMILES: COc1cc2c(-c3cnn4cc(N5C[C@H]6CC(C)C[C@H]6C5)cnc34)ccnc2cc1F]

or a pharmaceutically acceptable salt thereof.

In some embodiments, the compound of Formula I has a structure of Formula II-98:

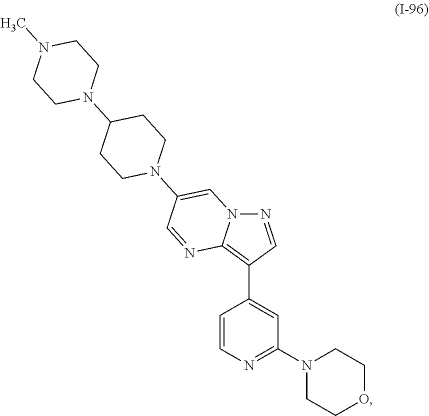 [SMILES: CN1CCN(C2CCN(c3cnc4c(-c5ccnc(N6CCOCC6)c5)cnn4c3)CC2)CC1]

or a pharmaceutically acceptable salt thereof.

In some embodiments, the compound of Formula I has a structure of Formula II-97:

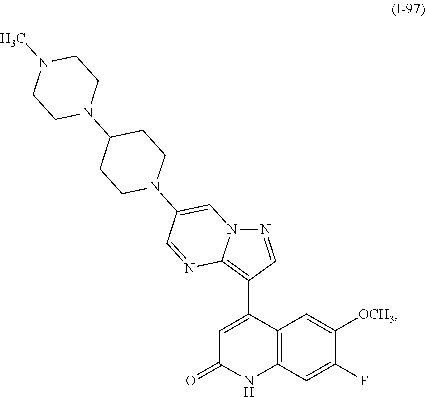 [SMILES: COc1cc2c(-c3cnn4cc(N5CCC(N6CCN(C)CC6)CC5)cnc34)cc(=O)[nH]c2cc1F]

or a pharmaceutically acceptable salt thereof.

In some embodiments, the compound of Formula I has a structure of Formula II-08:

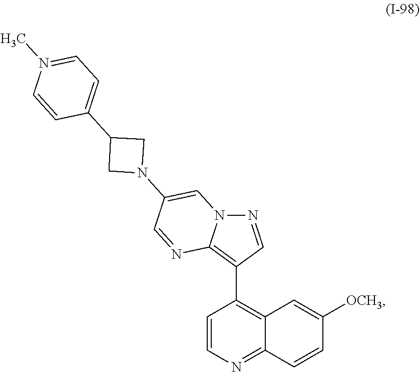

or a pharmaceutically acceptable salt thereof.

In some embodiments, the compound of Formula I has a structure of Formula II-09:

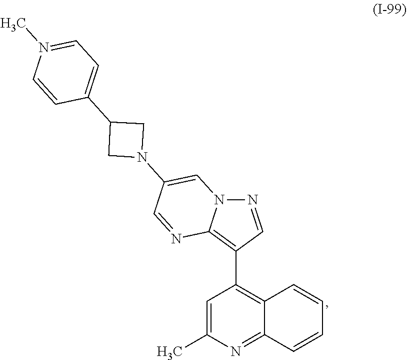

or a pharmaceutically acceptable salt thereof.

In some embodiments, the compound of Formula I has a structure of Formula I-100:

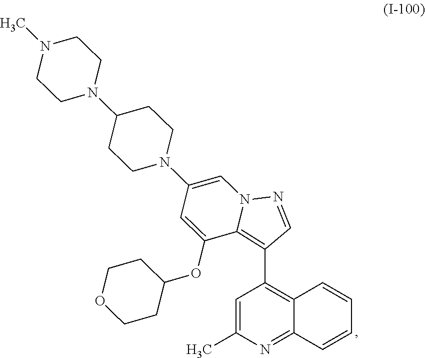 [SMILES: Cc1cc(-c2cnn3cc(N4CCC(N5CCN(C)CC5)CC4)cc(OC4CCOCC4)c23)c2ccccc2n1]

or a pharmaceutically acceptable salt thereof.

In some embodiments, the compound of Formula I has a structure of Formula II-101:

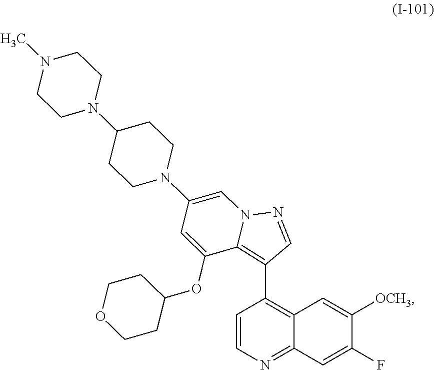 [SMILES: COc1cc2c(-c3cnn4cc(N5CCC(N6CCN(C)CC6)CC5)cc(OC5CCOCC5)c34)ccnc2cc1F]

or a pharmaceutically acceptable sail thereof.

In some embodiments, the compound of Formula I has a structure of Formula I-102:

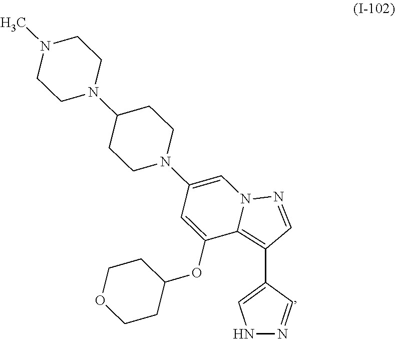 [SMILES: CN1CCN(C2CCN(c3cc(OC4CCOCC4)c4c(-c5cn[nH]c5)cnn4c3)CC2)CC1]

or a pharmaceutically acceptable salt thereof.

In some embodiments, the compound of Formula I has a structure of Formula II-103:

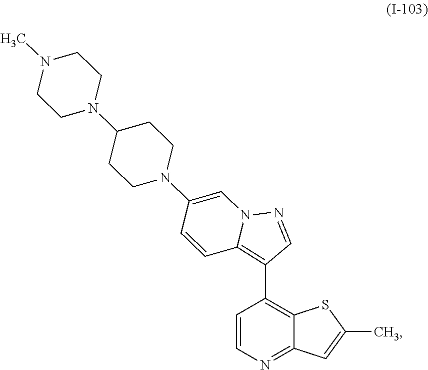 [SMILES: Cc1cc2nccc(-c3cnn4cc(N5CCC(N6CCN(C)CC6)CC5)ccc34)c2s1]

In some embodiments, the compound of Formula I has a structure of Formula I-104:

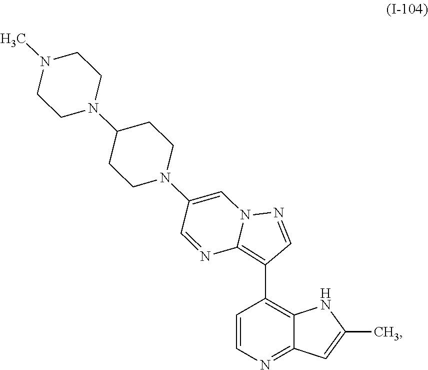 [SMILES: Cc1cc2nccc(-c3cnn4cc(N5CCC(N6CCN(C)CC6)CC5)cnc34)c2[nH]1]

or a pharmaceutically acceptable salt thereof.

In some embodiments, the compound of Formula I has a structure of Formula I-105:

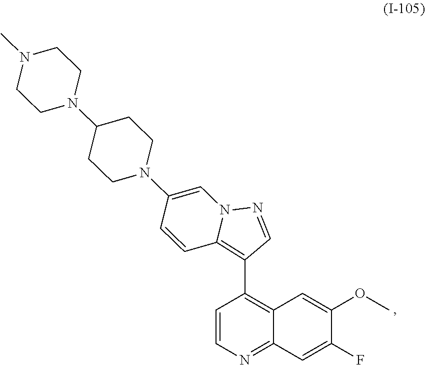

or a pharmaceutically acceptable salt thereof.

In some embodiments, the compound of Formula I has a structure of Formula II-106:

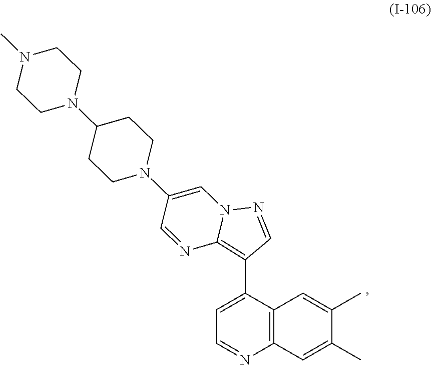

or a pharmaceutically acceptable salt thereof.

In some embodiments, the compound of Formula I has a structure of Formula II-107:

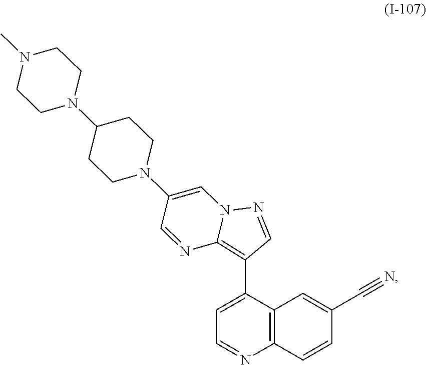 [SMILES: CN1CCN(C2CCN(c3cnc4c(-c5ccnc6ccc(C#N)cc56)cnn4c3)CC2)CC1]

or a pharmaceutically acceptable salt thereof.

In some embodiments, the compound of Formula I has a structure of Formula II-108:

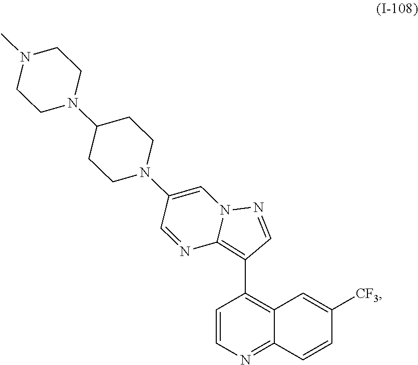 [SMILES: CN1CCN(C2CCN(c3cnc4c(-c5ccnc6ccc(C(F)(F)F)cc56)cnn4c3)CC2)CC1]

or a pharmaceutically acceptable salt thereof.

In some embodiments, the compound of Formula I has a structure of Formula II-109:

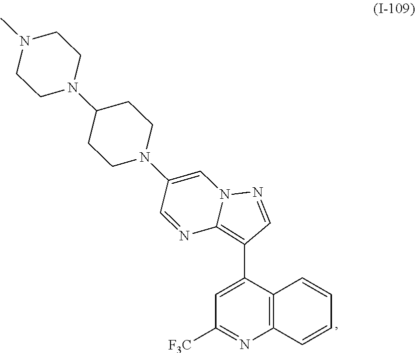 [SMILES: CN1CCN(C2CCN(c3cnc4c(-c5cc(C(F)(F)F)nc6ccccc56)cnn4c3)CC2)CC1]

In some embodiments, the compound of Formula I has a structure of Formula I-110:

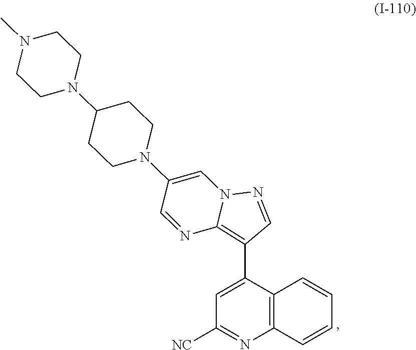 [SMILES: CN1CCN(C2CCN(c3cnc4c(-c5cc(C#N)nc6ccccc56)cnn4c3)CC2)CC1]

or a pharmaceutically acceptable salt thereof.

In some embodiments, the compound of Formula I has a structure of Formula I-111:

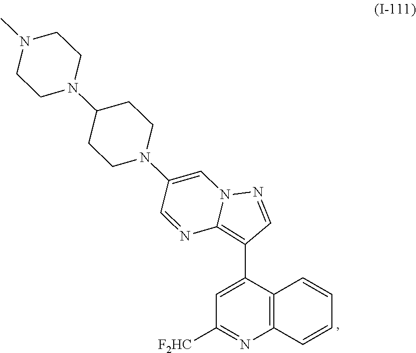 [SMILES: CN1CCN(C2CCN(c3cnc4c(-c5cc(C(F)F)nc6ccccc56)cnn4c3)CC2)CC1]

or a pharmaceutically acceptable sail thereof.

In some embodiments, the compound of Formula I has a structure of Formula I-112:

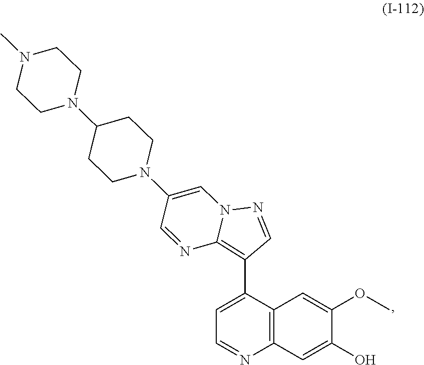

In some embodiments, the compound of Formula I has a structure of Formula II-113:

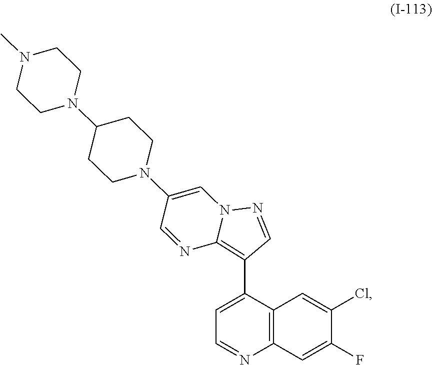 [SMILES: CN1CCN(C2CCN(c3cnc4c(-c5ccnc6cc(F)c(Cl)cc56)cnn4c3)CC2)CC1]

or a pharmaceutically acceptable salt thereof.

In some embodiments, the compound of Formula I has a structure of Formula I-114:

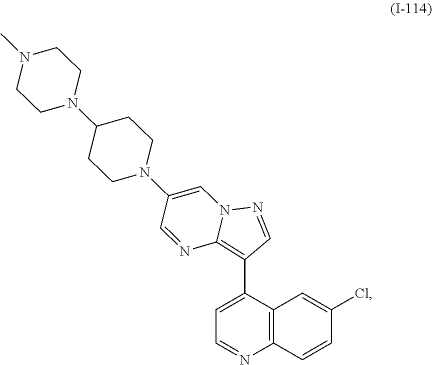 [SMILES: CN1CCN(C2CCN(c3cnc4c(-c5ccnc6ccc(Cl)cc56)cnn4c3)CC2)CC1]

or a pharmaceutically acceptable salt thereof.

In some embodiments, the compound of Formula I has a structure of Formula II-115:

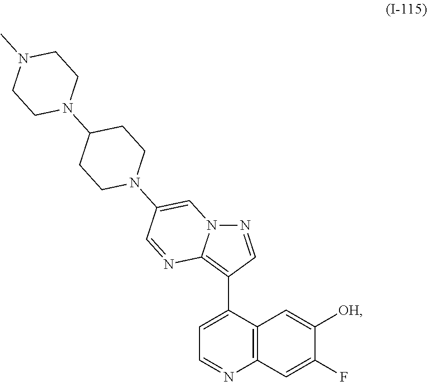 [SMILES: CN1CCN(C2CCN(c3cnc4c(-c5ccnc6cc(F)c(O)cc56)cnn4c3)CC2)CC1]

or a pharmaceutically acceptable salt thereof.

In some embodiments, the compound of Formula I has a structure of Formula I-116:

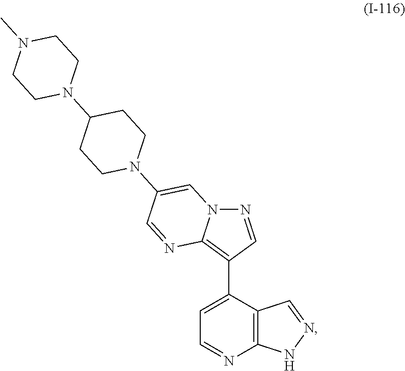 [SMILES: CN1CCN(C2CCN(c3cnc4c(-c5ccnc6[nH]ncc56)cnn4c3)CC2)CC1]

or a pharmaceutically acceptable salt thereof.

In some embodiments, the compound of Formula I has a structure of Formula I-117:

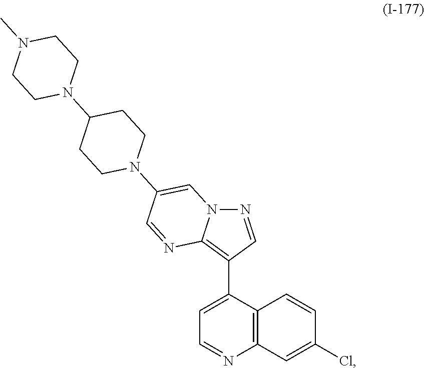 [SMILES: CN1CCN(C2CCN(c3cnc4c(-c5ccnc6cc(Cl)ccc56)cnn4c3)CC2)CC1]

or a pharmaceutically acceptable salt thereof.

In some embodiments, the compound of Formula I has a structure of Formula I-118:

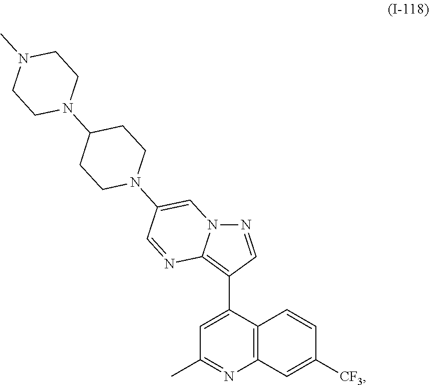 [SMILES: Cc1cc(-c2cnn3cc(N4CCC(N5CCN(C)CC5)CC4)cnc23)c2ccc(C(F)(F)F)cc2n1]

In some embodiments, the compound of Formula I has a structure of Formula II-119:

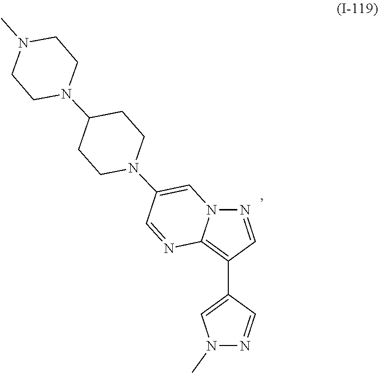 [SMILES: CN1CCN(C2CCN(c3cnc4c(-c5cnn(C)c5)cnn4c3)CC2)CC1]

or a pharmaceutically acceptable salt thereof.

In some embodiments, the compound of Formula I has a structure of Formula I-120:

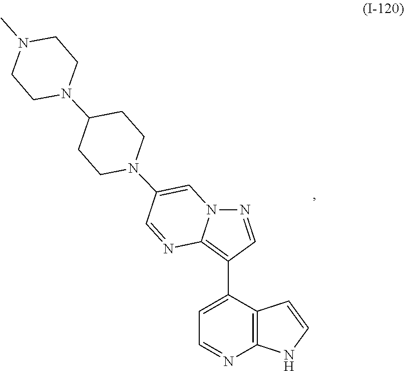 [SMILES: CN1CCN(C2CCN(c3cnc4c(-c5ccnc6[nH]ccc56)cnn4c3)CC2)CC1]

or a pharmaceutically acceptable salt thereof.

In some embodiments, the compound of Formula I has a structure of Formula I-121:

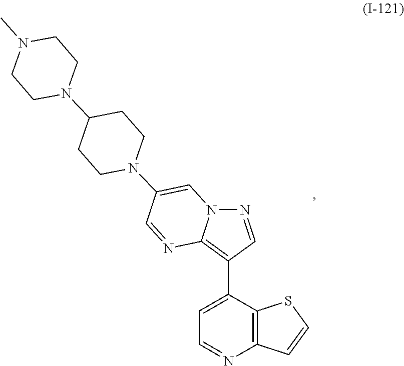 [SMILES: CN1CCN(C2CCN(c3cnc4c(-c5ccnc6ccsc56)cnn4c3)CC2)CC1]

In some embodiments, the compound of Formula I has a structure of Formula II-122:

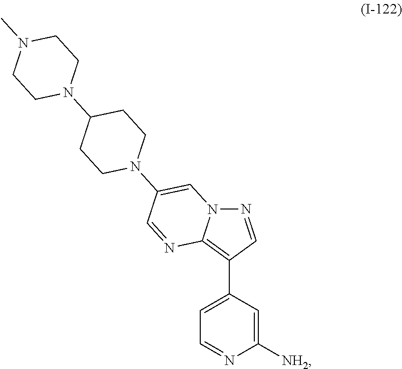 [SMILES: CN1CCN(C2CCN(c3cnc4c(-c5ccnc(N)c5)cnn4c3)CC2)CC1]

or a pharmaceutically acceptable salt thereof.

In some embodiments, the compound of Formula I has a structure of Formula I-123:

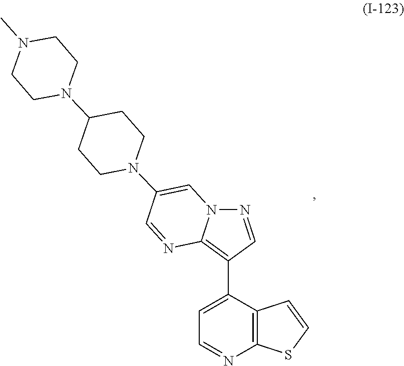 [SMILES: CN1CCN(C2CCN(c3cnc4c(-c5ccnc6sccc56)cnn4c3)CC2)CC1]

or a pharmaceutically acceptable salt thereof.

In some embodiments, the compound of Formula I has a structure of Formula II-124:

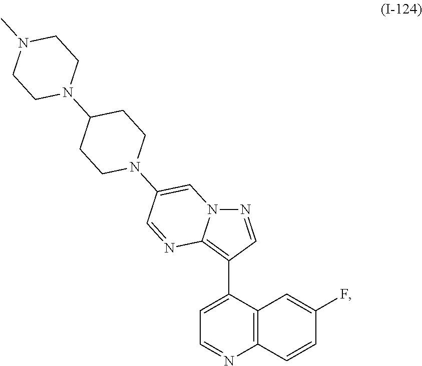 [SMILES: CN1CCN(C2CCN(c3cnc4c(-c5ccnc6ccc(F)cc56)cnn4c3)CC2)CC1]

In some embodiments, the compound of Formula I has a structure of Formula I-125:

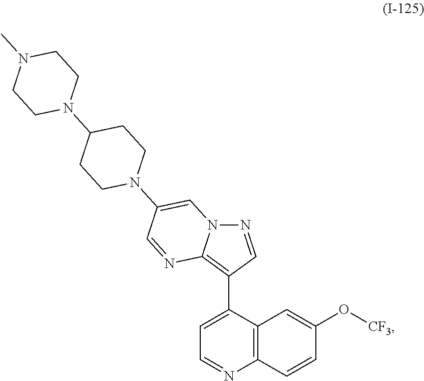 [SMILES: CN1CCN(C2CCN(c3cnc4c(-c5ccnc6ccc(OC(F)(F)F)cc56)cnn4c3)CC2)CC1]

or a pharmaceutically acceptable salt thereof.

In some embodiments, the compound of Formula I has a structure of Formula I-126:

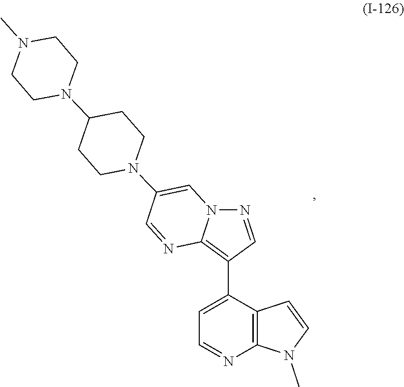 [SMILES: CN1CCN(C2CCN(c3cnc4c(-c5ccnc6c5ccn6C)cnn4c3)CC2)CC1]

or a pharmaceutically acceptable salt thereof.

In some embodiments, the compound of Formula I has a structure of Formula I-127:

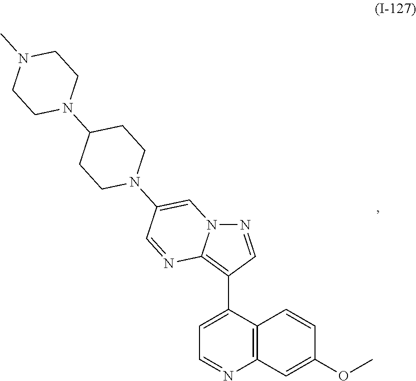

or a pharmaceutically acceptable salt thereof.

In some embodiments, the compound of Formula I has a structure of Formula I-128:

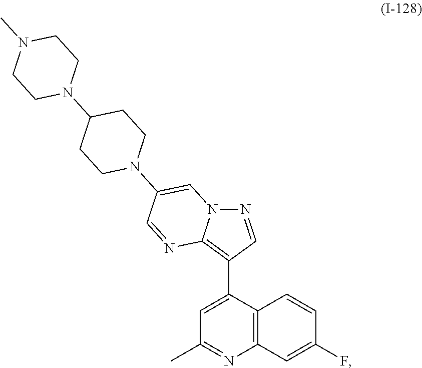 [SMILES: Cc1cc(-c2cnn3cc(N4CCC(N5CCN(C)CC5)CC4)cnc23)c2ccc(F)cc2n1]

or a pharmaceutically acceptable salt thereof.

In some embodiments, the compound of Formula I has a structure of Formula I-129:

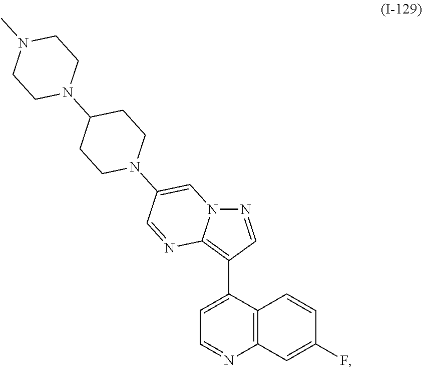 [SMILES: CN1CCN(C2CCN(c3cnc4c(-c5ccnc6cc(F)ccc56)cnn4c3)CC2)CC1]

or a pharmaceutically acceptable salt thereof.

In some embodiments, the compound of Formula I has a structure of Formula I-130:

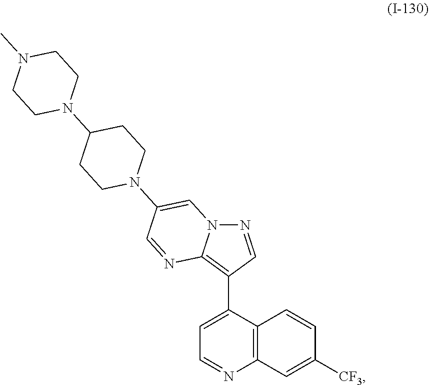 [SMILES: CN1CCN(C2CCN(c3cnc4c(-c5ccnc6cc(C(F)(F)F)ccc56)cnn4c3)CC2)CC1]

or a pharmaceutically acceptable salt thereof.

In some embodiments, the compound of Formula I has a structure of Formula II-131:

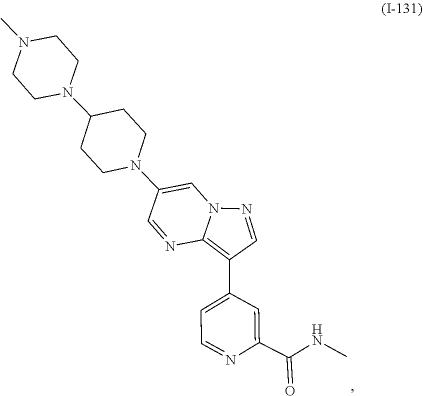

or a pharmaceutically acceptable salt thereof.

In some embodiments, the compound of Formula I has a structure of Formula I-132:

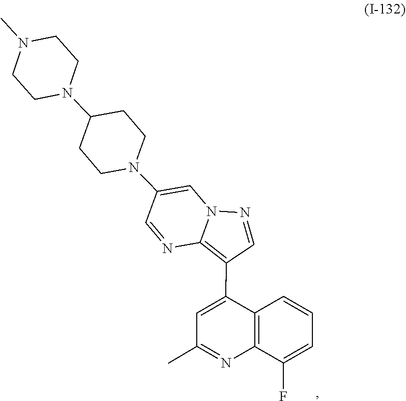 [SMILES: Cc1cc(-c2cnn3cc(N4CCC(N5CCN(C)CC5)CC4)cnc23)c2cccc(F)c2n1]

or a pharmaceutically acceptable sail thereof.

In some embodiments, the compound of Formula I has a structure of Formula I-133:

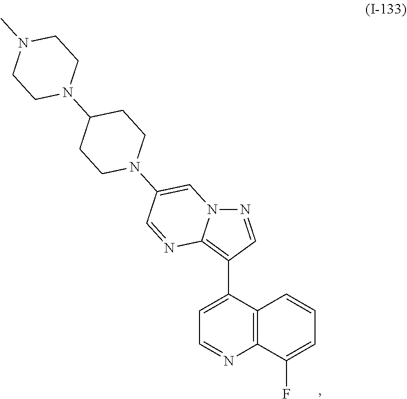 [SMILES: CN1CCN(C2CCN(c3cnc4c(-c5ccnc6c(F)cccc56)cnn4c3)CC2)CC1]

or a pharmaceutically acceptable salt thereof.

In some embodiments, the compound of Formula I has a structure of Formula II-134:

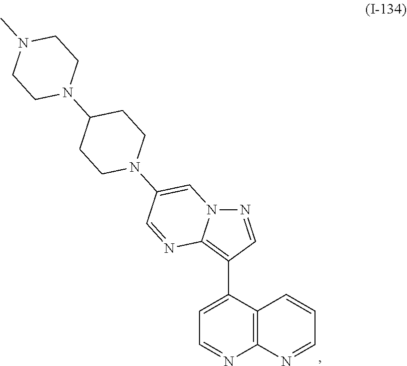 [SMILES: CN1CCN(C2CCN(c3cnc4c(-c5ccnc6ncccc56)cnn4c3)CC2)CC1]

or a pharmaceutically acceptable salt thereof.

In some embodiments, the compound of Formula I has a structure of Formula I-135:

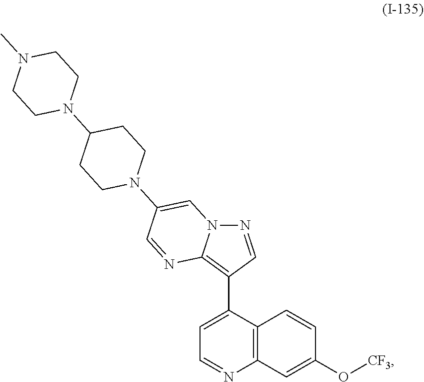 [SMILES: CN1CCN(C2CCN(c3cnc4c(-c5ccnc6cc(OC(F)(F)F)ccc56)cnn4c3)CC2)CC1]

or a pharmaceutically acceptable salt thereof.

In some embodiments, the compound of Formula I has a structure of Formula I-138:

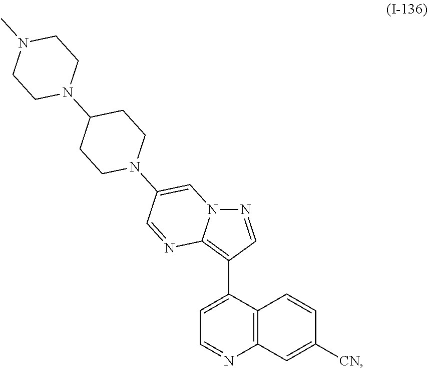 [SMILES: CN1CCN(C2CCN(c3cnc4c(-c5ccnc6cc(C#N)ccc56)cnn4c3)CC2)CC1]

In some embodiments, the compound of Formula I has a structure of Formula I-137:

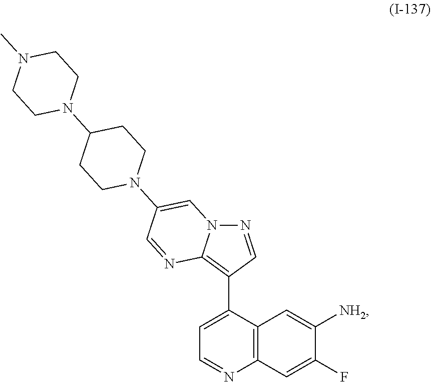 [SMILES: CN1CCN(C2CCN(c3cnc4c(-c5ccnc6cc(F)c(N)cc56)cnn4c3)CC2)CC1]

or a pharmaceutically acceptable salt thereof.

In some embodiments, the compound of Formula I has a structure of Formula I-138:

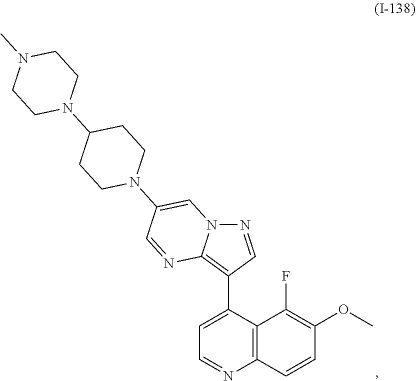

or a pharmaceutically acceptable salt thereof.

In some embodiments, the compound of Formula I has a structure of Formula I-139:

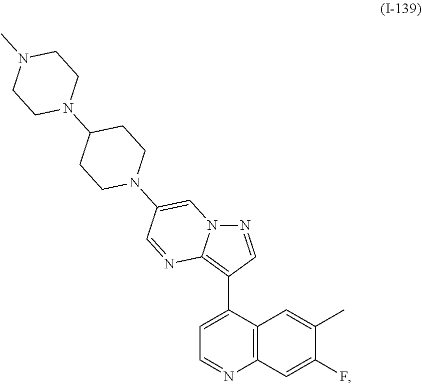 [SMILES: Cc1cc2c(-c3cnn4cc(N5CCC(N6CCN(C)CC6)CC5)cnc34)ccnc2cc1F]

In some embodiments, the compound of Formula I has a structure of Formula II-140:

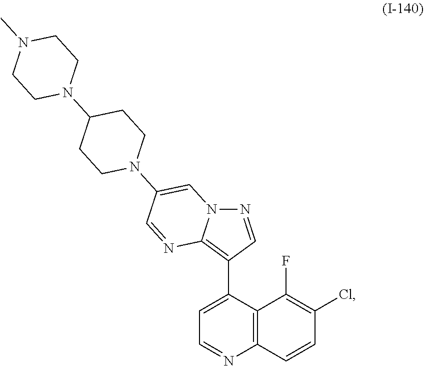 [SMILES: CN1CCN(C2CCN(c3cnc4c(-c5ccnc6ccc(Cl)c(F)c56)cnn4c3)CC2)CC1]

or a pharmaceutically acceptable salt thereof.

In some embodiments, the compound of Formula I has a structure of Formula I-141:

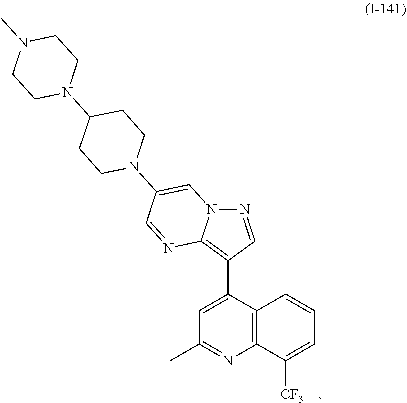 [SMILES: Cc1cc(-c2cnn3cc(N4CCC(N5CCN(C)CC5)CC4)cnc23)c2cccc(C(F)(F)F)c2n1]

or a pharmaceutically acceptable salt thereof.

In some embodiments, the compound of Formula I has a structure of Formula I-142:

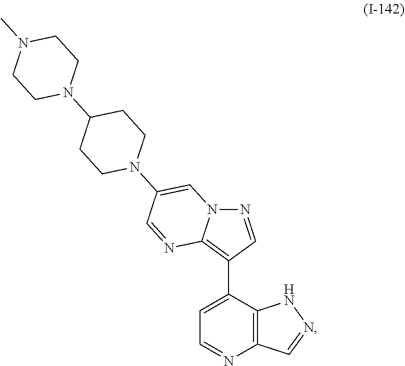 [SMILES: CN1CCN(C2CCN(c3cnc4c(-c5ccnc6cn[nH]c56)cnn4c3)CC2)CC1]

In some embodiments, the compound of Formula I has a structure of Formula I-143:

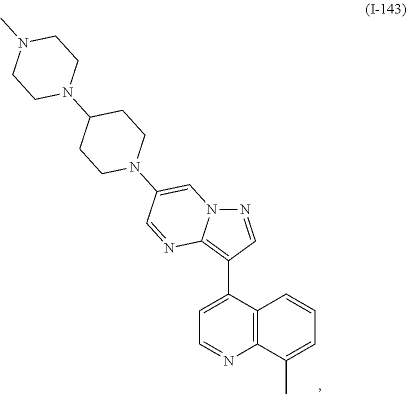 [SMILES: Cc1cccc2c(-c3cnn4cc(N5CCC(N6CCN(C)CC6)CC5)cnc34)ccnc12]

or a pharmaceutically acceptable sail thereof.

In some embodiments, the compound of Formula I has a structure of Formula II-144:

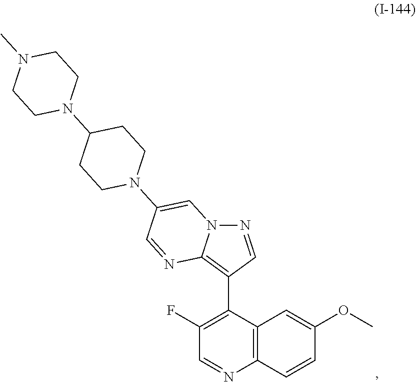

or a pharmaceutically acceptable salt thereof.

In some embodiments, the compound of Formula I has a structure of Formula I-145:

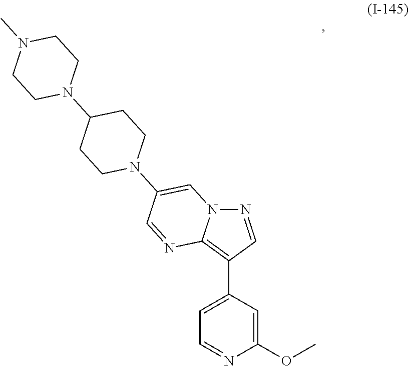

or a pharmaceutically acceptable salt thereof.

In some embodiments, the compound of Formula I has a structure of Formula II-146:

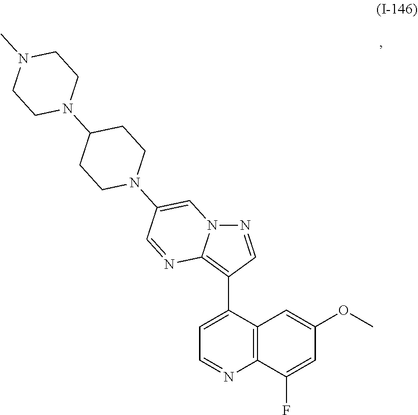

or a pharmaceutically acceptable salt thereof.

In some embodiments, the compound of Formula I has a structure of Formula II-147:

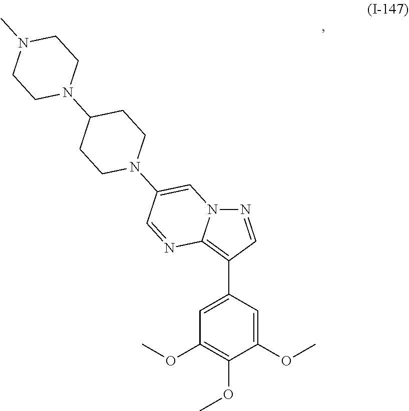

or a pharmaceutically acceptable salt thereof.

In some embodiments, the compound of Formula I has a structure of Formula II-148:

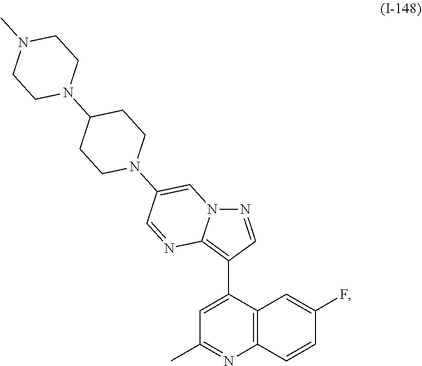 [SMILES: Cc1cc(-c2cnn3cc(N4CCC(N5CCN(C)CC5)CC4)cnc23)c2cc(F)ccc2n1]

or a pharmaceutically acceptable salt thereof.

In some embodiments, the compound of Formula I has a structure of Formula II-149:

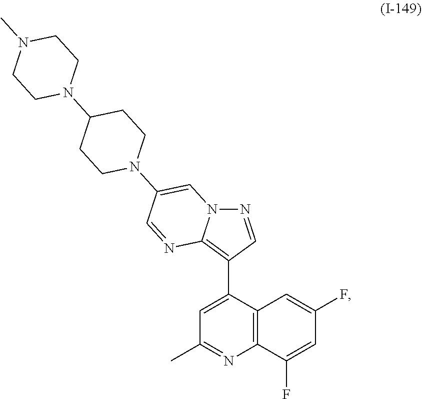 [SMILES: Cc1cc(-c2cnn3cc(N4CCC(N5CCN(C)CC5)CC4)cnc23)c2cc(F)cc(F)c2n1]

or a pharmaceutically acceptable sail thereof.

In some embodiments, the compound of Formula I has a structure of Formula I-150:

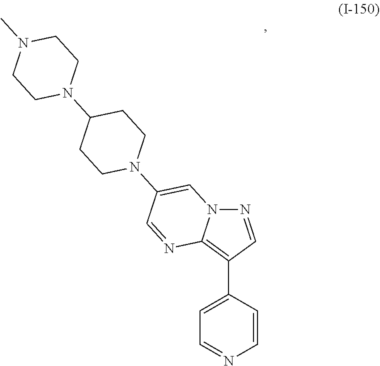 [SMILES: CN1CCN(C2CCN(c3cnc4c(-c5ccncc5)cnn4c3)CC2)CC1]

or a pharmaceutically acceptable salt thereof.

In some embodiments, the compound of Formula I has a structure of Formula II-151:

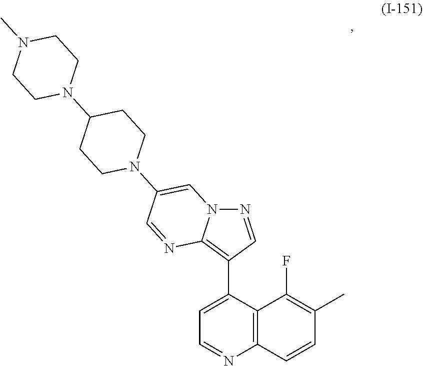

or a pharmaceutically acceptable salt thereof.

In some embodiments, the compound of Formula I has a structure of Formula I-152:

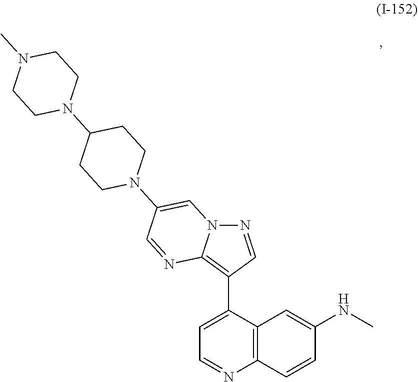

or a pharmaceutically acceptable salt thereof.

In some embodiments, the compound of Formula I has a structure of Formula I-153:

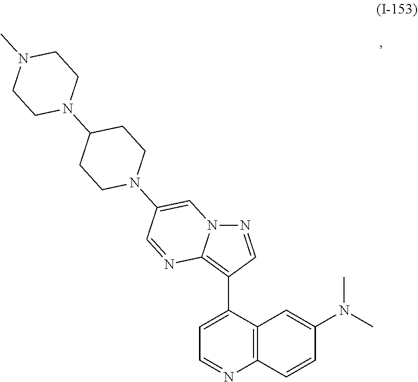

or a pharmaceutically acceptable salt thereof.

In some embodiments, the compound of Formula I has a structure of Formula II-154:

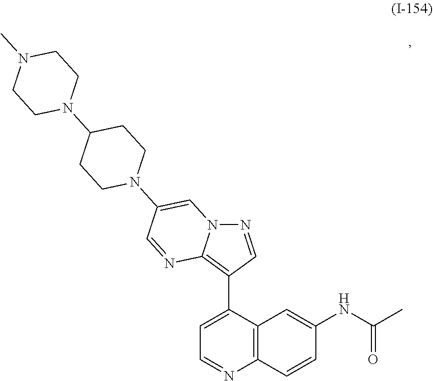 [SMILES: CC(=O)Nc1ccc2nccc(-c3cnn4cc(N5CCC(N6CCN(C)CC6)CC5)cnc34)c2c1]

or a pharmaceutically acceptable salt thereof.

In some embodiments, the compound of Formula I has a structure of Formula I-155:

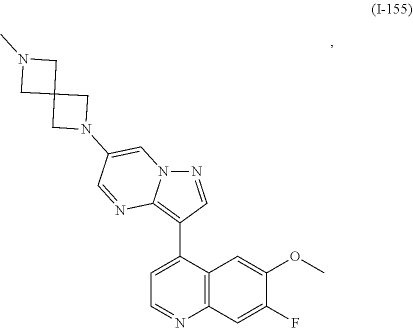

or a pharmaceutically acceptable salt thereof.

In some embodiments, the compound of Formula I has a structure of Formula I-156:

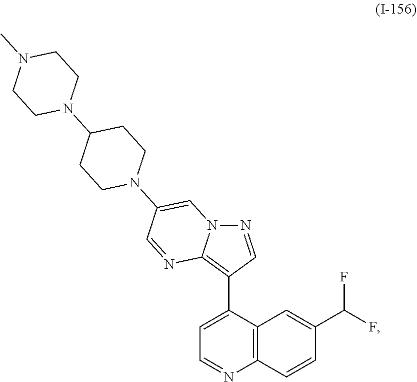 [SMILES: CN1CCN(C2CCN(c3cnc4c(-c5ccnc6ccc(C(F)F)cc56)cnn4c3)CC2)CC1]

or a pharmaceutically acceptable salt thereof.

In some embodiments, the compound of Formula I has a structure of Formula I-157:

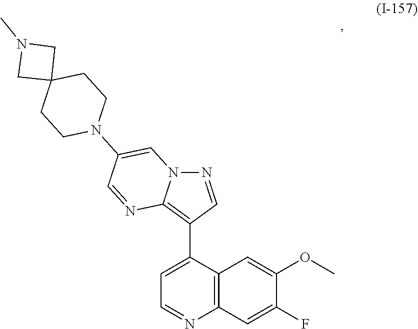

In some embodiments, the compound of Formula I has a structure of Formula I-158:

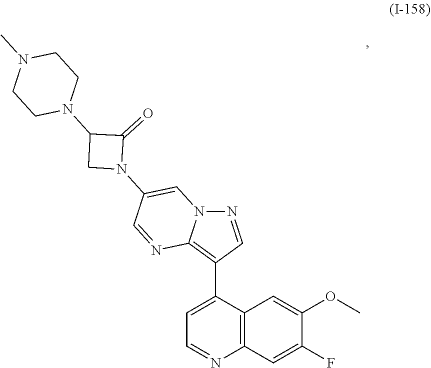

or a pharmaceutically acceptable salt thereof.

In some embodiments, the compound of Formula I has a structure of Formula I-159:

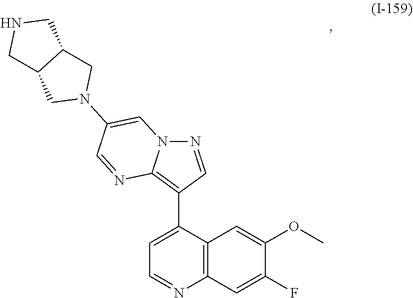 [SMILES: COc1cc2c(-c3cnn4cc(N5C[C@H]6CNC[C@H]6C5)cnc34)ccnc2cc1F]

or a pharmaceutically acceptable salt thereof.

In some embodiments, the compound of Formula I has a structure of Formula I-160:

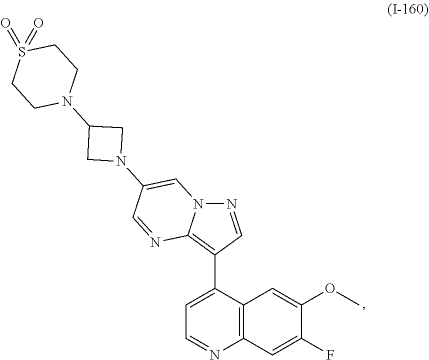 [SMILES: COc1cc2c(-c3cnn4cc(N5CC(N6CCS(=O)(=O)CC6)C5)cnc34)ccnc2cc1F]

or a pharmaceutically acceptable salt thereof.

In some embodiments, the compound of Formula I has a structure of Formula II-161:

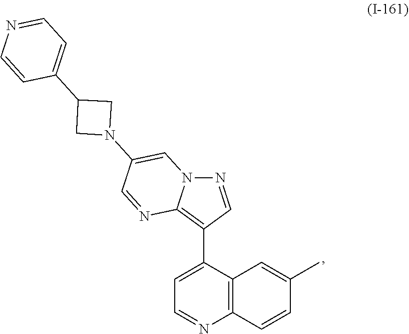 [SMILES: Cc1ccc2nccc(-c3cnn4cc(N5CC(c6ccncc6)C5)cnc34)c2c1]

or a pharmaceutically acceptable salt thereof.

In some embodiments, the compound of Formula I has a structure of Formula I-162:

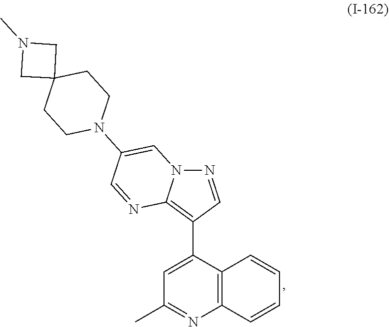 [SMILES: Cc1cc(-c2cnn3cc(N4CCC5(CC4)CN(C)C5)cnc23)c2ccccc2n1]

or a pharmaceutically acceptable salt thereof.

In some embodiments, the compound of Formula I has a structure of Formula II-163:

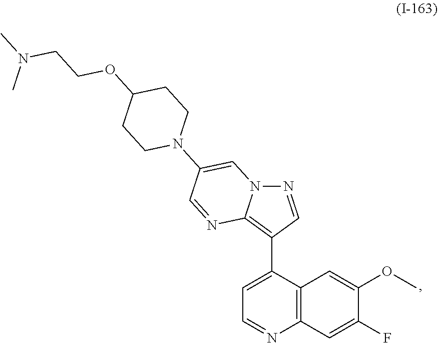

or a pharmaceutically acceptable salt thereof.

In some embodiments, the compound of Formula I has a structure of Formula I-164:

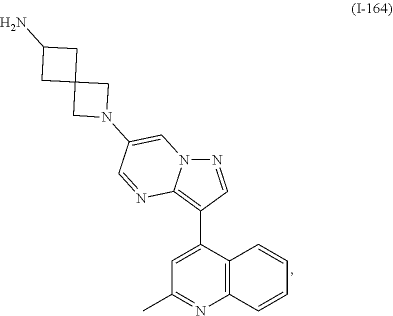 [SMILES: Cc1cc(-c2cnn3cc(N4CC5(CC(N)C5)C4)cnc23)c2ccccc2n1]

or a pharmaceutically acceptable salt thereof.

In some embodiments, the compound of Formula I has a structure of Formula II-165:

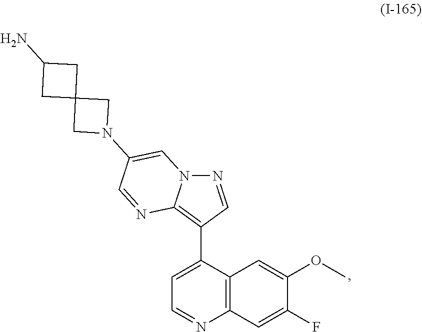 [SMILES: COc1cc2c(-c3cnn4cc(N5CC6(CC(N)C6)C5)cnc34)ccnc2cc1F]

or a pharmaceutically acceptable salt thereof.

In some embodiments, the compound of Formula I has a structure of Formula I-166:

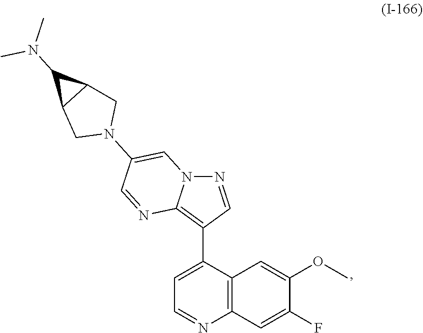

or a pharmaceutically acceptable salt thereof.

In some embodiments, the compound of Formula I has a structure of Formula I-167:

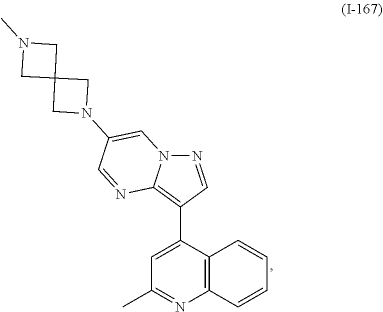 [SMILES: Cc1cc(-c2cnn3cc(N4CC5(CN(C)C5)C4)cnc23)c2ccccc2n1]

or a pharmaceutically acceptable salt thereof.

In some embodiments, the compound of Formula I has a structure of Formula II-168:

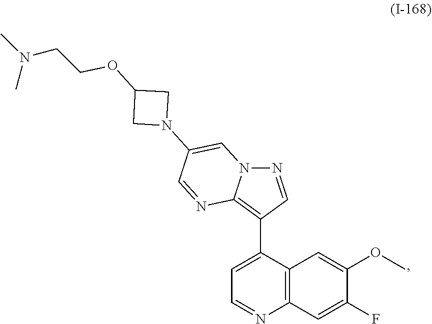

or a pharmaceutically acceptable salt thereof.

In some embodiments, the compound of Formula I has a structure of Formula II-169:

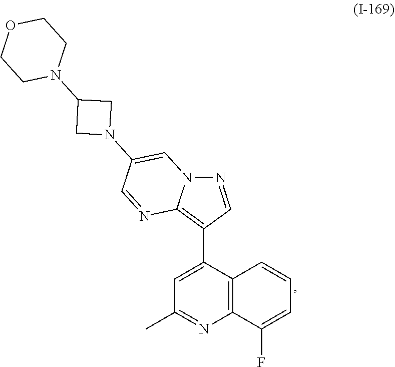 [SMILES: Cc1cc(-c2cnn3cc(N4CC(N5CCOCC5)C4)cnc23)c2cccc(F)c2n1]

or a pharmaceutically acceptable salt thereof.

In some embodiments, the compound of Formula I has a structure of Formula I-170:

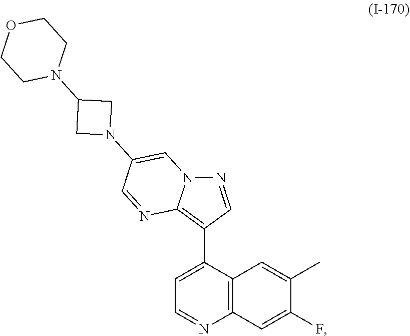 [SMILES: Cc1cc2c(-c3cnn4cc(N5CC(N6CCOCC6)C5)cnc34)ccnc2cc1F]

or a pharmaceutically acceptable salt thereof.

In some embodiments, the compound of Formula I has a structure of Formula I-171:

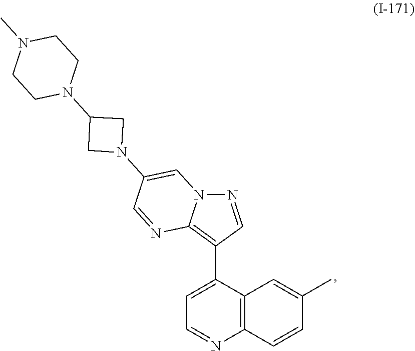

or a pharmaceutically acceptable salt thereof.

In some embodiments, the compound of Formula I has a structure of Formula I-172:

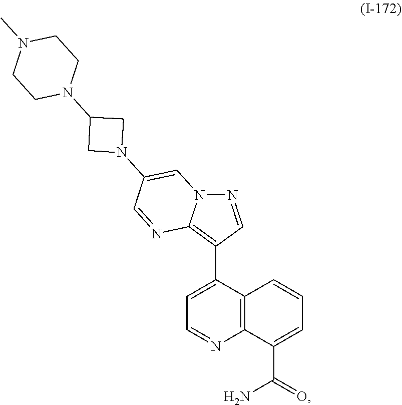 [SMILES: CN1CCN(C2CN(c3cnc4c(-c5ccnc6c(C(N)=O)cccc56)cnn4c3)C2)CC1]

or a pharmaceutically acceptable salt thereof.

In some embodiments, the compound of Formula I has a structure of Formula II-173:

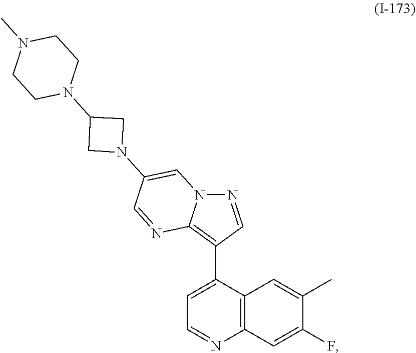 [SMILES: Cc1cc2c(-c3cnn4cc(N5CC(N6CCN(C)CC6)C5)cnc34)ccnc2cc1F]

or a pharmaceutically acceptable salt thereof.

In some embodiments, the compound of Formula I has a structure of Formula I-174:

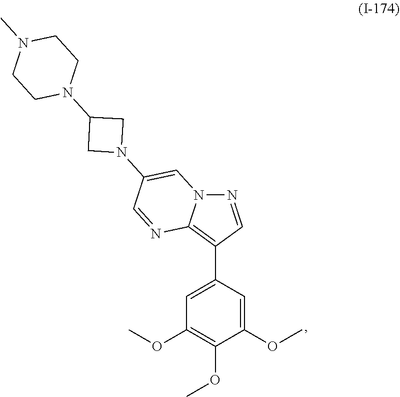

or a pharmaceutically acceptable salt thereof.

In some embodiments, the compound of Formula I has a structure of Formula II-175:

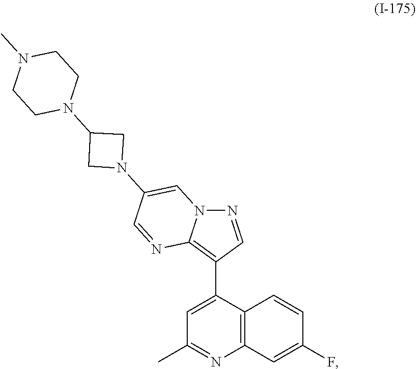 [SMILES: Cc1cc(-c2cnn3cc(N4CC(N5CCN(C)CC5)C4)cnc23)c2ccc(F)cc2n1]

or a pharmaceutically acceptable salt thereof.

In some embodiments, the compound of Formula I has a structure of Formula II-176:

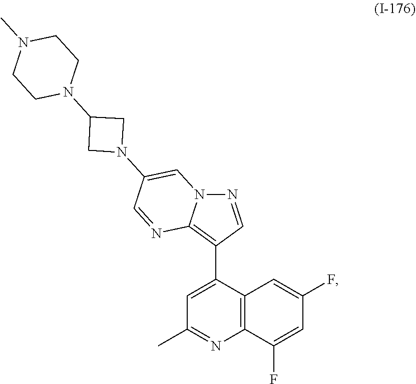 [SMILES: Cc1cc(-c2cnn3cc(N4CC(N5CCN(C)CC5)C4)cnc23)c2cc(F)cc(F)c2n1]

or a pharmaceutically acceptable salt thereof.

In some embodiments, the compound of Formula I has a structure of Formula I-177:

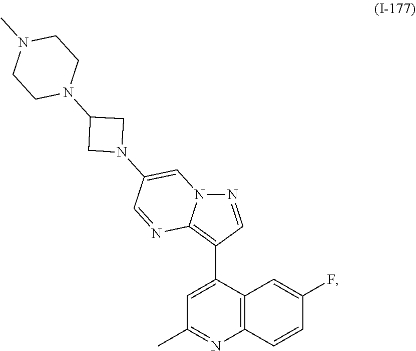 [SMILES: Cc1cc(-c2cnn3cc(N4CC(N5CCN(C)CC5)C4)cnc23)c2cc(F)ccc2n1]

or a pharmaceutically acceptable salt thereof.

In some embodiments, the compound of Formula I has a structure of Formula I-178:

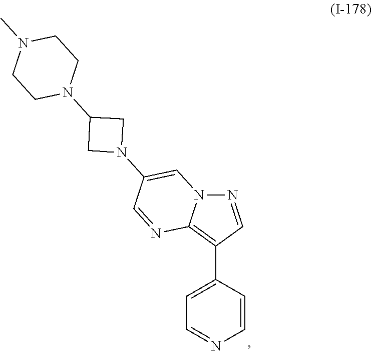 [SMILES: CN1CCN(C2CN(c3cnc4c(-c5ccncc5)cnn4c3)C2)CC1]

or a pharmaceutically acceptable salt thereof.

In some embodiments, the compound of Formula I has a structure of Formula II-179:

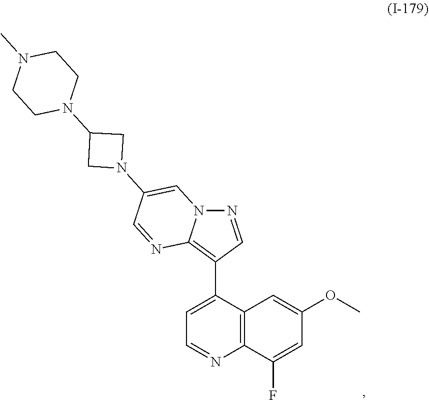

or a pharmaceutically acceptable sail thereof.

In some embodiments, the compound of Formula I has a structure of Formula II-180:

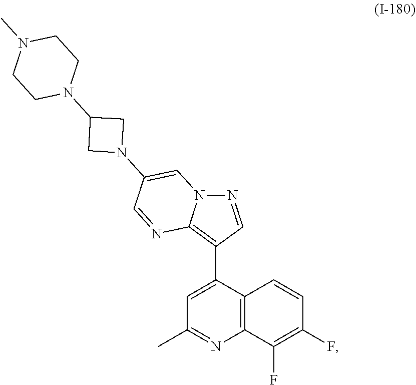 [SMILES: Cc1cc(-c2cnn3cc(N4CC(N5CCN(C)CC5)C4)cnc23)c2ccc(F)c(F)c2n1]

In some embodiments, the compound of Formula I has a structure of Formula II-181:

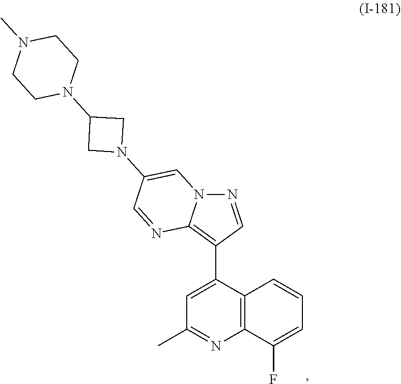 [SMILES: Cc1cc(-c2cnn3cc(N4CC(N5CCN(C)CC5)C4)cnc23)c2cccc(F)c2n1]

or a pharmaceutically acceptable salt thereof.

In some embodiments, the compound of Formula I has a structure of Formula II-182:

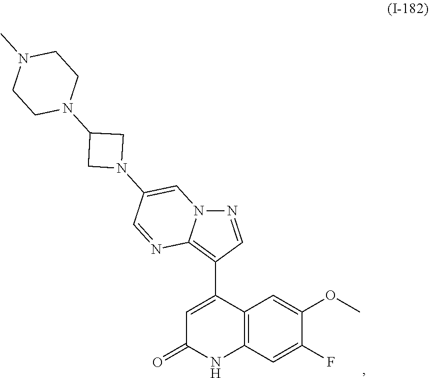

or a pharmaceutically acceptable salt thereof.

In some embodiments, the compound of Formula I has a structure of Formula II-183:

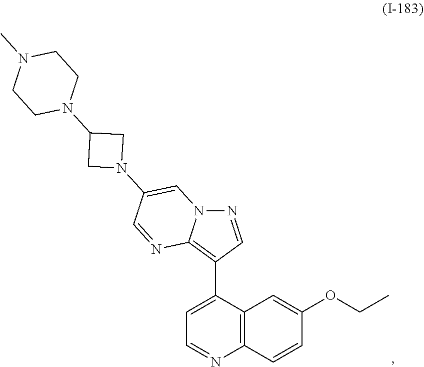 [SMILES: CCOc1ccc2nccc(-c3cnn4cc(N5CC(N6CCN(C)CC6)C5)cnc34)c2c1]

or a pharmaceutically acceptable salt thereof.

In some embodiments, the compound of Formula I has a structure of Formula II-184:

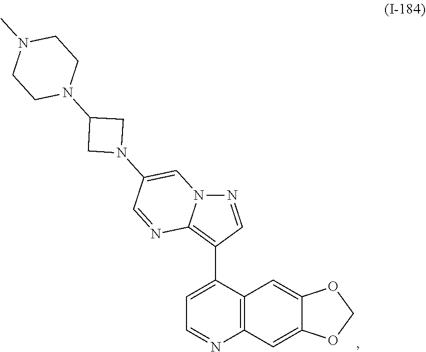 [SMILES: CN1CCN(C2CN(c3cnc4c(-c5ccnc6cc7c(cc56)OCO7)cnn4c3)C2)CC1]

or a pharmaceutically acceptable salt thereof.

In some embodiments, the compound of Formula I has a structure of Formula II-185:

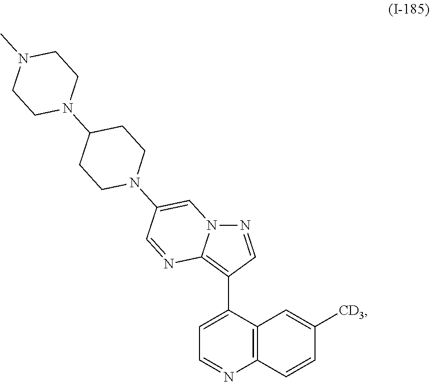 [SMILES: [2H]C([2H])([2H])c1ccc2nccc(-c3cnn4cc(N5CCC(N6CCN(C)CC6)CC5)cnc34)c2c1]

or a pharmaceutically acceptable salt thereof.

In some embodiments, the compound of Formula I has a structure of Formula I-186:

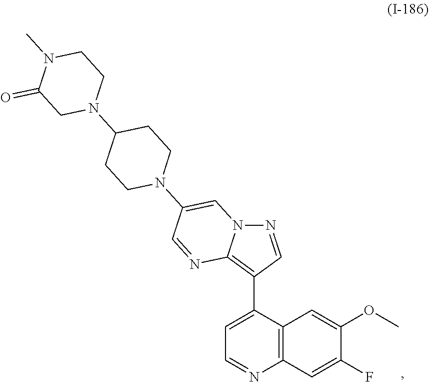 [SMILES: COc1cc2c(-c3cnn4cc(N5CCC(N6CCN(C)C(=O)C6)CC5)cnc34)ccnc2cc1F]

or a pharmaceutically acceptable salt thereof.

In some embodiments, the compound of Formula I has a structure of Formula II-187:

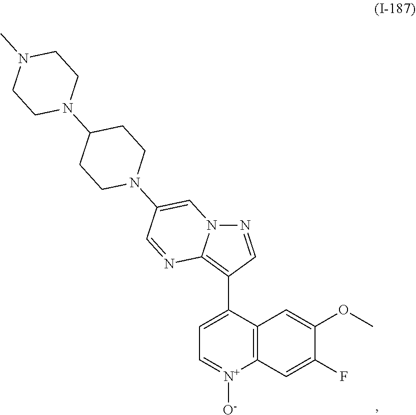

or a pharmaceutically acceptable salt thereof.

In some embodiments, the compound of Formula I has a structure of Formula I-188:

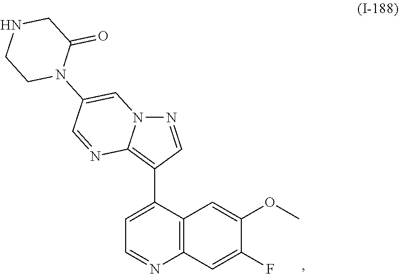 [SMILES: COc1cc2c(-c3cnn4cc(N5CCNCC5=O)cnc34)ccnc2cc1F]

or a pharmaceutically acceptable salt thereof.

In some embodiments, the compound of Formula I has a structure of Formula I-189:

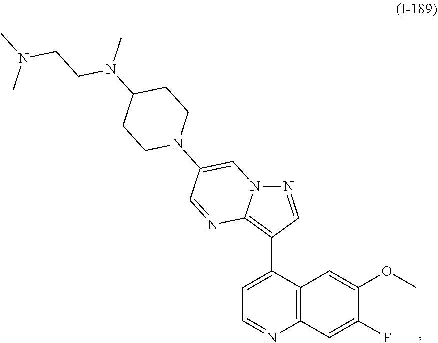

or a pharmaceutically acceptable salt thereof.

In some embodiments, the compound of Formula I has a structure of Formula I-190:

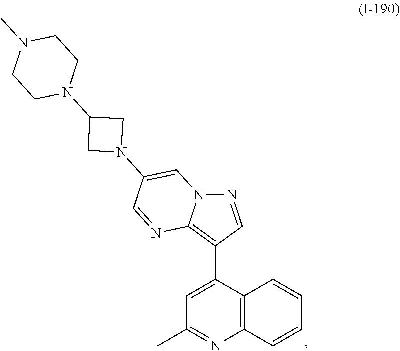 [SMILES: Cc1cc(-c2cnn3cc(N4CC(N5CCN(C)CC5)C4)cnc23)c2ccccc2n1]

or a pharmaceutically acceptable salt thereof.

In some embodiments, the compound of Formula I has a structure of Formula I-191:

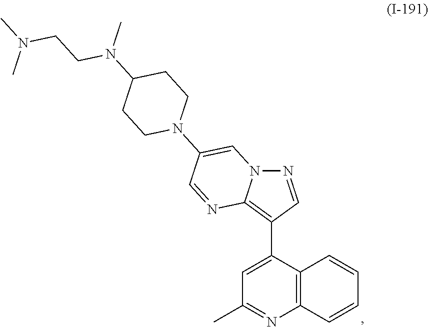 [SMILES: Cc1cc(-c2cnn3cc(N4CCC(N(C)CCN(C)C)CC4)cnc23)c2ccccc2n1]

or a pharmaceutically acceptable salt thereof.

In some embodiments, the compound of Formula I has a structure of Formula I-192:

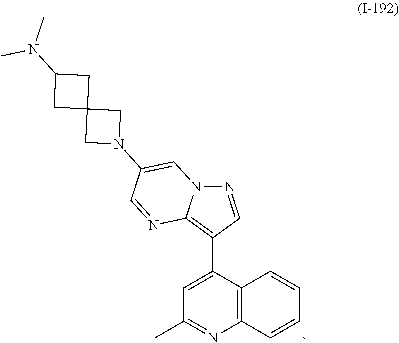 [SMILES: Cc1cc(-c2cnn3cc(N4CC5(CC(N(C)C)C5)C4)cnc23)c2ccccc2n1]

or a pharmaceutically acceptable salt thereof.

In some embodiments, the compound of Formula I has a structure of Formula I-193:

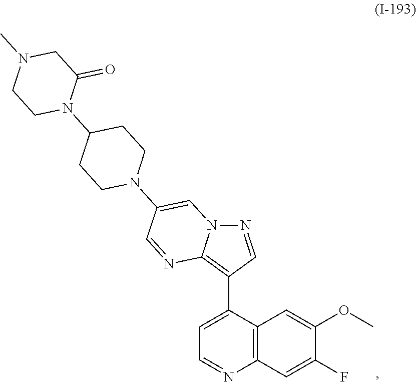

or a pharmaceutically acceptable salt thereof.

In some embodiments, the compound of Formula I has a structure of Formula I-194:

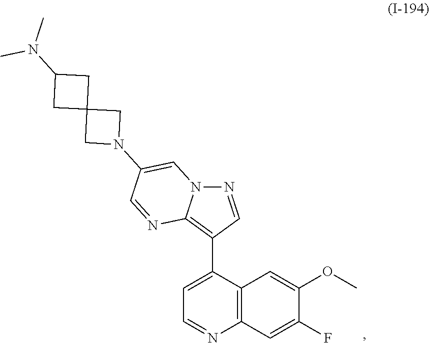

or a pharmaceutically acceptable salt thereof.

In some embodiments, the compound of Formula I has a structure of Formula II-195:

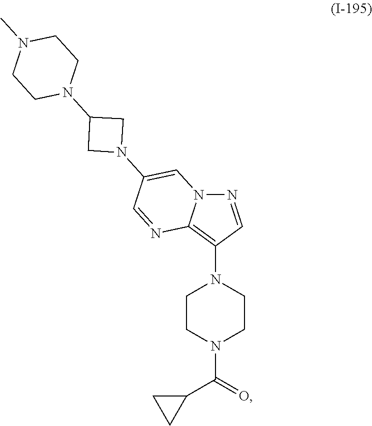 [SMILES: CN1CCN(C2CN(c3cnc4c(N5CCN(C(=O)C6CC6)CC5)cnn4c3)C2)CC1]

or a pharmaceutically acceptable salt thereof.

In some embodiments, the compound of Formula I has a structure of Formula II-196:

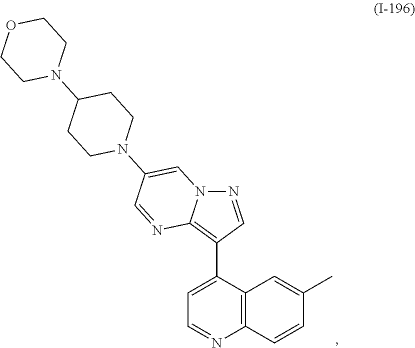 [SMILES: Cc1ccc2nccc(-c3cnn4cc(N5CCC(N6CCOCC6)CC5)cnc34)c2c1]

or a pharmaceutically acceptable salt thereof.

In some embodiments, the compound of Formula I has a structure of Formula I-197:

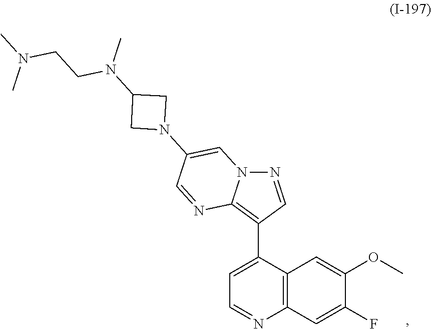

or a pharmaceutically acceptable salt thereof.

In some embodiments, the compound of Formula I has a structure of Formula II-198:

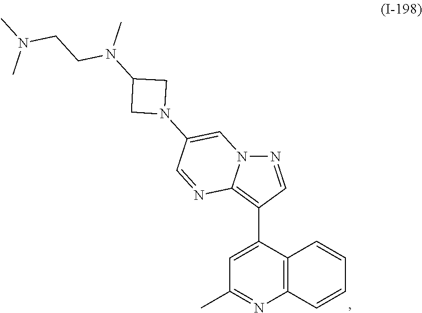 [SMILES: Cc1cc(-c2cnn3cc(N4CC(N(C)CCN(C)C)C4)cnc23)c2ccccc2n1]

or a pharmaceutically acceptable salt thereof.

In some embodiments, the compound of Formula I has a structure of Formula I-199:

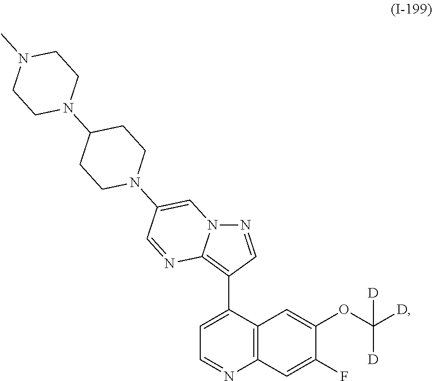

or a pharmaceutically acceptable salt thereof.

In some embodiments, the compound of Formula I has a structure of Formula II-200:

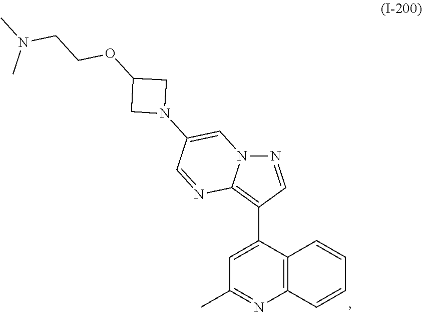 [SMILES: Cc1cc(-c2cnn3cc(N4CC(OCCN(C)C)C4)cnc23)c2ccccc2n1]

or a pharmaceutically acceptable salt thereof.

Additional compounds of Formula I are described US Patent Application Publication No. 2020/0179389, which is incorporated herein by reference.

In some embodiments, the ALK2 inhibitor is a compound of Formula II:

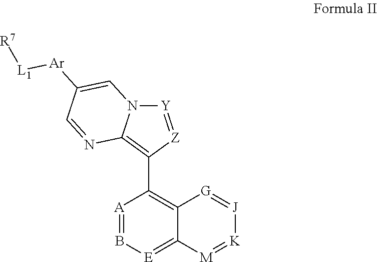

or a pharmaceutically acceptable salt thereof, wherein
X and Y are independently selected from CR¹⁵ and N, preferably both N;
Z is selected from CR³′ and N, preferably CR³′, most preferably CH;
Ar is a substituted or unsubstituted aryl ring or a substituted or unsubstituted heteroaryl ring;
L₁ is absent or selected from substituted or unsubstituted alkyl, substituted or unsubstituted cycloalkyl, substituted or unsubstituted cycloalkylalkyl, cycloalkyl-heteroalkyl, substituted or unsubstituted heterocyclyl, substituted or unsubstituted heterocyclylalkyl, substituted or unsubstituted heterocyclyl-heteroalkyl, and substituted or unsubstituted heteroalkyl; and
J and K are both absent or, independently for each occurrence, are each CR¹⁶;
A is CR¹⁶;
B and E are each independently CR¹⁷;
if J and K are absent, then G is R¹⁸ and M is R¹⁷; if J and K are not absent, then G is CR¹⁸ and M is CR¹⁷;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from

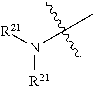

and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido, preferably H;
R¹⁶, independently for each occurrence, is selected from H, OH, halogen, cyano, carboxyl, and substituted or unsubstituted acyl, alkanol, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkoxy, alkylthio, acyloxy, amino, acylamino, carbamate, amido, amidino, sulfonyl, sulfoxido, sulfamoyl, or sulfonamide;
R¹⁷, independently for each occurrence, is selected from R¹⁶ and —R²², —NH₂, —NHR²², —N(R²²)₂, halogen, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²)═NH, —C(NHR²²)═NR²², —C(N(R²²)₂)—NH, —C(N(R²²)₂)—NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²,

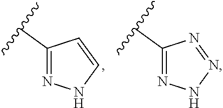 [SMILES: *c1cc[nH]n1 ; *c1nn[nH]n1]

—CH(OH)R²², —C(OH)(R²²)₂, —CH(NH₂)(R²²), —CH(NHR²²)(R²²), —CH(N(R²²)₂)(R²²), pyrazol-3-yl, pyrazol-4-yl, and —OR²², provided that at least one R¹⁷ is —R²², —NH₂, —NHR²², —N(R²²)₂, halogen, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)═NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²,

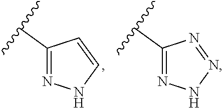 [SMILES: *c1cc[nH]n1 ; *c1nn[nH]n1]

—CH(OH)R²²—C(OH)(R²²)₂, —CH(NH₂)(R²²), —CH(NHR²²)(R²²), —CH(N(R²²)₂)(R²²), pyrazol-3-yl, pyrazol-4-yl, or —OR²²;
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide, preferably from H and substituted or unsubstituted alkyl, aralkyl, cycloalkyl, heterocyclyl, aryl, heteroaryl, heteroaralkyl, cycloalkylalkyl, heterocyclylalkyl, more preferably from H and substituted or unsubstituted alkyl, and most preferably from H and lower alkyl, such as methyl or ethyl; and
R²², independently for each occurrence, is selected from lower alkyl (e.g., CH₃ or CF₃) and cycloalkyl (preferably cyclopropyl or cyclobutyl).

In some embodiments, the ALK2 inhibitor is a compound of Formula II or a pharmaceutically acceptable salt thereof, wherein
X and Y are each N;
Z is CR³;
Ar is a substituted or unsubstituted aryl ring or a substituted or unsubstituted heteroaryl ring;
L₁ is absent or selected from substituted or unsubstituted cycloalkyl, substituted or unsubstituted cycloalkylalkyl, cycloalkyl-heteroalkyl, substituted or unsubstituted heterocyclyl, substituted or unsubstituted heterocyclylalkyl, substituted or unsubstituted heterocyclylheteroalkyl, and

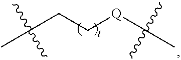

wherein Q is selected from CR¹⁰′R¹¹, NR¹², O, S, S(O), and SO₂; R¹⁰ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido; R¹² is selected from H and substituted or unsubstituted alkyl, cycloalkyl, heterocyclyl, heterocyclylalkyl, amino, acylamino, carbamate, amido, amidino, sulfonyl, sulfamoyl, or sulfonamide; and t is an integer selected from 0, 2, 3, and 4, wherein any CH₂ subunit of L₁ is optionally substituted with one or two lower alkyl groups, or represents a carbon atom in a 3-5-membered cycloalkyl or heterocyclyl ring; and
J and K are both absent or, independently for each occurrence, are each CR¹⁶;
A is CR 16;
B and E are each independently CR¹⁷;
if J and K are absent, then G is R¹⁶ and M is R¹⁷; if J and K are not absent, then G is CR¹⁶ and M is CR¹⁷;
R³ is H;
R⁷ is selected from

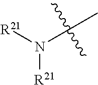

and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, OH, cyano, carboxyl, and substituted or unsubstituted acyl, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkylthio, acyloxy, amino, acylamino, carbamate, amido, amidino, sulfonyl, sulfoxido, sulfamoyl, or sulfonamide; R¹⁷, independently for each occurrence, is selected from R¹⁶ and —R²², —NH₂, —NHR²², —N(R²²)₂, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)═NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²,

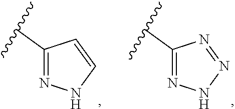 [SMILES: *c1cc[nH]n1 ; *c1nn[nH]n1]

—CH(OH)R²², —C(OH)(R²²)₂, —CH(NH₂)(R²²), —CH(NHR²²)(R²²), —CH(N(R²²)₂)(R²²), pyrazol-3-yl, pyrazol-4-yl, and —OR²², provided that at least one R¹⁷ is —R²², —NH₂, —NHR²², —N(R²²)₂, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)—NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²²

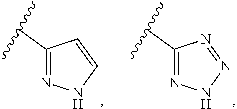 [SMILES: *c1cc[nH]n1 ; *c1nn[nH]n1]

—CH(OH)R²², —C(OH)(R²²)₂, —CH(NH₂)(R²²), —CH(NHR²²)(R²²), —CH(N(R²²)₂)(R²²), pyrazol-3-yl, or pyrazol-4-yl,
where at least one R¹⁷ represents a moiety selected from—CO₂H, —CONH₂, —CH₂OH, —CN, —C(O)CH₃, —CH(OH) CH₃, —C(OH)(CH₃)₂, —C(O)CF₃, —CH(NH₂) CF₃, —SO₂CH₃, —SO₂NH₂ and

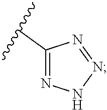 [SMILES: *c1nn[nH]n1]

R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide; and
R²², independently for each occurrence, is selected from lower alkyl and cycloalkyl;
wherein at least one R¹⁶ or one R¹⁷ is not H.

In some embodiments, the ALK2 inhibitor is a compound of Formula II or a pharmaceutically acceptable salt thereof, wherein
X and Y are each N;
Z is CR³;
Ar is a substituted or unsubstituted aryl ring or a substituted or unsubstituted heteroaryl ring;
L₁ is absent or

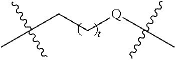

wherein Q is selected from CR¹⁰′R¹¹, NR¹², O, S, S(O), and SO₂; R¹⁰′ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido; R¹² is selected from H and substituted or unsubstituted alkyl, cycloalkyl, heterocyclyl, heterocyclylalkyl, amino, acylamino, carbamate, amido, amidino, sulfonyl, sulfamoyl, or sulfonamide; and t is selected from 0, 2, 3, and 4, wherein any CH₂ subunit of L₁ is optionally substituted with one or two lower alkyl groups, or represents a carbon atom in a 3-5-membered cycloalkyl or heterocyclyl ring; and
J and K are both absent or, independently for each occurrence, are each CR¹⁶;
A and B, independently for each occurrence, are CR¹⁶; E is CR¹⁷;
if J and K are absent, then G and M are each independently R¹⁸; if J and K are not absent, then G and M are each independently CR¹⁷;
R³ is H;
R⁷ is

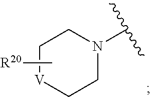

V is NR³⁰;
R²⁰ is absent or represents from 1-6 substituents on the ring to which it is attached, independently selected from substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfoxido, sulfamoyl, and sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, OH, cyano, carboxyl, and substituted or unsubstituted acyl, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkylthio, acyloxy, amino, acylamino, carbamate, amido, amidino, sulfonyl, sulfoxido, sulfamoyl, sulfonamide, tetrazolyl, or trifluoromethylacyl;
R¹⁷, independently for each occurrence, is selected from R¹⁶ and H, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃,

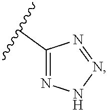 [SMILES: *c1nn[nH]n1]

—CH(OH) CH₃, —C(O)CF₃, and —OCH₃, provided that at least one R¹⁷ is H, —CO₂H, —CONH₂, —CONHCH₃, —CON(CH₃)₂, —C(NH₂)—N(OH), —C(NH₂)═NH, —CN, —CH₂OH, —SO₂NH₂, —CH₂NH₂, —C(O)CH₃,

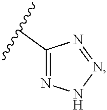 [SMILES: *c1nn[nH]n1]

—CH(OH) CH₃, or —C(O)CF₃; and
R³⁰, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide;
wherein at least one R¹⁶ or one R¹⁷ is not H.

In other embodiments, the ALK2 inhibitor is a compound of Formula II or a pharmaceutically acceptable salt thereof, wherein
X and Y are independently selected from CR¹⁵ and N, preferably both N;
Z is selected from CR³′ and N, preferably CR³′, most preferably CH;
Ar is a substituted or unsubstituted aryl ring (e.g., a substituted or unsubstituted phenyl ring) or a substituted or unsubstituted heteroaryl ring (e.g., a pyridyl or pyrimidyl ring);
L₁ is absent or selected from substituted or unsubstituted alkyl and heteroalkyl; and
J and K are both absent or, independently for each occurrence, are each CR¹⁶;
A and B, independently for each occurrence, are CR¹⁶; E is CR¹⁷;
if J and K are absent, then G and M are each independently R¹⁶; if J and K are not absent, then G and M are each independently CR¹⁷;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from

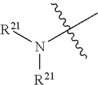

and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, D, OH, halogen, cyano, carboxyl, and substituted or unsubstituted acyl, alkanol, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkoxy, alkylthio, acyloxy, amino, acylamino, carbamate, amido, amidino, sulfonyl, sulfoxido, sulfamoyl, sulfonamide, tetrazolyl, or trifluoromethylacyl;
R¹⁷, independently for each occurrence, is selected from R¹⁶ and H, D, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃,

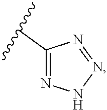 [SMILES: *c1nn[nH]n1]

—CH(OH) CH₃, —C(O)CF₃, and —OCH₃, provided that at least one R¹⁷ is H, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃,

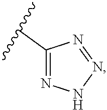 [SMILES: *c1nn[nH]n1]

—CH(OH) CH₃, —C(O)CF₃, or —OCH₃;
and

R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide.

Compounds of Formula II may be synthesized by methods known in the art, e.g., those described in U.S. Pat. No. 10,513,521, which is incorporated herein by reference.

In some embodiments, the compound of Formula II has a structure of Formula II-1:

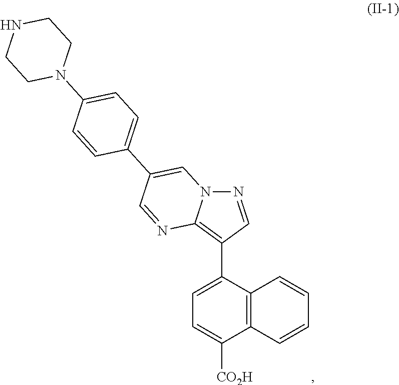 [SMILES: O=C(O)c1ccc(-c2cnn3cc(-c4ccc(N5CCNCC5)cc4)cnc23)c2ccccc12]

or a pharmaceutically acceptable salt thereof.

In some embodiments, the compound of Formula II has a structure of Formula II-2:

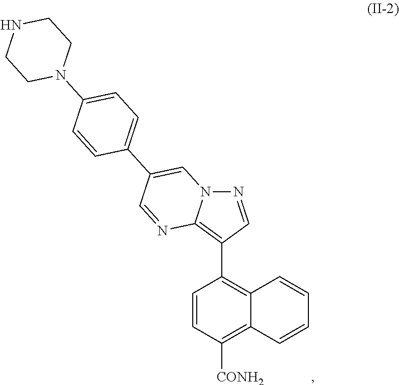 [SMILES: NC(=O)c1ccc(-c2cnn3cc(-c4ccc(N5CCNCC5)cc4)cnc23)c2ccccc12]

or a pharmaceutically acceptable salt thereof.

In some embodiments, the compound of Formula II has a structure of Formula II-3:

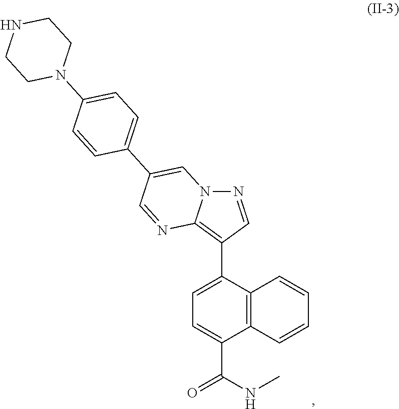 [SMILES: CNC(=O)c1ccc(-c2cnn3cc(-c4ccc(N5CCNCC5)cc4)cnc23)c2ccccc12]

In some embodiments, the compound of Formula II has a structure of Formula II-4:

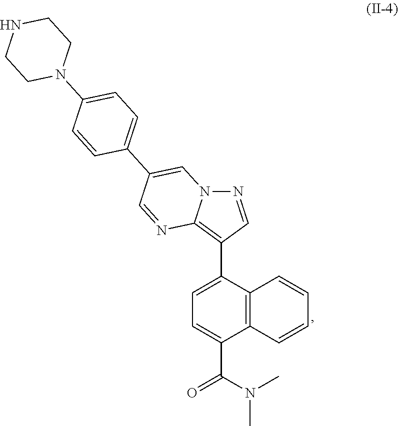 [SMILES: CN(C)C(=O)c1ccc(-c2cnn3cc(-c4ccc(N5CCNCC5)cc4)cnc23)c2ccccc12]

or a pharmaceutically acceptable salt thereof.

In some embodiments, the compound of Formula II has a structure of Formula II-5:

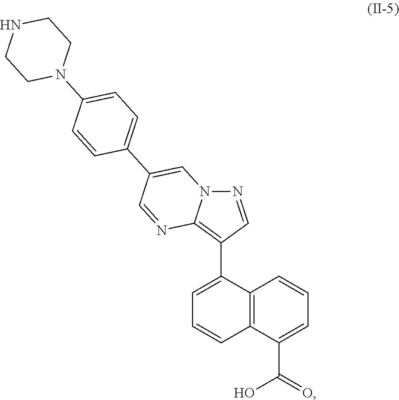 [SMILES: O=C(O)c1cccc2c(-c3cnn4cc(-c5ccc(N6CCNCC6)cc5)cnc34)cccc12]

or a pharmaceutically acceptable salt thereof.

In some embodiments, the compound of Formula II has a structure of Formula II-6:

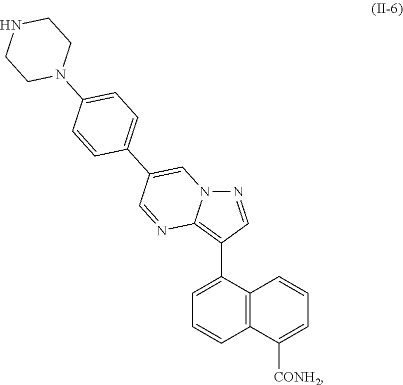 [SMILES: NC(=O)c1cccc2c(-c3cnn4cc(-c5ccc(N6CCNCC6)cc5)cnc34)cccc12]

or a pharmaceutically acceptable salt thereof.

In some embodiments, the compound of Formula II has a structure of Formula II-7:

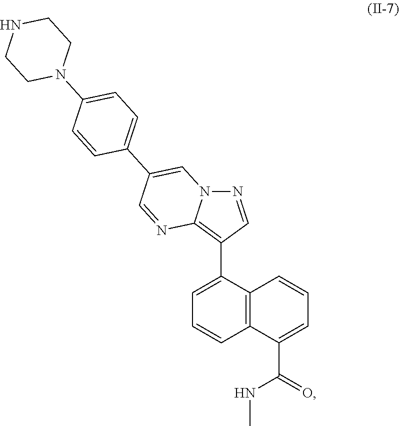 [SMILES: CNC(=O)c1cccc2c(-c3cnn4cc(-c5ccc(N6CCNCC6)cc5)cnc34)cccc12]

or a pharmaceutically acceptable salt thereof.

In some embodiments, the compound of Formula II has a structure of Formula II-8:

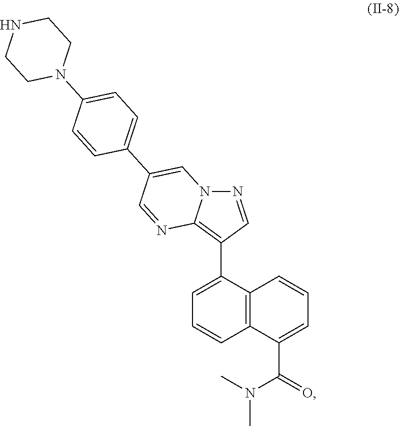 [SMILES: CN(C)C(=O)c1cccc2c(-c3cnn4cc(-c5ccc(N6CCNCC6)cc5)cnc34)cccc12]

or a pharmaceutically acceptable salt thereof.

In some embodiments, the compound of Formula II has a structure of Formula II-9:

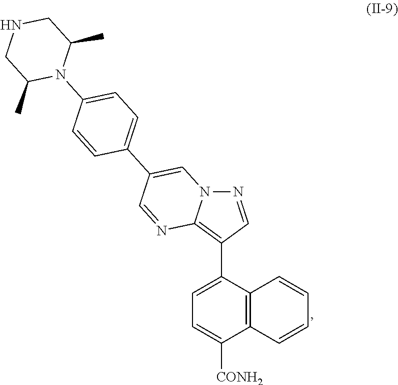 [SMILES: C[C@@H]1CNC[C@H](C)N1c1ccc(-c2cnc3c(-c4ccc(C(N)=O)c5ccccc45)cnn3c2)cc1]

In some embodiments, the compound of Formula II has a structure of Formula II-10:

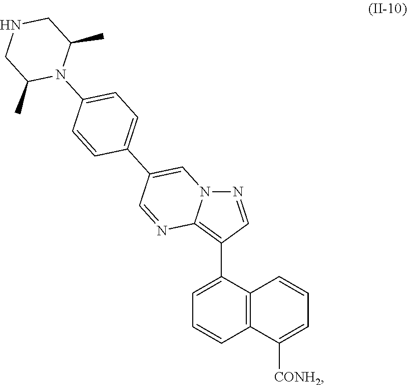 [SMILES: C[C@@H]1CNC[C@H](C)N1c1ccc(-c2cnc3c(-c4cccc5c(C(N)=O)cccc45)cnn3c2)cc1]

or a pharmaceutically acceptable salt thereof.

In some embodiments, the compound of Formula II has a structure of Formula II-11:

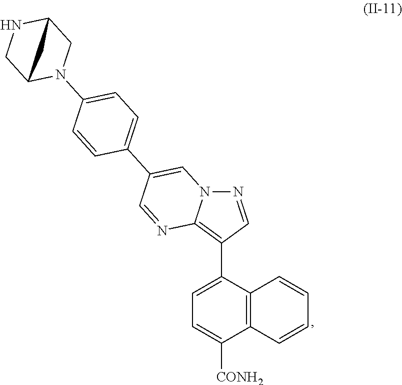 [SMILES: NC(=O)c1ccc(-c2cnn3cc(-c4ccc(N5C[C@@H]6C[C@H]5CN6)cc4)cnc23)c2ccccc12]

or a pharmaceutically acceptable salt thereof.

In some embodiments, the compound of Formula II has a structure of Formula II-12:

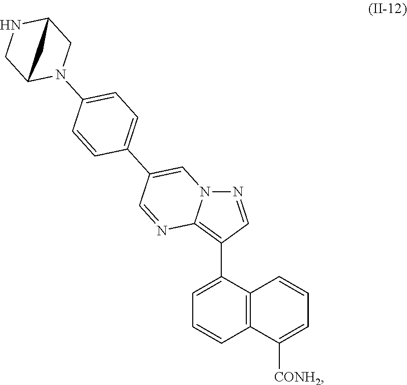 [SMILES: NC(=O)c1cccc2c(-c3cnn4cc(-c5ccc(N6C[C@@H]7C[C@H]6CN7)cc5)cnc34)cccc12]

In some embodiments, the compound of Formula II has a structure of Formula I-13:

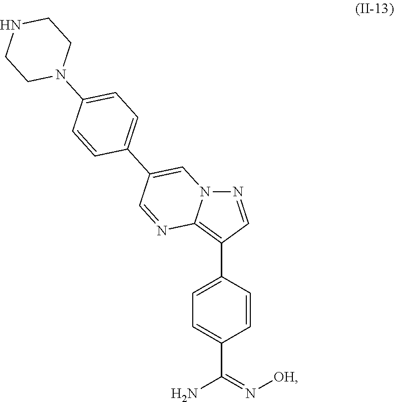 [SMILES: N/C(=N/O)c1ccc(-c2cnn3cc(-c4ccc(N5CCNCC5)cc4)cnc23)cc1]

or a pharmaceutically acceptable salt thereof.

In some embodiments, the compound of Formula II has a structure of Formula II-14:

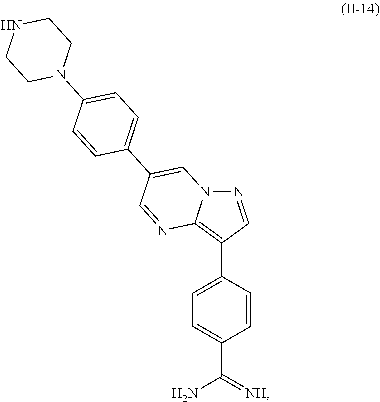 [SMILES: N=C(N)c1ccc(-c2cnn3cc(-c4ccc(N5CCNCC5)cc4)cnc23)cc1]

or a pharmaceutically acceptable salt thereof.

In some embodiments, the compound of Formula II has a structure of Formula II-15:

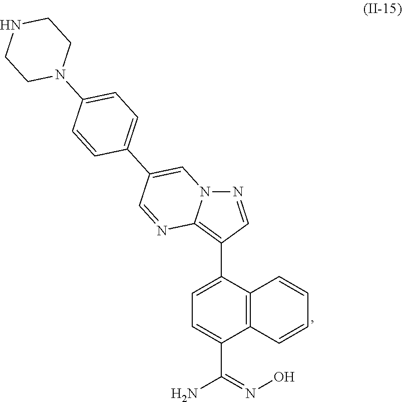 [SMILES: N/C(=N/O)c1ccc(-c2cnn3cc(-c4ccc(N5CCNCC5)cc4)cnc23)c2ccccc12]

In some embodiments, the compound of Formula II has a structure of Formula II-16:

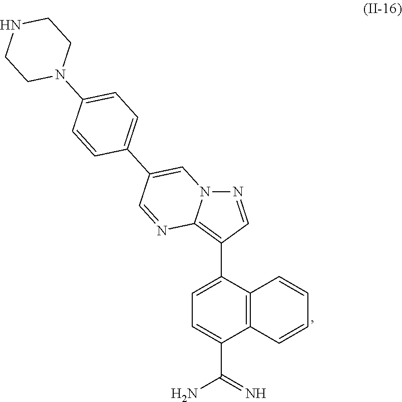 [SMILES: N=C(N)c1ccc(-c2cnn3cc(-c4ccc(N5CCNCC5)cc4)cnc23)c2ccccc12]

or a pharmaceutically acceptable salt thereof.

In some embodiments, the compound of Formula II has a structure of Formula II-17:

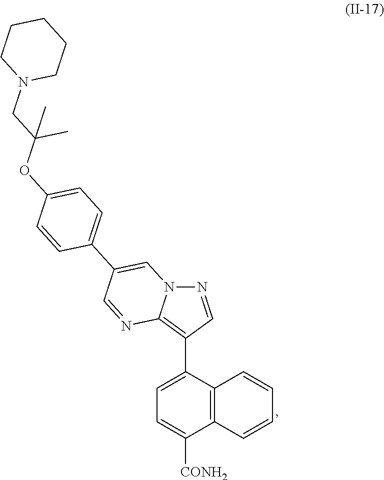 [SMILES: CC(C)(CN1CCCCC1)Oc1ccc(-c2cnc3c(-c4ccc(C(N)=O)c5ccccc45)cnn3c2)cc1]

or a pharmaceutically acceptable salt thereof.

In some embodiments, the compound of Formula II has a structure of Formula II-18:

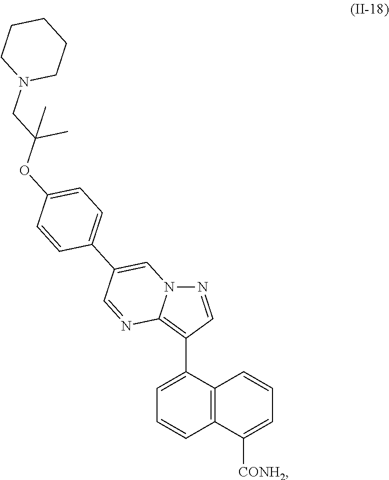 [SMILES: CC(C)(CN1CCCCC1)Oc1ccc(-c2cnc3c(-c4cccc5c(C(N)=O)cccc45)cnn3c2)cc1]

or a pharmaceutically acceptable salt thereof.

In some embodiments, the compound of Formula II has a structure of Formula II-19:

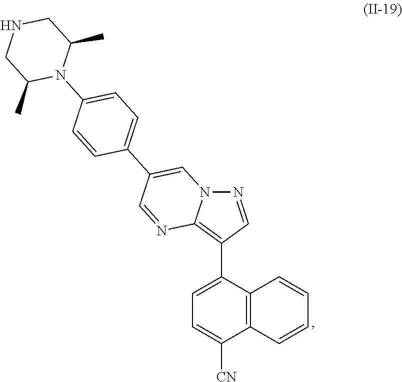 [SMILES: C[C@@H]1CNC[C@H](C)N1c1ccc(-c2cnc3c(-c4ccc(C#N)c5ccccc45)cnn3c2)cc1]

or a pharmaceutically acceptable salt thereof.

In some embodiments, the compound of Formula II has a structure of Formula II-20:

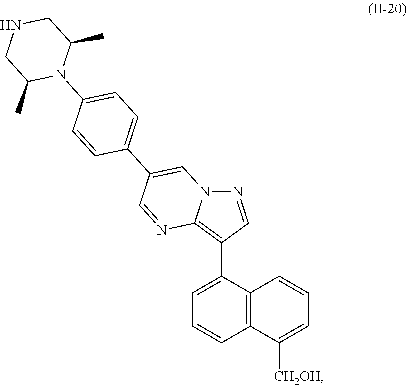 [SMILES: C[C@@H]1CNC[C@H](C)N1c1ccc(-c2cnc3c(-c4cccc5c(CO)cccc45)cnn3c2)cc1]

or a pharmaceutically acceptable salt thereof.

In some embodiments, the compound of Formula II has a structure of Formula II-21:

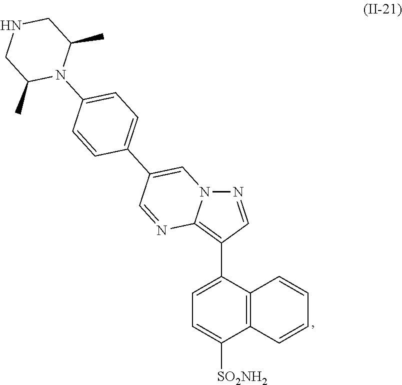 [SMILES: C[C@@H]1CNC[C@H](C)N1c1ccc(-c2cnc3c(-c4ccc(S(N)(=O)=O)c5ccccc45)cnn3c2)cc1]

In some embodiments, the compound of Formula II has a structure of Formula II-22:

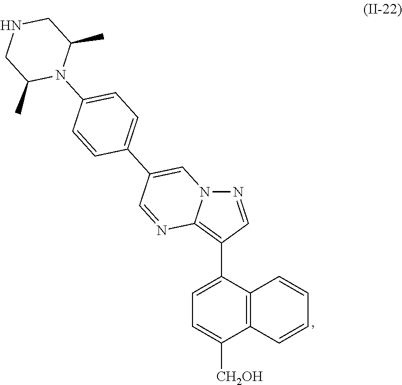 [SMILES: C[C@@H]1CNC[C@H](C)N1c1ccc(-c2cnc3c(-c4ccc(CO)c5ccccc45)cnn3c2)cc1]

or a pharmaceutically acceptable salt thereof.

In some embodiments, the compound of Formula II has a structure of Formula I-23:

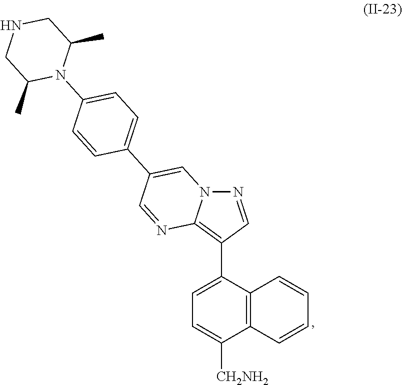

or a pharmaceutically acceptable salt thereof.

In some embodiments, the compound of Formula II has a structure of Formula II-24:

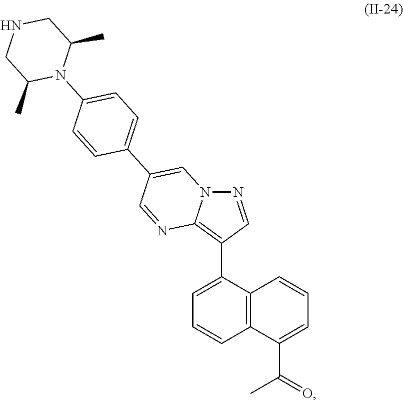 [SMILES: CC(=O)c1cccc2c(-c3cnn4cc(-c5ccc(N6[C@H](C)CNC[C@@H]6C)cc5)cnc34)cccc12]

or a pharmaceutically acceptable salt thereof.

In some embodiments, the compound of Formula II has a structure of Formula II-25:

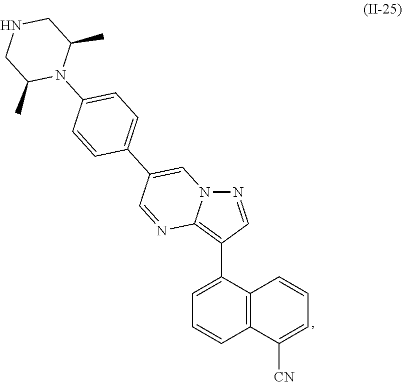 [SMILES: C[C@@H]1CNC[C@H](C)N1c1ccc(-c2cnc3c(-c4cccc5c(C#N)cccc45)cnn3c2)cc1]

or a pharmaceutically acceptable salt thereof.

In some embodiments, the compound of Formula II has a structure of Formula II-26:

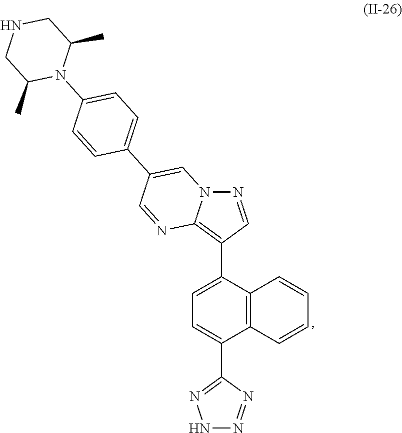 [SMILES: C[C@@H]1CNC[C@H](C)N1c1ccc(-c2cnc3c(-c4ccc(-c5nn[nH]n5)c5ccccc45)cnn3c2)cc1]

or a pharmaceutically acceptable salt thereof.

In some embodiments, the compound of Formula II has a structure of Formula NI-27:

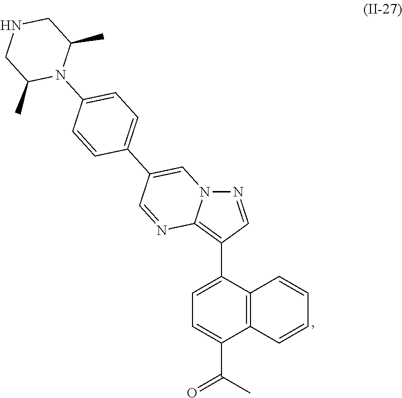 [SMILES: CC(=O)c1ccc(-c2cnn3cc(-c4ccc(N5[C@H](C)CNC[C@@H]5C)cc4)cnc23)c2ccccc12]

or a pharmaceutically acceptable salt thereof.

In some embodiments, the compound of Formula II has a structure of Formula II-28:

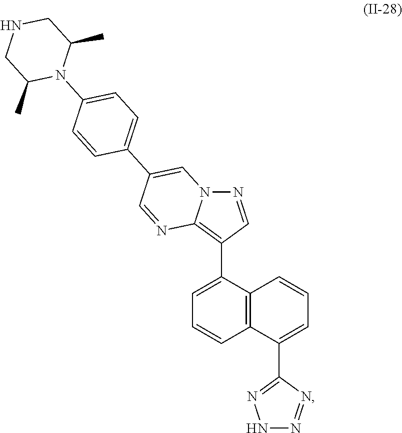 [SMILES: C[C@@H]1CNC[C@H](C)N1c1ccc(-c2cnc3c(-c4cccc5c(-c6nn[nH]n6)cccc45)cnn3c2)cc1]

or a pharmaceutically acceptable salt thereof.

In some embodiments, the compound of Formula II has a structure of Formula II-29:

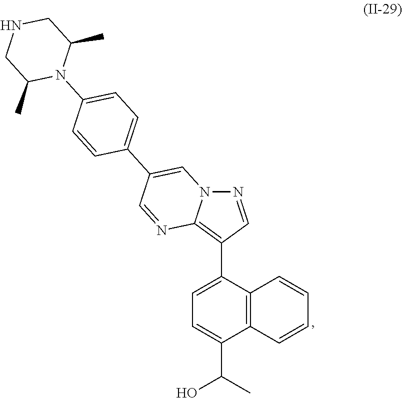 [SMILES: CC(O)c1ccc(-c2cnn3cc(-c4ccc(N5[C@H](C)CNC[C@@H]5C)cc4)cnc23)c2ccccc12]

or a pharmaceutically acceptable salt thereof.

In some embodiments, the compound of Formula II has a structure of Formula II-30:

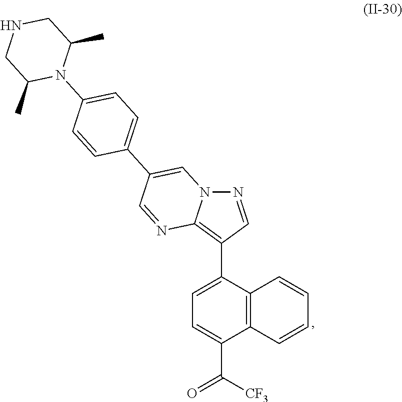 [SMILES: C[C@@H]1CNC[C@H](C)N1c1ccc(-c2cnc3c(-c4ccc(C(=O)C(F)(F)F)c5ccccc45)cnn3c2)cc1]

In some embodiments, the compound of Formula II has a structure of Formula II-31:

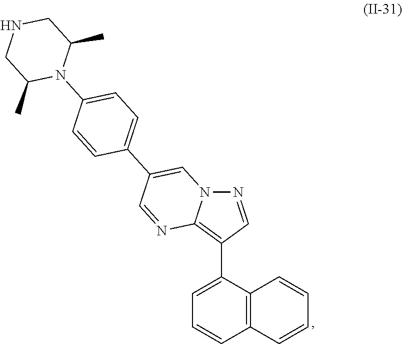 [SMILES: C[C@@H]1CNC[C@H](C)N1c1ccc(-c2cnc3c(-c4cccc5ccccc45)cnn3c2)cc1]

or a pharmaceutically acceptable salt thereof.

In some embodiments, the compound of Formula II has a structure of Formula II-32:

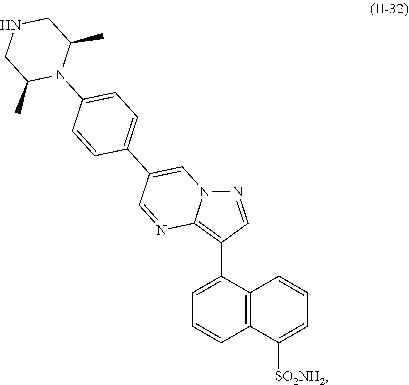 [SMILES: C[C@@H]1CNC[C@H](C)N1c1ccc(-c2cnc3c(-c4cccc5c(S(N)(=O)=O)cccc45)cnn3c2)cc1]

or a pharmaceutically acceptable salt thereof.

In some embodiments, the compound of Formula II has a structure of Formula II-33:

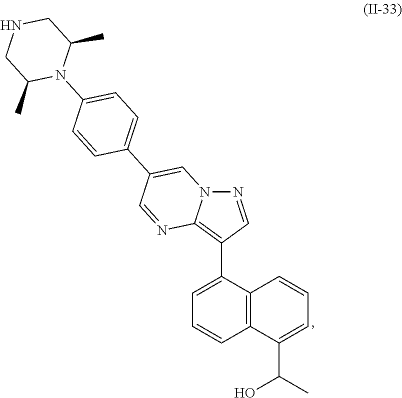 [SMILES: CC(O)c1cccc2c(-c3cnn4cc(-c5ccc(N6[C@H](C)CNC[C@@H]6C)cc5)cnc34)cccc12]

or a pharmaceutically acceptable salt thereof.

In some embodiments, the compound of Formula II has a structure of Formula II-34:

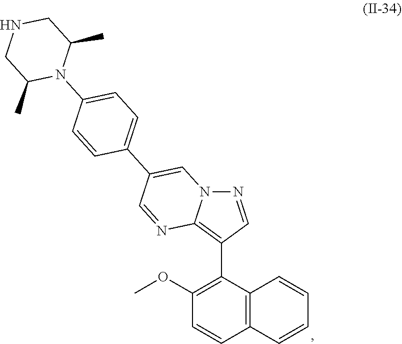 [SMILES: COc1ccc2ccccc2c1-c1cnn2cc(-c3ccc(N4[C@H](C)CNC[C@@H]4C)cc3)cnc12]

or a pharmaceutically acceptable salt thereof.

In some embodiments, the compound of Formula II has a structure of Formula II-35:

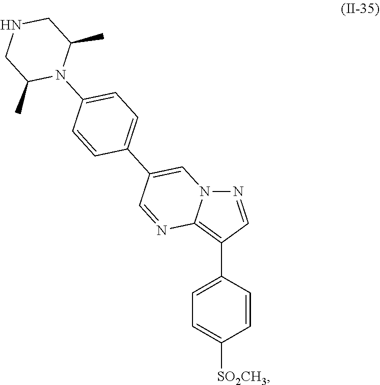 [SMILES: C[C@@H]1CNC[C@H](C)N1c1ccc(-c2cnc3c(-c4ccc(S(C)(=O)=O)cc4)cnn3c2)cc1]

or a pharmaceutically acceptable salt thereof.

In some embodiments, the compound of Formula II has a structure of Formula II-36:

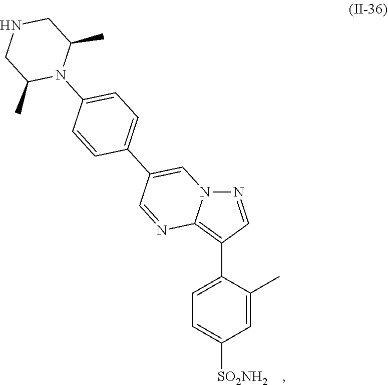 [SMILES: Cc1cc(S(N)(=O)=O)ccc1-c1cnn2cc(-c3ccc(N4[C@H](C)CNC[C@@H]4C)cc3)cnc12]

or a pharmaceutically acceptable salt thereof.

In some embodiments, the compound of Formula II has a structure of Formula II-37:

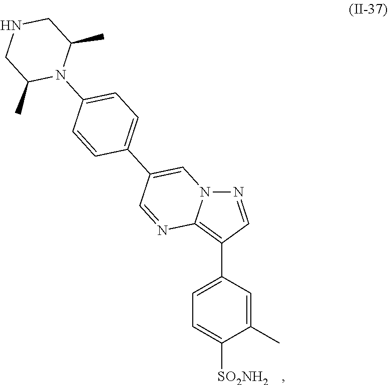 [SMILES: Cc1cc(-c2cnn3cc(-c4ccc(N5[C@H](C)CNC[C@@H]5C)cc4)cnc23)ccc1S(N)(=O)=O]

In some embodiments, the compound of Formula II has a structure of Formula II-38:

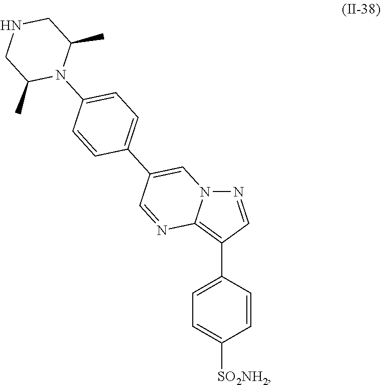 [SMILES: C[C@@H]1CNC[C@H](C)N1c1ccc(-c2cnc3c(-c4ccc(S(N)(=O)=O)cc4)cnn3c2)cc1]

or a pharmaceutically acceptable salt thereof.

In some embodiments, the compound of Formula II has a structure of Formula II-39:

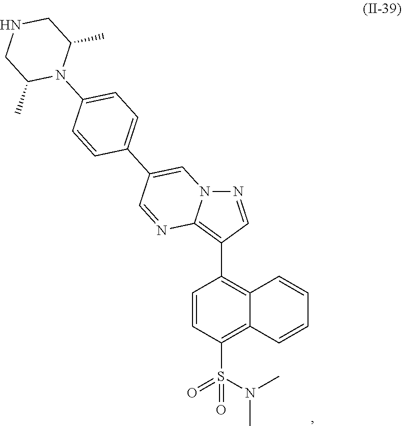 [SMILES: C[C@@H]1CNC[C@H](C)N1c1ccc(-c2cnc3c(-c4ccc(S(=O)(=O)N(C)C)c5ccccc45)cnn3c2)cc1]

or a pharmaceutically acceptable salt thereof.

In some embodiments, the compound of Formula II has a structure of Formula II-40:

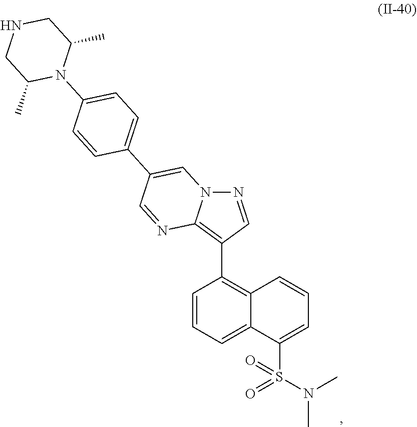 [SMILES: C[C@@H]1CNC[C@H](C)N1c1ccc(-c2cnc3c(-c4cccc5c(S(=O)(=O)N(C)C)cccc45)cnn3c2)cc1]

In some embodiments, the compound of Formula II has a structure of Formula II-41:

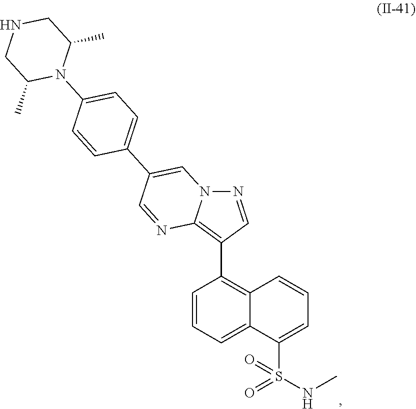 [SMILES: CNS(=O)(=O)c1cccc2c(-c3cnn4cc(-c5ccc(N6[C@H](C)CNC[C@@H]6C)cc5)cnc34)cccc12]

or a pharmaceutically acceptable salt thereof.

In some embodiments, the compound of Formula II has a structure of Formula II-42:

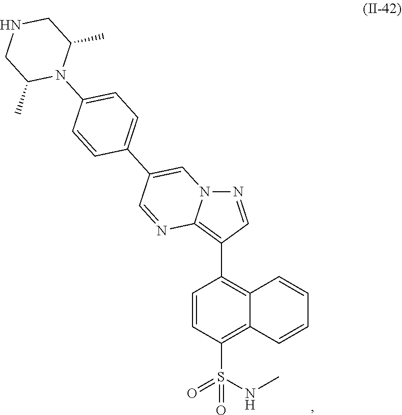 [SMILES: CNS(=O)(=O)c1ccc(-c2cnn3cc(-c4ccc(N5[C@H](C)CNC[C@@H]5C)cc4)cnc23)c2ccccc12]

or a pharmaceutically acceptable salt thereof.

In some embodiments, the compound of Formula II has a structure of Formula II-43:

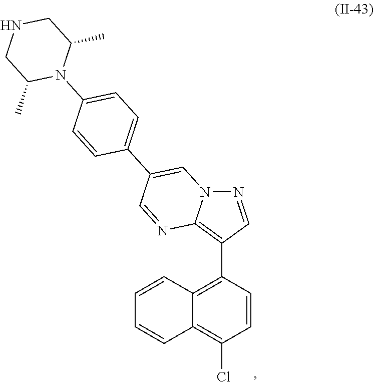 [SMILES: C[C@@H]1CNC[C@H](C)N1c1ccc(-c2cnc3c(-c4ccc(Cl)c5ccccc45)cnn3c2)cc1]

or a pharmaceutically acceptable salt thereof.

In some embodiments, the compound of Formula II has a structure of Formula II-44:

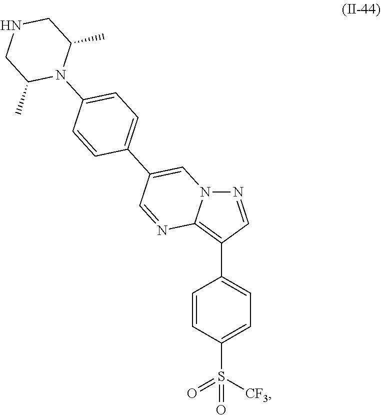 [SMILES: C[C@@H]1CNC[C@H](C)N1c1ccc(-c2cnc3c(-c4ccc(S(=O)(=O)C(F)(F)F)cc4)cnn3c2)cc1]

or a pharmaceutically acceptable salt thereof.

In some embodiments, the compound of Formula II has a structure of Formula II-45:

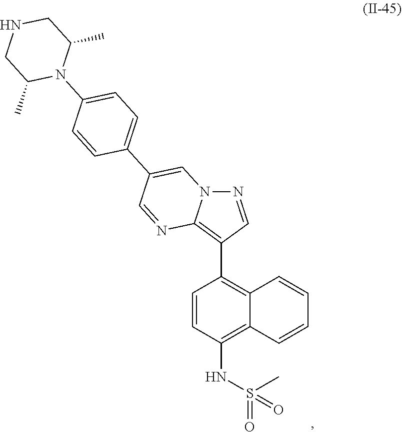 [SMILES: C[C@@H]1CNC[C@H](C)N1c1ccc(-c2cnc3c(-c4ccc(NS(C)(=O)=O)c5ccccc45)cnn3c2)cc1]

or a pharmaceutically acceptable salt thereof.

In some embodiments, the compound of Formula II has a structure of Formula II-46:

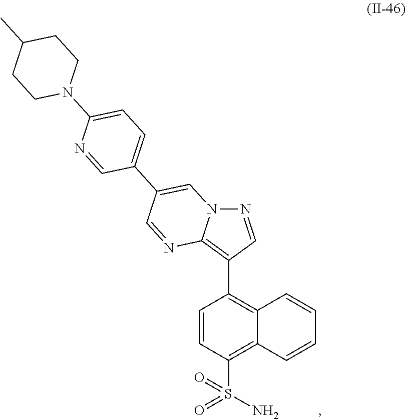 [SMILES: CC1CCN(c2ccc(-c3cnc4c(-c5ccc(S(N)(=O)=O)c6ccccc56)cnn4c3)cn2)CC1]

or a pharmaceutically acceptable salt thereof.

In some embodiments, the compound of Formula II has a structure of Formula II-47:

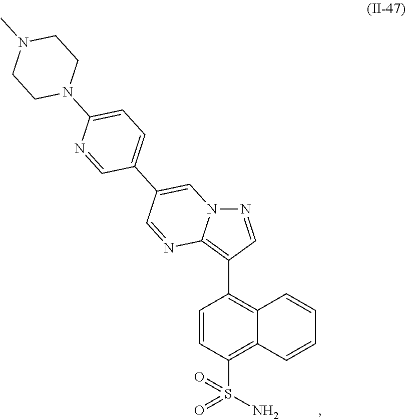 [SMILES: CN1CCN(c2ccc(-c3cnc4c(-c5ccc(S(N)(=O)=O)c6ccccc56)cnn4c3)cn2)CC1]

or a pharmaceutically acceptable salt thereof.

In some embodiments, the compound of Formula II has a structure of Formula II-48:

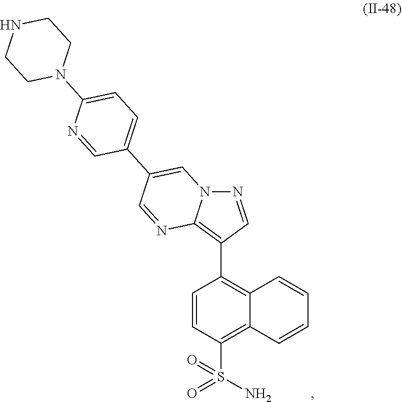 [SMILES: NS(=O)(=O)c1ccc(-c2cnn3cc(-c4ccc(N5CCNCC5)nc4)cnc23)c2ccccc12]

or a pharmaceutically acceptable salt thereof.

In some embodiments, the compound of Formula II has a structure of Formula II-49:

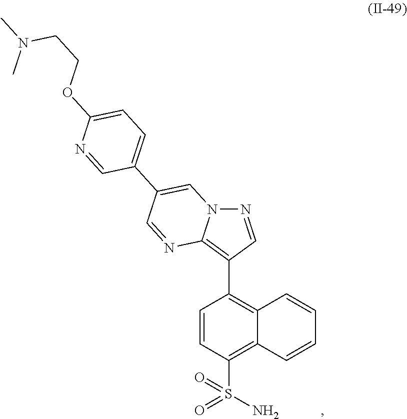 [SMILES: CN(C)CCOc1ccc(-c2cnc3c(-c4ccc(S(N)(=O)=O)c5ccccc45)cnn3c2)cn1]

or a pharmaceutically acceptable salt thereof.

In some embodiments, the compound of Formula II has a structure of Formula II-50:

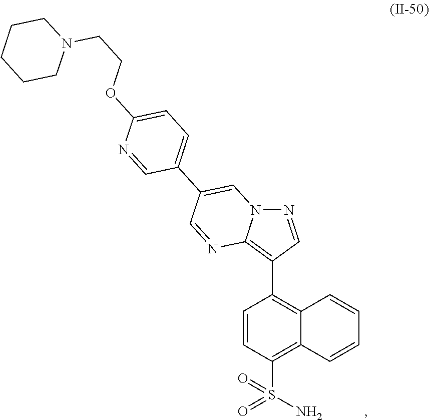 [SMILES: NS(=O)(=O)c1ccc(-c2cnn3cc(-c4ccc(OCCN5CCCCC5)nc4)cnc23)c2ccccc12]

or a pharmaceutically acceptable salt thereof.

In some embodiments, the compound of Formula II has a structure of Formula II-51a:

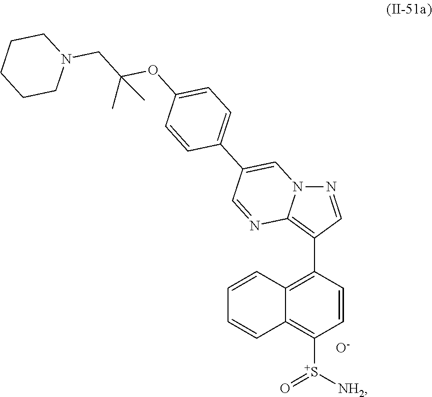

or a pharmaceutically acceptable salt thereof.

In some embodiments, the compound of Formula II has a structure of Formula II-51b:

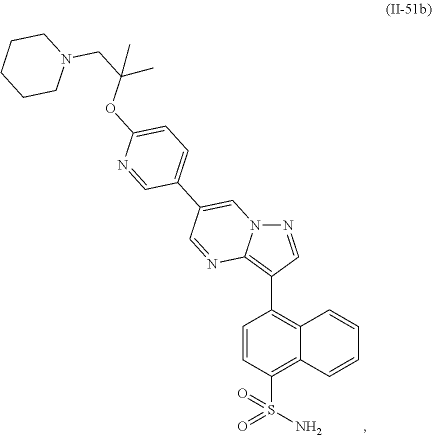 [SMILES: CC(C)(CN1CCCCC1)Oc1ccc(-c2cnc3c(-c4ccc(S(N)(=O)=O)c5ccccc45)cnn3c2)cn1]

In some embodiments, the compound of Formula II has a structure of Formula II-52:

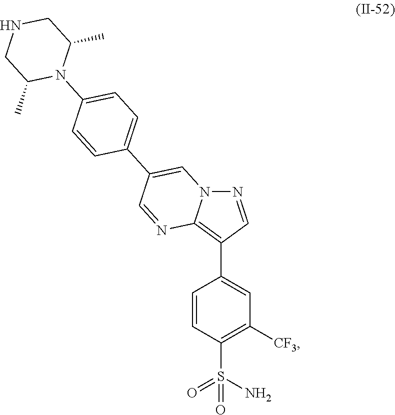 [SMILES: C[C@@H]1CNC[C@H](C)N1c1ccc(-c2cnc3c(-c4ccc(S(N)(=O)=O)c(C(F)(F)F)c4)cnn3c2)cc1]

or a pharmaceutically acceptable salt thereof.

In some embodiments, the compound of Formula II has a structure of Formula II-53:

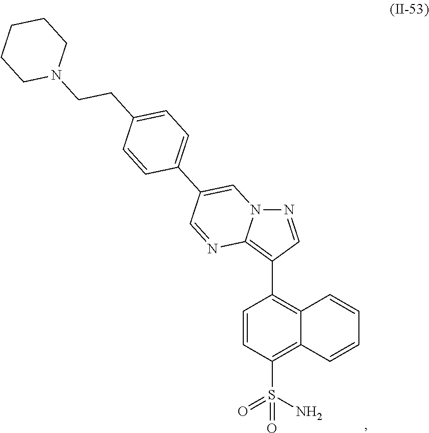 [SMILES: NS(=O)(=O)c1ccc(-c2cnn3cc(-c4ccc(CCN5CCCCC5)cc4)cnc23)c2ccccc12]

or a pharmaceutically acceptable salt thereof.

In some embodiments, the compound of Formula II has a structure of Formula II-54:

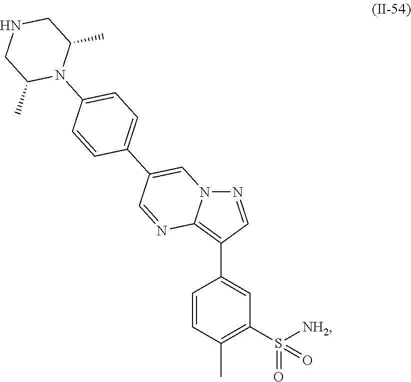 [SMILES: Cc1ccc(-c2cnn3cc(-c4ccc(N5[C@H](C)CNC[C@@H]5C)cc4)cnc23)cc1S(N)(=O)=O]

or a pharmaceutically acceptable salt thereof.

In some embodiments, the compound of Formula II has a structure of Formula NI-55:

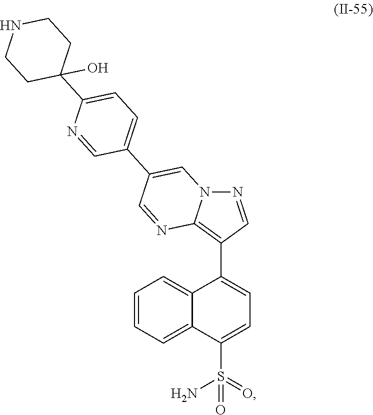 [SMILES: NS(=O)(=O)c1ccc(-c2cnn3cc(-c4ccc(C5(O)CCNCC5)nc4)cnc23)c2ccccc12]

or a pharmaceutically acceptable salt thereof.

In some embodiments, the compound of Formula II has a structure of Formula II-56:

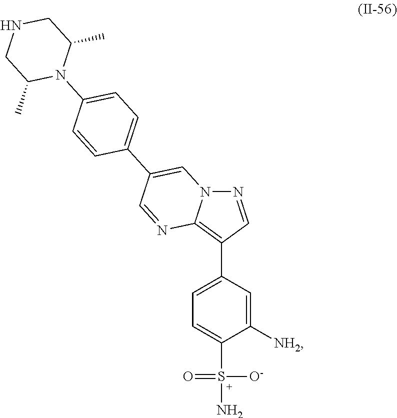

or a pharmaceutically acceptable salt thereof.

In some embodiments, the compound of Formula II has a structure of Formula II-57:

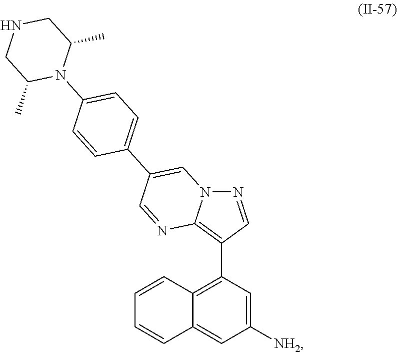 [SMILES: C[C@@H]1CNC[C@H](C)N1c1ccc(-c2cnc3c(-c4cc(N)cc5ccccc45)cnn3c2)cc1]

or a pharmaceutically acceptable salt thereof.

In some embodiments, the compound of Formula II has a structure of Formula II-58:

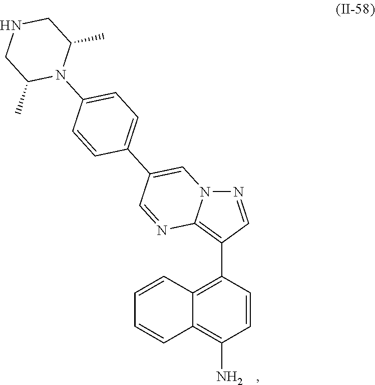 [SMILES: C[C@@H]1CNC[C@H](C)N1c1ccc(-c2cnc3c(-c4ccc(N)c5ccccc45)cnn3c2)cc1]

or a pharmaceutically acceptable salt thereof.

In some embodiments, the compound of Formula II has a structure of Formula II-59:

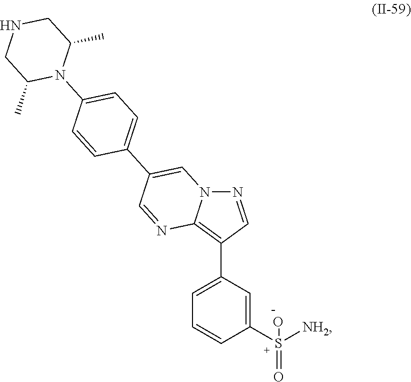

or a pharmaceutically acceptable salt thereof.

In some embodiments, the compound of Formula II has a structure of Formula II-00:

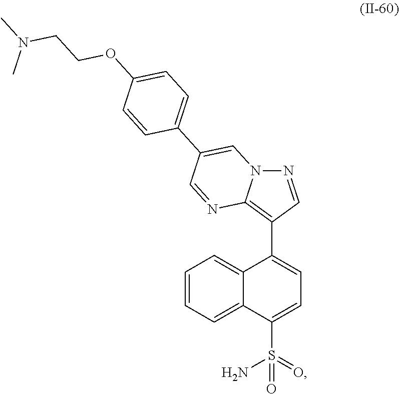 [SMILES: CN(C)CCOc1ccc(-c2cnc3c(-c4ccc(S(N)(=O)=O)c5ccccc45)cnn3c2)cc1]

In some embodiments, the compound of Formula II has a structure of Formula II-61:

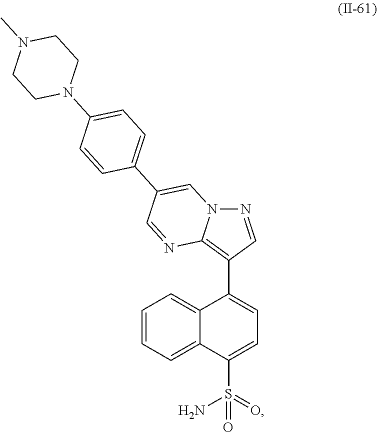 [SMILES: CN1CCN(c2ccc(-c3cnc4c(-c5ccc(S(N)(=O)=O)c6ccccc56)cnn4c3)cc2)CC1]

or a pharmaceutically acceptable salt thereof.

In some embodiments, the compound of Formula II has a structure of Formula II-62:

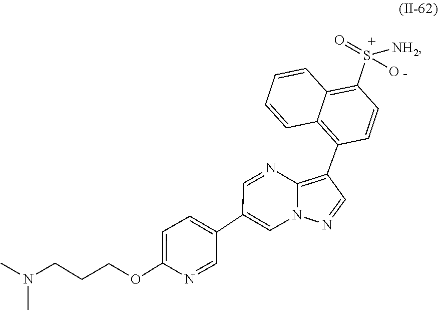

or a pharmaceutically acceptable salt thereof.

In some embodiments, the compound of Formula II has a structure of Formula II-63:

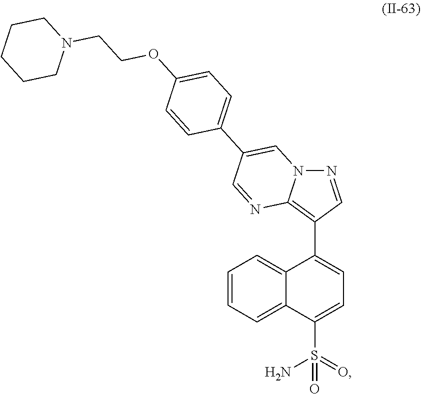 [SMILES: NS(=O)(=O)c1ccc(-c2cnn3cc(-c4ccc(OCCN5CCCCC5)cc4)cnc23)c2ccccc12]

In some embodiments, the compound of Formula II has a structure of Formula II-84:

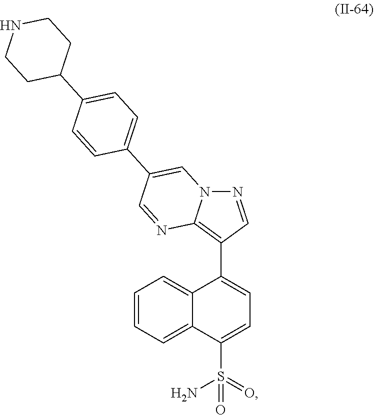 [SMILES: NS(=O)(=O)c1ccc(-c2cnn3cc(-c4ccc(C5CCNCC5)cc4)cnc23)c2ccccc12]

or a pharmaceutically acceptable salt thereof.

In some embodiments, the compound of Formula II has a structure of Formula II-65:

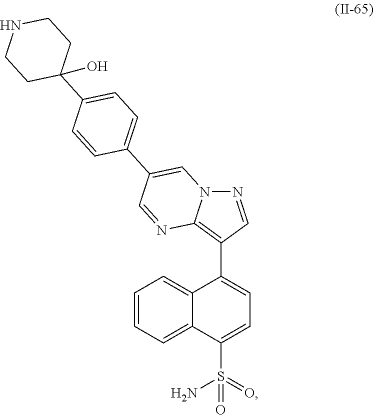 [SMILES: NS(=O)(=O)c1ccc(-c2cnn3cc(-c4ccc(C5(O)CCNCC5)cc4)cnc23)c2ccccc12]

or a pharmaceutically acceptable salt thereof.

In some embodiments, the compound of Formula II has a structure of Formula II-66:

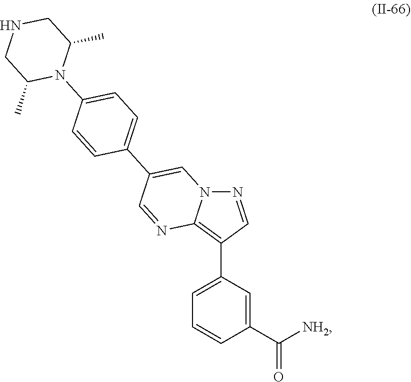 [SMILES: C[C@@H]1CNC[C@H](C)N1c1ccc(-c2cnc3c(-c4cccc(C(N)=O)c4)cnn3c2)cc1]

In some embodiments, the compound of Formula II has a structure of Formula II-67:

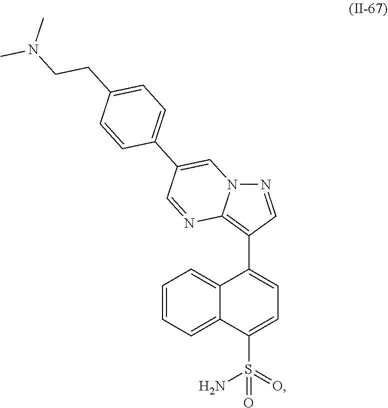 [SMILES: CN(C)CCc1ccc(-c2cnc3c(-c4ccc(S(N)(=O)=O)c5ccccc45)cnn3c2)cc1]

or a pharmaceutically acceptable salt thereof.

In some embodiments, the compound of Formula II has a structure of Formula II-68:

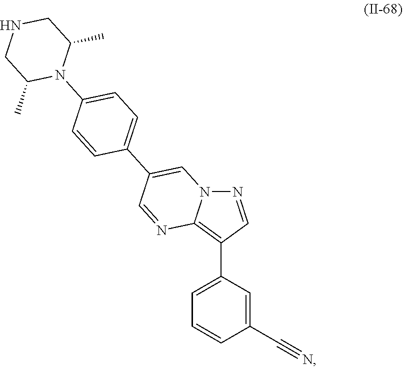 [SMILES: C[C@@H]1CNC[C@H](C)N1c1ccc(-c2cnc3c(-c4cccc(C#N)c4)cnn3c2)cc1]

or a pharmaceutically acceptable salt thereof.

In some embodiments, the compound of Formula II has a structure of Formula II-69:

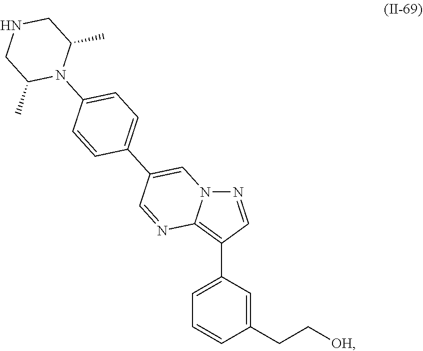 [SMILES: C[C@@H]1CNC[C@H](C)N1c1ccc(-c2cnc3c(-c4cccc(CCO)c4)cnn3c2)cc1]

In some embodiments, the compound of Formula II has a structure of Formula II-70:

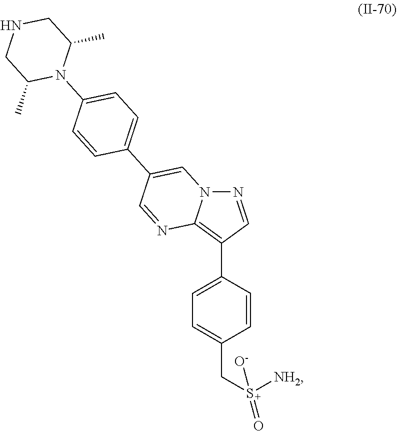

or a pharmaceutically acceptable salt thereof.

In some embodiments, the compound of Formula II has a structure of Formula I-71:

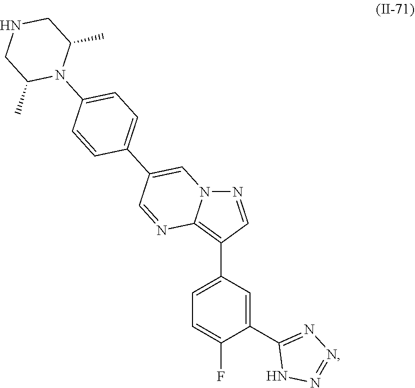 [SMILES: C[C@@H]1CNC[C@H](C)N1c1ccc(-c2cnc3c(-c4ccc(F)c(-c5nnn[nH]5)c4)cnn3c2)cc1]

or a pharmaceutically acceptable salt thereof.

In some embodiments, the compound of Formula II has a structure of Formula II-72:

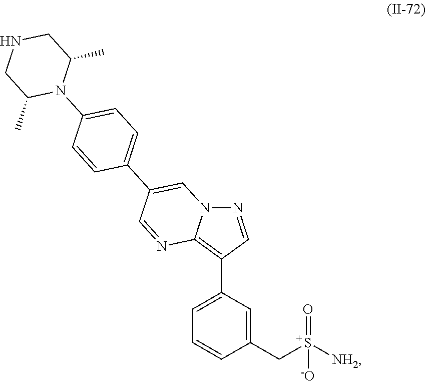

or a pharmaceutically acceptable salt thereof.

In some embodiments, the compound of Formula II has a structure of Formula NI-73:

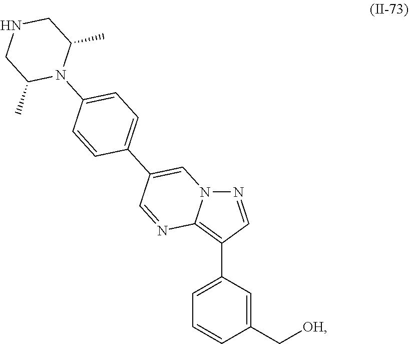 [SMILES: C[C@@H]1CNC[C@H](C)N1c1ccc(-c2cnc3c(-c4cccc(CO)c4)cnn3c2)cc1]

or a pharmaceutically acceptable salt thereof.

In some embodiments, the compound of Formula II has a structure of Formula II-74:

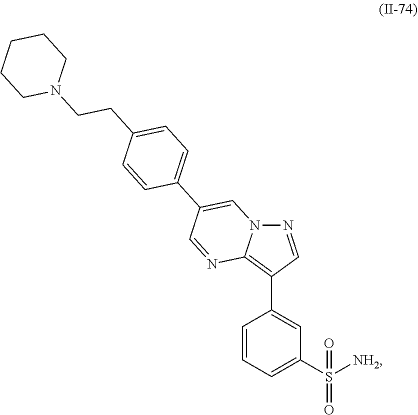 [SMILES: NS(=O)(=O)c1cccc(-c2cnn3cc(-c4ccc(CCN5CCCCC5)cc4)cnc23)c1]

or a pharmaceutically acceptable salt thereof.

In some embodiments, the compound of Formula II has a structure of Formula II-75:

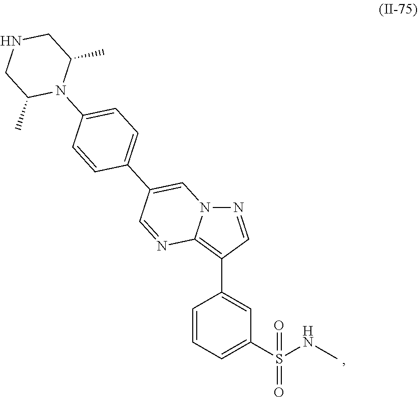 [SMILES: CNS(=O)(=O)c1cccc(-c2cnn3cc(-c4ccc(N5[C@H](C)CNC[C@@H]5C)cc4)cnc23)c1]

or a pharmaceutically acceptable salt thereof.

In some embodiments, the compound of Formula II has a structure of Formula II-78:

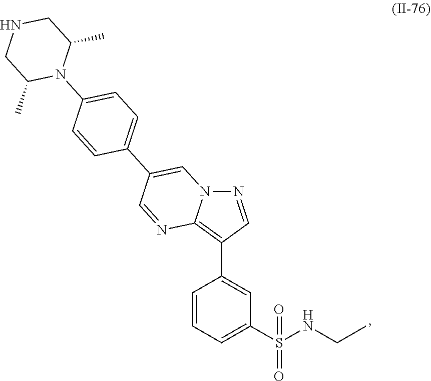 [SMILES: CCNS(=O)(=O)c1cccc(-c2cnn3cc(-c4ccc(N5[C@H](C)CNC[C@@H]5C)cc4)cnc23)c1]

or a pharmaceutically acceptable salt thereof.

In some embodiments, the compound of Formula II has a structure of Formula II-77:

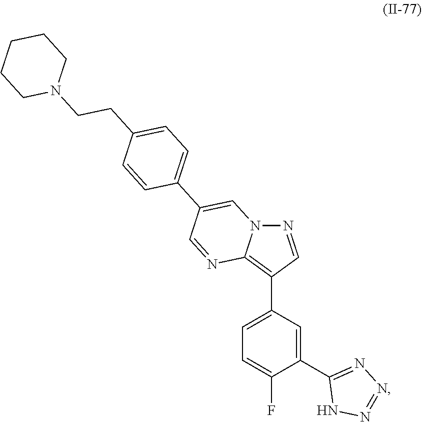 [SMILES: Fc1ccc(-c2cnn3cc(-c4ccc(CCN5CCCCC5)cc4)cnc23)cc1-c1nnn[nH]1]

or a pharmaceutically acceptable salt thereof.

In some embodiments, the compound of Formula II has a structure of Formula II-78:5

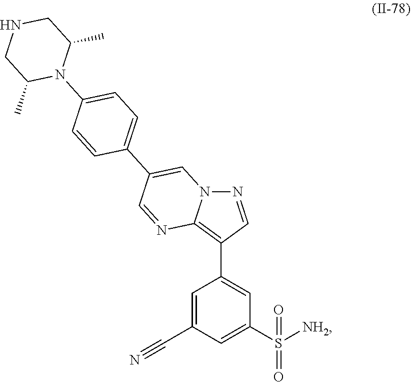 [SMILES: C[C@@H]1CNC[C@H](C)N1c1ccc(-c2cnc3c(-c4cc(C#N)cc(S(N)(=O)=O)c4)cnn3c2)cc1]

or a pharmaceutically acceptable salt thereof.

In some embodiments, the compound of Formula II has a structure of Formula II-79:

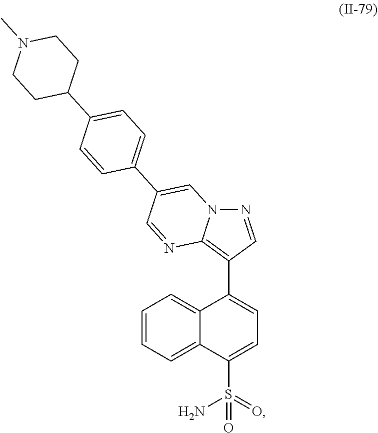 [SMILES: CN1CCC(c2ccc(-c3cnc4c(-c5ccc(S(N)(=O)=O)c6ccccc56)cnn4c3)cc2)CC1]

or a pharmaceutically acceptable salt thereof.

In some embodiments, the compound of Formula II has a structure of Formula II-80:

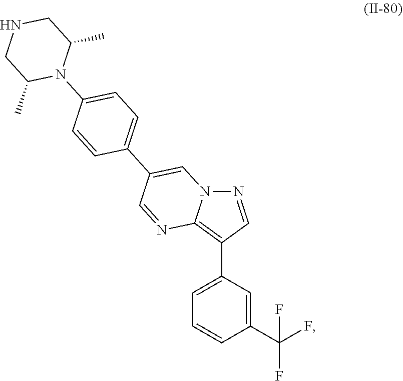 [SMILES: C[C@@H]1CNC[C@H](C)N1c1ccc(-c2cnc3c(-c4cccc(C(F)(F)F)c4)cnn3c2)cc1]

or a pharmaceutically acceptable salt thereof.

In some embodiments, the compound of Formula II has a structure of Formula II-81:

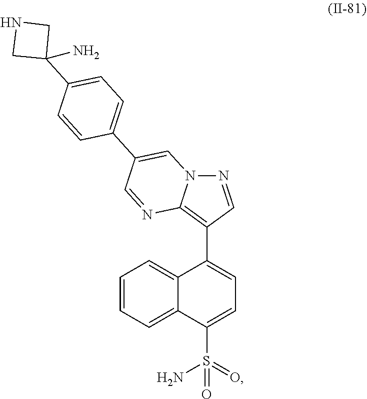 [SMILES: NC1(c2ccc(-c3cnc4c(-c5ccc(S(N)(=O)=O)c6ccccc56)cnn4c3)cc2)CNC1]

or a pharmaceutically acceptable salt thereof.

In some embodiments, the compound of Formula II has a structure of Formula II-82:

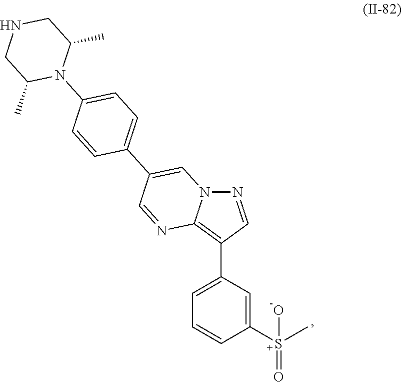

or a pharmaceutically acceptable salt thereof.

In some embodiments, the compound of Formula II has a structure of Formula II-83:

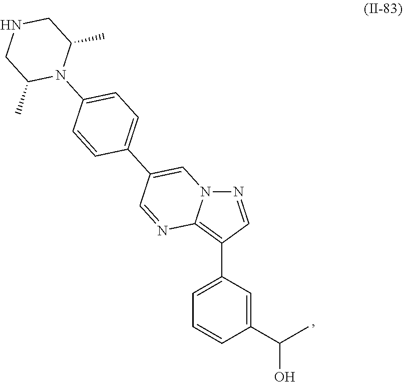 [SMILES: CC(O)c1cccc(-c2cnn3cc(-c4ccc(N5[C@H](C)CNC[C@@H]5C)cc4)cnc23)c1]

or a pharmaceutically acceptable salt thereof.

In some embodiments, the compound of Formula II has a structure of Formula II-84:

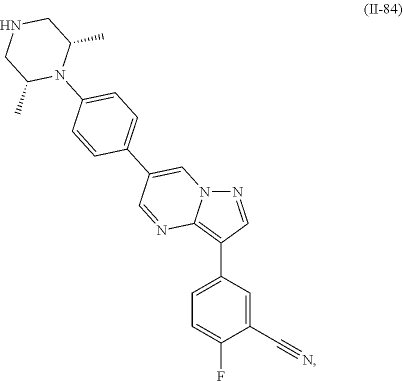 [SMILES: C[C@@H]1CNC[C@H](C)N1c1ccc(-c2cnc3c(-c4ccc(F)c(C#N)c4)cnn3c2)cc1]

In some embodiments, the compound of Formula II has a structure of Formula II-85:

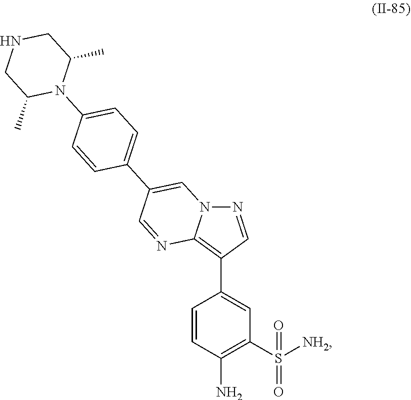 [SMILES: C[C@@H]1CNC[C@H](C)N1c1ccc(-c2cnc3c(-c4ccc(N)c(S(N)(=O)=O)c4)cnn3c2)cc1]

or a pharmaceutically acceptable salt thereof.

In some embodiments, the compound of Formula II has a structure of Formula II-86:

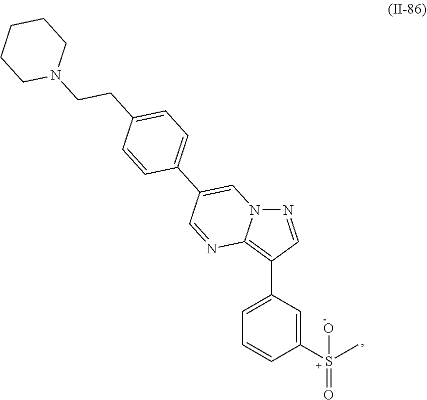

or a pharmaceutically acceptable salt thereof.

In some embodiments, the compound of Formula II has a structure of Formula II-87:

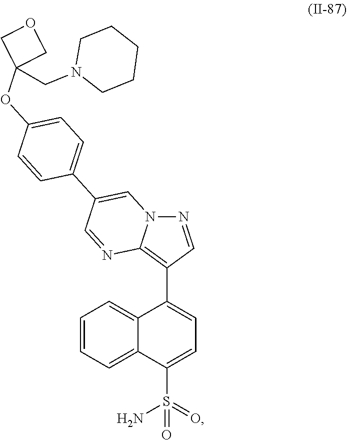 [SMILES: NS(=O)(=O)c1ccc(-c2cnn3cc(-c4ccc(OC5(CN6CCCCC6)COC5)cc4)cnc23)c2ccccc12]

or a pharmaceutically acceptable salt thereof.

In some embodiments, the compound of Formula II has a structure of Formula II-88:

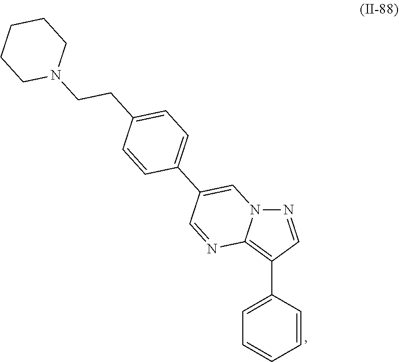 [SMILES: c1ccc(-c2cnn3cc(-c4ccc(CCN5CCCCC5)cc4)cnc23)cc1]

or a pharmaceutically acceptable salt thereof.

In some embodiments, the compound of Formula II has a structure of Formula II-89:

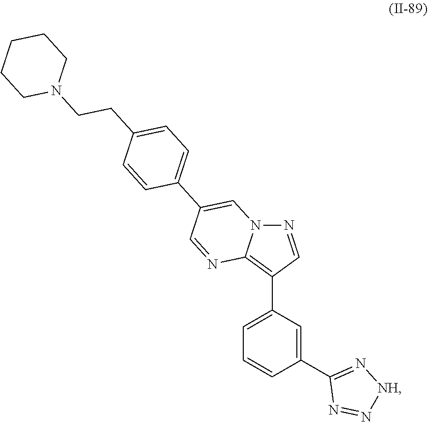 [SMILES: c1cc(-c2nn[nH]n2)cc(-c2cnn3cc(-c4ccc(CCN5CCCCC5)cc4)cnc23)c1]

or a pharmaceutically acceptable salt thereof.

In some embodiments, the compound of Formula II has a structure of Formula II-80:

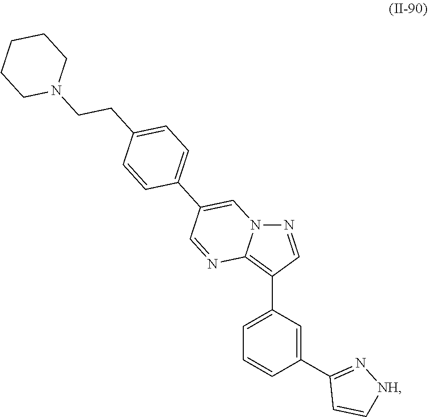 [SMILES: c1cc(-c2cc[nH]n2)cc(-c2cnn3cc(-c4ccc(CCN5CCCCC5)cc4)cnc23)c1]

or a pharmaceutically acceptable salt thereof.

In some embodiments, the compound of Formula II has a structure of Formula II-91:

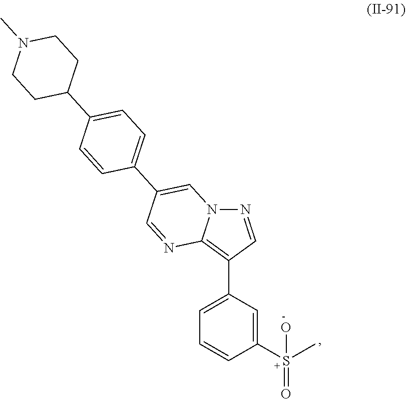

or a pharmaceutically acceptable salt thereof.

In some embodiments, the compound of Formula II has a structure of Formula II-92:

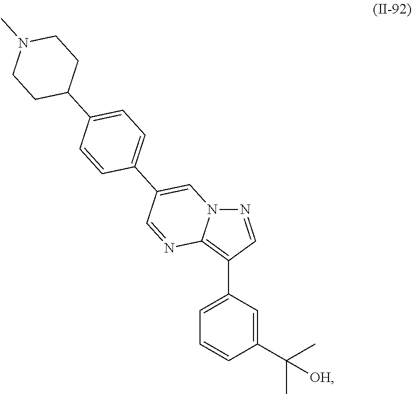 [SMILES: CN1CCC(c2ccc(-c3cnc4c(-c5cccc(C(C)(C)O)c5)cnn4c3)cc2)CC1]

or a pharmaceutically acceptable salt thereof.

In some embodiments, the compound of Formula II has a structure of Formula II-93:

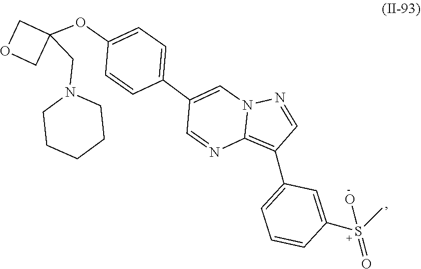

or a pharmaceutically acceptable salt thereof.

In some embodiments, the compound of Formula II has a structure of Formula II-94:

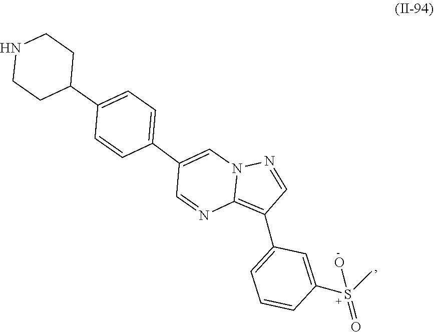

or a pharmaceutically acceptable salt thereof.

In some embodiments, the compound of Formula II has a structure of Formula II-95:

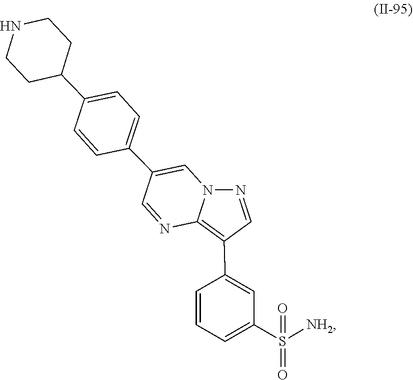 [SMILES: NS(=O)(=O)c1cccc(-c2cnn3cc(-c4ccc(C5CCNCC5)cc4)cnc23)c1]

or a pharmaceutically acceptable salt thereof.

In some embodiments, the compound of Formula II has a structure of Formula II-96:

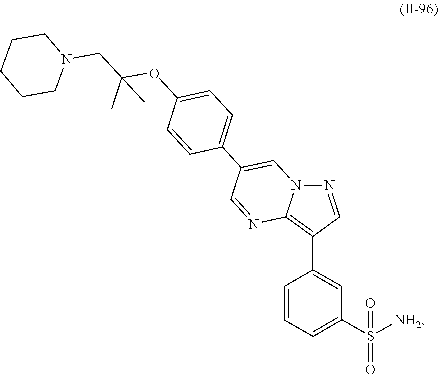 [SMILES: CC(C)(CN1CCCCC1)Oc1ccc(-c2cnc3c(-c4cccc(S(N)(=O)=O)c4)cnn3c2)cc1]

or a pharmaceutically acceptable salt thereof.

In some embodiments, the compound of Formula II has a structure of Formula II-97:

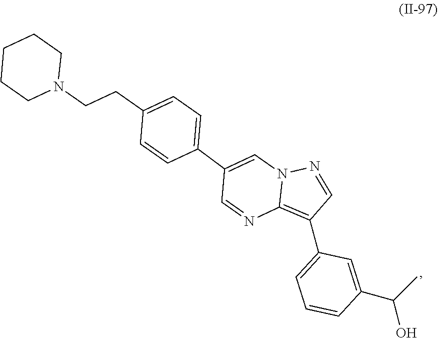 [SMILES: CC(O)c1cccc(-c2cnn3cc(-c4ccc(CCN5CCCCC5)cc4)cnc23)c1]

or a pharmaceutically acceptable salt thereof.

In some embodiments, the compound of Formula II has a structure of Formula II-98:

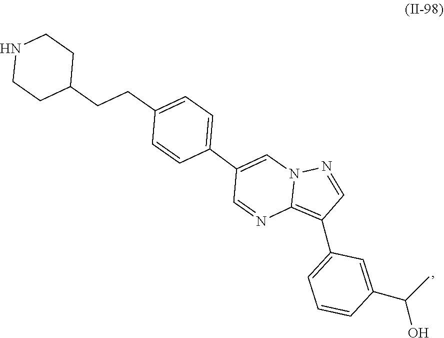 [SMILES: CC(O)c1cccc(-c2cnn3cc(-c4ccc(CCC5CCNCC5)cc4)cnc23)c1]

or a pharmaceutically acceptable salt thereof.

In some embodiments, the compound of Formula II has a structure of Formula II-99:

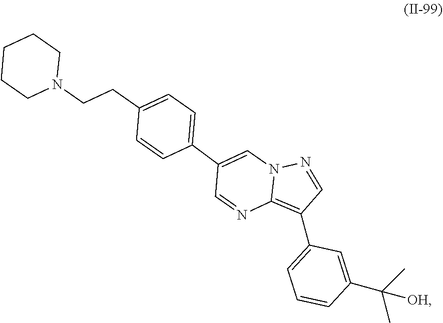 [SMILES: CC(C)(O)c1cccc(-c2cnn3cc(-c4ccc(CCN5CCCCC5)cc4)cnc23)c1]

or a pharmaceutically acceptable salt thereof.

In some embodiments, the compound of Formula II has a structure of Formula II-100:

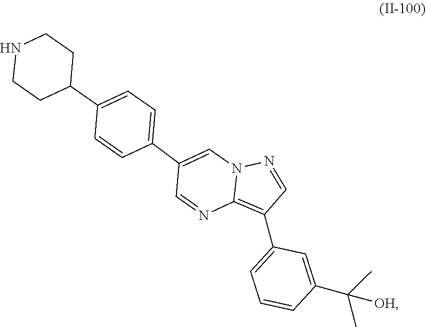 [SMILES: CC(C)(O)c1cccc(-c2cnn3cc(-c4ccc(C5CCNCC5)cc4)cnc23)c1]

or a pharmaceutically acceptable salt thereof.

In some embodiments, the compound of Formula II has a structure of Formula II-101:

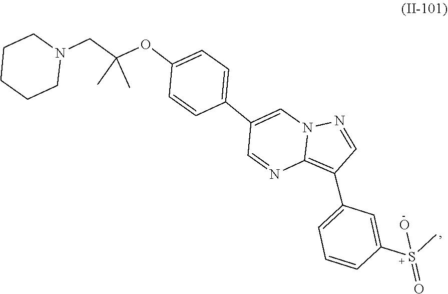

or a pharmaceutically acceptable salt thereof.

In some embodiments, the compound of Formula II has a structure of Formula II-102:

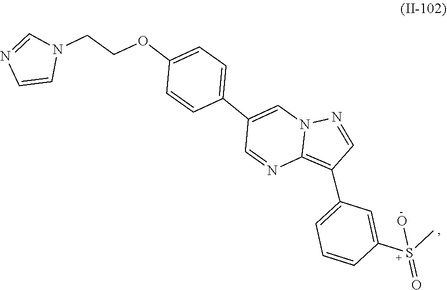

or a pharmaceutically acceptable salt thereof.

In some embodiments, the compound of Formula II has a structure of Formula II-103:

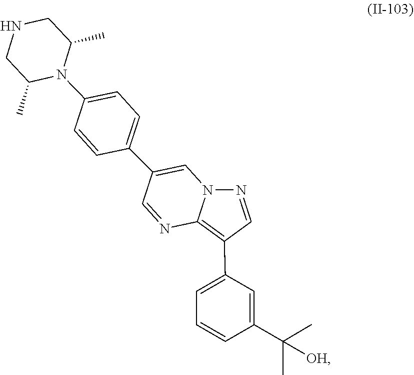 [SMILES: C[C@@H]1CNC[C@H](C)N1c1ccc(-c2cnc3c(-c4cccc(C(C)(C)O)c4)cnn3c2)cc1]

or a pharmaceutically acceptable salt thereof.

In some embodiments, the compound of Formula II has a structure of Formula II-104:

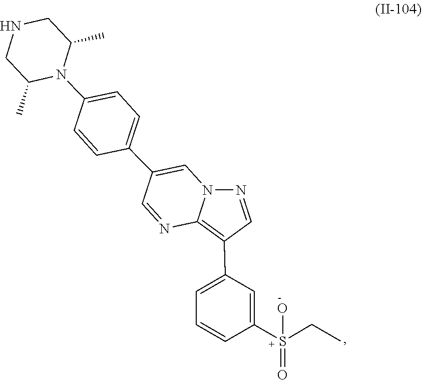

or a pharmaceutically acceptable salt thereof.

In some embodiments, the compound of Formula II has a structure of Formula II-105:

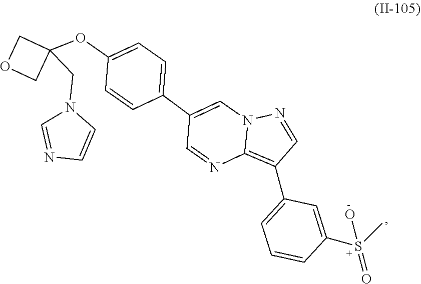

or a pharmaceutically acceptable salt thereof.

In some embodiments, the compound of Formula II has a structure of Formula II-106:

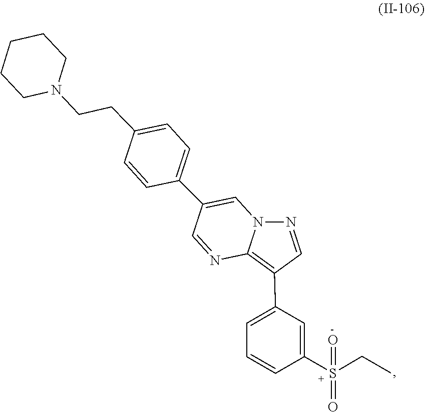 [SMILES: CCS(=O)(=O)c1cccc(-c2cnn3cc(-c4ccc(CCN5CCCCC5)cc4)cnc23)c1]

or a pharmaceutically acceptable salt thereof.

In some embodiments, the compound of Formula II has a structure of Formula II-107:

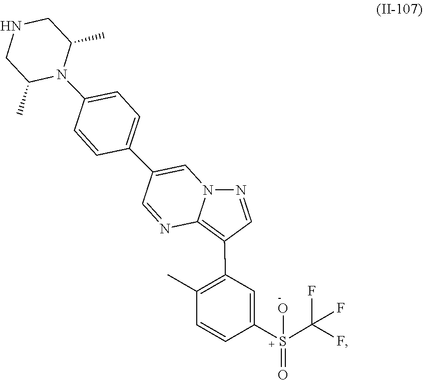

or a pharmaceutically acceptable salt thereof.

In some embodiments, the compound of Formula II has a structure of Formula II-108:

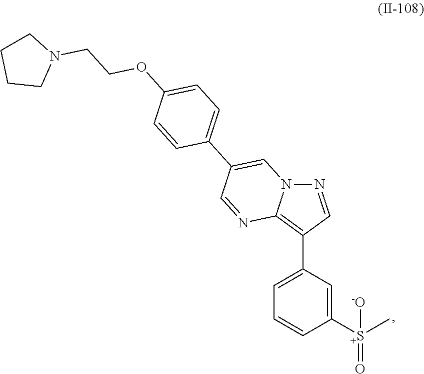

or a pharmaceutically acceptable salt thereof.

In some embodiments, the compound of Formula II has a structure of Formula II-109:

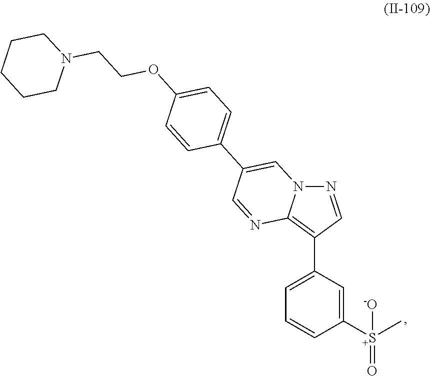

or a pharmaceutically acceptable salt thereof.

In some embodiments, the compound of Formula II has a structure of Formula II-110:

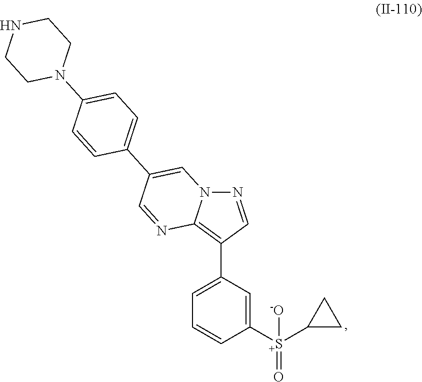

or a pharmaceutically acceptable salt thereof.

In some embodiments, the compound of Formula II has a structure of Formula II-111:

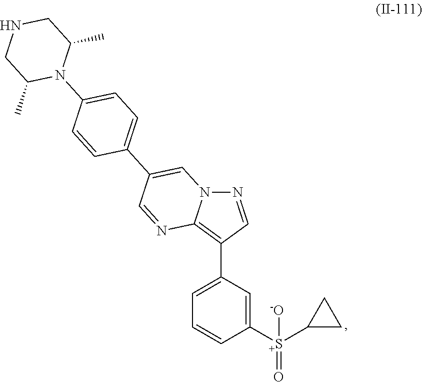

or a pharmaceutically acceptable salt thereof.

In some embodiments, the compound of Formula II has a structure of Formula II-112:

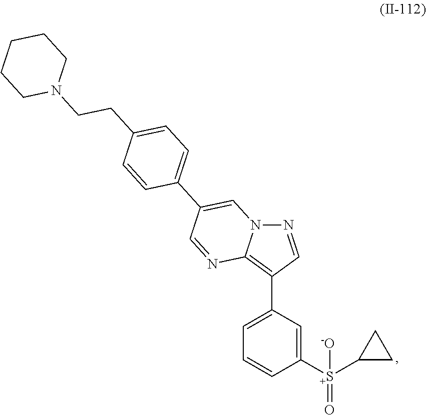

or a pharmaceutically acceptable salt thereof.

In some embodiments, the compound of Formula II has a structure of Formula I-113:

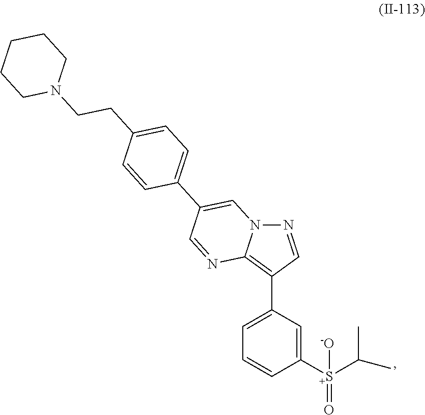

In some embodiments, the compound of Formula II has a structure of Formula II-114:

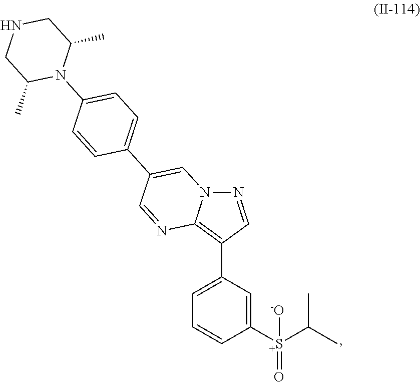

or a pharmaceutically acceptable salt thereof.

In some embodiments, the compound of Formula II has a structure of Formula II-115:

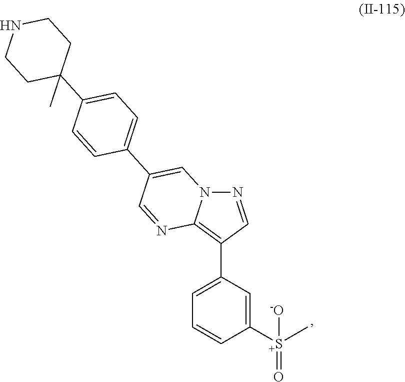

or a pharmaceutically acceptable salt thereof.

In some embodiments, the compound of Formula II has a structure of Formula II-116:

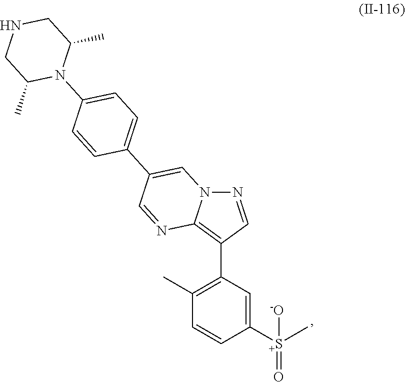

or a pharmaceutically acceptable salt thereof.

In some embodiments, the compound of Formula II has a structure of Formula II-117:

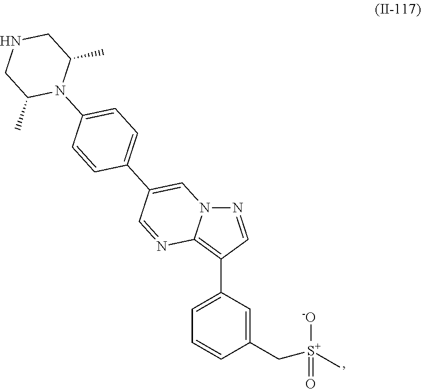

or a pharmaceutically acceptable sail thereof.

In some embodiments, the compound of Formula II has a structure of Formula II-118:

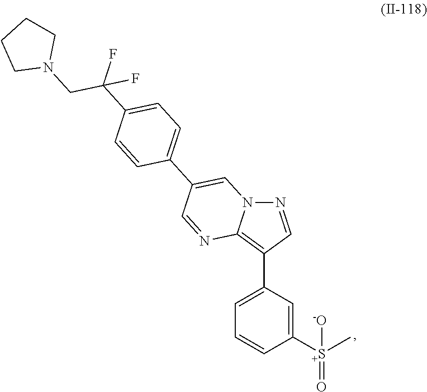

or a pharmaceutically acceptable salt thereof.

In some embodiments, the compound of Formula II has a structure of Formula II-119:

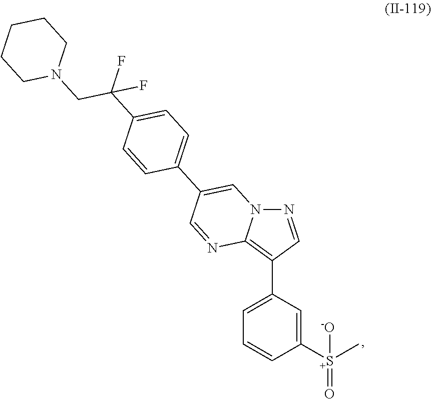

In some embodiments, the compound of Formula II has a structure of Formula II-120:

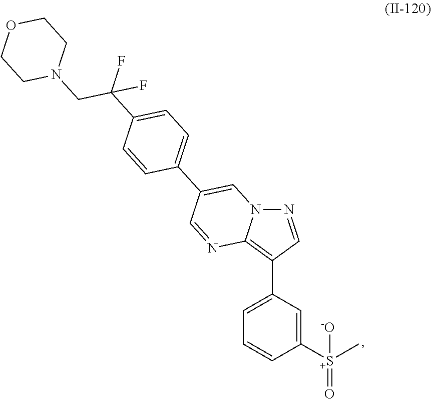

or a pharmaceutically acceptable salt thereof.

In some embodiments, the compound of Formula II has a structure of Formula II-121:

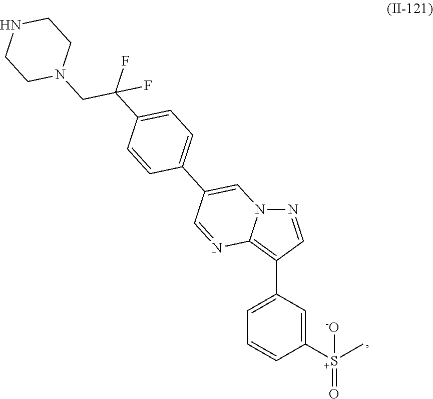

or a pharmaceutically acceptable salt thereof.

In some embodiments, the compound of Formula II has a structure of Formula II-122:

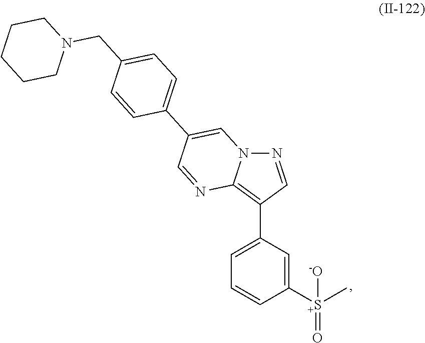

In some embodiments, the compound of Formula II has a structure of Formula II-123:

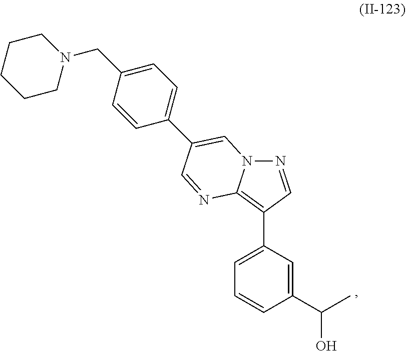 [SMILES: CC(O)c1cccc(-c2cnn3cc(-c4ccc(CN5CCCCC5)cc4)cnc23)c1]

or a pharmaceutically acceptable salt thereof.

In some embodiments, the compound of Formula II has a structure of Formula II-124:

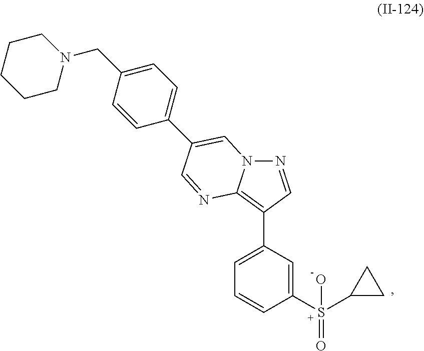

or a pharmaceutically acceptable salt thereof.

In some embodiments, the compound of Formula II has a structure of Formula II-125:

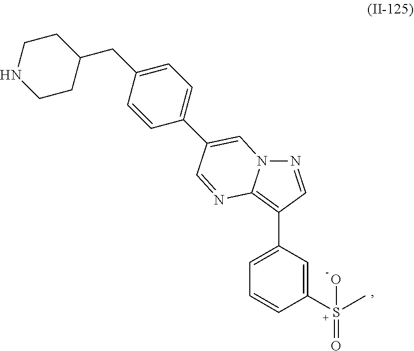

or a pharmaceutically acceptable salt thereof.

In some embodiments, the compound of Formula II has a structure of Formula II-128:

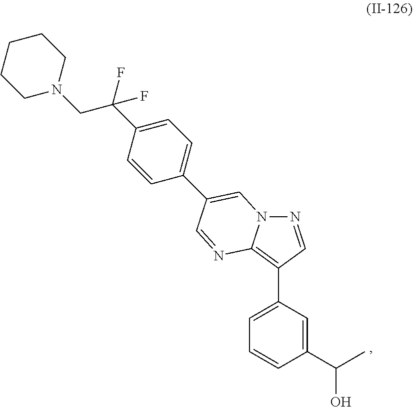 [SMILES: CC(O)c1cccc(-c2cnn3cc(-c4ccc(C(F)(F)CN5CCCCC5)cc4)cnc23)c1]

or a pharmaceutically acceptable salt thereof.

In some embodiments, the compound of Formula II has a structure of Formula II-127:

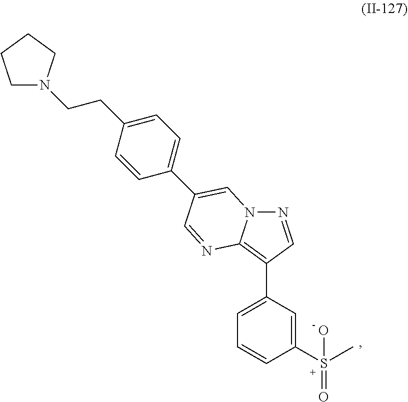

or a pharmaceutically acceptable salt thereof.

In some embodiments, the compound of Formula II has a structure of Formula II-128:

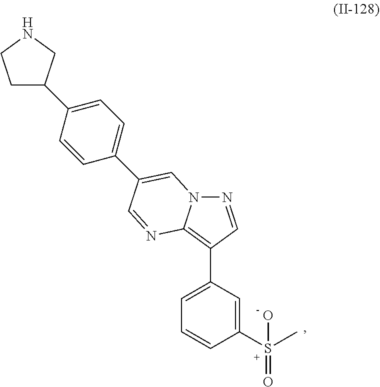

or a pharmaceutically acceptable salt thereof.

In some embodiments, the compound of Formula II has a structure of Formula II-129:

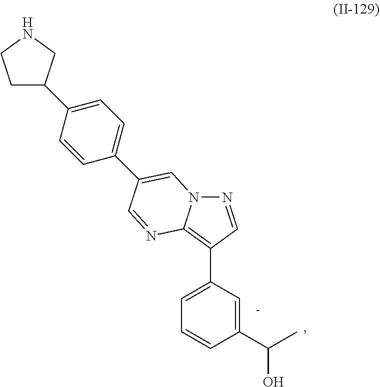 [SMILES: CC(O)c1cccc(-c2cnn3cc(-c4ccc(C5CCNC5)cc4)cnc23)c1]

or a pharmaceutically acceptable salt thereof.

In some embodiments, the compound of Formula II has a structure of Formula II-130:

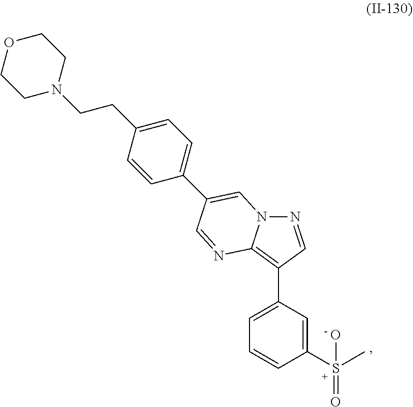

or a pharmaceutically acceptable salt thereof.

In some embodiments, the compound of Formula II has a structure of Formula II-131:

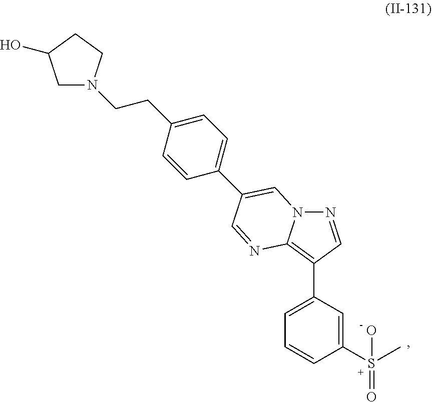

or a pharmaceutically acceptable salt thereof.

In some embodiments, the compound of Formula II has a structure of Formula II-132:

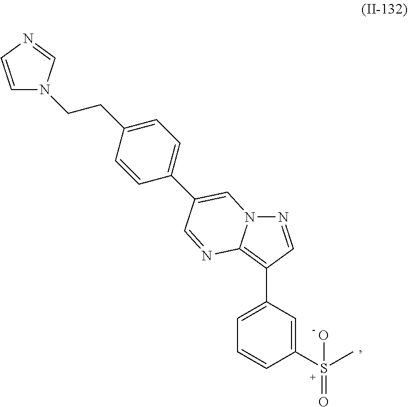

or a pharmaceutically acceptable salt thereof.

In some embodiments, the compound of Formula II has a structure of Formula II-133:

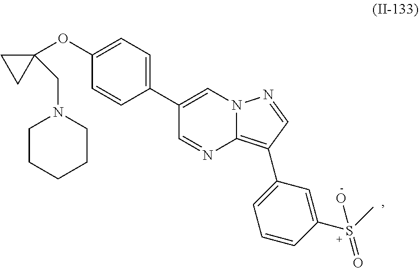

or a pharmaceutically acceptable salt thereof.

In some embodiments, the compound of Formula II has a structure of Formula II-134:

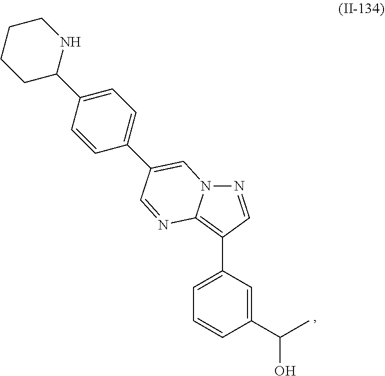 [SMILES: CC(O)c1cccc(-c2cnn3cc(-c4ccc(C5CCCCN5)cc4)cnc23)c1]

or a pharmaceutically acceptable salt thereof.

In some embodiments, the compound of Formula II has a structure of Formula II-135:

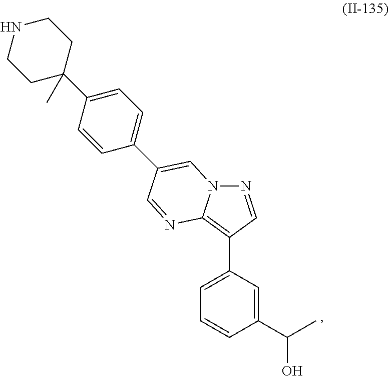 [SMILES: CC(O)c1cccc(-c2cnn3cc(-c4ccc(C5(C)CCNCC5)cc4)cnc23)c1]

or a pharmaceutically acceptable salt thereof.

In some embodiments, the compound of Formula II has a structure of Formula II-138:

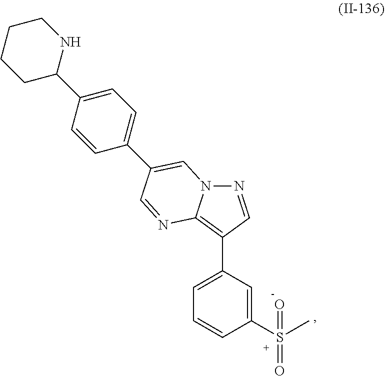 [SMILES: CS(=O)(=O)c1cccc(-c2cnn3cc(-c4ccc(C5CCCCN5)cc4)cnc23)c1]

In some embodiments, the compound of Formula II has a structure of Formula II-137:

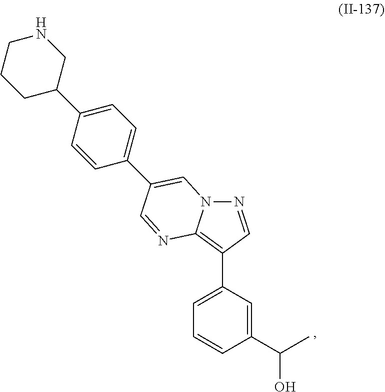 [SMILES: CC(O)c1cccc(-c2cnn3cc(-c4ccc(C5CCCNC5)cc4)cnc23)c1]

or a pharmaceutically acceptable salt thereof.

In some embodiments, the compound of Formula II has a structure of Formula II-138:

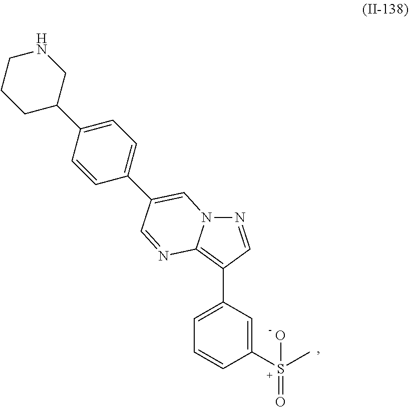

or a pharmaceutically acceptable salt thereof.

In some embodiments, the compound of Formula II has a structure of Formula II-139:

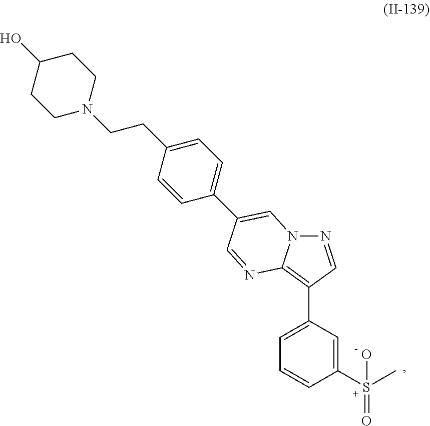

or a pharmaceutically acceptable salt thereof.

In some embodiments, the compound of Formula II has a structure of Formula II-140:

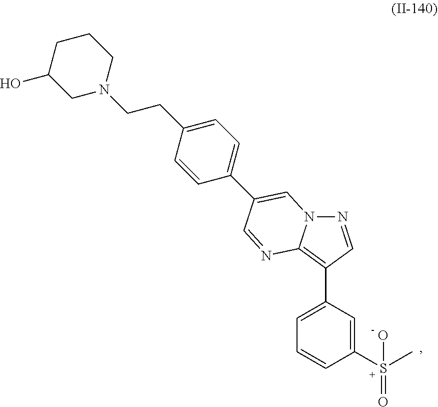

or a pharmaceutically acceptable salt thereof.

In some embodiments, the compound of Formula II has a structure of Formula II-141:

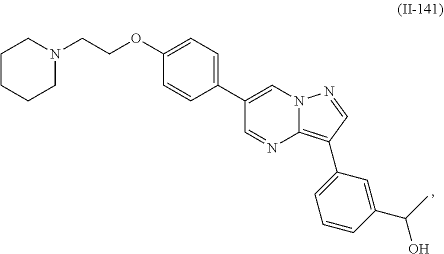 [SMILES: CC(O)c1cccc(-c2cnn3cc(-c4ccc(OCCN5CCCCC5)cc4)cnc23)c1]

or a pharmaceutically acceptable salt thereof.

In some embodiments, the compound of Formula II has a structure of Formula II-142:

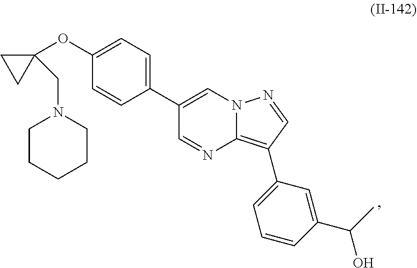 [SMILES: CC(O)c1cccc(-c2cnn3cc(-c4ccc(OC5(CN6CCCCC6)CC5)cc4)cnc23)c1]

or a pharmaceutically acceptable salt thereof.

In some embodiments, the compound of Formula II has a structure of Formula II-143:

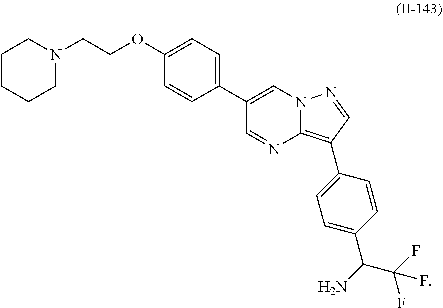 [SMILES: NC(c1ccc(-c2cnn3cc(-c4ccc(OCCN5CCCCC5)cc4)cnc23)cc1)C(F)(F)F]

or a pharmaceutically acceptable salt thereof.

In some embodiments, the compound of Formula II has a structure of Formula II-144:

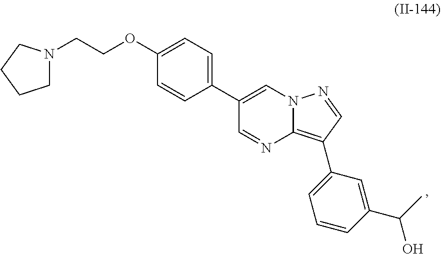 [SMILES: CC(O)c1cccc(-c2cnn3cc(-c4ccc(OCCN5CCCC5)cc4)cnc23)c1]

or a pharmaceutically acceptable salt thereof.

In some embodiments, the compound of Formula II has a structure of Formula II-145:

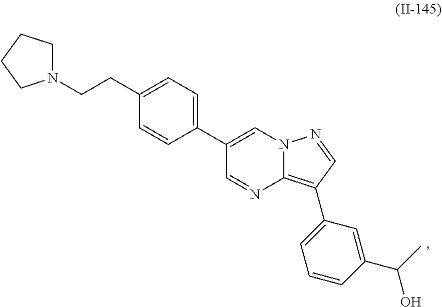 [SMILES: CC(O)c1cccc(-c2cnn3cc(-c4ccc(CCN5CCCC5)cc4)cnc23)c1]

or a pharmaceutically acceptable salt thereof.

In some embodiments, the compound of Formula II has a structure of Formula II-146:

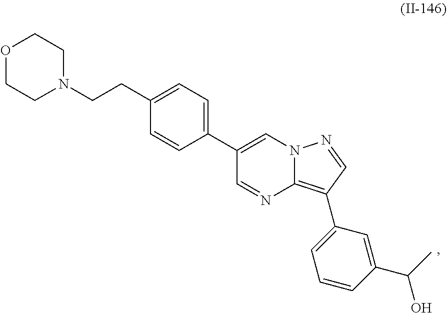 [SMILES: CC(O)c1cccc(-c2cnn3cc(-c4ccc(CCN5CCOCC5)cc4)cnc23)c1]

or a pharmaceutically acceptable salt thereof.

In some embodiments, the compound of Formula II has a structure of Formula II-147:

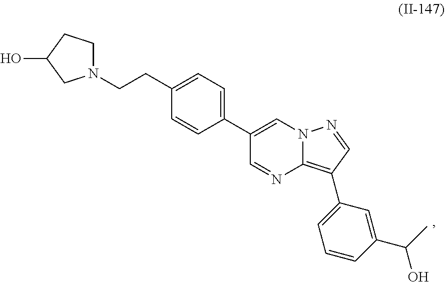 [SMILES: CC(O)c1cccc(-c2cnn3cc(-c4ccc(CCN5CCC(O)C5)cc4)cnc23)c1]

or a pharmaceutically acceptable salt thereof.

In some embodiments, the compound of Formula II has a structure of Formula II-148:

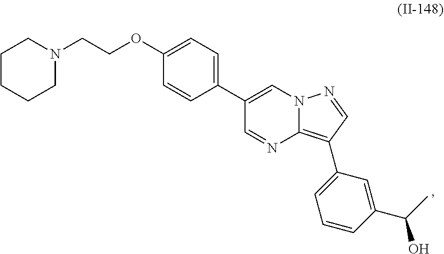 [SMILES: C[C@@H](O)c1cccc(-c2cnn3cc(-c4ccc(OCCN5CCCCC5)cc4)cnc23)c1]

or a pharmaceutically acceptable salt thereof.

In some embodiments, the compound of Formula II has a structure of Formula II-149:

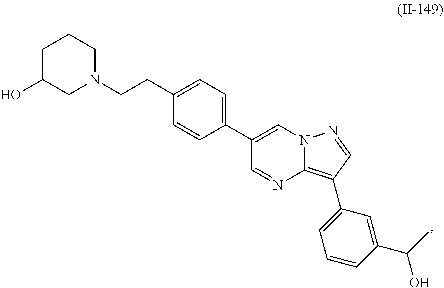 [SMILES: CC(O)c1cccc(-c2cnn3cc(-c4ccc(CCN5CCCC(O)C5)cc4)cnc23)c1]

or a pharmaceutically acceptable salt thereof.

In some embodiments, the compound of Formula II has a structure of Formula II-150:

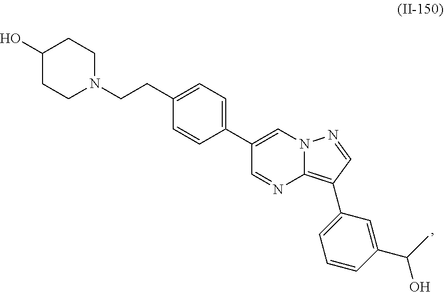 [SMILES: CC(O)c1cccc(-c2cnn3cc(-c4ccc(CCN5CCC(O)CC5)cc4)cnc23)c1]

or a pharmaceutically acceptable salt thereof.

In some embodiments, the compound of Formula II has a structure of Formula II-151:

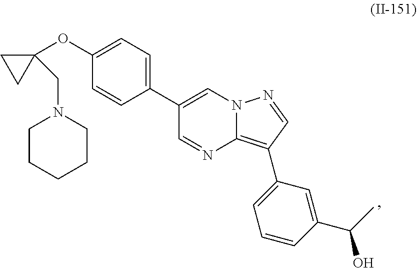 [SMILES: C[C@@H](O)c1cccc(-c2cnn3cc(-c4ccc(OC5(CN6CCCCC6)CC5)cc4)cnc23)c1]

or a pharmaceutically acceptable salt thereof.

In some embodiments, the compound of Formula II has a structure of Formula II-152:

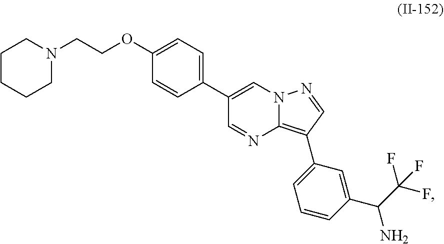 [SMILES: NC(c1cccc(-c2cnn3cc(-c4ccc(OCCN5CCCCC5)cc4)cnc23)c1)C(F)(F)F]

or a pharmaceutically acceptable salt thereof.

In some embodiments, the compound of Formula II has a structure of Formula II-153:

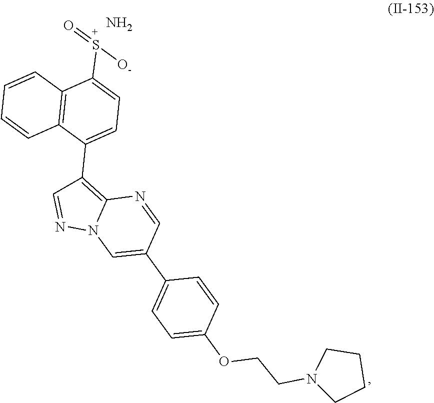

or a pharmaceutically acceptable salt thereof.

In some embodiments, the compound of Formula II has a structure of Formula II-154:

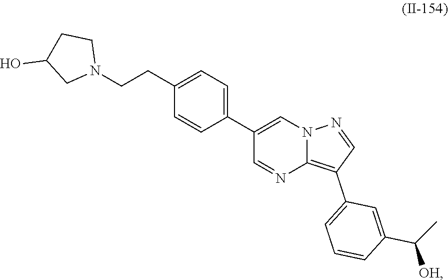 [SMILES: C[C@@H](O)c1cccc(-c2cnn3cc(-c4ccc(CCN5CCC(O)C5)cc4)cnc23)c1]

or a pharmaceutically acceptable salt thereof.

In some embodiments, the compound of Formula II has a structure of Formula II-155:

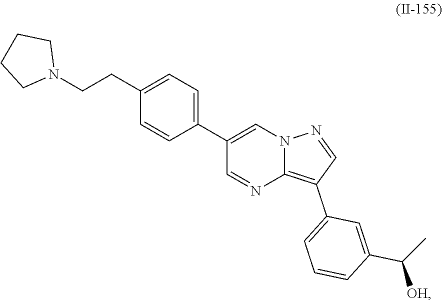 [SMILES: C[C@@H](O)c1cccc(-c2cnn3cc(-c4ccc(CCN5CCCC5)cc4)cnc23)c1]

or a pharmaceutically acceptable salt thereof.

In some embodiments, the compound of Formula II has a structure of Formula II-156:

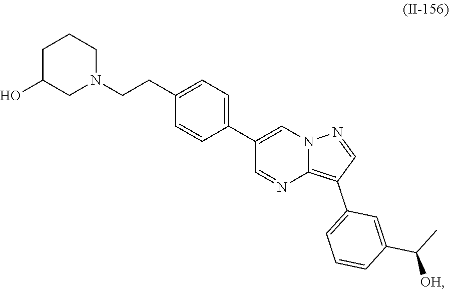 [SMILES: C[C@@H](O)c1cccc(-c2cnn3cc(-c4ccc(CCN5CCCC(O)C5)cc4)cnc23)c1]

or a pharmaceutically acceptable salt thereof.

In some embodiments, the compound of Formula II has a structure of Formula II-157:

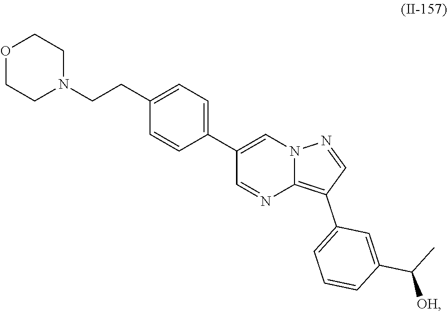 [SMILES: C[C@@H](O)c1cccc(-c2cnn3cc(-c4ccc(CCN5CCOCC5)cc4)cnc23)c1]

or a pharmaceutically acceptable salt thereof.

In some embodiments, the compound of Formula II has a structure of Formula II-158:

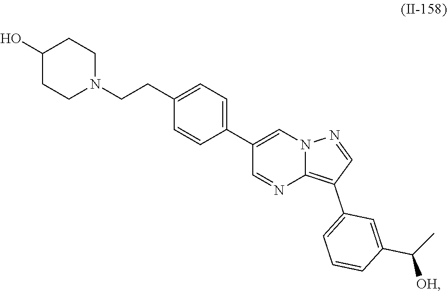 [SMILES: C[C@@H](O)c1cccc(-c2cnn3cc(-c4ccc(CCN5CCC(O)CC5)cc4)cnc23)c1]

or a pharmaceutically acceptable salt thereof.

In some embodiments, the compound of Formula II has a structure of Formula II-159:

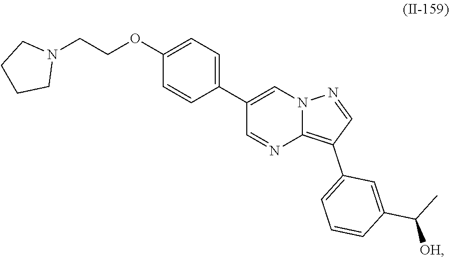 [SMILES: C[C@@H](O)c1cccc(-c2cnn3cc(-c4ccc(OCCN5CCCC5)cc4)cnc23)c1]

or a pharmaceutically acceptable salt thereof.

In some embodiments, the compound of Formula II has a structure of Formula II-160:

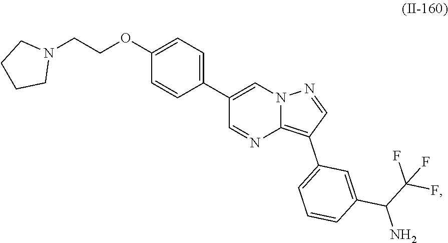 [SMILES: NC(c1cccc(-c2cnn3cc(-c4ccc(OCCN5CCCC5)cc4)cnc23)c1)C(F)(F)F]

or a pharmaceutically acceptable salt thereof.

In some embodiments, the compound of Formula II has a structure of Formula II-161:

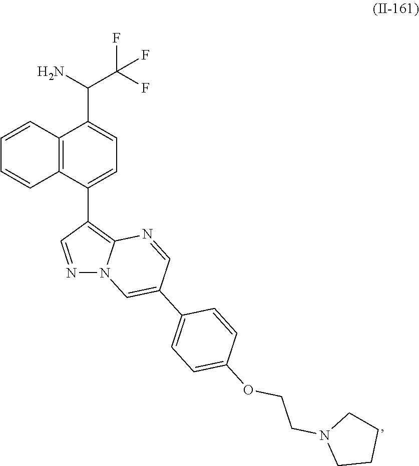 [SMILES: NC(c1ccc(-c2cnn3cc(-c4ccc(OCCN5CCCC5)cc4)cnc23)c2ccccc12)C(F)(F)F]

or a pharmaceutically acceptable salt thereof.

In some embodiments, the compound of Formula II has a structure of Formula II-162:

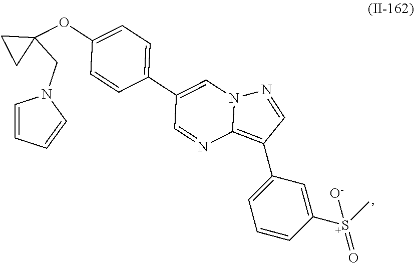 [SMILES: C[S+](=O)([O-])c1cccc(-c2cnn3cc(-c4ccc(OC5(Cn6cccc6)CC5)cc4)cnc23)c1]

or a pharmaceutically acceptable salt thereof.

In some embodiments, the compound of Formula II has a structure of Formula II-163:

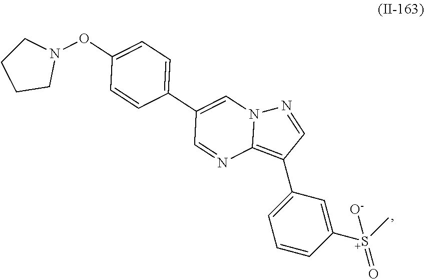 [SMILES: C[S+](=O)([O-])c1cccc(-c2cnn3cc(-c4ccc(ON5CCCC5)cc4)cnc23)c1]

or a pharmaceutically acceptable salt thereof.

In some embodiments, the compound of Formula II has a structure of Formula II-164:

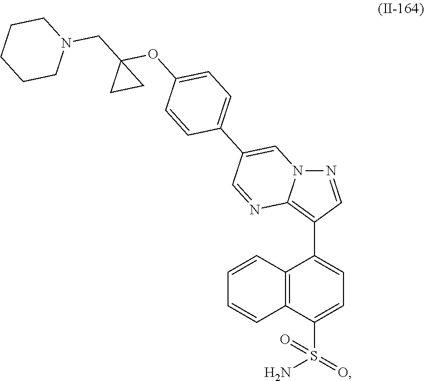 [SMILES: NS(=O)(=O)c1ccc(-c2cnn3cc(-c4ccc(OC5(CN6CCCCC6)CC5)cc4)cnc23)c2ccccc12]

or a pharmaceutically acceptable salt thereof.

In some embodiments, the compound of Formula II has a structure of Formula II-165:

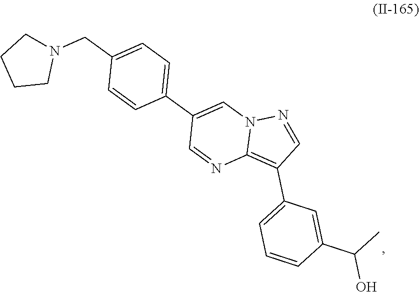 [SMILES: CC(O)c1cccc(-c2cnn3cc(-c4ccc(CN5CCCC5)cc4)cnc23)c1]

or a pharmaceutically acceptable salt thereof.

In some embodiments, the compound of Formula II has a structure of Formula II-166:

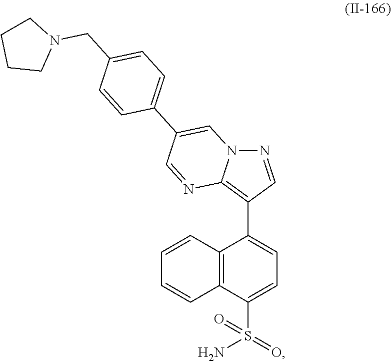 [SMILES: NS(=O)(=O)c1ccc(-c2cnn3cc(-c4ccc(CN5CCCC5)cc4)cnc23)c2ccccc12]

or a pharmaceutically acceptable salt thereof.

In some embodiments, the compound of Formula II has a structure of Formula II-167:

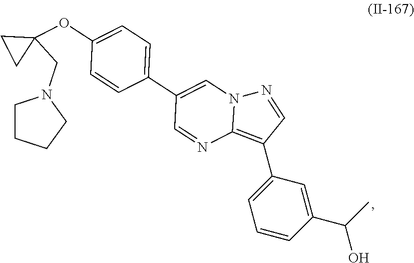 [SMILES: CC(O)c1cccc(-c2cnn3cc(-c4ccc(OC5(CN6CCCC6)CC5)cc4)cnc23)c1]

or a pharmaceutically acceptable salt thereof.

In some embodiments, the compound of Formula II has a structure of Formula II-168:

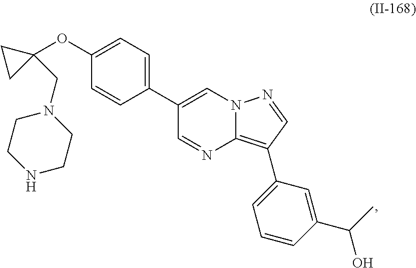 [SMILES: CC(O)c1cccc(-c2cnn3cc(-c4ccc(OC5(CN6CCNCC6)CC5)cc4)cnc23)c1]

or a pharmaceutically acceptable salt thereof.

In some embodiments, the compound of Formula II has a structure of Formula II-169:

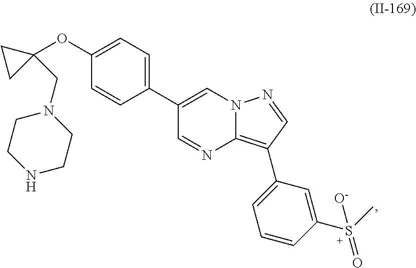

or a pharmaceutically acceptable salt thereof.

In some embodiments, the compound of Formula II has a structure of Formula II-170:

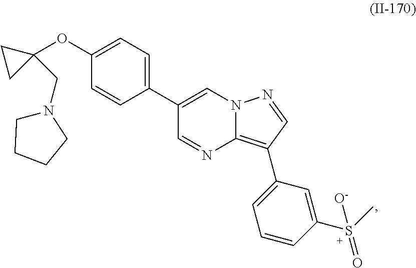

or a pharmaceutically acceptable salt thereof.

In some embodiments, the compound of Formula II has a structure of Formula II-171:

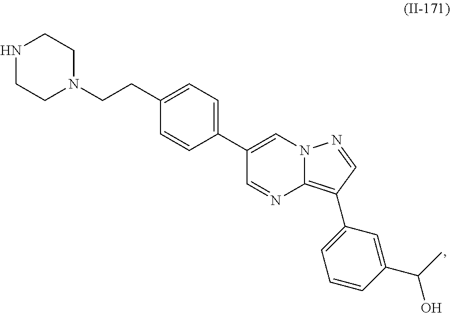 [SMILES: CC(O)c1cccc(-c2cnn3cc(-c4ccc(CCN5CCNCC5)cc4)cnc23)c1]

or a pharmaceutically acceptable salt thereof.

In some embodiments, the compound of Formula II has a structure of Formula II-172:

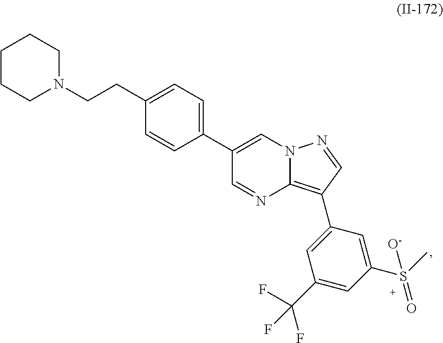

or a pharmaceutically acceptable salt thereof.

In some embodiments, the compound of Formula II has a structure of Formula II-173:

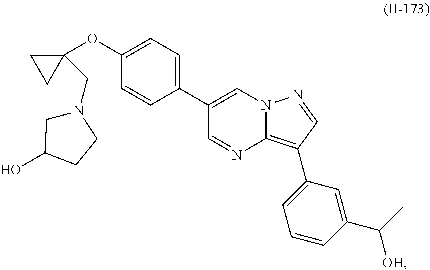 [SMILES: CC(O)c1cccc(-c2cnn3cc(-c4ccc(OC5(CN6CCC(O)C6)CC5)cc4)cnc23)c1]

or a pharmaceutically acceptable salt thereof.

In some embodiments, the compound of Formula II has a structure of Formula II-174:

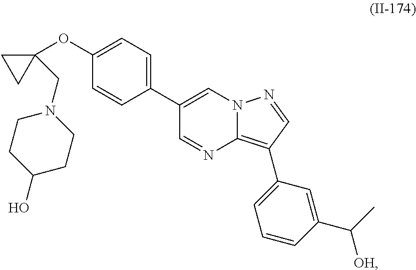 [SMILES: CC(O)c1cccc(-c2cnn3cc(-c4ccc(OC5(CN6CCC(O)CC6)CC5)cc4)cnc23)c1]

or a pharmaceutically acceptable salt thereof.

In some embodiments, the compound of Formula II has a structure of Formula II-175:

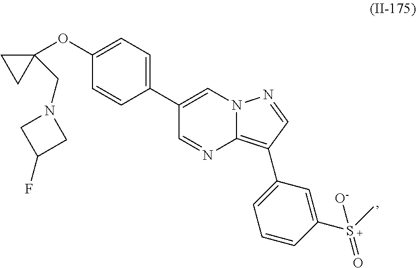

or a pharmaceutically acceptable salt thereof.

In some embodiments, the compound of Formula II has a structure of Formula II-176:

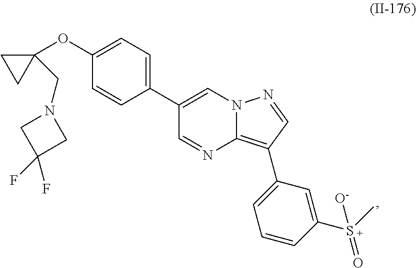

or a pharmaceutically acceptable salt thereof.

In some embodiments, the compound of Formula II has a structure of Formula II-177:

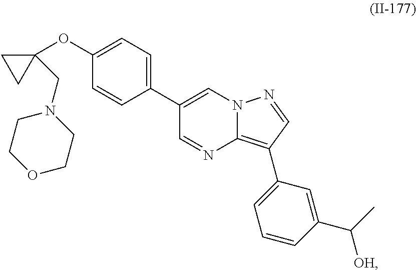 [SMILES: CC(O)c1cccc(-c2cnn3cc(-c4ccc(OC5(CN6CCOCC6)CC5)cc4)cnc23)c1]

or a pharmaceutically acceptable salt thereof.

In some embodiments, the compound of Formula II has a structure of Formula II-178:

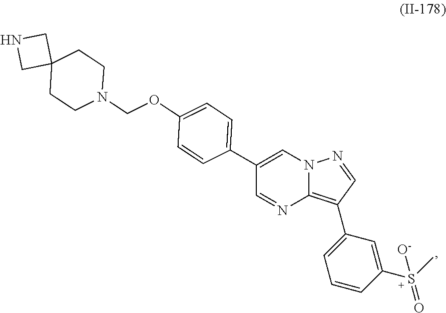

or a pharmaceutically acceptable salt thereof.

In some embodiments, the compound of Formula II has a structure of Formula II-179:

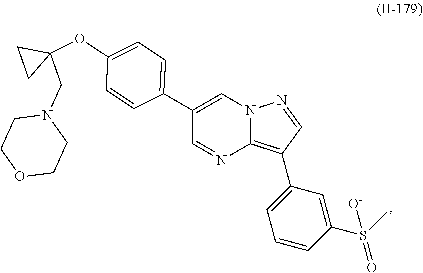

or a pharmaceutically acceptable salt thereof.

In some embodiments, the compound of Formula II has a structure of Formula II-180:

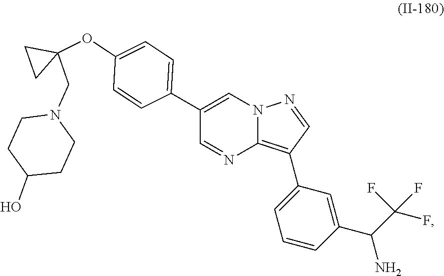 [SMILES: NC(c1cccc(-c2cnn3cc(-c4ccc(OC5(CN6CCC(O)CC6)CC5)cc4)cnc23)c1)C(F)(F)F]

or a pharmaceutically acceptable salt thereof.

In some embodiments, the compound of Formula II has a structure of Formula II-181:

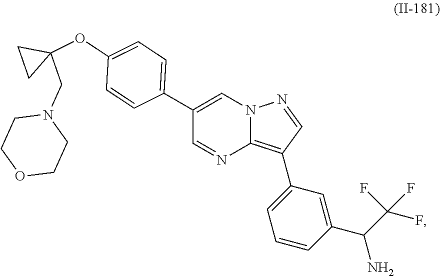 [SMILES: NC(c1cccc(-c2cnn3cc(-c4ccc(OC5(CN6CCOCC6)CC5)cc4)cnc23)c1)C(F)(F)F]

or a pharmaceutically acceptable salt thereof.

In some embodiments, the compound of Formula II has a structure of Formula II-182:

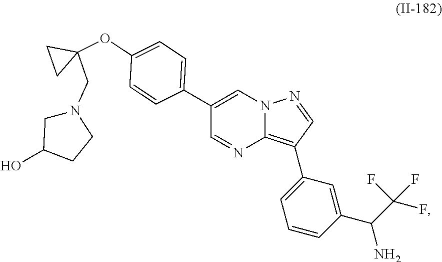 [SMILES: NC(c1cccc(-c2cnn3cc(-c4ccc(OC5(CN6CCC(O)C6)CC5)cc4)cnc23)c1)C(F)(F)F]

or a pharmaceutically acceptable salt thereof.

In some embodiments, the compound of Formula II has a structure of Formula II-183:

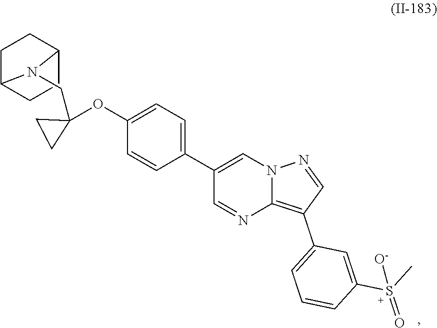

or a pharmaceutically acceptable salt thereof.

In some embodiments, the compound of Formula II has a structure of Formula II-184:

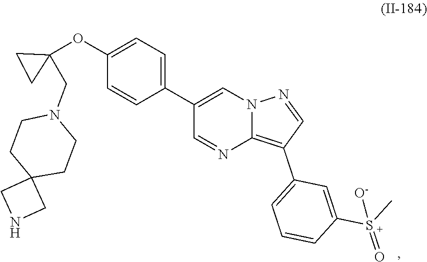

or a pharmaceutically acceptable salt thereof.

In some embodiments, the compound of Formula II has a structure of Formula II-185:

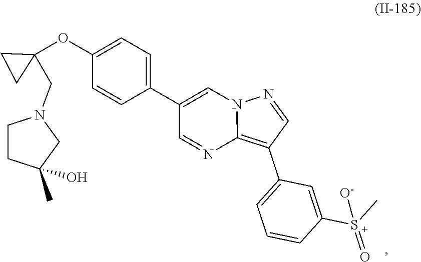

or a pharmaceutically acceptable salt thereof.

In some embodiments, the compound of Formula II has a structure of Formula II-186:

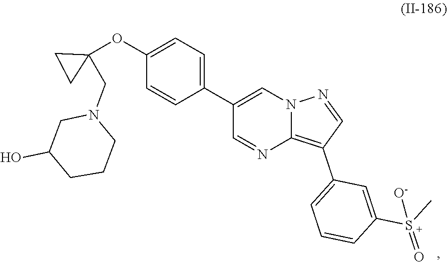

or a pharmaceutically acceptable salt thereof.

In some embodiments, the compound of Formula II has a structure of Formula II-187:

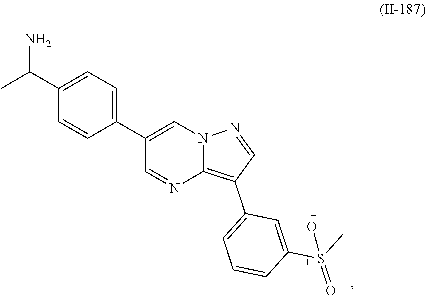

or a pharmaceutically acceptable salt thereof.

In some embodiments, the compound of Formula II has a structure of Formula II-188:

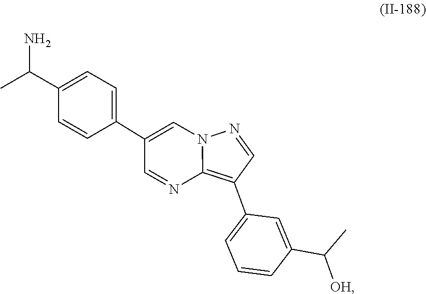 [SMILES: CC(N)c1ccc(-c2cnc3c(-c4cccc(C(C)O)c4)cnn3c2)cc1]

or a pharmaceutically acceptable salt thereof.

In some embodiments, the compound of Formula II has a structure of Formula II-189:

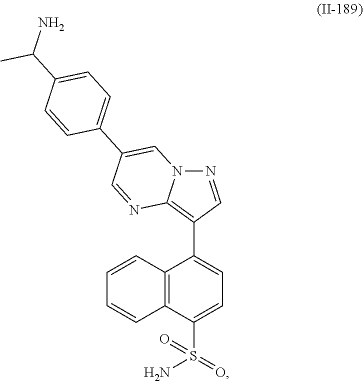 [SMILES: CC(N)c1ccc(-c2cnc3c(-c4ccc(S(N)(=O)=O)c5ccccc45)cnn3c2)cc1]

or a pharmaceutically acceptable salt thereof.

In some embodiments, the compound of Formula II has a structure of Formula II-190:

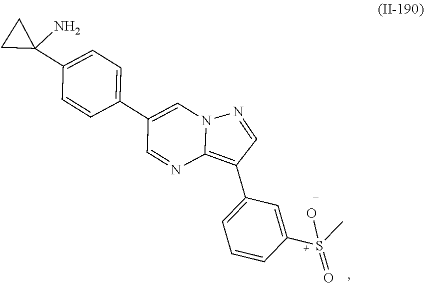

or a pharmaceutically acceptable salt thereof.

In some embodiments, the compound of Formula II has a structure of Formula II-191:

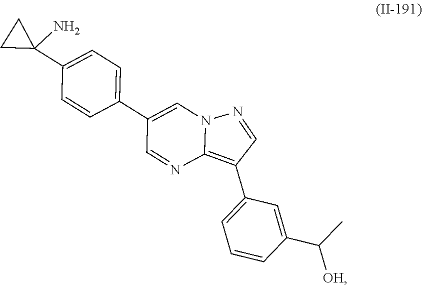 [SMILES: CC(O)c1cccc(-c2cnn3cc(-c4ccc(C5(N)CC5)cc4)cnc23)c1]

or a pharmaceutically acceptable salt thereof.

In some embodiments, the compound of Formula II has a structure of Formula II-192:

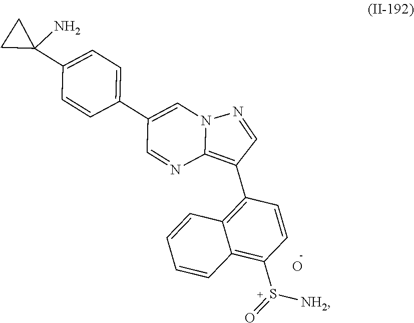

or a pharmaceutically acceptable salt thereof.

In some embodiments, the compound of Formula II has a structure of Formula II-193:

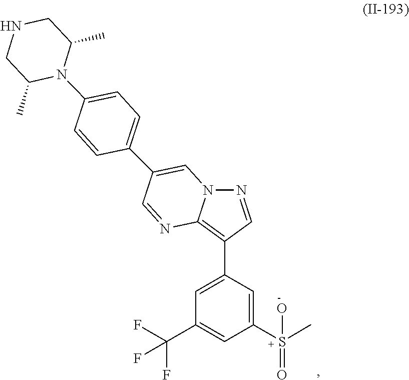

or a pharmaceutically acceptable salt thereof.

In some embodiments, the compound of Formula II has a structure of Formula II-194:

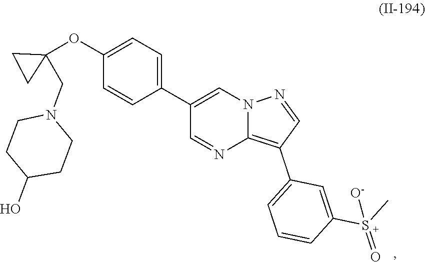

or a pharmaceutically acceptable salt thereof.

In some embodiments, the compound of Formula II has a structure of Formula II-195:

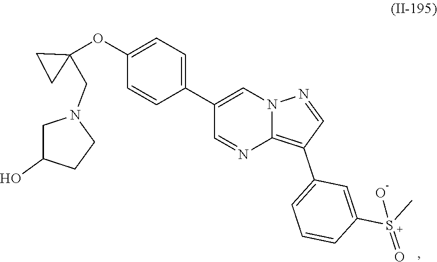

or a pharmaceutically acceptable salt thereof.

In some embodiments, the compound of Formula II has a structure of Formula II-196:

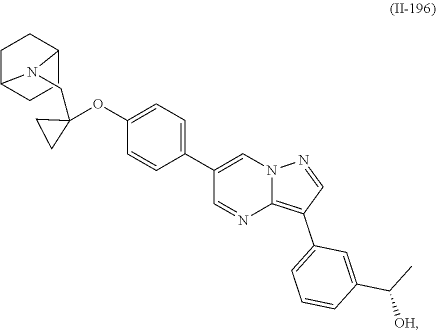 [SMILES: C[C@H](O)c1cccc(-c2cnn3cc(-c4ccc(OC5(CN6C7CCC6CC7)CC5)cc4)cnc23)c1]

or a pharmaceutically acceptable salt thereof.

In some embodiments, the compound of Formula II has a structure of Formula II-197:

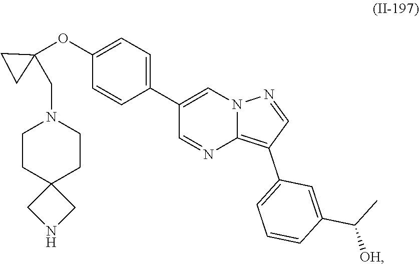 [SMILES: C[C@H](O)c1cccc(-c2cnn3cc(-c4ccc(OC5(CN6CCC7(CC6)CNC7)CC5)cc4)cnc23)c1]

or a pharmaceutically acceptable salt thereof.

In some embodiments, the compound of Formula II has a structure of Formula II-198:

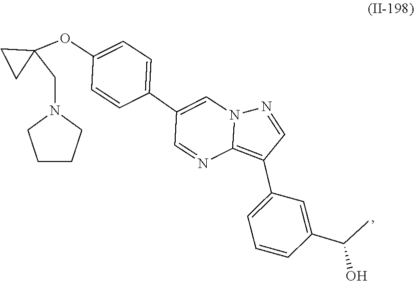 [SMILES: C[C@H](O)c1cccc(-c2cnn3cc(-c4ccc(OC5(CN6CCCC6)CC5)cc4)cnc23)c1]

or a pharmaceutically acceptable salt thereof.

In some embodiments, the compound of Formula II has a structure of Formula II-199:

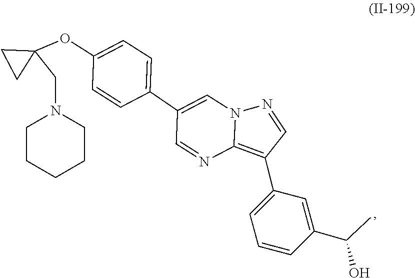 [SMILES: C[C@H](O)c1cccc(-c2cnn3cc(-c4ccc(OC5(CN6CCCCC6)CC5)cc4)cnc23)c1]

or a pharmaceutically acceptable salt thereof.

In some embodiments, the compound of Formula II has a structure of Formula II-200:

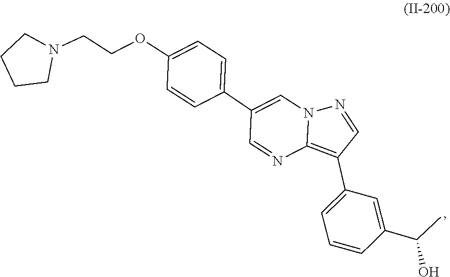 [SMILES: C[C@H](O)c1cccc(-c2cnn3cc(-c4ccc(OCCN5CCCC5)cc4)cnc23)c1]

or a pharmaceutically acceptable salt thereof.

In some embodiments, the compound of Formula II has a structure of Formula II-201:

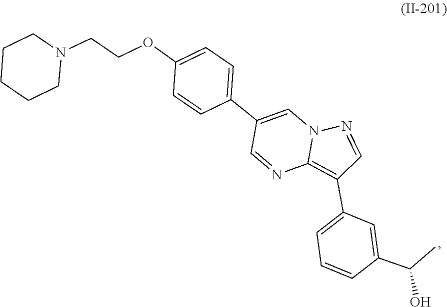 [SMILES: C[C@H](O)c1cccc(-c2cnn3cc(-c4ccc(OCCN5CCCCC5)cc4)cnc23)c1]

or a pharmaceutically acceptable salt thereof.

In some embodiments, the compound of Formula II has a structure of Formula II-202:

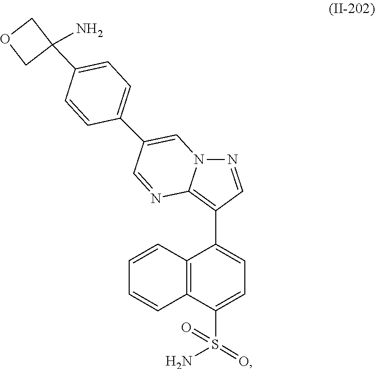 [SMILES: NC1(c2ccc(-c3cnc4c(-c5ccc(S(N)(=O)=O)c6ccccc56)cnn4c3)cc2)COC1]

or a pharmaceutically acceptable salt thereof.

In some embodiments, the compound of Formula II has a structure of Formula II-203:

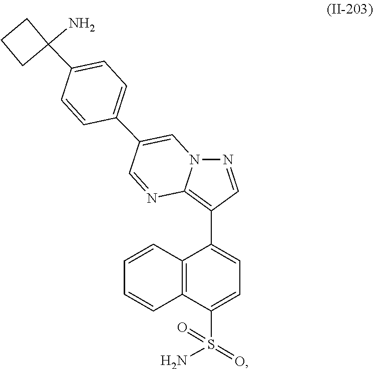 [SMILES: NC1(c2ccc(-c3cnc4c(-c5ccc(S(N)(=O)=O)c6ccccc56)cnn4c3)cc2)CCC1]

or a pharmaceutically acceptable salt thereof.

In some embodiments, the compound of Formula II has a structure of Formula II-204:

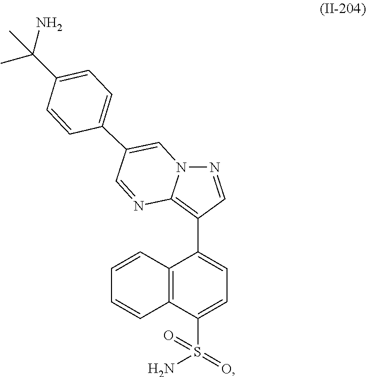 [SMILES: CC(C)(N)c1ccc(-c2cnc3c(-c4ccc(S(N)(=O)=O)c5ccccc45)cnn3c2)cc1]

In some embodiments, the compound of Formula II has a structure of Formula II-205:

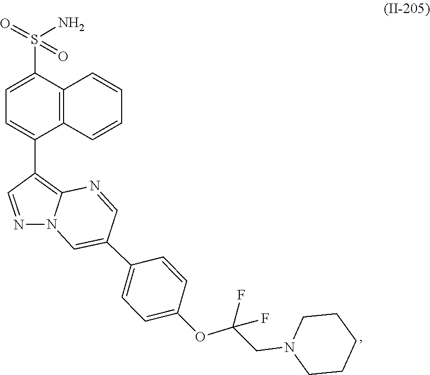 [SMILES: NS(=O)(=O)c1ccc(-c2cnn3cc(-c4ccc(OC(F)(F)CN5CCCCC5)cc4)cnc23)c2ccccc12]

or a pharmaceutically acceptable salt thereof.

In some embodiments, the compound of Formula II has a structure of Formula II-206:

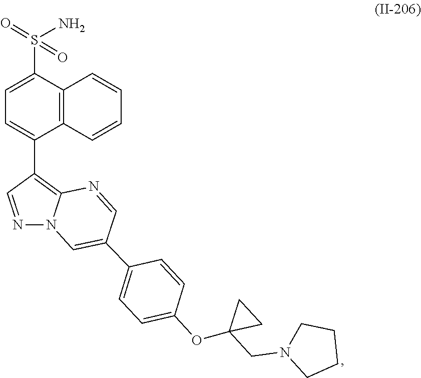 [SMILES: NS(=O)(=O)c1ccc(-c2cnn3cc(-c4ccc(OC5(CN6CCCC6)CC5)cc4)cnc23)c2ccccc12]

or a pharmaceutically acceptable salt thereof.

In some embodiments, the ALK2 inhibitor is a compound of Formula II or a pharmaceutically acceptable salt thereof, wherein
X and Y are independently selected from CR¹⁵ and N, preferably both N;
Z is selected from CR³′ and N, preferably CR³′, most preferably CH;
Ar is a phenyl ring substituted with at least one non-protium (1H) substituent or a substituted or unsubstituted heteroaryl ring;
L₁ is absent or selected from substituted or unsubstituted alkyl and heteroalkyl; and
G, J, K, and M are all absent or, independently for each occurrence, are selected from CR¹⁶ and N;
A, B, and E, independently for each occurrence, are selected from CR¹⁶ and N; provided that no more than three (and preferably no more than two) of A, B, E, G, J, K, and M are N, and at least one of E and M is N, and that if G, J, K, and M are absent then the carbon atom adjacent to E and M is optionally substituted with R¹⁶;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from H, hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido; and
R¹⁶, independently for each occurrence, is absent or is selected from H (including, and in certain embodiments preferably, D), OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, or sulfonamide.

In some embodiments, the ALK2 inhibitor is a compound of Formula II or a pharmaceutically acceptable salt thereof, wherein
X and Y are independently selected from CR¹⁵ and N, preferably both N;
Z is selected from CR³ and N, preferably CR³′, most preferably CH;
Ar is selected from substituted or unsubstituted aryl and heteroaryl;
L₁ is absent or selected from substituted or unsubstituted alkyl and heteroalkyl; and
G, J, K, and M are all absent or, independently for each occurrence, are selected from CR¹⁶ and N;
A, B, and E, independently for each occurrence, are selected from CR¹⁶ and N;
provided that no more than three (and preferably no more than two) of A, B, E, G, J, K, and M are N, and at least one of E and M is N, and that if G, J, K, and M are absent then the carbon atom adjacent to E and M is optionally substituted with R¹⁶;
R³′ is selected from H, halogen, cyano, and substituted or unsubstituted alkyl, cycloalkyl, acylamino, carbamate, sulfonyl, sulfoxido, sulfamoyl, or sulfonamido;
R⁷ is selected from H, hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido; and
R¹⁶, independently for each occurrence, is absent or is selected from H (including, and in certain embodiments preferably, D), OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, and sulfonamide; wherein B is C—R²⁵ when E is N or K is C—R²⁵ when M is N or both such that at least one of B and K is C—R²⁵, where
R²⁵ is selected from deuterium, halogen (preferably fluorine or chlorine), hydroxyl, lower alkyl (preferably methyl), and lower alkoxy (preferably methoxy), such as deuterium, fluorine, chlorine, methyl, ethyl, hydroxy, or methoxy.

In some embodiments, the ALK2 inhibitor is a compound of Formula II or a pharmaceutically acceptable salt thereof, wherein
X and Y are independently selected from CR¹⁵ and N;
Z is selected from CR³′ and N;
Ar is selected from substituted or unsubstituted aryl and heteroaryl;
L₁ is absent or selected from substituted or unsubstituted alkyl and heteroalkyl;
G, J, K, and M are all absent or, independently for each occurrence, are selected from CR¹⁶ and N;
A, B, and E, independently for each occurrence, are selected from CR¹⁶ and N;
provided that:
no more than three of A, B, E, G, J, K, and M are N,
at least one of E and M is N, and
that if G, J, K, and M are absent, then the carbon atom drawn as connected to variable M is optionally substituted with R¹⁶;
R³ is selected from H, halogen, cyano, and substituted or unsubstituted alkyl, cycloalkyl, acylamino, carbamate, sulfonyl, sulfoxido, sulfamoyl, and sulfonamido;
R⁷ is selected from hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, and sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, and sulfonamido; and
R¹⁶, independently for each occurrence, is absent or is selected from H, OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, and sulfonamide;
provided that:
i) if Ar is a phenyl ring, it is substituted with at least one non-protium (1H) substituent;
ii) B is C—R²⁵ when E is N, or K is C—R²⁵ when M is N, or both, such that at least one of B and K is C—R²⁵, wherein
R²⁵ is selected from deuterium, halogen, hydroxyl, lower alkyl, and lower alkoxy; and/or iii) R⁷ is

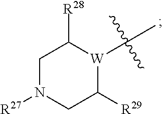

W is N, CH, or CCH₃;
R²⁷ is selected from H and substituted or unsubstituted alkyl, acyl, and ester; and
R²⁸ and R²⁹ are each independently H or alkyl, or
R²⁸ forms a one- or two-carbon bridge to the carbon atom adjacent to R²⁹ and NR²⁷; wherein either W is CH or CCH₃, or R²⁸ and R²⁹ are not both H.

Compounds of Formula II may be synthesized by methods known in the art, e.g., those described in U.S. Pat. Nos. 10,017,516 and 9,682,983, which are incorporated herein by reference.

In some embodiments, the compound of Formula II has a structure of Formula II-207:

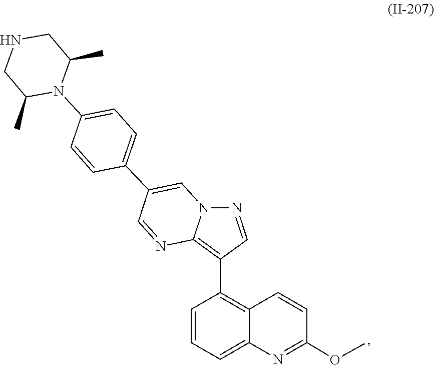 [SMILES: COc1ccc2c(-c3cnn4cc(-c5ccc(N6[C@H](C)CNC[C@@H]6C)cc5)cnc34)cccc2n1]

or a pharmaceutically acceptable salt thereof.

In some embodiments, the compound of Formula II has a structure of Formula II-208:

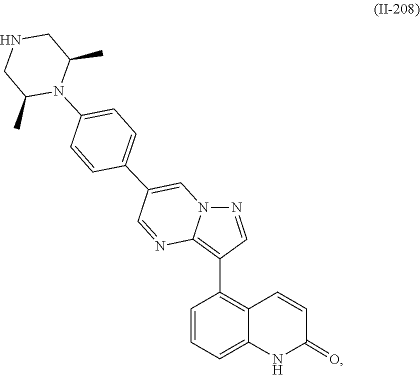 [SMILES: C[C@@H]1CNC[C@H](C)N1c1ccc(-c2cnc3c(-c4cccc5[nH]c(=O)ccc45)cnn3c2)cc1]

or a pharmaceutically acceptable salt thereof.

In some embodiments, the compound of Formula II has a structure of Formula II-209:

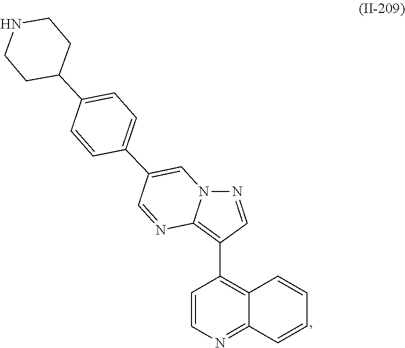 [SMILES: c1ccc2c(-c3cnn4cc(-c5ccc(C6CCNCC6)cc5)cnc34)ccnc2c1]

or a pharmaceutically acceptable salt thereof.

In some embodiments, the compound of Formula II has a structure of Formula II-210:

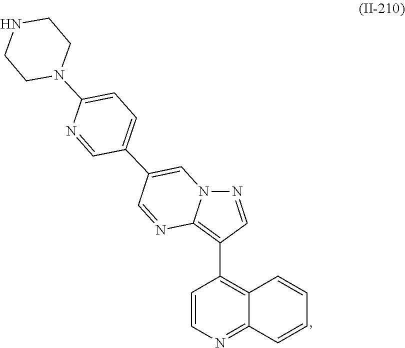 [SMILES: c1ccc2c(-c3cnn4cc(-c5ccc(N6CCNCC6)nc5)cnc34)ccnc2c1]

or a pharmaceutically acceptable salt thereof.

In some embodiments, the compound of Formula II has a structure of Formula II-211:

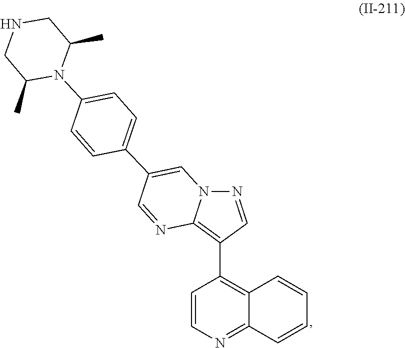 [SMILES: C[C@@H]1CNC[C@H](C)N1c1ccc(-c2cnc3c(-c4ccnc5ccccc45)cnn3c2)cc1]

or a pharmaceutically acceptable salt thereof.

In some embodiments, the compound of Formula II has a structure of Formula II-212:

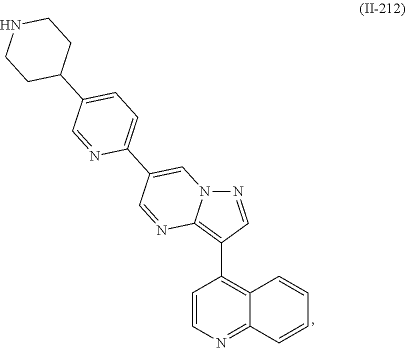 [SMILES: c1ccc2c(-c3cnn4cc(-c5ccc(C6CCNCC6)cn5)cnc34)ccnc2c1]

or a pharmaceutically acceptable salt thereof.

In some embodiments the compound of Formula II has a structure of Formula II-213:

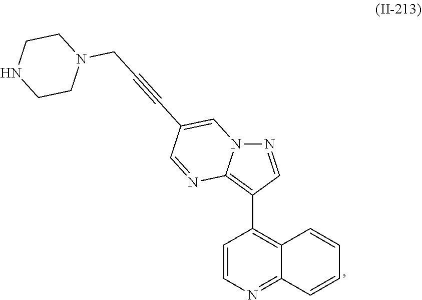 [SMILES: C(#Cc1cnc2c(-c3ccnc4ccccc34)cnn2c1)CN1CCNCC1]

or a pharmaceutically acceptable salt thereof.

In some embodiments, the compound of Formula II has a structure of Formula II-214:

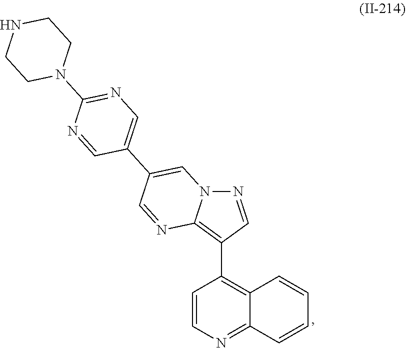 [SMILES: c1ccc2c(-c3cnn4cc(-c5cnc(N6CCNCC6)nc5)cnc34)ccnc2c1]

or a pharmaceutically acceptable salt thereof.

In some embodiments, the compound of Formula II has a structure of Formula II-215:

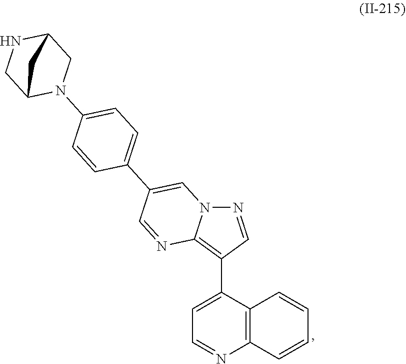 [SMILES: c1ccc2c(-c3cnn4cc(-c5ccc(N6C[C@@H]7C[C@H]6CN7)cc5)cnc34)ccnc2c1]

or a pharmaceutically acceptable salt thereof.

In some embodiments, the compound of Formula II has a structure of Formula II-216:

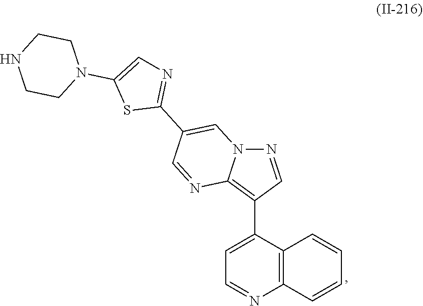 [SMILES: c1ccc2c(-c3cnn4cc(-c5ncc(N6CCNCC6)s5)cnc34)ccnc2c1]

or a pharmaceutically acceptable salt thereof.

In some embodiments the compound of Formula II has a structure of Formula II-217:5

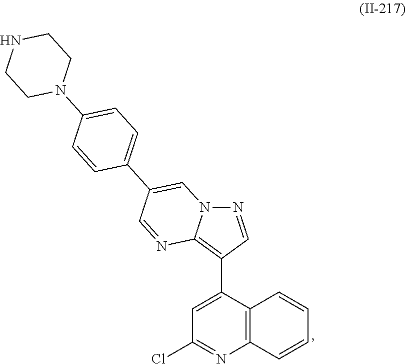 [SMILES: Clc1cc(-c2cnn3cc(-c4ccc(N5CCNCC5)cc4)cnc23)c2ccccc2n1]

or a pharmaceutically acceptable salt thereof.

In some embodiments, the compound of Formula II has a structure of Formula II-218:

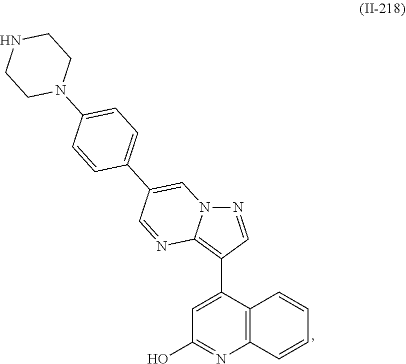 [SMILES: Oc1cc(-c2cnn3cc(-c4ccc(N5CCNCC5)cc4)cnc23)c2ccccc2n1]

or a pharmaceutically acceptable salt thereof.

In some embodiments, the compound of Formula II has a structure of Formula II-219:

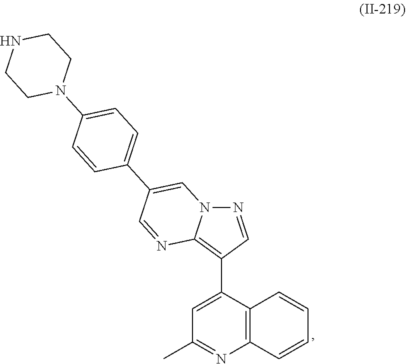 [SMILES: Cc1cc(-c2cnn3cc(-c4ccc(N5CCNCC5)cc4)cnc23)c2ccccc2n1]

or a pharmaceutically acceptable salt thereof.

In some embodiments, the compound of Formula II has a structure of Formula II-220:

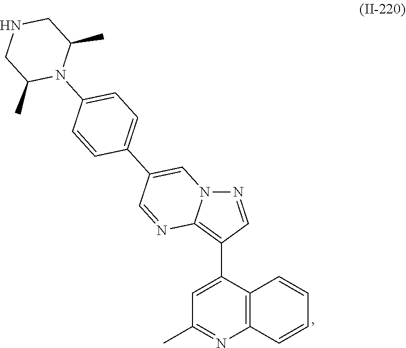 [SMILES: Cc1cc(-c2cnn3cc(-c4ccc(N5[C@H](C)CNC[C@@H]5C)cc4)cnc23)c2ccccc2n1]

or a pharmaceutically acceptable salt thereof.

In some embodiments, the compound of Formula II has a structure of Formula II-221:

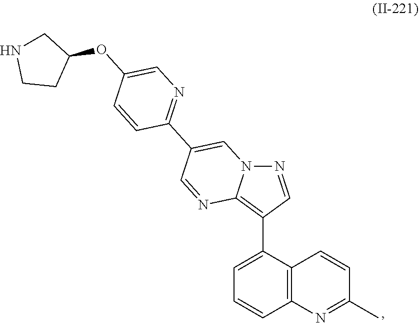 [SMILES: Cc1ccc2c(-c3cnn4cc(-c5ccc(O[C@H]6CCNC6)cn5)cnc34)cccc2n1]

or a pharmaceutically acceptable salt thereof.

In some embodiments, the compound of Formula II has a structure of Formula II-222:

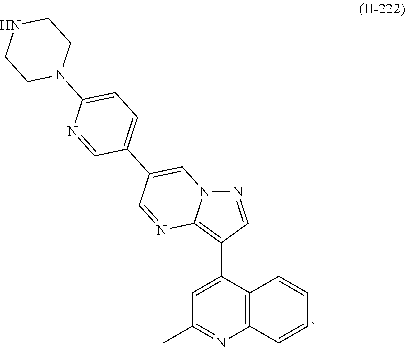 [SMILES: Cc1cc(-c2cnn3cc(-c4ccc(N5CCNCC5)nc4)cnc23)c2ccccc2n1]

or a pharmaceutically acceptable salt thereof.

In some embodiments, the compound of Formula II has a structure of Formula II-223:

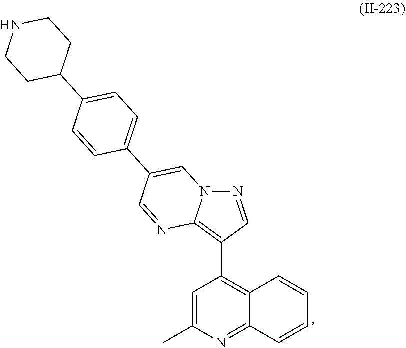 [SMILES: Cc1cc(-c2cnn3cc(-c4ccc(C5CCNCC5)cc4)cnc23)c2ccccc2n1]

or a pharmaceutically acceptable salt thereof.

In some embodiments, the compound of Formula II has a structure of Formula II-224:

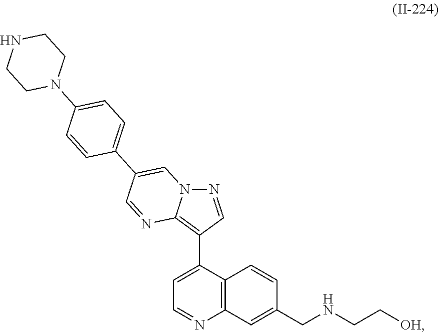 [SMILES: OCCNCc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-225:

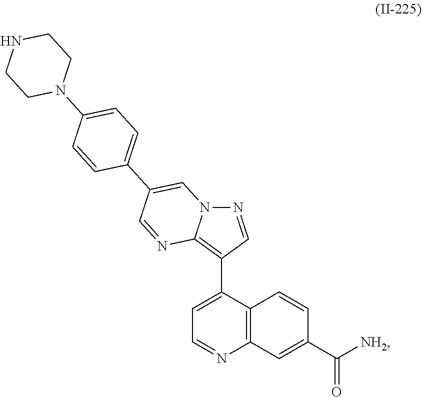 [SMILES: NC(=O)c1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-226:

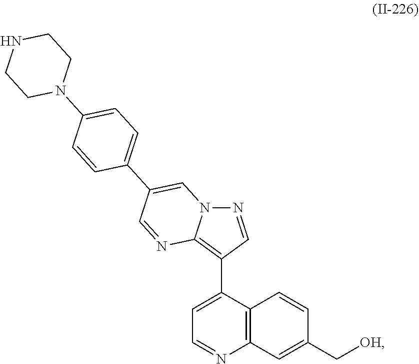 [SMILES: OCc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-227:

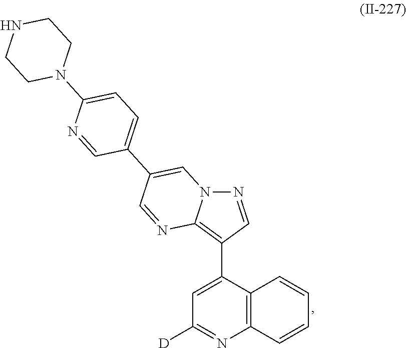 [SMILES: [2H]c1cc(-c2cnn3cc(-c4ccc(N5CCNCC5)nc4)cnc23)c2ccccc2n1]

or a pharmaceutically acceptable salt thereof.

In some embodiments, the compound of Formula II has a structure of Formula II-228:

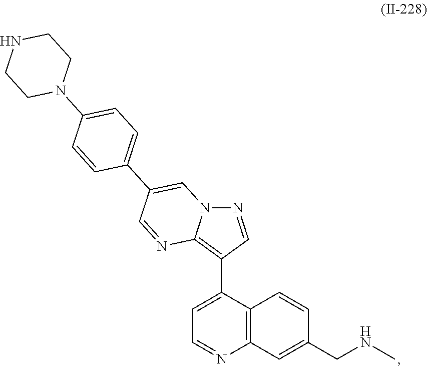 [SMILES: CNCc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-229:

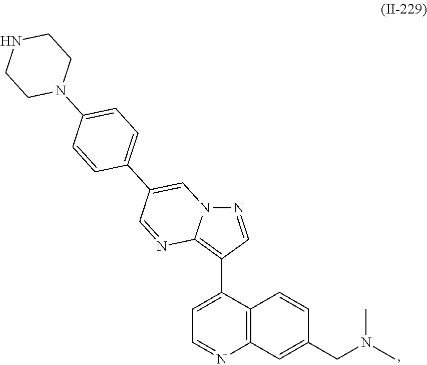 [SMILES: CN(C)Cc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-230:

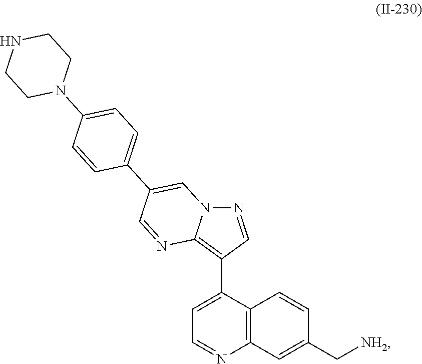 [SMILES: NCc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-231:

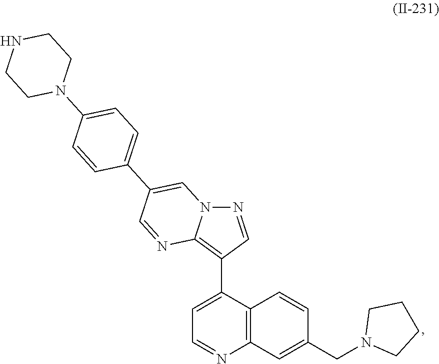 [SMILES: c1cc(-c2cnn3cc(-c4ccc(N5CCNCC5)cc4)cnc23)c2ccc(CN3CCCC3)cc2n1]

or a pharmaceutically acceptable salt thereof.

In some embodiments, the compound of Formula II has a structure of Formula II-232:

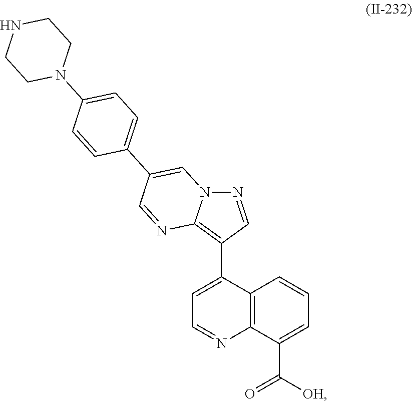 [SMILES: O=C(O)c1cccc2c(-c3cnn4cc(-c5ccc(N6CCNCC6)cc5)cnc34)ccnc12]

or a pharmaceutically acceptable salt thereof.

In some embodiments, the compound of Formula II has a structure of Formula II-233:

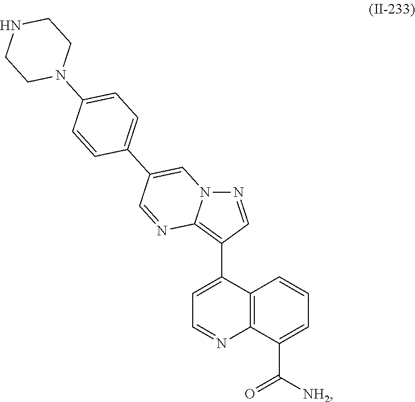 [SMILES: NC(=O)c1cccc2c(-c3cnn4cc(-c5ccc(N6CCNCC6)cc5)cnc34)ccnc12]

or a pharmaceutically acceptable salt thereof.

In some embodiments, the compound of Formula II has a structure of Formula II-234:

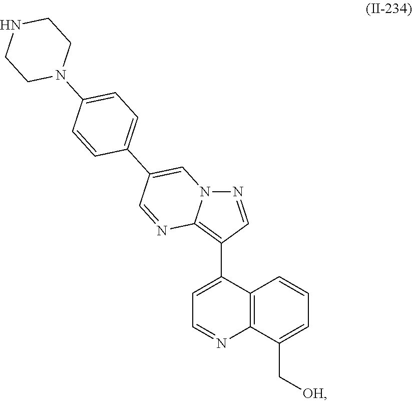 [SMILES: OCc1cccc2c(-c3cnn4cc(-c5ccc(N6CCNCC6)cc5)cnc34)ccnc12]

or a pharmaceutically acceptable salt thereof.

In some embodiments, the compound of Formula II has a structure of Formula II-235:

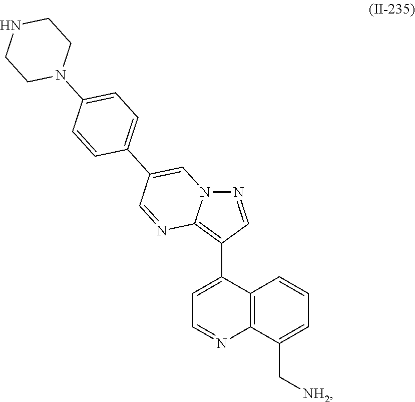 [SMILES: NCc1cccc2c(-c3cnn4cc(-c5ccc(N6CCNCC6)cc5)cnc34)ccnc12]

or a pharmaceutically acceptable salt thereof.

In some embodiments, the compound of Formula II has a structure of Formula II-236

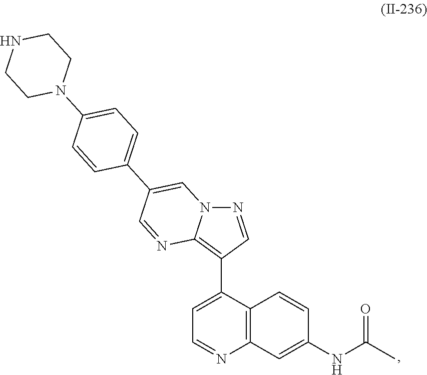 [SMILES: CC(=O)Nc1ccc2c(-c3cnn4cc(-c5ccc(N6CCNCC6)cc5)cnc34)ccnc2c1]

In some embodiments, the compound of Formula II has a structure of Formula II-237:

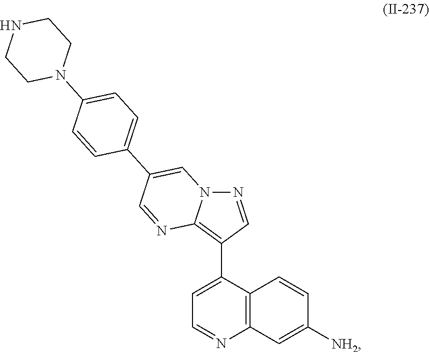 [SMILES: Nc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-238:

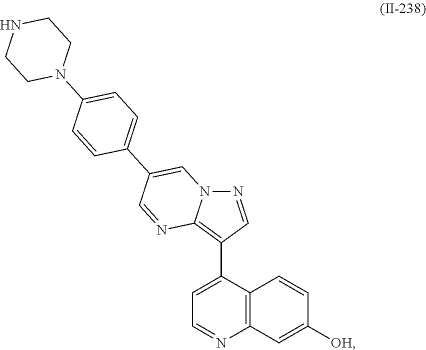 [SMILES: Oc1ccc2c(-c3cnn4cc(-c5ccc(N6CCNCC6)cc5)cnc34)ccnc2c1]

or a pharmaceutically acceptable salt thereof.

In some embodiments, the compound of Formula II has a structure of Formula II-239:

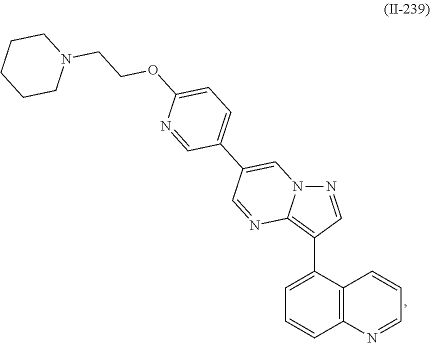 [SMILES: c1cc(-c2cnn3cc(-c4ccc(OCCN5CCCCC5)nc4)cnc23)c2cccnc2c1]

or a pharmaceutically acceptable salt thereof.

In some embodiments, the compound of Formula II has a structure of Formula II-240:

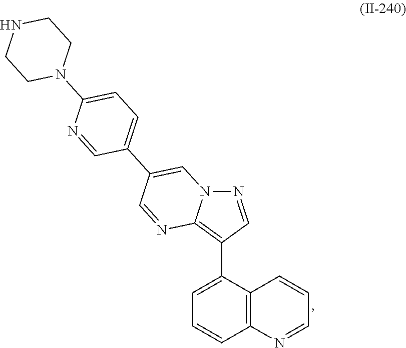 [SMILES: c1cc(-c2cnn3cc(-c4ccc(N5CCNCC5)nc4)cnc23)c2cccnc2c1]

or a pharmaceutically acceptable salt thereof.

In some embodiments, the compound of Formula II has a structure of Formula II-241:

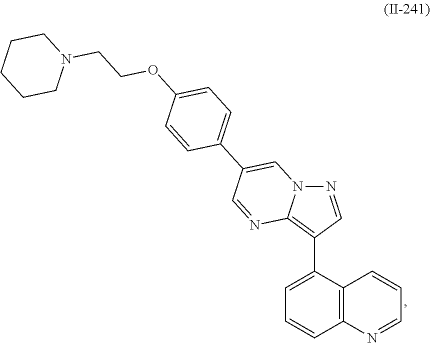 [SMILES: c1cc(-c2cnn3cc(-c4ccc(OCCN5CCCCC5)cc4)cnc23)c2cccnc2c1]

or a pharmaceutically acceptable salt thereof.

In some embodiments, the compound of Formula II has a structure of Formula II-242:

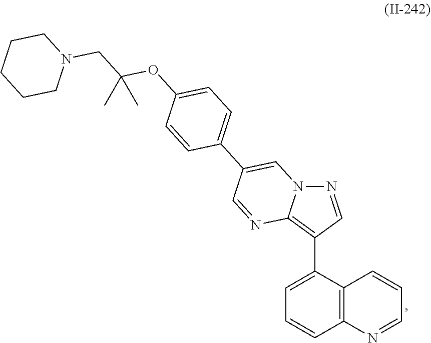 [SMILES: CC(C)(CN1CCCCC1)Oc1ccc(-c2cnc3c(-c4cccc5ncccc45)cnn3c2)cc1]

or a pharmaceutically acceptable salt thereof.

In some embodiments, the compound of Formula II has a structure of Formula II-243:

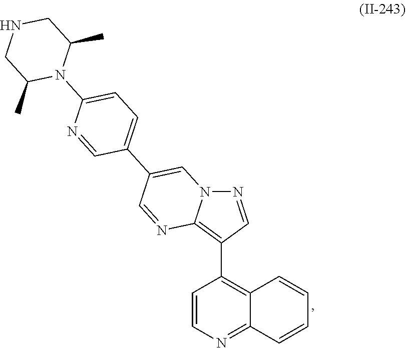 [SMILES: C[C@@H]1CNC[C@H](C)N1c1ccc(-c2cnc3c(-c4ccnc5ccccc45)cnn3c2)cn1]

or a pharmaceutically acceptable salt thereof.

In some embodiments, the compound of Formula II has a structure of Formula II-244:

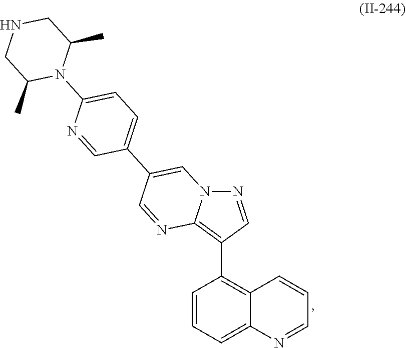 [SMILES: C[C@@H]1CNC[C@H](C)N1c1ccc(-c2cnc3c(-c4cccc5ncccc45)cnn3c2)cn1]

or a pharmaceutically acceptable salt thereof.

In some embodiments, the compound of Formula II has a structure of Formula II-245:

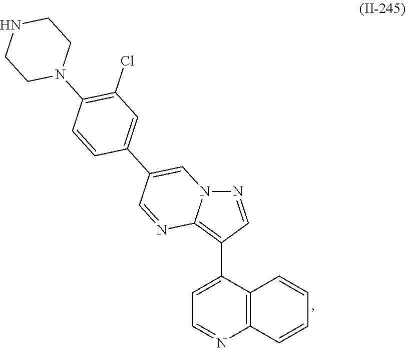 [SMILES: Clc1cc(-c2cnc3c(-c4ccnc5ccccc45)cnn3c2)ccc1N1CCNCC1]

or a pharmaceutically acceptable salt thereof.

In some embodiments, the compound of Formula II has a structure of Formula II-246:

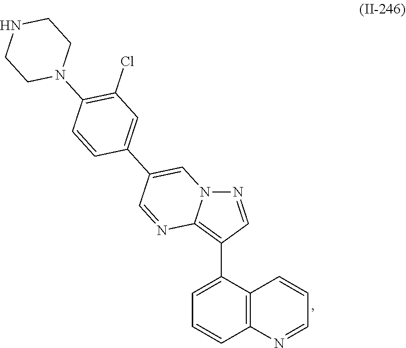 [SMILES: Clc1cc(-c2cnc3c(-c4cccc5ncccc45)cnn3c2)ccc1N1CCNCC1]

or a pharmaceutically acceptable salt thereof.

In some embodiments, the compound of Formula II has a structure of Formula II-247:

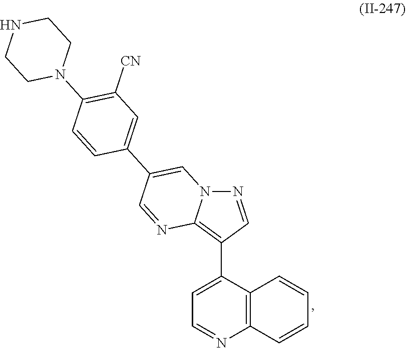 [SMILES: N#Cc1cc(-c2cnc3c(-c4ccnc5ccccc45)cnn3c2)ccc1N1CCNCC1]

or a pharmaceutically acceptable salt thereof.

In some embodiments, the compound of Formula II has a structure of Formula II-248:

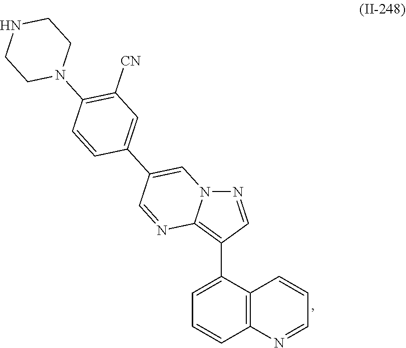 [SMILES: N#Cc1cc(-c2cnc3c(-c4cccc5ncccc45)cnn3c2)ccc1N1CCNCC1]

or a pharmaceutically acceptable salt thereof.

In some embodiments, the compound of Formula II has a structure of Formula II-249:

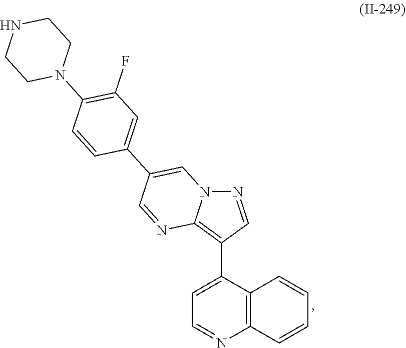 [SMILES: Fc1cc(-c2cnc3c(-c4ccnc5ccccc45)cnn3c2)ccc1N1CCNCC1]

or a pharmaceutically acceptable salt thereof.

In some embodiments, the compound of Formula II has a structure of Formula II-250:

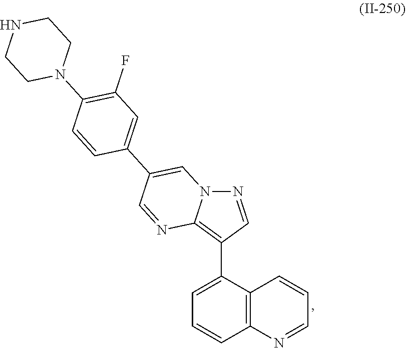 [SMILES: Fc1cc(-c2cnc3c(-c4cccc5ncccc45)cnn3c2)ccc1N1CCNCC1]

or a pharmaceutically acceptable salt thereof.

In some embodiments, the compound of Formula II has a structure of Formula II-251:

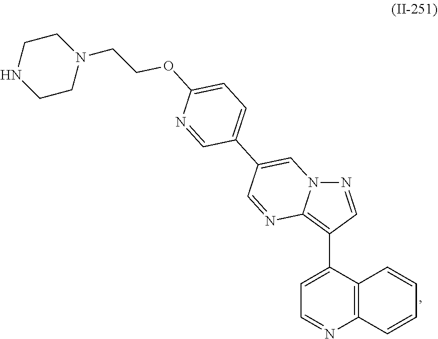 [SMILES: c1ccc2c(-c3cnn4cc(-c5ccc(OCCN6CCNCC6)nc5)cnc34)ccnc2c1]

or a pharmaceutically acceptable salt thereof.

In some embodiments, the compound of Formula II has a structure of Formula II-252:

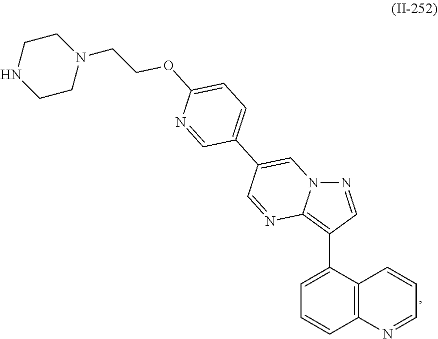 [SMILES: c1cc(-c2cnn3cc(-c4ccc(OCCN5CCNCC5)nc4)cnc23)c2cccnc2c1]

or a pharmaceutically acceptable salt thereof.

In some embodiments, the compound of Formula II has a structure of Formula II-253:

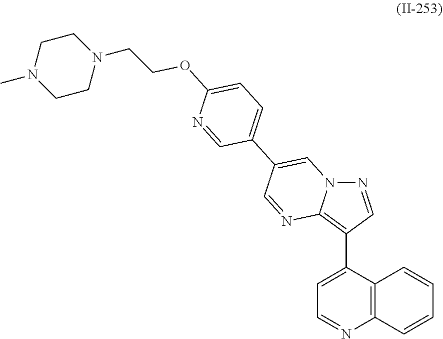 [SMILES: CN1CCN(CCOc2ccc(-c3cnc4c(-c5ccnc6ccccc56)cnn4c3)cn2)CC1]

or a pharmaceutically acceptable salt thereof.

In some embodiments the compound of Formula II has a structure of Formula II-254:

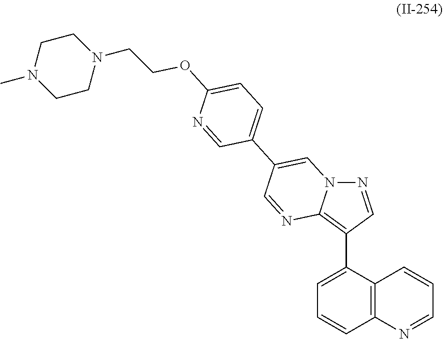 [SMILES: CN1CCN(CCOc2ccc(-c3cnc4c(-c5cccc6ncccc56)cnn4c3)cn2)CC1]

or a pharmaceutically acceptable salt thereof.

In some embodiments, the compound of Formula II has a structure of Formula II-255:

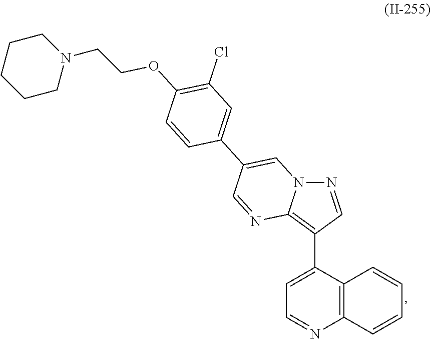 [SMILES: Clc1cc(-c2cnc3c(-c4ccnc5ccccc45)cnn3c2)ccc1OCCN1CCCCC1]

or a pharmaceutically acceptable salt thereof.

In some embodiments, the compound of Formula II has a structure of Formula II-256:

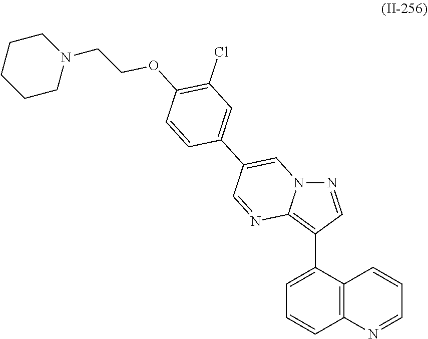 [SMILES: Clc1cc(-c2cnc3c(-c4cccc5ncccc45)cnn3c2)ccc1OCCN1CCCCC1]

or a pharmaceutically acceptable salt thereof.

In some embodiments, the compound of Formula II has a structure of Formula II-257:

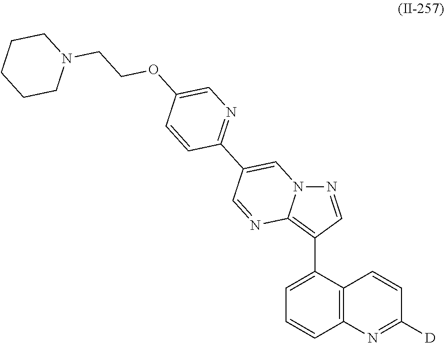 [SMILES: [2H]c1ccc2c(-c3cnn4cc(-c5ccc(OCCN6CCCCC6)cn5)cnc34)cccc2n1]

or a pharmaceutically acceptable salt thereof.

In some embodiments, the compound of Formula II has a structure of Formula II-258:

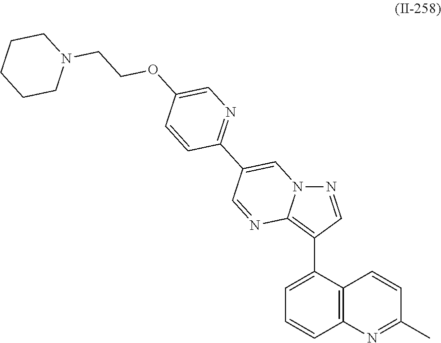 [SMILES: Cc1ccc2c(-c3cnn4cc(-c5ccc(OCCN6CCCCC6)cn5)cnc34)cccc2n1]

or a pharmaceutically acceptable salt thereof.

In some embodiments, the compound of Formula II has a structure of Formula II-259:

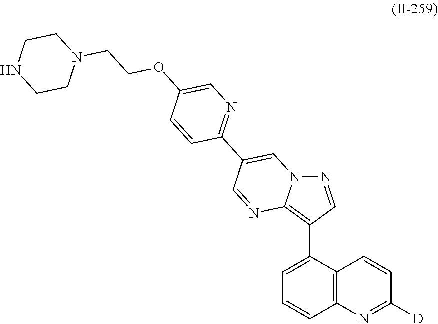 [SMILES: [2H]c1ccc2c(-c3cnn4cc(-c5ccc(OCCN6CCNCC6)cn5)cnc34)cccc2n1]

or a pharmaceutically acceptable salt thereof.

In some embodiments, the compound of Formula II has a structure of Formula II-260:

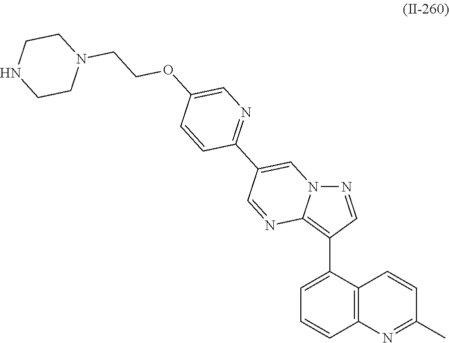 [SMILES: Cc1ccc2c(-c3cnn4cc(-c5ccc(OCCN6CCNCC6)cn5)cnc34)cccc2n1]

or a pharmaceutically acceptable salt thereof.

In some embodiments, the compound of Formula II has a structure of Formula II-261:

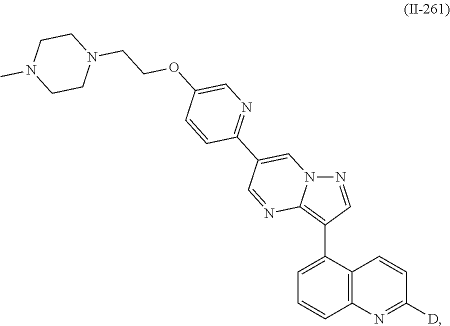

or a pharmaceutically acceptable salt thereof.

In some embodiments, the compound of Formula II has a structure of Formula II-282:

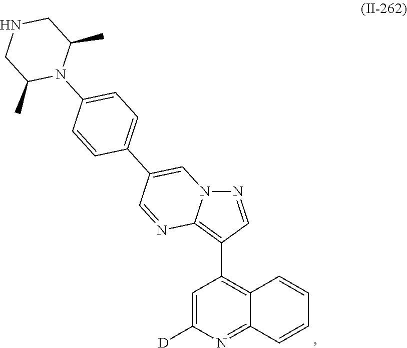 [SMILES: [2H]c1cc(-c2cnn3cc(-c4ccc(N5[C@H](C)CNC[C@@H]5C)cc4)cnc23)c2ccccc2n1]

or a pharmaceutically acceptable salt thereof.

In some embodiments, the compound of Formula II has a structure of Formula II-263:

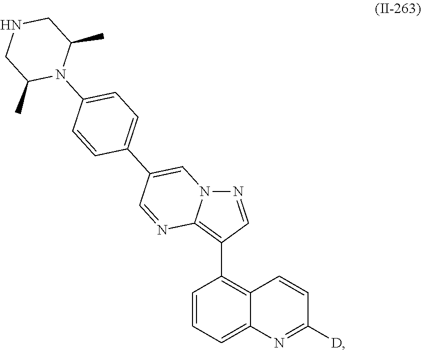

or a pharmaceutically acceptable salt thereof.

In some embodiments, the compound of Formula II has a structure of Formula II-264:

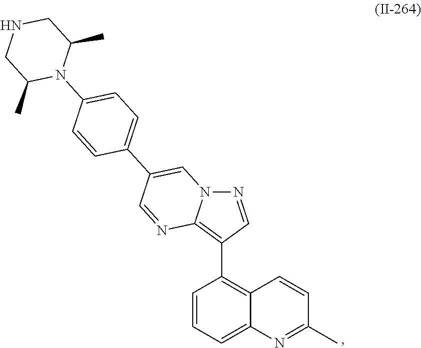 [SMILES: Cc1ccc2c(-c3cnn4cc(-c5ccc(N6[C@H](C)CNC[C@@H]6C)cc5)cnc34)cccc2n1]

or a pharmaceutically acceptable salt thereof.

In some embodiments, the compound of Formula II has a structure of Formula II-265:

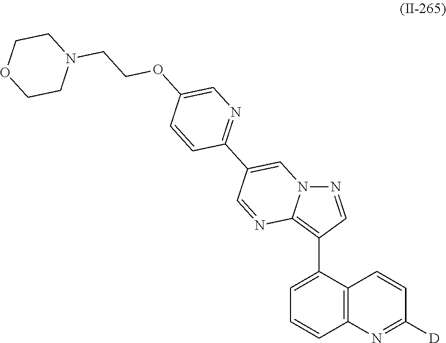 [SMILES: [2H]c1ccc2c(-c3cnn4cc(-c5ccc(OCCN6CCOCC6)cn5)cnc34)cccc2n1]

or a pharmaceutically acceptable salt thereof.

In some embodiments, the compound of Formula II has a structure of Formula II-266:

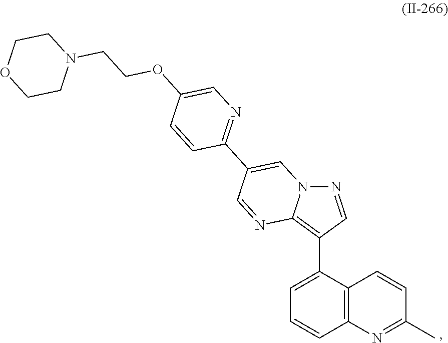 [SMILES: Cc1ccc2c(-c3cnn4cc(-c5ccc(OCCN6CCOCC6)cn5)cnc34)cccc2n1]

or a pharmaceutically acceptable salt thereof.

In some embodiments, the compound of Formula II has a structure of Formula II-267:

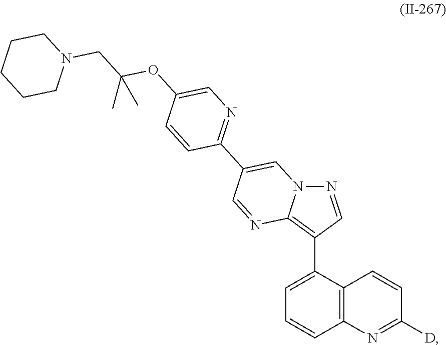

or a pharmaceutically acceptable salt thereof.

In some embodiments, the compound of Formula II has a structure of Formula II-268:

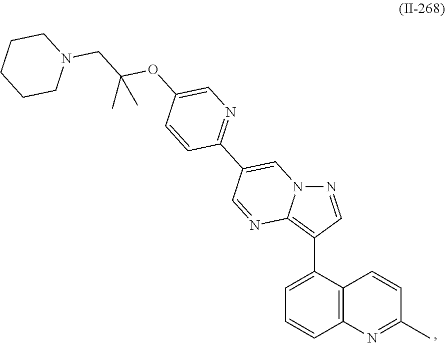 [SMILES: Cc1ccc2c(-c3cnn4cc(-c5ccc(OC(C)(C)CN6CCCCC6)cn5)cnc34)cccc2n1]

or a pharmaceutically acceptable salt thereof.

In some embodiments, the compound of Formula II has a structure of Formula II-269:

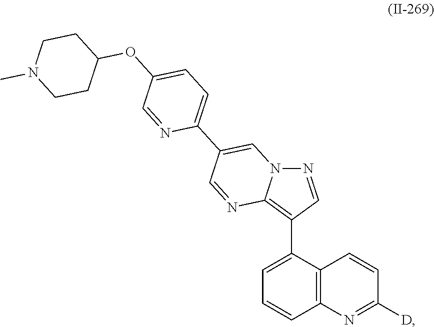

or a pharmaceutically acceptable salt thereof.

In some embodiments, the compound of Formula II has a structure of Formula II-270:

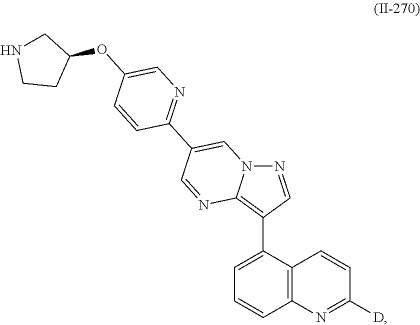

or a pharmaceutically acceptable salt thereof.

In some embodiments, the compound of Formula II has a structure of Formula II-271:

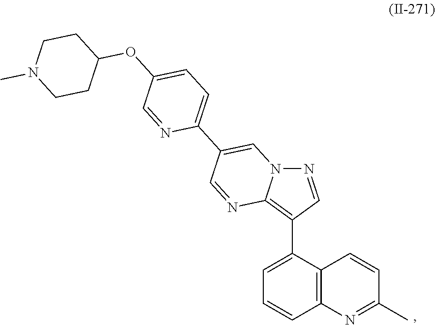

or a pharmaceutically acceptable salt thereof.

In some embodiments, the compound of Formula II has a structure of Formula II-272:

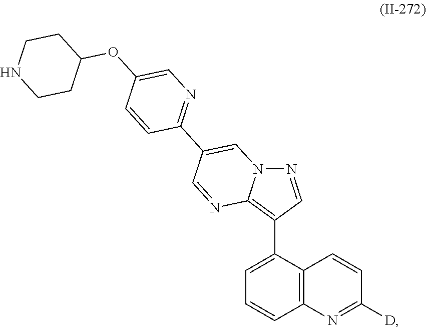

or a pharmaceutically acceptable salt thereof.

In some embodiments, the compound of Formula II has a structure of Formula II-273:

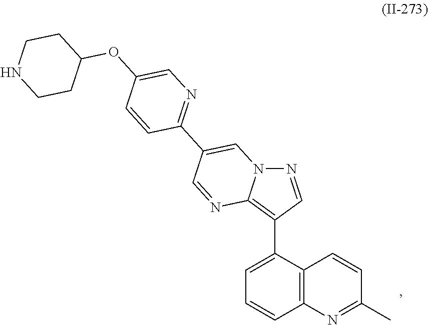 [SMILES: Cc1ccc2c(-c3cnn4cc(-c5ccc(OC6CCNCC6)cn5)cnc34)cccc2n1]

or a pharmaceutically acceptable salt thereof.

In some embodiments, the compound of Formula II has a structure of Formula II-274:

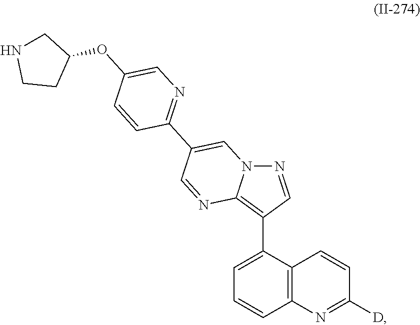

or a pharmaceutically acceptable salt thereof.

In some embodiments, the compound of Formula II has a structure of Formula II-275:

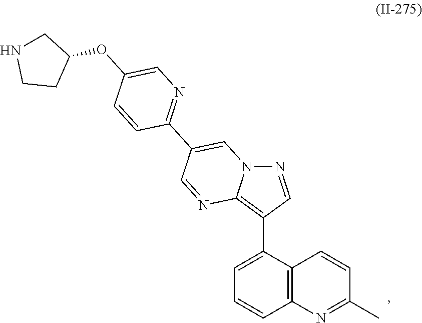 [SMILES: Cc1ccc2c(-c3cnn4cc(-c5ccc(O[C@@H]6CCNC6)cn5)cnc34)cccc2n1]

or a pharmaceutically acceptable salt thereof.

Additional compounds of Formula II are described U.S. Pat. Nos. 10,513,521, 10,017,516, and 9,682,983, which are incorporated herein by reference.

In some embodiments, the ALK2 inhibitor is a compound of Formula III:

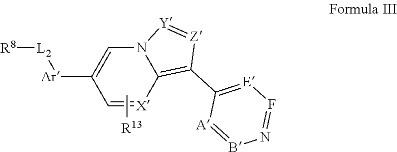

or a pharmaceutically acceptable salt thereof, wherein
X′ is selected from CR¹⁵′ and N;
Y′ is selected from CR¹⁵ and N;
Z′ is selected from CR²⁸ and N;
Ar′ is selected from substituted or unsubstituted aryl and heteroaryl, e.g., a six-membered ring, such as phenyl;
L₂ is absent or selected from substituted or unsubstituted alkyl and heteroalkyl;
A and B, independently for each occurrence, are selected from CR¹⁶ and N, preferably CR¹⁶, e.g., CH;
E and F, independently for each occurrence, are selected from CR⁵′ and N, preferably CR⁵;
preferably chosen such that no more than two of A, B, E, and F are N;
R²⁸ represents a substituent, e.g., selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido, e.g., lower alkyl;
R⁸ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido, e.g., substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, or sulfonamido, preferably substituted or unsubstituted heterocyclyl or heteroaryl;
R⁵, independently for each occurrence, represents a substituent, e.g., selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano), or two occurrences of R⁵ taken together with the atoms to which they are attached form a substituted or unsubstituted 5- or 6-membered cycloalkyl, heterocycloalkyl, aryl, or heteroaryl ring, preferably an aryl or heteroaryl ring, e.g., a substituted or unsubstituted benzo ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably substituted or unsubstituted alkyl, heteroalkyl, halogen, hydroxyl, alkoxyl, alkylthio, acyloxy, acylamino, carbamate, or cyano;
R¹⁵′, independently for each occurrence, represents a substituent, e.g., selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably H or substituted or unsubstituted alkyl, heteroalkyl, halogen, hydroxyl, alkoxyl, alkylthio, acyloxy, acylamino, carbamate, or cyano;
R¹⁶′, independently for each occurrence, represents a substituent, e.g., selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, preferably H or substituted or unsubstituted alkyl, alkenyl, heteroalkyl, halogen,

Compounds of Formula III may be synthesized by methods known in the art, e.g., those described in U.S. Pat. Nos. 8,507,501 and 9,045,484, which are incorporated herein by reference.

In some embodiments, a provided compound of Formula III has a structure of Formula III-a:

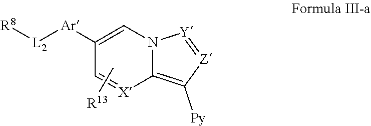

or a pharmaceutically acceptable salt thereof,
wherein
X′ is selected from CR¹⁵′ and N;
Y′ is selected from CR¹⁵ and N;
Z′ is selected from CR²⁶ and N;
Ar′ is selected from substituted or unsubstituted aryl and heteroaryl, e.g., a six-membered ring, such as phenyl;
L₂ is absent or selected from substituted or unsubstituted alkyl and heteroalkyl;
Py is substituted or unsubstituted 4-pyridinyl or 4-quinolinyl, e.g., optionally substituted with substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, amino, acylamino, carbamate, amido, amidino, cyano, sulfonyl, sulfoxido, sulfamoyl, or sulfonamido; and
R²⁶ represents a substituent, e.g., selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido, e.g., lower alkyl;
R⁸ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido, e.g., substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, or sulfonamido, preferably substituted or unsubstituted heterocyclyl or heteroaryl;
R⁵′, independently for each occurrence, represents a substituent, e.g., selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano), or two occurrences of R²⁶ taken together with the atoms to which they are attached form a substituted or unsubstituted 5- or 6-membered cycloalkyl, heterocycloalkyl, aryl, or heteroaryl ring, preferably an aryl or heteroaryl ring, e.g., a substituted or unsubstituted benzo ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxide, sulfamoyl, or sulfonamido, preferably substituted or unsubstituted alkyl, heteroalkyl, halogen, hydroxyl, alkoxyl, alkylthio, acyloxy, acylamino, carbamate, or cyano;
R¹⁵′, independently for each occurrence, represents a substituent, e.g., selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably H or substituted or unsubstituted alkyl, heteroalkyl, halogen, hydroxyl, alkoxyl, alkylthio, acyloxy, acylamino, carbamate, or cyano;
R¹⁶′, independently for each occurrence, represents a substituent, e.g., selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, or cyano.

In some embodiments, the compound of Formula III has a structure of Formula III-b:

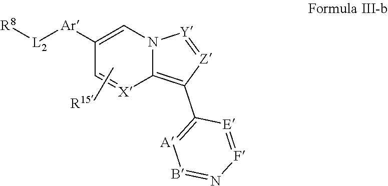

or a pharmaceutically acceptable salt thereof, wherein
X′ and Y′ are each N;
Z′ is CR²⁶;
Ar′ is substituted or unsubstituted phenyl;
L₂ is absent or selected from substituted or unsubstituted alkyl and heteroalkyl;
A′ and B′ are both CR¹⁶; E′ and F′ are both CR⁵′ and both occurrences of R⁵ taken together with E′ and F′ form a substituted or unsubstituted 5- or 6-membered cycloalkyl, heterocycloalkyl, aryl, or heteroaryl ring;
R²⁶ is selected from H and substituted or unsubstituted alkyl;
R⁸ is selected from H and substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R¹⁵′, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido; and
R¹⁶′, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In some embodiments, the compound of Formula III has a structure of Formula III-b, or a pharmaceutically acceptable salt thereof, wherein
X′ and Y′ are each N;
Z′ is CR²⁸;
Ar′ is selected from substituted or unsubstituted aryl and heteroaryl;
L₂ is absent or selected from substituted or unsubstituted alkyl and heteroalkyl;
A′ and B′ are both CR¹⁶″;
E′ and F′ are both CR⁵′ and both occurrences of R⁵ taken together with E′ and F′ form a substituted or unsubstituted 5- or 6-membered cycloalkyl, heterocycloalkyl, aryl, or heteroaryl ring;
R²⁶ is selected from H and substituted or unsubstituted alkyl;
R⁸ is selected from H and substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R¹⁵′, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido;
R¹⁶′, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In some embodiments, the compound of Formula III has a structure of Formula III-1:

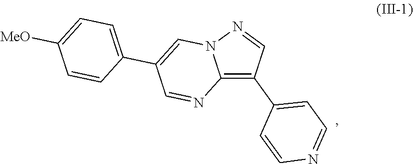 [SMILES: COc1ccc(-c2cnc3c(-c4ccncc4)cnn3c2)cc1]

or a pharmaceutically acceptable salt thereof.

In some embodiments, the compound of Formula III has a structure of Formula III-2:

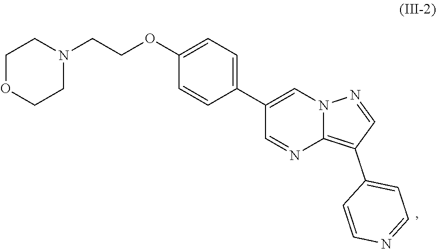 [SMILES: c1cc(-c2cnn3cc(-c4ccc(OCCN5CCOCC5)cc4)cnc23)ccn1]

or a pharmaceutically acceptable salt thereof.

In some embodiments, the compound of Formula III has a structure of Formula III-3:

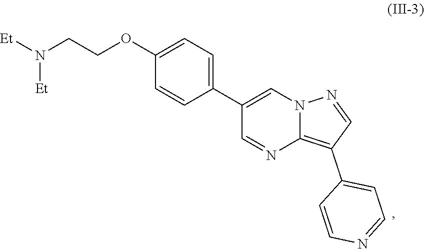 [SMILES: CCN(CC)CCOc1ccc(-c2cnc3c(-c4ccncc4)cnn3c2)cc1]

or a pharmaceutically acceptable salt thereof.

In some embodiments, the compound of Formula III has a structure of Formula III-4:

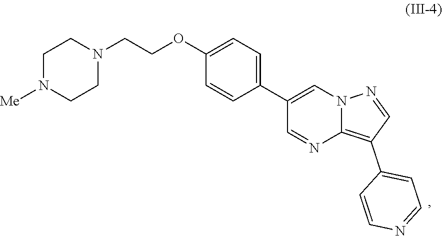 [SMILES: CN1CCN(CCOc2ccc(-c3cnc4c(-c5ccncc5)cnn4c3)cc2)CC1]

or a pharmaceutically acceptable salt thereof.

In some embodiments, the compound of Formula III has a structure of Formula III-5:

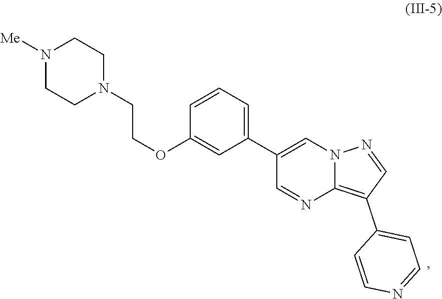 [SMILES: CN1CCN(CCOc2cccc(-c3cnc4c(-c5ccncc5)cnn4c3)c2)CC1]

or a pharmaceutically acceptable salt thereof.

In some embodiments, the compound of Formula III has a structure of Formula III-6:

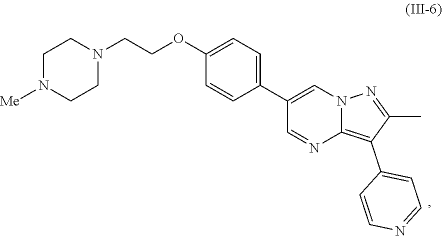 [SMILES: Cc1nn2cc(-c3ccc(OCCN4CCN(C)CC4)cc3)cnc2c1-c1ccncc1]

or a pharmaceutically acceptable salt thereof.

In some embodiments, the compound of Formula III has a structure of Formula III-7:

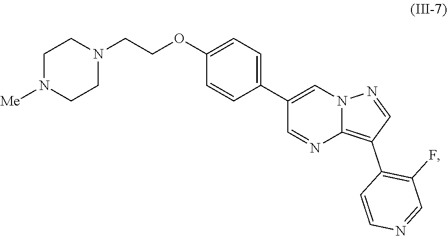 [SMILES: CN1CCN(CCOc2ccc(-c3cnc4c(-c5ccncc5F)cnn4c3)cc2)CC1]

or a pharmaceutically acceptable salt thereof.

In some embodiments, the compound of Formula III has a structure of Formula III-8:

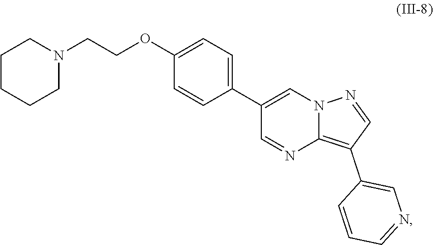 [SMILES: c1cncc(-c2cnn3cc(-c4ccc(OCCN5CCCCC5)cc4)cnc23)c1]

or a pharmaceutically acceptable salt thereof.

In some embodiments, the compound of Formula III has a structure of Formula III-9:

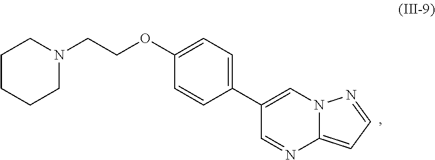 [SMILES: c1cc2ncc(-c3ccc(OCCN4CCCCC4)cc3)cn2n1]

or a pharmaceutically acceptable salt thereof.

In some embodiments, the compound of Formula III has a structure of Formula III-10:

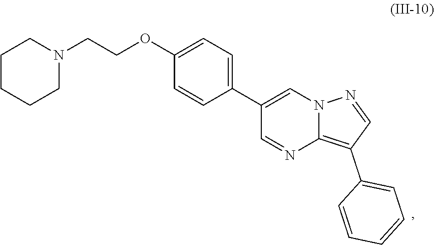 [SMILES: c1ccc(-c2cnn3cc(-c4ccc(OCCN5CCCCC5)cc4)cnc23)cc1]

or a pharmaceutically acceptable salt thereof.

In some embodiments, the compound of Formula III has a structure of Formula III-11:

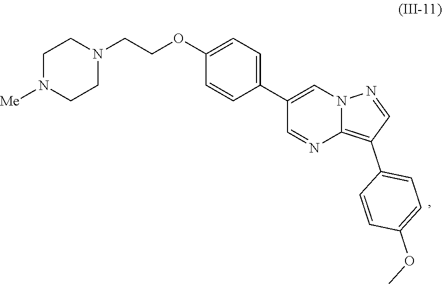 [SMILES: COc1ccc(-c2cnn3cc(-c4ccc(OCCN5CCN(C)CC5)cc4)cnc23)cc1]

or a pharmaceutically acceptable salt thereof.

In some embodiments, the compound of Formula III has a structure of Formula III-12:

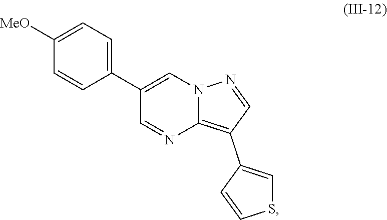 [SMILES: COc1ccc(-c2cnc3c(-c4ccsc4)cnn3c2)cc1]

or a pharmaceutically acceptable salt thereof.

In some embodiments, the compound of Formula III has a structure of Formula III-13:

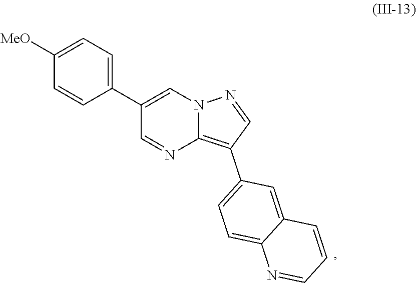 [SMILES: COc1ccc(-c2cnc3c(-c4ccc5ncccc5c4)cnn3c2)cc1]

or a pharmaceutically acceptable salt thereof.

In some embodiments, the compound of Formula III has a structure of Formula III-14:

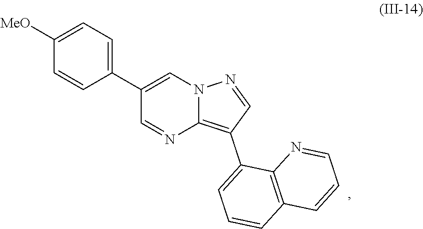 [SMILES: COc1ccc(-c2cnc3c(-c4cccc5cccnc45)cnn3c2)cc1]

or a pharmaceutically acceptable salt thereof.

In some embodiments, the compound of Formula III has a structure of Formula III-15:

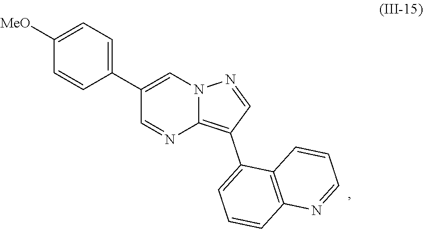 [SMILES: COc1ccc(-c2cnc3c(-c4cccc5ncccc45)cnn3c2)cc1]

or a pharmaceutically acceptable salt thereof.

In some embodiments, the compound of Formula III has a structure of Formula III-16:

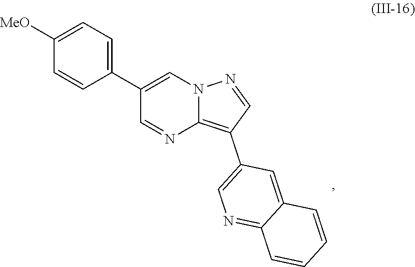 [SMILES: COc1ccc(-c2cnc3c(-c4cnc5ccccc5c4)cnn3c2)cc1]

or a pharmaceutically acceptable salt thereof.

In some embodiments, the compound of Formula III has a structure of Formula III-17:

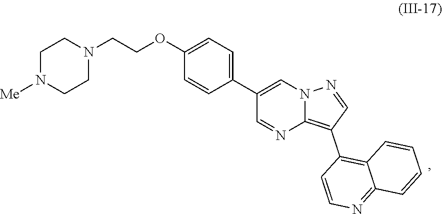 [SMILES: CN1CCN(CCOc2ccc(-c3cnc4c(-c5ccnc6ccccc56)cnn4c3)cc2)CC1]

or a pharmaceutically acceptable salt thereof.

In some embodiments, the compound of Formula III has a structure of Formula III-18:

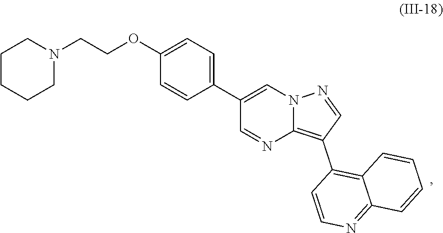 [SMILES: c1ccc2c(-c3cnn4cc(-c5ccc(OCCN6CCCCC6)cc5)cnc34)ccnc2c1]

or a pharmaceutically acceptable salt thereof.

In some embodiments, the compound of Formula III has a structure of Formula III-19:

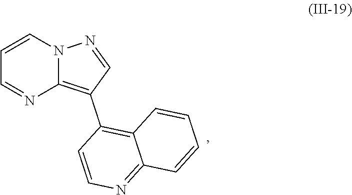 [SMILES: c1ccc2c(-c3cnn4cccnc34)ccnc2c1]

or a pharmaceutically acceptable salt thereof.

In some embodiments, the compound of Formula III has a structure of Formula III-20:

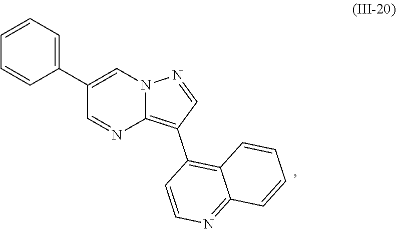 [SMILES: c1ccc(-c2cnc3c(-c4ccnc5ccccc45)cnn3c2)cc1]

or a pharmaceutically acceptable salt thereof.

In some embodiments, the compound of Formula III has a structure of Formula III-21:

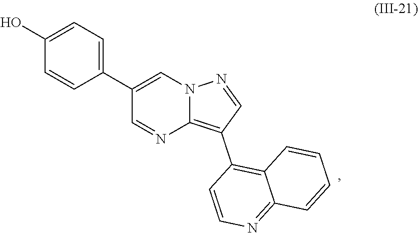 [SMILES: Oc1ccc(-c2cnc3c(-c4ccnc5ccccc45)cnn3c2)cc1]

or a pharmaceutically acceptable salt thereof.

In some embodiments, the compound of Formula III has a structure of Formula III-22:

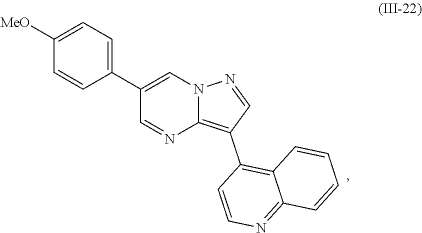 [SMILES: COc1ccc(-c2cnc3c(-c4ccnc5ccccc45)cnn3c2)cc1]

or a pharmaceutically acceptable salt thereof.

In some embodiments, the compound of Formula III has a structure of Formula III-23:

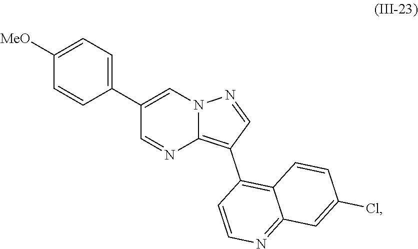 [SMILES: COc1ccc(-c2cnc3c(-c4ccnc5cc(Cl)ccc45)cnn3c2)cc1]

or a pharmaceutically acceptable salt thereof.

In some embodiments, the compound of Formula III has a structure of Formula III-24:

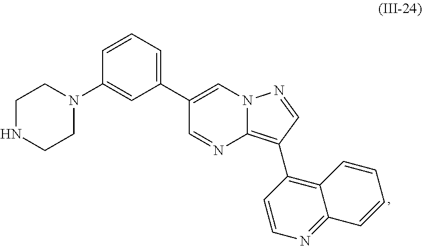 [SMILES: c1cc(-c2cnc3c(-c4ccnc5ccccc45)cnn3c2)cc(N2CCNCC2)c1]

or a pharmaceutically acceptable salt thereof.

In some embodiments, the compound of Formula III has a structure of Formula III-25:

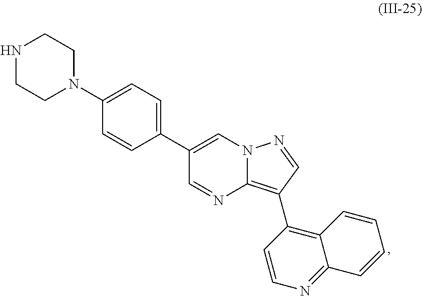 [SMILES: c1ccc2c(-c3cnn4cc(-c5ccc(N6CCNCC6)cc5)cnc34)ccnc2c1]

or a pharmaceutically acceptable salt thereof.

In some embodiments, the compound of Formula III has a structure of Formula III-26:

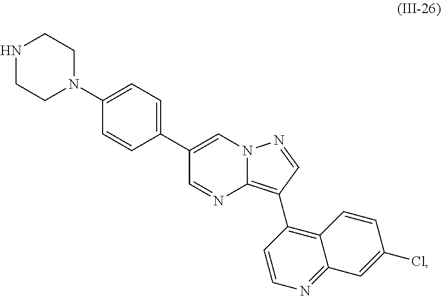 [SMILES: Clc1ccc2c(-c3cnn4cc(-c5ccc(N6CCNCC6)cc5)cnc34)ccnc2c1]

or a pharmaceutically acceptable salt thereof.

In some embodiments, the compound of Formula III has a structure of Formula III-27:

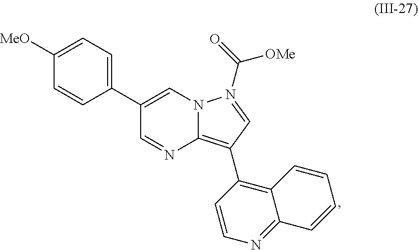

or a pharmaceutically acceptable salt thereof.

In some embodiments, the compound of Formula III has a structure of Formula III-28:

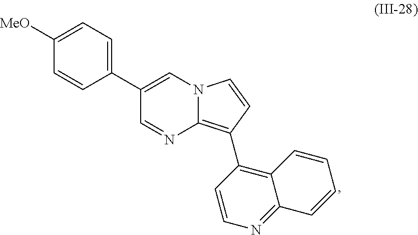 [SMILES: COc1ccc(-c2cnc3c(-c4ccnc5ccccc45)ccn3c2)cc1]

or a pharmaceutically acceptable salt thereof.

In some embodiments, the compound of Formula III has a structure of Formula III-29:

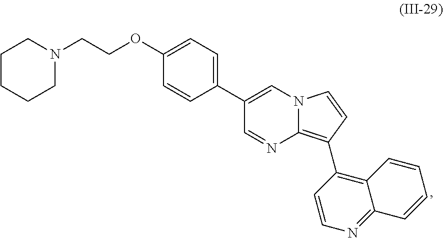 [SMILES: c1ccc2c(-c3ccn4cc(-c5ccc(OCCN6CCCCC6)cc5)cnc34)ccnc2c1]

or a pharmaceutically acceptable salt thereof.

In some embodiments, the compound of Formula III has a structure of Formula III-30:

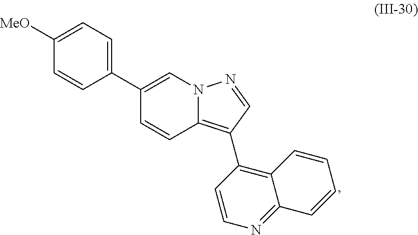 [SMILES: COc1ccc(-c2ccc3c(-c4ccnc5ccccc45)cnn3c2)cc1]

or a pharmaceutically acceptable salt thereof.

In some embodiments, the compound of Formula III has a structure of Formula III-31:

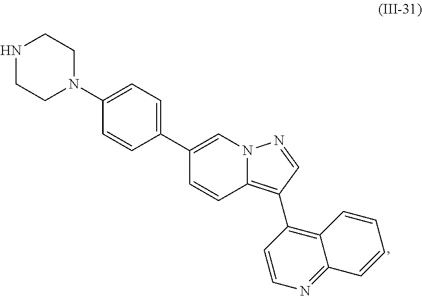 [SMILES: c1ccc2c(-c3cnn4cc(-c5ccc(N6CCNCC6)cc5)ccc34)ccnc2c1]

or a pharmaceutically acceptable salt thereof.

In some embodiments, the compound of Formula III has a structure of Formula III-32:

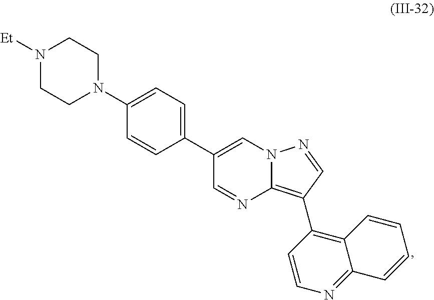 [SMILES: CCN1CCN(c2ccc(-c3cnc4c(-c5ccnc6ccccc56)cnn4c3)cc2)CC1]

or a pharmaceutically acceptable salt thereof.

In some embodiments, the compound of Formula III has a structure of Formula III-33:

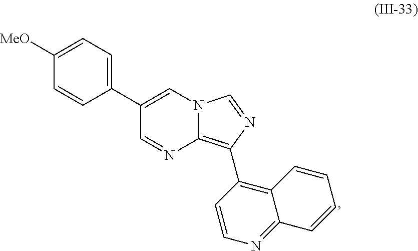 [SMILES: COc1ccc(-c2cnc3c(-c4ccnc5ccccc45)ncn3c2)cc1]

or a pharmaceutically acceptable salt thereof.

In some embodiments, the compound of Formula III has a structure of Formula III-34:

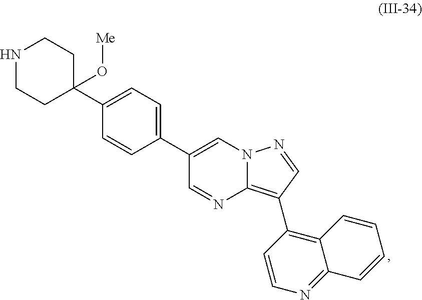 [SMILES: COC1(c2ccc(-c3cnc4c(-c5ccnc6ccccc56)cnn4c3)cc2)CCNCC1]

or a pharmaceutically acceptable salt thereof.

In some embodiments, the compound of Formula III has a structure of Formula III-35:

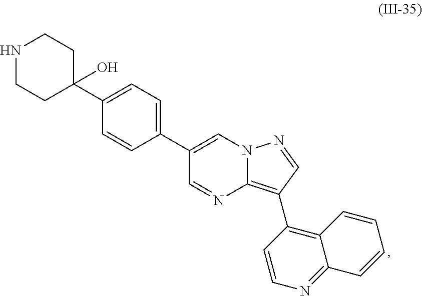 [SMILES: OC1(c2ccc(-c3cnc4c(-c5ccnc6ccccc56)cnn4c3)cc2)CCNCC1]

or a pharmaceutically acceptable salt thereof.

Additional compounds of Formula III are described U.S. Pat. Nos. 8,507,501 and 9,045,484, which are incorporated herein by reference.

In some embodiments, the ALK2 inhibitor is Compound 1:

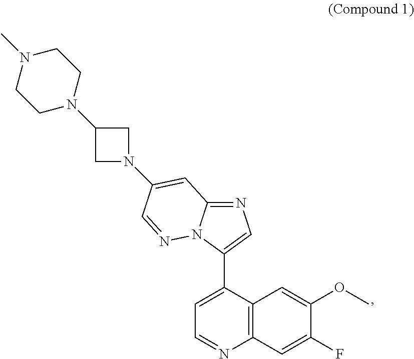

or a pharmaceutically acceptable salt thereof.

Compound 1 may be synthesized by methods known in the art, e.g., those described in US Patent Application Publication No. 2020/0179389, which is incorporated herein by reference.

In some embodiments, the ALK2 inhibitor is Compound 2:

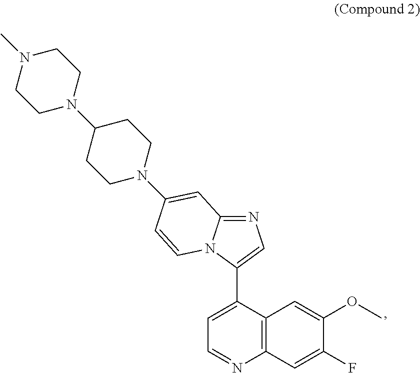

or a pharmaceutically acceptable salt thereof.

Compound 2 may be synthesized by methods known in the art, e.g., those described in US Patent Application Publication No. 2020/0179389, which is incorporated herein by reference.

In some embodiments, the ALK2 inhibitor is Compound 3:

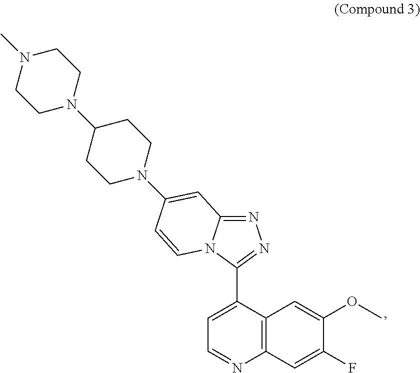

or a pharmaceutically acceptable salt thereof.

Compound 3 may be synthesized by methods known in the art, e.g., those described in US Patent Application Publication No. 2020/0179389, which is incorporated herein by reference.

In some embodiments, the ALK2 inhibitor is Compound 4:

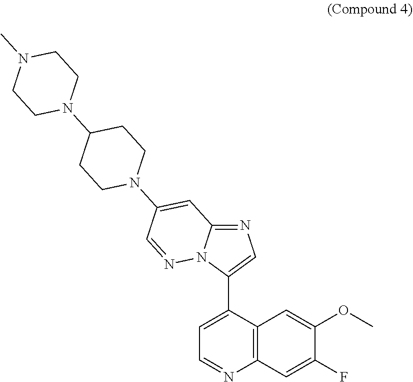

or a pharmaceutically acceptable salt thereof.

Compound 4 may be synthesized by methods known in the art, e.g., those described in US Patent Application Publication No. 2020/0179389, which is incorporated herein by reference.

In some embodiments, the ALK2 inhibitor is Compound 5:

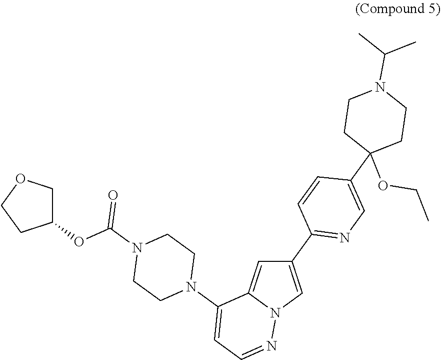 [SMILES: CCOC1(c2ccc(-c3cc4c(N5CCN(C(=O)O[C@@H]6CCOC6)CC5)ccnn4c3)nc2)CCN(C(C)C)CC1]

or a pharmaceutically acceptable salt thereof. Compound 5 may be synthesized by methods known in the art, e.g., those described in U.S. Pat. No. 10,233,186 and International Patent Application Publication No. WO2021067670A₁, which are incorporated herein by reference. In some embodiments, the compound is a crystalline compound of Compound 5, or a salt thereof. Crystalline compounds of Compound 5 can be synthesized by methods known in the art, e.g., those described in International Patent Application Publication No. WO2021030386A₁, which is incorporated herein by reference. In some embodiments, Compound 5 is administered as a succinate salt, a hydrochloride salt, or a fumarate salt, such as those described in International Patent Application Publication No. WO2021030386A₁. Additional ALK2 inhibitors that can be used in the methods described herein are described in US Patent Application Publication No. 2020/0331908 and U.S. Pat. No. 10,233,186, which are incorporated herein by reference.

In some embodiments, the ALK2 inhibitor is Compound 6:

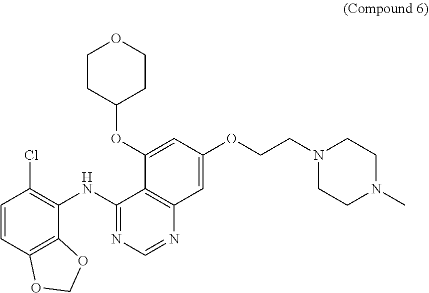 [SMILES: CN1CCN(CCOc2cc(OC3CCOCC3)c3c(Nc4c(Cl)ccc5c4OCO5)ncnc3c2)CC1]

or a pharmaceutically acceptable salt thereof. Compound 6 is also known as Saracatinib and AZD530.

In some embodiments, the ALK2 inhibitor is Compound 7:

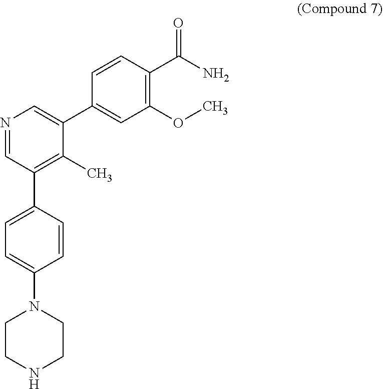 [SMILES: COc1cc(-c2cncc(-c3ccc(N4CCNCC4)cc3)c2C)ccc1C(N)=O]

or a pharmaceutically acceptable salt thereof. Compound 7 is also known as M₄K₂₁₄₉ and can be synthesized according to the methods described in Ensan et al., J. Med. Chem 63:4978-4996, 2020.

In some embodiments, the ALK2 inhibitor is a compound selected from the group consisting of:
(R)-3-(6-(3-Methylpiperazin-1-yl)pyridin-3-yl)-5-(piperidin-1-yl)-1H-pyrazolo[4,3-b]pyridine;
(R)-3-(6-(3-Methylpiperazin-1-yl)pyridin-3-yl)-5-(piperidin-1-yl)-1H-pyrazolo[3,4-c]pyridine;
(R)-3-(6-(3-methylpiperazin-1-yl)pyridin-3-yl)-5-(piperidin-1-yl)-1H-pyrazolo[4,3-d]pyrimidine;
3-(6-((R)-3-methylpiperazin-1-yl)pyridin-3-yl)-5-(2-methylpiperidin-1-yl)-1H-pyrazolo[4,3-d]pyrimidine;
5-(cis-2,6-Dimethylpiperazin-1-yl)-3-(6-((R)-3-methylpiperazin-1-yl)pyridin-3-yl)-1H-pyrazolo[4,3-d]pyrimidine;
   (3R,5S)—N,N,3,5-tetramethyl-4-(3-(6-((R)-3-methylpiperazin-1-yl)pyridin-3-yl)-1H-pyrazolo[4,3-d]pyrimidin-5-yl)piperazine-1-carboxamide;
5-((2R,6S)-2,6-dimethylpiperidin-1-yl)-3-(6-((R)-3-methylpiperazin-1-yl)pyridin-3-yl)-1H-pyrazolo[4,3-d]pyrimidine;
   (3R,5S)-3,5-dimethyl-4-(3-(6-((R)-3-methylpiperazin-1-yl)pyridin-3-yl)-1H-pyrazolo[4,3-d]pyrimidin-5-yl)morpholine;
   ((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)(pyrrolidin-1-yl)methanone;
5-(7-azabicyclo[2.2.1]heptan-7-yl)-3-(6-((R)-3-methylpiperazin-1-yl)pyridin-3-yl)-1H-pyrazolo[4,3-d]pyrimidine;
   (1R,5S,6S)-8-(3-(6-((R)-3-methylpiperazin-1-yl)pyridin-3-yl)-1H-pyrazolo[4,3-d]pyrimidin-5-yl)-8-azabicyclo[3.2.1]octan-6-ol;
1-((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)-2-methylpropan-1-one;
1-((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)-2-methoxyethan-1-one;
   (3R,5S)—N-isopropyl-3,5-dimethyl-4-(3-(6-((R)-3-methylpiperazin-1-yl)pyridin-3-yl)-1H-pyrazolo[4,3-d]pyrimidin-5-yl)piperazine-1-carboxamide;
   (3R,5S)—N,N,3,5-tetramethyl-4-(3-(6-((R)-3-methylpiperazin-1-yl)pyridin-3-yl)-1H-pyrazolo[4,3-d]pyrimidin-5-yl)piperazine-I-carboxamide;
   ((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)(pyrrolidin-1-yl)methanone;
   ((3R,5S)-3,5-dimethyl-4-(3-(6-((R)-3-methylpiperazin-1-yl)pyridin-3-yl)-1H-pyrazolo[4,3-d]pyrimidin-5-yl)piperazin-1-yl)(3-fluoropyrrolidin-1-yl)methanone;
   Ethyl (3R,5S)-3,5-dimethyl-4-(3-(6-((R)-3-methylpiperazin-1-yl)pyridin-3-yl)-1H-pyrazolo[4,3-d]pyrimidin-5-yl)piperazine-1-carboxylate;
2-Fluoroethyl (3R,5S)-3,5-dimethyl-4-(3-(6-((R)-3-methylpiperazin-1-yl)pyridin-3-yl)-1H-pyrazolo[4,3-d]pyrimidin-5-yl)piperazine-1-carboxylate;
   Cyclopropyl((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)methanone;
   ((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)(pyrrolidin-I-yl)methanone;
4-((1R,5S)-8-(3-(6-((R)-3-methylpiperazin-1-yl)pyridin-3-yl)-1H-pyrazolo[4,3-d]pyrimidin-5-yl)-3,8-diazabicyclo[3.2.1]octan-3-yl)benzonitrile;
5-((2R,6S)-2,6-dimethylpiperidin-1-yl)-3-(4-(4-methylpiperazin-1-yl)phenyl)-1H-pyrazolo[4,3-d]pyrimidine;
   Methyl (3R,5S)-3,5-dimethyl-4-(3-(6-((R)-3-methylpiperazin-1-yl)pyridin-3-yl)-1H-pyrazolo[4,3-d]pyrimidin-5-yl)piperazine-1-carboxylate;
   (R)-5-(1-Methyl-1,4,6,7-tetrahydro-5H-imidazo[4,5-c]pyridin-5-yl)-3-(6-(3-methylpiperazin-1-yl)pyridin-3-yl)-1H-pyrazolo[4,3-d]pyrimidine;
   (R)-5-(3-Methyl-5,6-dihydroimidazo[1,2-a]pyrazin-7 (8H)-yl)-3-(6-(3-methylpiperazin-1-yl)pyridin-3-yl)-1H-pyrazolo[4,3-d]pyrimidine;
   (5-(3-(6-((R)-3-Methylpiperazin-1-yl)pyridin-3-yl)-1H-pyrazolo[4,3-d]pyrimidin-5-yl)-2,5-diazabicyclo[2.2.2]octan-2-yl)(pyrrolidin-1-yl)methanone;
   ((1S,4S)-5-(3-(6-((R)-3-Methylpiperazin-1-yl)pyridin-3-yl)-1H-pyrazolo[4,3-d]pyrimidin-5-yl)-2,5-diazabicyclo[2.2.1]heptan-2-yl)(pyrrolidin-1-yl)methanone;
   Methyl (3R,5S)-3,5-dimethyl-4-(3-(4-(4-methylpiperazin-1-yl)phenyl)-1H-pyrazolo[4,3-d]pyrimidin-5-yl)piperazine-1-carboxylate;
   Methyl (3R,5S)-4-(3-(3-fluoro-4-(4-methylpiperazin-1-yl)phenyl)-1H-pyrazolo[4,3-d]pyrimidin-5-yl)-3,5-dimethylpiperazine-1-carboxylate;
   Methyl (3R,5S)-3,5-dimethyl-4-(3-(6-(4-methylpiperazin-1-yl)pyridin-3-yl)-1H-pyrazolo[4,3-d]pyrimidin-5-yl)piperazine-1-carboxylate;
   Methyl (3R,5S)-4-(3-(4-(4-ethylpiperazin-1-yl)-3-methylphenyl)-1H-pyrazolo[4,3-d]pyrimidin-5-yl)-3,5-dimethylpiperazine-1-carboxylate;
   Methyl (3R,5S)-3,5-dimethyl-4-(3-(4-(methylcarbamoyl)phenyl)-1H-pyrazolo[4,3-d]pyrimidin-5-yl)piperazine-1-carboxylate;
   Methyl (3R,5S)-3,5-dimethyl-4-(3-(4-(1-methylpyrrolidin-3-yl)phenyl)-1H-pyrazolo[4,3-d]pyrimidin-5-yl)piperazine-1-carboxylate;
   Methyl (3R,5S)-4-(3-(4-(1-(2-hydroxyethyl)pyrrolidin-3-yl)phenyl)-1H-pyrazolo[4,3-d]pyrimidin-5-yl)-3,5-dimethylpiperazine-1-carboxylate;
   Methyl (3R,5S)-3,5-dimethyl-4-(3-(4-(1-methylpyrrolidin-3-yl)phenyl)-1H-pyrazolo[4,3-d]pyrimidin-5-yl)piperazine-1-carboxylate;
   Methyl (3R,5S)-4-(3-(4-(4-(1-hydroxypropan-2-yl)piperazin-1-yl)phenyl)-1H-pyrazolo[4,3-d]pyrimidin-5-yl)-3,5-dimethylpiperazine-1-carboxylate;
   Methyl (3R,5S)-3,5-dimethyl-4-(3-(4-(4-(tetrahydro-2H-pyran-4-yl)piperazin-1-yl)phenyl)-1H-pyrazolo[4,3-d]pyrimidin-5-yl)piperazine-1-carboxylate;
2-(4-(4-(5-((2R,6S)-4-(Methoxycarbonyl)-2,6-dimethylpiperazin-1-yl)-1H-pyrazolo[4,3-d]pyrimidin-3-yl)phenyl)piperazin-1-yl)-2-methylpropanoic acid;
   Methyl (3R,5S)-3,5-dimethyl-4-(3-(6-((R)-2-methylmorpholino)pyridin-3-yl)-1H-pyrazolo[4,3-d]pyrimidin-5-yl)piperazine-1-carboxylate;
   Methyl (3R,5S)-4-(3-(6-((R)-3,4-dimethylpiperazin-1-yl)pyridin-3-yl)-1H-pyrazolo[4,3-d]pyrimidin-5-yl)-3,5-dimethylpiperazine-1-carboxylate;
   Methyl (3R,5S)-4-(3-(4-(4-hydroxypiperidin-1-yl)phenyl)-1H-pyrazolo[4,3-d]pyrimidin-5-yl)-3,5-dimethylpiperazine-1-carboxylate;
   Methyl (3R,5S)-4-(3-(4-((7S,8aR)-7-hydroxyhexahydropyrrolo[1,2-a]pyrazin-2 (1H)-yl)phenyl)-1H-pyrazolo[4,3-d]pyrimidin-5-yl)-3,5-dimethylpiperazine-1-carboxylate;
1-Methyl-4-(3-(1-methyl-1H-pyrazol-4-yl)-1H-pyrazolo[4,3-d]pyrimidin-5-yl)piperazin-2-one; 1-Cyclopropyl-4-(3-(1-methyl-1H-pyrazol-4-yl)-1H-pyrazolo[4,3-d]pyrimidin-5-yl)piperazin-2-one; 1,3-Dimethyl-4-(3-(1-methyl-1H-pyrazol-4-yl)-1H-pyrazolo[4,3-d]pyrimidin-5-yl)piperazin-2-one;
3-Ethyl-1-methyl-4-(3-(1-methyl-1H-pyrazol-4-yl)-1H-pyrazolo[4,3-d]pyrimidin-5-yl)piperazin-2-one;
6-(3-(1-Methyl-1H-pyrazol-4-yl)-1H-pyrazolo[4,3-d]pyrimidin-5-yl)-5,6,7,8-tetrahydro-1,6-naphthyridine;
3-(1-Methyl-1H-pyrazol-4-yl)-5-(1-(trifluoromethyl)-5,6-dihydroimidazo[1,5-a]pyrazin-7 (8H)-yl)-1H-pyrazolo[4,3-d]pyrimidine;
5-(3-(1-Methyl-1H-pyrazol-4-yl)-1H-pyrazolo[4,3-d]pyrimidin-5-yl)-4,5,6,7-tetrahydrothiazolo[5,4-c]pyridine;
3-(1-Methyl-1H-pyrazol-4-yl)-5-(8-methyl-5,6-dihydroimidazo[1,2-a]pyrazin-7 (8H)-yl)-1H-pyrazolo[4,3-d]pyrimidine;
3-(1-Methyl-1H-pyrazol-4-yl)-5-(1,4,5,7-tetrahydro-6H-pyrazolo[3,4-c]pyridin-6-yl)-1H-pyrazolo[4,3-d]pyrimidine;
9-(3-(1-Methyl-1H-pyrazol-4-yl)-1H-pyrazolo[4,3-d]pyrimidin-5-yl)-1,4,5,6,7,8-hexahydro-4,7-epiminocyclohepta[c]pyrazole;
1-(2,2-Difluoroethyl)-3-methyl-4-(3-(1-methyl-1H-pyrazol-4-yl)-1H-pyrazolo[4,3-d]pyrimidin-5-yl)piperazin-2-one;
1-Isopropyl-3-methyl-4-(3-(1-methyl-1H-pyrazol-4-yl)-1H-pyrazolo[4,3-d]pyrimidin-5-yl)piperazin-2-one;
1-(2-Methoxyethyl)-3-methyl-4-(3-(1-methyl-1H-pyrazol-4-yl)-1H-pyrazolo[4,3-d]pyrimidin-5-yl)piperazin-2-one;
3-Methyl-8-(3-(1-methyl-1H-pyrazol-4-yl)-1H-pyrazolo[4,3-d]pyrimidin-5-yl)-3,8-diazabicyclo[3.2.1]octan-2-one;
3-Ethyl-8-(3-(1-methyl-1H-pyrazol-4-yl)-1H-pyrazolo[4,3-d]pyrimidin-5-yl)-3,8-diazabicyclo[3.2.1]octan-2-one;
3-(2-Fluoroethyl)-8-(3-(1-methyl-1H-pyrazol-4-yl)-1H-pyrazolo[4,3-d]pyrimidin-5-yl)-3,8-diazabicyclo[3.2.1]octan-2-one;
3-Isopropyl-8-(3-(1-methyl-1H-pyrazol-4-yl)-1H-pyrazolo[4,3-d]pyrimidin-5-yl)-3,8-diazabicyclo[3.2.1]octan-2-one;
8-(3-(1-(2-Methoxyethyl)-1H-pyrazol-4-yl)-1H-pyrazolo[4,3-d]pyrimidin-5-yl)-3-methyl-3,8-diazabicyclo[3.2.1]octan-2-one;
3-Methyl-8-(3-(1-(tetrahydro-2H-pyran-4-yl)-1H-pyrazol-4-yl)-1H-pyrazolo[4,3-d]pyrimidin-5-yl)-3,8-diazabicyclo[3.2.1]octan-2-one;
8-(3-(1-((1R,4S)-4-Hydroxycyclohexyl)-1H-pyrazol-4-yl)-1H-pyrazolo[4,3-d]pyrimidin-5-yl)-3-methyl-3,8-diazabicyclo[3.2.1]octan-2-one;
   N,N-Dimethyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)piperidine-1-carboxamide;
   N, N-Dimethyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzamide;
8-(3-(1-(1-Isobutyrylpiperidin-4-yl)-1H-pyrazol-4-yl)-1H-pyrazolo[4,3-d]pyrimidin-5-yl)-3-methyl-3,8-diazabicyclo[3.2.1]octan-2-one;
3-Methyl-8-(3-(1-(2-morpholinoethyl)-1H-pyrazol-4-yl)-1H-pyrazolo[4,3-d]pyrimidin-5-yl)-3,8-diazabicyclo[3.2.1]octan-2-one;
3-Methyl-8-(3-(1-(pyridin-4-ylmethyl)-1H-pyrazol-4-yl)-1H-pyrazolo[4,3-d]pyrimidin-5-yl)-3,8-diazabicyclo[3.2.1]octan-2-one;
3-Methyl-8-(3-(6-(4-methylpiperazin-1-yl)pyridin-3-yl)-1H-pyrazolo[4,3-d]pyrimidin-5-yl)-3,8-diazabicyclo[3.2.1]octan-2-one;
8-(3-(1-Ethyl-1H-pyrazol-4-yl)-1H-pyrazolo[4,3-d]pyrimidin-5-yl)-3-methyl-3,8-diazabicyclo[3.2.1]octan-2-one;
8-(3-(1-Isopropyl-1H-pyrazol-4-yl)-1H-pyrazolo[4,3-d]pyrimidin-5-yl)-3-methyl-3,8-diazabicyclo[3.2.1]octan-2-one;
8-(3-(1-Cyclobutyl-1H-pyrazol-4-yl)-1H-pyrazolo[4,3-d]pyrimidin-5-yl)-3-methyl-3,8-diazabicyclo[3.2.1]octan-2-one;
8-(3-(1-Cyclopropyl-1H-pyrazol-4-yl)-1H-pyrazolo[4,3-d]pyrimidin-5-yl)-3-methyl-3,8-diazabicyclo[3.2.1]octan-2-one;
3-Methyl-8-(3-(1-(2,2,2-trifluoroethyl)-1H-pyrazol-4-yl)-1H-pyrazolo[4,3-d]pyrimidin-5-yl)-3,8-diazabicyclo[3.2.1]octan-2-one;
3-Methyl-8-(3-(1-(pyridin-4-yl)-1H-pyrazol-4-yl)-1H-pyrazolo[4,3-d]pyrimidin-5-yl)-3,8-diazabicyclo[3.2.1]octan-2-one;
3-Methyl-8-(3-(1-(pyridin-3-yl)-1H-pyrazol-4-yl)-1H-pyrazolo[4,3-d]pyrimidin-5-yl)-3,8-diazabicyclo[3.2.1]octan-2-one;
4-(4-(5-(3-Methyl-2-oxo-3,8-diazabicyclo[3.2.1]octan-8-yl)-1H-pyrazolo[4,3-d]pyrimidin-3-yl)-1H-pyrazol-1-yl)benzonitrile;
3-Methyl-8-(3-(1-(2-methylpyridin-4-yl)-1H-pyrazol-4-yl)-1H-pyrazolo[4,3-d]pyrimidin-5-yl)-3,8-diazabicyclo[3.2.1]octan-2-one;
8-(3-(1-(6-(Dimethylamino)pyridin-3-yl)-1H-pyrazol-4-yl)-1H-pyrazolo[4,3-d]pyrimidin-5-yl)-3-methyl-3,8-diazabicyclo[3.2.1]octan-2-one;
5-(4-(5-(3-Methyl-2-oxo-3,8-diazabicyclo[3.2.1]octan-8-yl)-1H-pyrazolo[4,3-d]pyrimidin-3-yl)-1H-pyrazol-1-yl) nicotinonitrile;
5-(4-(5-(3-Methyl-2-oxo-3,8-diazabicyclo[3.2.1]octan-8-yl)-1H-pyrazolo[4,3-d]pyrimidin-3-yl)-1H-pyrazol-1-yl)picolinonitrile;
8-(3-(1-(Imidazo[1,2-a]pyridin-6-yl)-1H-pyrazol-4-yl)-1H-pyrazolo[4,3-d]pyrimidin-5-yl)-3-methyl-3,8-diazabicyclo[3.2.1]octan-2-one;
2-Methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzonitrile;
3-Methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzonitrile;
2-Fluoro-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzonitrile;
   N, N-Dimethyl-4-(4-(5-(3-methyl-2-oxo-3,8-diazabicyclo[3.2.1]octan-8-yl)-1H-pyrazolo[4,3-d]pyrimidin-3-yl)-1H-pyrazol-I-yl)benzamide;
   N-Methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzamide;
2-Fluoro-N-methyl-4-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)benzamide;
   N-Methyl-5-(4-(5-(3-methyl-2-oxo-3,8-diazabicyclo[3.2.1]octan-8-yl)-1H-pyrazolo[4,3-d]pyrimidin-3-yl)-1H-pyrazol-1-yl)picolinamide;
4-(6-Methyl-7-(4-(piperazin-1-yl)phenyl)imidazo[1,2-b]pyridazin-3-yl)quinoline; Methyl 7-(4-(piperazin-1-yl)phenyl)-3-(quinolin-4-yl)imidazo[1,2-b]pyridazine-6-carboxylate;
   N-((1R,4R)-4-Hydroxy-4-methylcyclohexyl)-7-(4-(piperazin-1-yl)phenyl)-3-(quinolin-4-yl)imidazo[1,2-b]pyridazine-6-carboxamide;
   Ethyl 4-(7-(4-(piperazin-1-yl)phenyl)-3-(quinolin-4-yl)imidazo[1,2-b]pyridazine-6-carbonyl)piperazine-1-carboxylate;
7-(4-(Piperazin-1-yl)phenyl)-3-(quinolin-4-yl)imidazo[1,2-b]pyridazine-6-carboxamide;
7-(4-(piperazin-1-yl)phenyl)-3-(quinolin-4-yl)imidazo[1,2-b]pyridazine-6-carbonitrile;
   (7-(4-(Piperazin-1-yl)phenyl)-3-(quinolin-4-yl)imidazo[1,2-b]pyridazin-6-yl)methanol;
4-(6-(Methoxymethyl)-7-(4-(piperazin-1-yl)phenyl)imidazo[1,2-b]pyridazin-3-yl)quinoline;
4-(6-Methyl-7-(1-(piperidin-4-ylmethyl)-1H-pyrazol-4-yl)imidazo[1,2-b]pyridazin-3-yl)-7-(pyridin-4-yl)quinolone;
   (trans)-4-(4-(6-Methyl-3-(7-(pyridin-4-yl)quinolin-4-yl)imidazo[1,2-b]pyridazin-7-yl)-1H-pyrazol-1-yl)cyclohexan-1-ol;
2-(4-(4-(6-Methyl-3-(7-(pyridin-4-yl)quinolin-4-yl)imidazo[1,2-b]pyridazin-7-yl)phenyl)piperazin-1-yl)ethan-1-ol;
4-Methyl-1-(4-(6-methyl-3-(7-(pyridin-4-yl)quinolin-4-yl)imidazo[1,2-b]pyridazin-7-yl)phenyl)piperazin-2-one;
4-(6-Methyl-7-(3-(4-methylpiperazin-1-yl)phenyl)imidazo[1,2-b]pyridazin-3-yl)-7-(pyridin-4-yl)quinolone;
(4-(6-Methyl-3-(7-(pyridin-4-yl)quinolin-4-yl)imidazo[1,2-b]pyridazin-7-yl)phenyl)(4-methylpiperazin-1-yl)methanone;
(R)-4-(7-(3-fluoro-4-((2-methylpyrrolidin-1-yl)methyl)phenyl)-6-methylimidazo[1,2-b]pyridazin-3-yl)-7-(pyridin-4-yl)quinoline;
N-methyl-4-(6-methyl-7-(4-(piperazin-1-yl)phenyl)imidazo[1,2-b]pyridazin-3-yl)quinoline-7-carboxamide;
(4-(6-methyl-7-(4-(piperazin-1-yl)phenyl)imidazo[1,2-b]pyridazin-3-yl)quinolin-7-yl)(4-methylpiperazin-1-yl)methanone;
7-(1-methyl-1H-pyrazol-3-yl)-4-(6-methyl-7-(4-(piperazin-1-yl)phenyl)imidazo[1,2-b]pyridazin-3-yl)quinoline;
(4-(4-(6-methyl-7-(4-(piperazin-1-yl)phenyl)imidazo[1,2-b]pyridazin-3-yl)quinolin-7-yl)pyridin-2-yl)methanol;
4-(4-(6-methyl-7-(4-(piperazin-1-yl)phenyl)imidazo[1,2-b]pyridazin-3-yl)quinolin-7-yl)morpholine;
2-(4-(6-methyl-7-(4-(piperazin-1-yl)phenyl)imidazo[1,2-b]pyridazin-3-yl)quinolin-7-yl)-6-oxa-2-azaspiro[3.4]octane;
4-(6-methyl-7-(4-(piperazin-1-yl)phenyl)imidazo[1,2-b]pyridazin-3-yl)-N-(pyridin-3-yl)quinolin-7-amine;
1-(4-(4-(6-methyl-7-(4-(piperazin-1-yl)phenyl)imidazo[1,2-b]pyridazin-3-yl)quinolin-7-yl)piperazin-1-yl)ethan-1-one;
4-((4-(6-methyl-7-(4-(piperazin-1-yl)phenyl)imidazo[1,2-b]pyridazin-3-yl)quinolin-7-yl)methyl)morpholine;
(3,5-difluorophenyl)(4-(6-methyl-7-(4-(piperazin-1-yl)phenyl)imidazo[1,2-b]pyridazin-3-yl)quinolin-7-yl)methanol;
(4-(6-methyl-7-(4-(piperazin-1-yl)phenyl)imidazo[1,2-b]pyridazin-3-yl)quinolin-7-yl)methanol;
4-(6-methyl-7-(4-(piperazin-1-yl)phenyl)imidazo[1,2-b]pyridazin-3-yl)-1,8-naphthyridine;
5-(6-methyl-7-(4-(piperazin-1-yl)phenyl)imidazo[1,2-b]pyridazin-3-yl)quinoline-8-carbonitrile;
8-(6-methyl-7-(4-(piperazin-1-yl)phenyl)imidazo[1,2-b]pyridazin-3-yl)-2,3-dihydro-[1,4]dioxino[2,3-b]pyridine;
4-(6-methyl-7-(4-(piperazin-1-yl)phenyl)imidazo[1,2-b]pyridazin-3-yl)quinoline-7-carbonitrile;
N-(2-hydroxyethyl)-4-(6-methyl-7-(4-(piperazin-1-yl)phenyl)imidazo[1,2-b]pyridazin-3-yl)quinoline-8-carboxamide;
(4-(6-methyl-7-(1-(tetrahydro-2H-pyran-4-yl)-1H-pyrazol-4-yl)imidazo[1,2-b]pyridazin-3-yl)-2,3-dihydro-1H-pyrrolo[2,3-b]pyridin-1-yl)(pyridin-4-yl)methanone;
1-((1S,4S)-5-(4-(7-(1-((1R,4R)-4-hydroxycyclohexyl)-1H-pyrazol-4-yl)-6-methylimidazo[1,2-b]pyridazin-3-yl)quinolin-7-yl)-2,5-diazabicyclo[2.2.1]heptan-2-yl)ethan-1-one;
2-(4-(7-(1-((1R,4R)-4-hydroxycyclohexyl)-1H-pyrazol-4-yl)-6-methylimidazo[1,2-b]pyridazin-3-yl)quinolin-7-yl) hexahydropyrrolo[1,2-a]pyrazin-6 (2H)-one;
4-(6-methyl-7-(1-(piperidin-4-yl)-1H-pyrazol-4-yl)imidazo[1,2-a]pyridin-3-yl)quinoline;
2-fluoro-4-(6-methyl-7-(1-(piperidin-4-yl)-1H-pyrazol-4-yl)imidazo[1,2-a]pyridin-3-yl)benzamide;
(1R,4R)-4-(4-(6-Methyl-3-(7-(pyridin-2-yl)-1,8-naphthyridin-4-yl)imidazo[1,2-b]pyridazin-7-yl)-1H-pyrazol-1-yl)cyclohexan-1-ol;
5-(7-(4-(Azetidin-1-ylmethyl)phenyl)-6-methylimidazo[1,2-b]pyridazin-3-yl)-2-(pyridin-2-yl)-1,8-naphthyridine;
5-(7-(1-Ethyl-1H-pyrazol-4-yl)-6-methylimidazo[1,2-b]pyridazin-3-yl)-2-(pyridin-2-yl)-1,8-naphthyridine;
5-(7-(1-Isopropyl-1H-pyrazol-4-yl)-6-methylimidazo[1,2-b]pyridazin-3-yl)-2-(pyrimidin-2-yl)-1,8-naphthyridine;
(1R,4R)-4-(4-(6-Methyl-3-(7-(pyrazin-2-yl)-1,8-naphthyridin-4-yl)imidazo[1,2-b]pyridazin-7-yl)-1H-pyrazol-1-yl)cyclohexan-1-ol;
5-(7-(4-(Azetidin-1-ylmethyl)phenyl)-6-methylimidazo[1,2-b]pyridazin-3-yl)-2-(pyrazin-2-yl)-1,8-naphthyridine;
5-(7-(1-Isopropyl-1H-pyrazol-4-yl)-6-methylimidazo[1,2-b]pyridazin-3-yl)-2-(pyrazin-2-yl)-1,8-naphthyridine;
5-(6-Methyl-7-(1-(tetrahydro-2H-pyran-4-yl)-1H-pyrazol-4-yl)imidazo[1,2-b]pyridazin-3-yl)-2-(pyrazin-2-yl)-1,8-naphthyridine;
5-(7-(1-Isopropyl-1H-pyrazol-4-yl)-6-methylimidazo[1,2-b]pyridazin-3-yl)-2-(1-methyl-1H-pyrazol-3-yl)-1,8-naphthyridine;
5-(7-(4-(Azetidin-1-ylmethyl)phenyl)-6-methylimidazo[1,2-b]pyridazin-3-yl)-2-(1-methyl-1H-pyrazol-3-yl)-1,8-naphthyridine;
   N, N-dimethyl-4-(6-methyl-3-(7-(1-methyl-1H-pyrazol-3-yl)-1,8-naphthyridin-4-yl)imidazo[1,2-b]pyridazin-7-yl)benzamide;
5-(7-(4-(Azetidin-1-ylmethyl)phenyl)-6-methylimidazo[1,2-b]pyridazin-3-yl)-2-(5-methyl-1H-pyrazol-3-yl)-1,8-naphthyridine;
4-(4-(6-methyl-3-(7-(5-methyl-1H-pyrazol-3-yl)-1,8-naphthyridin-4-yl)imidazo[1,2-b]pyridazin-7-yl)benzyl)morpholine;
5-(7-(1-Isopropyl-1H-pyrazol-4-yl)-6-methylimidazo[1,2-b]pyridazin-3-yl)-2-(5-methyl-1H-pyrazol-3-yl)-1,8-naphthyridine;
5-(7-(1-Isopropyl-1H-pyrazol-4-yl)-6-methylimidazo[1,2-b]pyridazin-3-yl)-2-(1-methyl-1H-pyrazol-4-yl)-1,8-naphthyridine;
2-(1-Ethyl-1H-imidazol-4-yl)-5-(6-methyl-7-(4-(pyrrolidin-1-ylmethyl)phenyl)imidazo[1,2-b]pyridazin-3-yl)-1,8-naphthyridine;
4-(4-(3-(7-(1-Ethyl-1H-imidazol-4-yl)-1,8-naphthyridin-4-yl)-6-methylimidazo[1,2-b]pyridazin-7-yl)benzyl)morpholine;
1-(4-(3-(7-(1-Ethyl-1H-imidazol-4-yl)-1,8-naphthyridin-4-yl)-6-methylimidazo[1,2-b]pyridazin-7-yl)phenyl)-N,N-dimethylmethanamine;
2-(1-Ethyl-1H-imidazol-4-yl)-5-(7-(1-isopropyl-1H-pyrazol-4-yl)-6-methylimidazo[1,2-b]pyridazin-3-yl)-1,8-naphthyridine;
5-(7-(4-(Azetidin-1-ylmethyl)phenyl)-6-methylimidazo[1,2-b]pyridazin-3-yl)-2-(1-ethyl-1H-imidazol-4-yl)-1,8-naphthyridine;
5-(7-(4-((3,3-Difluoroazetidin-1-yl) yl)methyl)phenyl)-6-methylimidazo[1,2-b]pyridazin-3-yl)-2-(1-ethyl-1H-imidazol-4-yl)-1,8-naphthyridine;
5-(7-(4-((3,3-Difluoroazetidin-1-yl)methyl)phenyl)-6-methylimidazo[1,2-b]pyridazin-3-yl)-2-(1-ethyl-1H-imidazol-4-yl)-1,8-naphthyridine;
   (S)-4-(4-(3-(7-(1-ethyl-1H-imidazol-4-yl)-1,8-naphthyridin-4-yl)-6-methylimidazo[1,2-b]pyridazin-7-yl)benzyl)-2-methylmorpholine;
   (S)-(4-(4-(3-(7-(1-ethyl-1H-imidazol-4-yl)-1,8-naphthyridin-4-yl)-6-methylimidazo[1,2-b]pyridazin-7-yl)benzyl)morpholin-2-yl)methanol;
4-(6-methyl-7-(1-(piperidin-4-yl)-1H-pyrazol-4-yl)imidazo[1,2-a]pyridin-3-yl)quinolone;
   (R)-4-(7-(3-fluoro-4-((2-methylpyrrolidin-1-yl)methyl)phenyl)-6-methylimidazo[1,2-b]pyridazin-3-yl)-7-(pyridin-4-yl)quinolone;
1-((1S,4S)-5-(4-(7-(1-((1R,4R)-4-hydroxycyclohexyl)-1H-pyrazol-4-yl)-6-methylimidazo[1,2-b]pyridazin-3-yl)quinolin-7-yl)-2,5-diazabicyclo[2.2.1]heptan-2-yl)ethan-1-one;
7-(1-methyl-1H-pyrazol-3-yl)-4-(6-methyl-7-(4-(piperazin-1-yl)phenyl)imidazo[1,2-b]pyridazin-3-yl)quinolone;
4-(6-Methyl-7-(4-(piperazin-1-yl)phenyl)imidazo[1,2-b]pyridazin-3-yl)quinolone;
4-(6-(Methoxymethyl)-7-(4-(piperazin-1-yl)phenyl)imidazo[1,2-b]pyridazin-3-yl)quinolone;
2-(4-Methyl-2H-1,2,3-triazol-2-yl)-5-(6-methyl-7-(4-(pyrrolidin-1-ylmethyl)phenyl)imidazo[1,2-b]pyridazin-3-yl)-1,8-naphthyridine;
5-(7-(1-Isopropyl-1H-pyrazol-4-yl)-6-methylimidazo[1,2-b]pyridazin-3-yl)-2-(4-methyl-2H-1,2,3-triazol-2-yl)-1,8-naphthyridine;
2-(4-Methyl-2H-1,2,3-triazol-2-yl)-5-(6-methyl-7-(1-(tetrahydro-2H-pyran-4-yl)-1H-pyrazol-4-yl)imidazo[1,2-b]pyridazin-3-yl)-1,8-naphthyridine;
   N, N-dimethyl-1-(4-(6-methyl-3-(7-(4-methyl-2H-1,2,3-triazol-2-yl)-1,8-naphthyridin-4-yl)imidazo[1,2-b]pyridazin-7-yl)phenyl)methanamine;
4-(4-(6-Methyl-3-(7-(4-methyl-2H-1,2,3-triazol-2-yl)-1,8-naphthyridin-4-yl)imidazo[1,2-b]pyridazin-7-yl)benzyl)morpholine;
   N, N-dimethyl-1-(4-(6-methyl-3-(7-(4-methyl-2H-1,2,3-triazol-2-yl)-1,8-naphthyridin-4-yl)imidazo[1,2-b]pyridazin-7-yl)phenyl)methanamine;
   (S)-3-methyl-1-(4-(6-methyl-3-(7-(4-methyl-2H-1,2,3-triazol-2-yl)-1,8-naphthyridin-4-yl)imidazo[1,2-b]pyridazin-7-yl)benzyl)pyrrolidin-3-ol;
1-(4-(6-methyl-3-(7-(4-methyl-2H-1,2,3-triazol-2-yl)-1,8-naphthyridin-4-yl)imidazo[1,2-b]pyridazin-7-yl)benzyl)piperidin-4-ol;
4-methyl-1-(4-(6-methyl-3-(7-(4-methyl-2H-1,2,3-triazol-2-yl)-1,8-naphthyridin-4-yl)imidazo[1,2-b]pyridazin-7-yl)benzyl)piperidin-4-ol;
3-methyl-1-(4-(6-methyl-3-(7-(4-methyl-2H-1,2,3-triazol-2-yl)-1,8-naphthyridin-4-yl)imidazo[1,2-b]pyridazin-7-yl)benzyl) azetidin-3-ol;
2-(4-methyl-2H-1,2,3-triazol-2-yl)-5-(6-methyl-7-(4-((4-methylpiperazin-1-yl)methyl)phenyl)imidazo[1,2-b]pyridazin-3-yl)-1,8-naphthyridine;
1-(4-(3-(7-(1-Ethyl-1H-imidazol-4-yl)-1,8-naphthyridin-4-yl)-6-methylimidazo[1,2-b]pyridazin-7-yl)benzyl)piperidin-4-ol;
1-(4-(3-(7-(1-Ethyl-1H-imidazol-4-yl)-1,8-naphthyridin-4-yl)-6-methylimidazo[1,2-b]pyridazin-7-yl)benzyl)-4-methylpiperidin-4-ol;
   and pharmaceutically acceptable salts thereof.

Additional ALK2 inhibitors that can be used in the methods described herein are BCX9250, INCB00928, dorsomorphin, LDN-212854, LDN-193189, and LDN-214117 and the ALK2 inhibitors described in International Patent Application Publication Nos. WO2018232094A₁ and WO2020068729A₁, U.S. Pat. No. 11,111,247, and US Patent Application Publication Nos. US20200095250A₁, US20200199131A₁, and US20200331908A₁, and Nguyen et al., ACS Med. Chem. Lett. 13:1159-1164, 2022, which are incorporated herein by reference.

In some embodiments, the compound used in the methods and compositions described herein is a compound of Formula II-11:

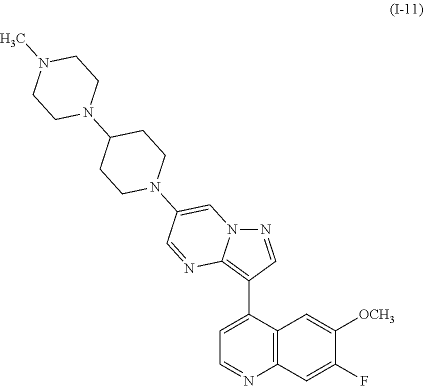 [SMILES: COc1cc2c(-c3cnn4cc(N5CCC(N6CCN(C)CC6)CC5)cnc34)ccnc2cc1F]

or a pharmaceutically acceptable salt thereof.

In some embodiments, the compound is a crystalline compound of Formula II-11, or a salt thereof. Crystalline compounds of Formula II-11 can be synthesized by methods known in the art, e.g., those described in U.S. Patent Application Publication No. US20210238185A₁, which is incorporated herein by reference.

In certain embodiments, a crystalline compound of Formula I-11 is not solvated (e.g., the crystal lattice does not comprise molecules of a solvent). In certain such embodiments, the crystalline compound of Formula I-11 is anhydrous, or substantially anhydrous.

In certain embodiments, the compound of Formula I-11 is in the form of a salt with an anion selected from chloride, bromide, succinate, xinafoate, citrate, malate, hemi-malate, tartrate, malonate, mesylate, phosphate, tosylate, sulfate, and bis-sulfate. In preferred embodiments, the compound of Formula I-11 is in the form of a succinate salt, such as a mono-succinate salt.

The compound of Formula II-11 or any crystalline compound described herein may be used in the manufacture of a medicament for the treatment of any diseases or conditions disclosed herein.

The mono-succinate salt of the compound of Formula II-11 exists at least as “Form A,” “Form B,” “Form C,” and “Form D,” as described U.S. Patent Application Publication No. US20210238185A₁. These different forms are understood as “polymorphs” herein.

A polymorph of the crystalline compound may be characterized by powder X-ray diffraction (XRPD). 0 represents the diffraction angle, measured in degrees. In certain embodiments, the diffractometer used in XRPD measures the diffraction angle as two times the diffraction angle θ. Thus, in certain embodiments, the diffraction patterns described herein refer to X-ray intensity measured against angle 2θ.

In certain embodiments, a first anhydrous crystalline form of a compound of Formula II-11 mono-succinate salt has 2θ values of about 7.05±0.2, 15.16±0.2, 21.05±0.2, 21.26±0.2, and 24.47±0.2. In further embodiments, an anhydrous crystalline compound of Formula II-11 mono-succinate salt has 20 values of about 3.58±0.2, 7.05±0.2, 13.8±0.2, 14.16±0.2, 15.16±0.2, 16.18±0.2, 16.80±0.2, 17.15±0.2, 17.69±0.2, 18.29±0.2, 18.84±0.2, 20.29±0.2, 21.05±0.2, 21.26±0.2, 22.68±0.2, 23.84±0.2, 24.47±0.2, 24.84±0.2, and 28.47±0.2. In yet further embodiments, the anhydrous crystalline compound of Formula I-11 mono-succinate salt has 20 values of about 3.58±0.2, 7.05±0.2, 10.59±0.2, 10.75±0.2, 13.80±0.2, 14.16±0.2, 15.16±0.2, 15.68±0.2, 16.18±0.2, 16.80±0.2, 17.15±0.2, 17.69±0.2, 17.97±0.2, 18.29±0.2, 18.59±0.2, 18.84±0.2, 19.27±0.2, 20.29±0.2, 21.05±0.2, 21.26±0.2, 21.56±0.2, 21.78±0.2, 22.68±0.2, 23.84±0.2, 24.47±0.2, 24.84±0.2, 25.15±0.2, 26.10±0.2, 27.12±0.2, 27.78±0.2, 28.47±0.2, and 29.06±0.2.

In certain embodiments, a second anhydrous crystalline form of a compound of Formula I-11 mono-succinate salt has 20 values of about 9.79±0.2, 13.05±0.2, 22.91±0.2, 23.60±0.2, and 26.25±0.2. In further embodiments, an anhydrous crystalline compound of Formula I-11 mono-succinate salt has 20 values of about 3.25±0.2, 9.79±0.2, 13.05±0.2, 16.75±0.2, 19.50±0.2, 22.91±0.2, 23.60±0.2, and 26.25±0.2. In yet further embodiments, an anhydrous crystalline compound of Formula II-11 mono-succinate salt has 20 values of about 3.25±0.2, 9.79±0.2, 13.05±0.2, 13.61±0.2, 14.39±0.2, 16.75±0.2, 18.50±0.2, 19.50±0.2, 22.91±0.2, 23.60±0.2, and 26.25±0.2.

In certain embodiments, the invention relates to a pharmaceutical composition comprising a crystalline compound of Formula II-11 mono-succinate salt and one or more pharmaceutically acceptable excipients. In certain embodiments, the pharmaceutical composition is selected from tablets, capsules, and suspensions.

### Pharmaceutical Compositions and Preparations

The ALK2 inhibitors described herein (e.g., a compound of Formula I, Formula II, or Formula III or of Compounds 1-7, BCX9250, or INCB00928 or a pharmaceutically acceptable salt thereof) can be incorporated into a vehicle for administration into a patient, such as a human patient suffering from FID or anemia (e.g., FID anemia) associated with MDS, MF, or MDS/MPN. In some embodiments, a pharmaceutical composition including an ALK2 inhibitor described herein may be used in combination with other agents (e.g., therapeutic biologics and/or small molecules) or compositions in a therapy. Pharmaceutical compositions containing ALK2 inhibitors can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions. In certain embodiments, the pharmaceutical preparations have a low enough pyrogen activity to be suitable for intravenous use in a human patient.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Exemplary pharmaceutically acceptable excipients also include, for example, binders, disintegrating agents, lubricants, corrigents, solubilizing agents, suspension aids, emulsifying agents, coating agents, cyclodextrins, and/or buffers. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

Mixtures of ALK2 inhibitors may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated.

The pharmaceutical compositions of the invention may be prepared in microcapsules, such as hydroxylmethylcellulose or gelatin-microcapsule and poly-(methylmethacrylate) microcapsule. The pharmaceutical compositions of the invention may also be prepared in other drug delivery systems such as liposomes, albumin microspheres, microemulsions, nanoparticles, and nanocapsules. Such techniques are described in Remington: The Science and Practice of Pharmacology 22nd edition, Allen, L. Ed. (2013). The pharmaceutical compositions to be used for in vivo administration must be sterile. This is readily accomplished by filtration through sterile filtration membranes.

The pharmaceutical compositions of the invention may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polylactides, copolymers of L-glutamic acid and y ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., a an ALK2 inhibitor described herein, included in the pharmaceutical preparations is such that a suitable dose within the designated range is provided (e.g., a dose within the range of 0.01-100 mg/kg of body weight).

Although the dosage will vary depending on the symptoms, age and body weight of the patient, the nature and severity of the disorder to be treated or prevented, the route of administration and the form of the drug, in general, a daily dosage of from 0.01 to 3000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The pharmaceutical compositions are administered in a manner compatible with the dosage formulation and in such amount as is therapeutically effective to result in an improvement or remediation of symptoms. The pharmaceutical compositions are administered in a variety of dosage forms, e.g., intravenous dosage forms, subcutaneous dosage forms, and oral dosage forms (e.g., ingestible solutions, drug release capsules). Pharmaceutical compositions that include an ALK2 inhibitor of the invention may be administered to a subject in need thereof, for example, one or more times (e.g., 1-10 times or more) daily, every other day, weekly, biweekly, monthly, bimonthly, quarterly, biannually, annually, or as medically necessary. Dosages may be provided in either a single or multiple dosage regimens. The timing between administrations may decrease as the medical condition improves or increase as the health of the patient declines.

In certain embodiments, the individual to which the composition is administered is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as an eye drop, through ophthalmic mucous membrane administration.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

In other cases, the compounds useful in the methods of the present invention may contain one or more acidic functional groups and, thus, are capable of forming pharmaceutically acceptable salts with pharmaceutically acceptable bases. These salts are the relatively non-toxic inorganic and organic base addition salts of a compound. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. Compositions or compounds may also be administered as a bolus, electuary or paste.

To prepare solid dosage forms for oral administration capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the compositions of the present invention can also include adjuvants such as wetting agents, lubricants, emulsifying and suspending agents such as sodium lauryl sulfate and magnesium stearate, or sweetening, flavoring, coloring, perfuming, preservative, or anti-oxidant agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams, or spray formulations containing such carriers as are known in the art to be appropriate.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required.

The ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compounds described herein can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions, and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of agents may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

These compounds may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compounds, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts. In general, the compositions of this invention may be provided in an aqueous solution containing about 0.1-30% w/v of a compound disclosed herein, among other substances, for parenteral administration. Typical dose ranges are from about 0.01 to about 50 mg/kg of body weight per day, given in 1 single or 2-4 divided doses. Each divided dose may contain the same or different compounds of the invention.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved.

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily. In some embodiments, the active compound will be administered once every other day.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn, or other metal salts.

The pharmaceutically acceptable acid addition salts can also exist as various solvates, such as with water, methanol, ethanol, dimethylformamide, dimethylsulfoxide, and the like. Mixtures of such solvates can also be prepared. The source of such solvate can be from the solvent of crystallization, inherent in the solvent of preparation or crystallization, or adventitious to such solvent. In some embodiments, a solvate of a disclosed compound can be a dimethylsulfoxide solvate.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Routes, Dosage, and Administration

Pharmaceutical compositions that include an ALK2 inhibitor as the therapeutic agent may be administered by a variety of routes, such as intravenous (e.g., by bolus or continuous infusion), parenteral, intradermal, transdermal, intramuscular, intranasal, subcutaneous, percutaneous, topical, intratracheal, intraperitoneal, intraarterial, intravascular, intrathecal, intracerebroventricular, inhalation, perfusion, lavage, and oral administration. The pharmaceutical composition may also be formulated for, or administered via, oral, ocular, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014). For ocular administration, the formulation may be delivered systemically, by injection (e.g., intraocular injection), or topically (e.g., as a solution, suspension, or ointment, such as by instillation (e.g., an eye drop)).

A pharmaceutical composition of the invention may include a dosage of an ALK2 inhibitor of the invention ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The dosage range for the agent depends upon the potency and includes amounts large enough to produce the desired effect, e.g., reduction in at least one symptom of FID or anemia (e.g., FID anemia) associated with MDS, MF, or MDS/MPN. The most suitable route and dosage for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. The dosage should not be so large as to cause unacceptable adverse side effects. The dosage can be determined by one of skill in the art and can also be adjusted by the individual physician in the event of any complication. Typically, the dosage ranges from 0.1 mg/kg body weight to 1 g/kg body weight. In some embodiments, the dosage range is from 0.1 mg/kg body weight to 1 g/kg body weight, from 0.1 mg/kg body weight to 500 mg/kg body weight, from 0.1 mg/kg body weight to 250 mg/kg body weight, from 0.1 mg/kg body weight to 100 mg/kg body weight, from 0.1 mg/kg body weight to 50 mg/kg body weight, from 0.1 mg/kg body weight to 10 mg/kg body weight, from 0.1 mg/kg body weight to 5 mg/kg body weight, from 0.1 mg/kg body weight to 1 mg/kg body weight, from 1 mg/kg to 100 mg/kg, from 5 mg/kg to 100 mg/kg, from 10 mg/kg to 100 mg/kg, from 15 mg/kg to 100 mg/kg, from 20 mg/kg to 100 mg/kg, from 25 mg/kg to 100 mg/kg, from 30 mg/kg to 100 mg/kg, from 40 mg/kg to 100 mg/kg, from 50 mg/kg to 100 mg/kg, from 60 mg/kg to 100 mg/kg, from 70 mg/kg to 100 mg/kg, from 75 mg/kg to 100 mg/kg, from 25 mg/kg to 50 mg/kg, from 50 mg/kg to 200 mg/kg, from 75 mg/kg to 250 mg/kg, from 100 mg/kg to 300 mg/kg, from 100 mg/kg to 200 mg/kg, from 100 mg/kg to 400 mg/kg, from 100 mg/kg to 500 mg/kg, from 100 mg/kg to 750 mg/kg from 200 mg/kg to 1000 mg/kg, from 300 mg/kg to 1000 mg/kg, from 400 mg/kg to 1000 mg/kg, from 500 mg/kg to 1000 mg/kg, from 600 mg/kg to 1000 mg/kg, from 700 mg/kg to 1000 mg/kg, from 800 mg/kg to 1000 mg/kg, from 900 mg/kg to 1000 mg/kg, from 250 mg/kg to 750 mg/kg, from 300 mg/kg to 600 mg/kg, or any range there between. For example, a pharmaceutical composition of the invention may include a dosage of an ALK2 inhibitor of the invention ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

In certain embodiments, the dose of the agent is at least. 1 mg/kg/day; in other embodiments the dose of the agent is at least 1 mg/kg/day, at least 10 mg/kg/day, at least 20 mg/kg/day, at least 25 mg/kg/day, at least 30 mg/kg/day, at least 40 mg/kg/day, at least 50 mg/kg/day, at least 60 mg/kg/day, at least 70 mg/kg/day, at least 80 mg/kg/day, at least 90 mg/kg/day, at least 100 mg/kg/day, at least 125 mg/kg/day, at least 150 mg/kg/day, at least 175 mg/kg/day, at least 200 mg/kg/day, at least 250 mg/kg/day, at least 300 mg/kg/day, at least 400 mg/kg/day, at least 500 mg/kg/day or more.

In some embodiments, the dosage range of the agent for use in a human subject is from 1 mg/day to 500 mg/day, from 1 mg/day to 450 mg/day, from 1 mg/day to 350 mg/day, from 1 mg/day to 300 mg/day, from 3 mg/day to 250 mg/day, from 5 mg/day to 250 mg/day, from 10 mg/day to 250 mg/day, from 15 mg/day to 200 mg/day, from 20 mg/day to 200 mg/day, from 25 mg/day to 200 mg/day, from 25 mg/day to 175 mg/day, from 25 mg/day to 150 mg/day, from 25 mg/day to 125 mg/day, from 25 mg/day to 100 mg/day, from 25 mg/day to 75 mg/day, from 25 mg/day to 50 mg/day, from 50 mg/day to 200 mg/day, from 75 mg/day to 200 mg/day, from 100 mg/day to 200 mg/day, from 125 mg/day to 200 mg/day, from 150 mg/day to 200 mg/day, from 175 mg/day to 200 mg/day, from 50 mg/day to 200 mg/day, from 50 mg/day to 175 mg/day, from 50 mg/day to 150 mg/day, from 50 mg/day to 100 mg/day, from 50 mg/day to 75 mg/day, from 75 mg/day to 200 mg/day, from 75 mg/day to 175 mg/day, from 75 mg/day to 150 mg/day, from 75 mg/day to 125 mg/day, from 75 mg/day to 100 mg/day, from 100 mg/day to 200 mg/day, from 100 mg/day to 175 mg/day, from 100 mg/day to 125 mg/day, from 125 mg/day to 200 mg/day, from 125 mg/day to 175 mg/day, from 125 mg/day to 150 mg/day, from 150 mg/day to 200 mg/day, from 150 mg/day to 175 mg/day, from 175 mg/day to 200 mg/day, or any range there between. In some embodiments, the dosage of the agent for use in a human subject is 1 mg/day, 3 mg/day, 5 mg/day, 10 mg/day, 15 mg/day, 20 mg/day, 25 mg/day, 30 mg/day, 35 mg/day, 40 mg/day, 45 mg/day, 50 mg/day, 60 mg/day, 70 mg/day, 80 mg/day, 90 mg/day, 100 mg/day, 125 mg/day, 150 mg/day, 175 mg/day, 200 mg/day, 225 mg/day, 250 mg/day, 275 mg/day, 300 mg/day, 325 mg/day, 350 mg/day, 375 mg/day, 400 mg/day, 425 mg/day, 450 mg/day, 475 mg/day, or 500 mg/day.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Pharmaceutical compositions that an ALK2 inhibitor of the invention may be administered to a subject in need thereof, for example, one or more times (e.g., 1-10 times or more) daily, every other day, weekly, biweekly, monthly, bimonthly, quarterly, biannually, annually, or as medically necessary. In some embodiments, the doses recited above are administered daily or every other day for several weeks or months. In some embodiments, pharmaceutical compositions that include an ALK2 inhibitor may be administered to a subject in need thereof daily, every other day, weekly, biweekly, monthly, bimonthly, or quarterly. Dosages may be provided in either a single or multiple dosage regimens. The timing between administrations may decrease as the medical condition improves or increase as the health of the patient declines. The duration of treatment depends upon the subject's clinical progress and responsiveness to therapy. Continuous, relatively low maintenance doses are contemplated after an initial higher therapeutic dose.

In some embodiments, the pharmaceutically acceptable formulation used to administer the active compound provides sustained delivery, such as “slow-release” of the active compound to a subject. For example, the formulation can deliver the agent or composition for at least one, two, three, or four weeks after the pharmaceutically acceptable formulation is administered to the subject. Preferably, a subject to be treated in accordance with the methods described herein is treated with the active composition for at least 30 days (either by repeated administration or by use of a sustained delivery system, or both).

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Methods of Treatment

The ALK2 inhibitors described herein (e.g., a compound of Formula I, Formula II, Formula III, Compounds 1-7, BCX9250, and INCB00928 or a pharmaceutically acceptable salt thereof, e.g., an effective amount of an ALK2 inhibitor described herein) can be used to can be used to treat a subject having or at risk of developing functional iron deficiency (FID) or anemia associated with myelodysplastic syndromes (MDS), myelofibrosis (MF), or myelodysplastic syndromes/myeloproliferative neoplasm (MDS/MPN) overlap syndromes. In some embodiments, the subject has FID anemia. In some embodiments, the subject has MDS (diagnosed according to the 2016 World Health Organization (WHO) classification) that is classified as very low, low, or intermediate risk MDS as determined by the Revised International Prognostic Scoring System (IPSS—R). In some embodiments, the subject with MDS (e.g., a subject with very low, low, or intermediate risk MDS) has a bone marrow blast percentage of less than five percent prior to treatment (e.g., for at least six months or more prior to treatment initiation with an ALK2 inhibitor described herein). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS—RS-SLD) and multilineage dysplasia (MDS-RS-MLD)), myelodysplastic syndrome associated with isolated del chromosome abnormality (MDS with isolated del (5q)), myelodysplastic syndrome with excess blasts (MDS-EB; which includes myelodysplastic syndrome with excess blasts-type 1 (MDS-EB-1) and myelodysplastic syndrome with excess blasts-type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the subject has primary myelofibrosis, post-polycythemia vera MF, or post-essential thrombocythemia MF (e.g., diagnosed according to the 2017 WHO criteria). In some embodiments, the subject with MF (e.g., a subject with primary myelofibrosis, post-polycythemia vera MF, or post-essential thrombocythemia MF) has a bone marrow and peripheral blood blast percentage that is less than two percent, or that is stable between two to five percent prior to treatment (e.g., for at least six months or more prior to treatment initiation with an ALK2 inhibitor described herein). In some embodiments, the MDS/MPN overlap syndrome is chronic myelomonocytic leukemia, juvenile myelomonocytic leukemia, atypical chronic myeloid leukemia, MDS/MPN with ring sideroblasts and thrombocytosis, or MDS/MPN unclassifiable. In some embodiments, the subject with an MDS/MPN overlap syndrome (e.g., an MDS/MPN overlap syndrome diagnosed according to the 2016 WHO classification) has a bone marrow blast percentage of less than five percent prior to treatment (e.g., for at least six months or more prior to treatment initiation with an ALK2 inhibitor described herein). The subject may be diagnosed as having anemia (e.g., anemia with iron-restricted erythropoiesis) based on having a hemoglobin level of less than 11.0 g/dl (e.g., in a subject that does not require red blood cell transfusions) or based on requiring one to three units of packed RBCs in the eight weeks prior to treatment initiation with an ALK2 inhibitor described herein. In some embodiments, the subject has a transferrin saturation of less than or equal to 20% prior to treatment initiation with an ALK2 inhibitor described herein. In some embodiments, the subject has a ferritin level of greater than 100 ng/ml and less than 1000 ng/ml prior to treatment initiation with an ALK2 inhibitor described herein. In some embodiments, the subject has reticulocyte hemoglobin less than or equal to 31.0 pg prior to treatment initiation with an ALK2 inhibitor described herein. In some embodiments, the subject has an Eastern Cooperative Oncology Group (ECOG) performance score of less than or equal to two. In some embodiments, the subject is identified as having anemia prior to administration of an ALK2 inhibitor described herein. In some embodiments, the method includes a step of identifying the subject as having anemia (e.g., by evaluating hemoglobin levels) prior to administration of an ALK2 inhibitor described herein. In some embodiments, the subject is identified as having FID prior to administration of an ALK2 inhibitor described herein. In some embodiments, the method includes a step of identifying the subject as having FID prior to administration of an ALK2 inhibitor described herein. The method can further include evaluating red blood cell, hemoglobin, hematocrit, and/or reticulocyte levels after administration of an ALK2 inhibitor described herein (e.g., 12 hours, 24 hours, 1, 2, 3, 4, 5, 6, or 7 days, 1, 2, 3, 4, 5, 6, 7, 8, 9, 10, 11, or 12 weeks, or 1, 2, 3, 4, 5, or 6 months or more after the start of treatment with an ALK2 inhibitor described herein). In some embodiments, the subject does not receive concurrent treatment with a moderate to strong CYP3A₄ inhibitor (e.g., a strong CYP3A₄ inhibitor, such as boceprevir, cobicistat, ritonavir alone or in combination with danoprevir, elvitegravir, indinavir, lopinavir, paritaprevir (and ombitasvir and/or dasabuvir), saquinavir, or tipranavir, itraconazole, ketoconazole, posaconazole, telaprevir, telithromycin, troleandomycin, (triacetyl) oleandomycin, and voriconazoleor; or a moderate CYP3A₄ inhibitor, such as aprepitant, ciprofloxacin, conivaptan, crizotinib, cyclosporine, diltiazem, dronedarone, erythromycin, fluconazole, fluvoxamine, imatinib, tofisopam, and verapamil) or a moderate to strong CYP3A₄ inducer (e.g., a strong CYP3A₄ inducer, such as apalutamide, carbamazepine, enzalutamide, mitotane, phenytoin, rifampin, and St. John's wort, or a moderate CYP3A₄ inducer, such as bosentan, efavirenz, etravirine, phenobarbital, and primidone). In some embodiments, the subject does not receive concurrent treatment with a drug that is a substrate of CYP2B₆ (e.g., bupropion or efavirenz) or MATE1 (e.g., metformin). In some embodiments, the subject does not receive concurrent treatment with an inhibitor of P-glycoprotein (e.g., amiodarone, carvedilol, clarithromycin, dronedarone, itraconazole, lapatinib, propafenone, quinidine, ranolazine, ritonavir alone or in combination with lopinavir, saquinavir, or tipranavir, telaprevir, or verapamilor) an inhibitor of breast cancer resistance protein (e.g., curcumin, cyclosporine A, or eltrombopag).

In some embodiments, the methods described herein (e.g., treatment with a compound of Formula I, Formula II, o Formula III or of Compounds 1-7, BCX9250, or INCB00928) increase serum iron, increase transferrin saturation, increase iron bioavailability (e.g., by mobilizing iron from storage tissue into the circulation), increase reticulocyte hemoglobin, promote the formation of hemoglobin-containing (e.g., hemoglobin-rich) red blood cells, reestablish iron homeostasis, and/or reduce serum hepcidin (e.g., hepcidin concentration in plasma). By increasing iron bioavailability (e.g., increasing iron supply in the bone marrow), the methods described herein may promote or increase erythropoiesis or the production of functional red blood cells. In some embodiments, the methods described herein increase mean corpuscular hemoglobin (e.g., in subjects who did not require RBC transfusions prior to treatment with the ALK2 inhibitors described herein). In some embodiments, the methods described herein increase absolute reticulocyte count. The methods described herein may also affect soluble transferrin receptor levels, red cell zinc protoporphyrin, and the percentage of hypochromic red cells. These changes may be observed in a subject treated with an ALK2 inhibitor described herein compared to measurements obtained prior to treatment or compared to measurements obtained from untreated or placebo-treated subjects having the same disease or condition (e.g., FID or anemia associated with MDS, MF, or an MDS/MPN overlap syndrome).

In some embodiments, treatment according to the methods described herein leads to an increase in reticulocyte hemoglobin of >2.0 pg from baseline pre-treatment measurements and/or a reticulocyte hemoglobin value of >32 pg (e.g., after 1, 2, 3, 4, 5, 6, 7, 8, 9, 10, 11, 12, or more weeks of treatment with an ALK2 inhibitor described herein). In some embodiments, treatment according to methods described herein leads to a mean hemoglobin increase of greater than or equal to 1.0 g/dL or 1.5 g/dL from baseline or pretreatment measurements and/or leads to hemoglobin levels that are in the non-anemic range, which is defined as >12.0 g/dL for women and >13.0 g/dL for men (e.g., after 1, 2, 3, 4, 5, 6, 7, 8, 9, 10, 11, 12, or more weeks of treatment with an ALK2 inhibitor described herein, e.g., in a subject who did not receive a red blood cell transfusion in the previous 28 days). In some embodiments, the methods described herein reduce the need of a subject (e.g., a subject who previously required RBC transfusions) for an RBC transfusion (e.g., reduce transfusion burden, for example, the subject no longer needs RBC transfusions, or the subject needs fewer or less frequent RBC transfusions than before treatment with an ALK2 inhibitor described herein). In some embodiments, the compositions and methods described herein promote RBC transfusion independence (e.g., in a subject who was previously transfusion-dependent) for at least four consecutive weeks or more, such as 4 weeks, 6, weeks, 8 weeks, 10 weeks, 12 weeks, 14 weeks, 16 weeks, 18 weeks, 20 weeks, 22 weeks, 24, weeks or more, during treatment with an ALK2 inhibitor described herein. In some embodiments, treatment according to the methods described herein improves patient-reported outcomes in the 36-item Short Form Health Survey (SF-36) and/or Functional Assessment of Chronic Illness Therapy-Fatigue (FACIT-Fatigue) questionnaires.

### Kits

The compositions described herein can be provided in a kit for use in treating FID or anemia (e.g., FID anemia) associated with MDS, MF, or MDS/MPN overlap syndromes. Compositions may include an ALK2 inhibitor and may be provided in unit dosage form, optionally in a pharmaceutically acceptable excipient (e.g., saline), in an amount sufficient to treat FID or anemia (e.g., FID anemia) associated with MDS, MF, or MDS/MPN overlap syndromes. The kit can further include a package insert that instructs a user of the kit, such as a physician, to perform the methods described herein. The kit may optionally include a syringe or other device for administering the composition.

### Examples

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1-Treatment of FID or Anemia Associated with Very Low, Low, or Intermediate Risk MDS by Administration of an ALK2 Inhibitor

According to the methods disclosed herein, a physician of skill in the art can treat a subject, such as a human patient, having FID and/or anemia associated with a low, very low, or intermediate risk myelodysplastic syndrome so as to increase hemoglobin levels, increase reticulocyte hemoglobin, increase reticulocytes, increase serum iron, increase transferrin saturation, increase iron bioavailability, reduce hepcidin concentration in plasma, increase mean corpuscular hemoglobin, reduce transfusion burden, or promote transfusion independence. The method of treatment can include diagnosing or identifying a subject as a candidate for treatment using the IPSS—R and/or by measuring hemoglobin levels. To treat the subject, a physician of skill in the art can administer to the subject a composition containing an ALK2 inhibitor described herein (e.g., a compound of any one of Formulas I-III or any one of Compounds 1-7, BCX9250, or INCB00928, or a pharmaceutically acceptable salt thereof, such as a compound of Formula I-11 or Formula II-42 or a pharmaceutically acceptable salt thereof). The composition containing the ALK2 inhibitor may be administered to the subject, for example, by oral administration. The ALK2 inhibitor is administered in a therapeutically effective amount, such as from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 60, 70, 80, 90, 100, 150, 200, 250, 300, 350, 400, 450, or 500 mg/kg) or 1 mg/day to 500 mg/day (e.g., 1, 5, 10, 30, 40, 50, 60, 70, 80, 90, 100, 110, 120, 130, 140, 150, 160, 170, 180, 190, 200, 250, 300, 350, 400, 450, or 500 mg/day). In some embodiments, the ALK2 inhibitor is administered bimonthly, once a month, once every two weeks, once a week, once every other day, or once a day. The ALK2 inhibitor is administered in an amount sufficient to increase hemoglobin levels, increase reticulocyte hemoglobin, increase reticulocytes, increase serum iron, increase transferrin saturation, increase iron bioavailability, reduce hepcidin concentration in plasma, increase mean corpuscular hemoglobin, reduce transfusion burden, or promote transfusion independence.

Following administration of the composition to a patient, a practitioner of skill in the art can monitor the patient's improvement in response to the therapy by a variety of methods. For example, a physician can monitor the patient's hemoglobin levels, reticulocyte hemoglobin, reticulocyte count, serum iron, transferrin saturation, or hepcidin concentration using a blood test. A finding that the patient's hemoglobin levels, reticulocyte hemoglobin, reticulocyte count, serum iron, or transferrin saturation are increased and/or that the patient's hepcidin levels are decreased following administration of the composition compared to test results prior to administration of the composition indicates that the patient is responding favorably to the treatment. Subsequent doses can be determined and administered as needed.

### Example 2-Treatment of FID or Anemia Associated with Primary Myelofibrosis, Post-Polycythemia Vera MF, or Post-Essential Thrombocythemia MF by Administration of an ALK2 Inhibitor

According to the methods disclosed herein, a physician of skill in the art can treat a subject, such as a human patient, having FID and/or anemia associated with primary myelofibrosis, post-polycythemia vera MF, or post-essential thrombocythemia MF so as to increase hemoglobin levels, increase reticulocyte hemoglobin, increase reticulocytes, increase serum iron, increase transferrin saturation, increase iron bioavailability, reduce hepcidin concentration in plasma, increase mean corpuscular hemoglobin, reduce transfusion burden, or promote transfusion independence. The method of treatment can include diagnosing or identifying a subject as a candidate for treatment using the 2017 WHO criteria and/or by measuring hemoglobin levels. To treat the subject, a physician of skill in the art can administer to the subject a composition containing an ALK2 inhibitor described herein (e.g., a compound of any one of Formulas I-III or any one of Compounds 1-7, BCX9250, or INCB00928, or a pharmaceutically acceptable salt thereof, such as a compound of Formula I-11 or Formula II-42 or a pharmaceutically acceptable salt thereof). The composition containing the ALK2 inhibitor may be administered to the subject, for example, by oral administration. The ALK2 inhibitor is administered in a therapeutically effective amount, such as from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 60, 70, 80, 90, 100, 150, 200, 250, 300, 350, 400, 450, or 500 mg/kg) or 1 mg/day to 500 mg/day (e.g., 1, 5, 10, 20 30, 40, 50, 60, 70, 80, 90, 100, 110, 120, 130, 140, 150, 160, 170, 180, 190, 200, 250, 300, 350, 400, 450, or 500 mg/day). In some embodiments, the ALK2 inhibitor is administered bimonthly, once a month, once every two weeks, once a week, once every other day, or once a day. The ALK2 inhibitor is administered in an amount sufficient to increase hemoglobin levels, increase reticulocyte hemoglobin, increase reticulocytes, increase serum iron, increase transferrin saturation, increase iron bioavailability, reduce hepcidin concentration in plasma, increase mean corpuscular hemoglobin, reduce transfusion burden, or promote transfusion independence.

Following administration of the composition to a patient, a practitioner of skill in the art can monitor the patient's improvement in response to the therapy by a variety of methods. For example, a physician can monitor the patient's hemoglobin levels, reticulocyte hemoglobin, reticulocyte count, serum iron, transferrin saturation, or hepcidin concentration using a blood test. A finding that the patient's hemoglobin levels, reticulocyte hemoglobin, reticulocyte count, serum iron, or transferrin saturation are increased and/or that the patient's hepcidin levels are decreased following administration of the composition compared to test results prior to administration of the composition indicates that the patient is responding favorably to the treatment. Subsequent doses can be determined and administered as needed.

### Example 3-Treatment of FID or Anemia Associated with Chronic Myelomonocytic Leukemia by Administration of an ALK2 Inhibitor

According to the methods disclosed herein, a physician of skill in the art can treat a subject, such as a human patient, having FID and/or anemia associated with chronic myelomonocytic leukemia so as to increase hemoglobin levels, increase reticulocyte hemoglobin, increase reticulocytes, increase serum iron, increase transferrin saturation, increase iron bioavailability, reduce hepcidin concentration in plasma, increase mean corpuscular hemoglobin, reduce transfusion burden, or promote transfusion independence. The method of treatment can include diagnosing or identifying a subject as a candidate for treatment using the 2016 WHO classification and/or by measuring hemoglobin levels. To treat the subject, a physician of skill in the art can administer to the subject a composition containing an ALK2 inhibitor described herein (e.g., a compound of any one of Formulas I-III or any one of Compounds 1-7, BCX9250, or INCB00928, or a pharmaceutically acceptable salt thereof, such as a compound of Formula I-11 or Formula II-42 or a pharmaceutically acceptable salt thereof). The composition containing the ALK2 inhibitor may be administered to the subject, for example, by oral administration. The ALK2 inhibitor is administered in a therapeutically effective amount, such as from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 60, 70, 80, 90, 100, 150, 200, 250, 300, 350, 400, 450, or 500 mg/kg) or 1 mg/day to 500 mg/day (e.g., 1, 5, 10, 20 30, 40, 50, 60, 70, 80, 90, 100, 110, 120, 130, 140, 150, 160, 170, 180, 190, 200, 250, 300, 350, 400, 450, or 500 mg/day). In some embodiments, the ALK2 inhibitor is administered bimonthly, once a month, once every two weeks, once a week, once every other day, or once a day. The ALK2 inhibitor is administered in an amount sufficient to increase hemoglobin levels, increase reticulocyte hemoglobin, increase reticulocytes, increase serum iron, increase transferrin saturation, increase iron bioavailability, reduce hepcidin concentration in plasma, increase mean corpuscular hemoglobin, reduce transfusion burden, or promote transfusion independence.

Following administration of the composition to a patient, a practitioner of skill in the art can monitor the patient's improvement in response to the therapy by a variety of methods. For example, a physician can monitor the patient's hemoglobin levels, reticulocyte hemoglobin, reticulocyte count, serum iron, transferrin saturation, or hepcidin concentration using a blood test. A finding that the patient's hemoglobin levels, reticulocyte hemoglobin, reticulocyte count, serum iron, or transferrin saturation are increased and/or that the patient's hepcidin levels are decreased following administration of the composition compared to test results prior to administration of the composition indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth. All publications, patents, and patent applications are herein incorporated by reference in their 10 entirety to the same extent as if each individual publication, patent or patent application was specifically and individually indicated to be incorporated by reference in its entirety.

Other embodiments are within the following claims.

1. A method of treating a subject having or at risk of developing functional iron deficiency associated with a very low, low, or intermediate risk myelodysplastic syndrome (MDS), comprising administering to the subject a therapeutically effective amount of a small molecule ALK2 inhibitor or a pharmaceutically acceptable salt thereof.

2. The method of claim 1, wherein the myelodysplastic syndrome is myelodysplastic syndrome with unilineage dysplasia, myelodysplastic syndrome with multilineage dysplasia, myelodysplastic syndrome with ring sideroblasts, myelodysplastic syndrome with isolated del (5q), myelodysplastic syndrome with excess blasts, or myelodysplastic syndrome unclassifiable.

3. The method of claim 1 or 2, wherein the myelodysplastic syndrome is a ring sideroblast positive myelodysplastic syndrome.

4. The method of claim 1 or 2, wherein the myelodysplastic syndrome is a non-ring sideroblast myelodysplastic syndrome.

5. A method of treating a subject having or at risk of developing functional iron deficiency associated with primary myelofibrosis (PMF), post-polycythemia vera myelofibrosis (post-PV MF), or post-essential thrombocythemia myelofibrosis (post-ET MF), comprising administering to the subject a therapeutically effective amount of a small molecule ALK2 inhibitor or a pharmaceutically acceptable salt thereof.

6. A method of treating a subject having or at risk of developing functional iron deficiency associated with chronic myelomonocytic leukemia, comprising administering to the subject a therapeutically effective amount of a small molecule ALK2 inhibitor or a pharmaceutically acceptable salt thereof.

7. A method of treating a subject having or at risk of developing functional iron deficiency associated with juvenile myelomonocytic leukemia, comprising administering to the subject a therapeutically effective amount of a small molecule ALK2 inhibitor or a pharmaceutically acceptable salt thereof.

8. A method of treating a subject having or at risk of developing functional iron deficiency associated with atypical chronic myeloid leukemia, comprising administering to the subject a therapeutically effective amount of a small molecule ALK2 inhibitor or a pharmaceutically acceptable salt thereof.

9. A method of treating a subject having or at risk of developing functional iron deficiency associated with myelodysplastic/myeloproliferative neoplasm, unclassifiable (MDS/MPN-U), comprising administering to the subject a therapeutically effective amount of a small molecule ALK2 inhibitor or a pharmaceutically acceptable salt thereof.

10. The method of any one of claims 1-9, wherein the subject has or is at risk of developing anemia.

11. The method of any one of claims 1-10, wherein the subject does not receive concurrent treatment with a moderate to strong CYP3A₄ inhibitor or inducer, a drug that is a substrate of CYP2B₆ or MATE1, an inhibitor of P-glycoprotein, or an inhibitor of breast cancer resistance protein.

12. The method of any one of claims 1-11, wherein the method reduces the subject's transfusion burden.

13. The method of any one of claims 1-12, wherein the subject achieves transfusion independence for at least eight consecutive weeks during treatment.

14. The method of any one of claims 1-13, wherein the method increases mean hemoglobin by greater than or equal to 1.0 g/dL.

15. The method of any one of claims 1-14, wherein the method increases the subject's hemoglobin levels to be in a non-anemic range.

16. The method of any one of claims 1-15, wherein the method increases reticulocyte hemoglobin by greater than 2.0 pg from baseline or increases the subject's reticulocyte hemoglobin to greater than 32 pg.

17. The method of any one of claims 1-16, wherein the small molecule ALK2 inhibitor or a pharmaceutically acceptable salt thereof is administered in an amount sufficient to increase serum iron, increase transferrin saturation, increase iron bioavailability, increase reticulocyte hemoglobin, promote formation of hemoglobin-containing red blood cells, reestablish iron homeostasis, reduce hepcidin concentration in plasma, increase mean corpuscular hemoglobin, increase absolute reticulocyte count, or treat anemia.

18. The method of any one of claims 1-17, wherein the small molecule ALK2 inhibitor is a compound of:
i) Formula I 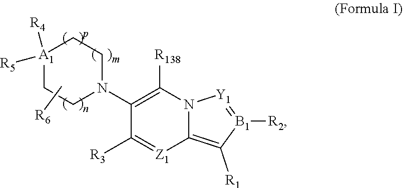
wherein
R₁ is hydrogen or an optionally substituted substituent selected from cycloalkyl, aryl, heteroaryl, and heterocyclyl;
R₂ is optionally absent, hydrogen, CN, NO₂, or an optionally substituted substituent selected from alkyl, alkoxy, heterocyclyloxy, heteroaryloxy, aryloxy, cycloalkyloxy, carbonyl, amino, amido, sulfonyl, sulfonamido, cycloalkyl, aryl, heterocyclyl, and heteroaryl;
R₃ is hydrogen, CN, NO₂, or an optionally substituted substituent selected from alkyl, alkoxy, heterocyclyloxy, heteroaryloxy, aryloxy, cycloalkyloxy, carbonyl, amino, amido, sulfonyl, sulfonamido, cycloalkyl, aryl, heterocyclyl, and heteroaryl;
R₄ is optionally absent, hydrogen, O—, halo, CN, NO₂, hydroxy, or an optionally substituted substituent selected from alkyl, alkenyl, alkynyl, carbonyl, cycloalkyl, aryl, alkoxy, aryloxy, cycloalkyloxy, amino, amido, alkoxycarbonyl, carboxy, sulfonyl, sulfonamido, thio, heterocyclyl, heterocyclyloxy, heteroaryl, and heteroaryloxy;
R₅ is optionally absent, hydrogen, halo, hydroxy, or optionally substituted alkyl;
R₁₃₈ is hydrogen, halo, hydroxy, or an optionally substituted substituent selected from alkyl, carbonyl, alkoxy, thio, amino, amido, heterocyclyl, aryl, and heteroaryl;
R₆ is independently one or more of hydrogen, halo, CN, NO₂, hydroxy, or an optionally substituted substituent selected from alkyl, alkenyl, alkynyl, alkoxy, heterocyclyloxy, heteroaryloxy, aryloxy, cycloalkyloxy, amino, amido, carbonyl, alkoxycarbonyl, carboxy, sulfonyl, sulfonamido, thio, cycloalkyl, aryl, heterocyclyl, and heteroaryl and oxo; B₁, is C or N; Y₁ is N or CR₁₃₉, wherein R₁₃₉ is hydrogen, halo, hydroxy, or an optionally substituted substituent selected from alkyl, carbonyl, alkoxy, thio, amino, amido, heterocyclyl, aryl, and heteroaryl; Z₁ is N or CR₁₄₀, wherein R₁₄₀ is hydrogen, halo, hydroxy, or an optionally substituted substituent selected from alkyl, carbonyl, alkoxy, thio, amino, amido, heterocyclyl, aryl, or heteroaryl; A₁ is C, N, O, C(O), S, SO, or SO₂; m is 0, 1, 2, or 3; n is 0, 1, 2, or 3; and p is 0 or 1; wherein optionally any two or more of R₄, R₅, or R₆ may be joined together to form one or more rings;
ii) Formula II 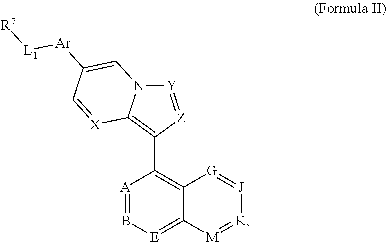
wherein:
a) X and Y are independently selected from CR₁₅ and N;
Z is selected from CR₃ and N;
Ar is a substituted or unsubstituted aryl ring or a substituted or unsubstituted heteroaryl ring;
L₁ is absent or selected from substituted or unsubstituted alkyl, substituted or unsubstituted cycloalkyl, substituted or unsubstituted cycloalkylalkyl, cycloalkyl-heteroalkyl, substituted or unsubstituted heterocyclyl, substituted or unsubstituted heterocyclylalkyl, substituted or unsubstituted heterocyclyl-heteroalkyl, and substituted or unsubstituted heteroalkyl; and
J and K are both absent or, independently for each occurrence, are each CR¹⁶;
A is CR¹⁶;
B and E are each independently CR¹⁷;
if J and K are absent, then G is R¹⁶ and M is R¹⁷; if J and K are not absent, then G is CR¹⁶ and M is CR¹⁷;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from 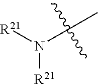
and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, OH, halogen, cyano, carboxyl, and substituted or unsubstituted acyl, alkanol, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkoxy, alkylthio, acyloxy, amino, acylamino, carbamate, amido, amidino, sulfonyl, sulfoxido, sulfamoyl, or sulfonamide;
R¹⁷, independently for each occurrence, is selected from R¹⁶ and —R²², —NH₂, —NHR²², —N(R²²)₂, halogen, —CO₂H, —CO₂R²², —CONH₂, —CONHR²², —CON(R²²)₂, —C(NH₂)═N(OH), —C(NHR²²)═N(OH), —C(N(R²²)₂)═N(OH), —C(NH₂)═NH, —C(NHR²²)═NH, —C(NHR²²)═NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²², 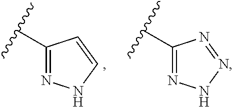 [SMILES: *c1cc[nH]n1 ; *c1nn[nH]n1]
—CH(OH)R²², —C(OH)(R²²)₂, —CH(NH₂)(R²²), —CH(NHR²²)(R²²), —CH(N(R²²)₂)(R²²), pyrazol-3-yl, pyrazol-4-yl, and —OR²², provided that at least one R¹⁷ is —R²², —NH₂, —NHR²², —N(R²²)₂, halogen, —CO₂H, —CO₂R²², —CONH₂, —CONHR²², —CON(R²²)₂, —C(NH₂)═N(OH), —C(NHR²²)—N(OH), —C(N(R²²)₂)═N(OH), —C(NH₂)═NH, —C(NHR²²)═NH, —C(NHR²²)═NR²², —C(N(R²²)₂)═NH, —C(N(R²²)₂)═NR²², —CN, —CH₂CH₂OH, —CH₂OH, —CH₂SO₂NH₂, —CH₂SO₂NHR²², —CH₂SO₂N(R²²)₂, —SO₂NH₂, —SO₂NHR²², —SO₂N(R²²)₂, —NHSO₂R²², —SO₂R²², —CH₂SO₂R²², —CH₂NH₂, —CH₂NHR²², —CH₂N(R²²)₂, —C(O)R²², 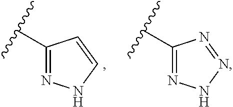 [SMILES: *c1cc[nH]n1 ; *c1nn[nH]n1]
—CH(OH)R²²_C(OH)(R²²)₂, —CH(NH₂)(R²²), —CH(NHR²²)(R²²), —CH(N(R²²)₂)(R²²), pyrazol-3-yl, pyrazol-4-yl, or —OR²²;
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide; and
R²², independently for each occurrence, is selected from lower alkyl and cycloalkyl;
b) X and Y are independently selected from CR¹⁵ and N;
Z is selected from CR³ and N;
Ar is a substituted or unsubstituted aryl ring or a substituted or unsubstituted heteroaryl ring;
L₁ is absent or selected from substituted or unsubstituted alkyl and heteroalkyl; and
J and K are both absent or, independently for each occurrence, are each CR¹⁶;
A and B, independently for each occurrence, are CR¹⁶;
E is CR¹⁷;
if J and K are absent, then G and M are each independently R¹⁸; if J and K are not absent, then G and M are each independently CR¹⁷;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from 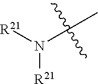
and a nitrogen-containing heterocyclyl or heteroaryl ring;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido;
R¹⁶, independently for each occurrence, is selected from H, D, OH, halogen, cyano, carboxyl, and substituted or unsubstituted acyl, alkanol, alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkylamino, aminoalkyl, alkoxy, alkylthio, acyloxy, amino, acylamino, carbamate, amido, amidino, sulfonyl, sulfoxido, sulfamoyl, sulfonamide, tetrazolyl, or trifluoromethylacyl;
R¹⁷, independently for each occurrence, is selected from R¹⁶ and H, D, —CO₂H, —CONH₂, —CONHCH₃, —CON(CH₃)₂, —C(NH₂)═N(OH), —C(NH₂)═NH, —CN, —CH₂OH, —SO₂NH₂, —CH₂NH₂, —C(O)CH₃, 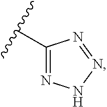 [SMILES: *c1nn[nH]n1]
—CH(OH) CH₃, —C(O)CF₃, and —OCH₃, provided that at least one R¹⁷ is H, —CO₂H, —CONH₂, —CONHCH₃, —CON(CH₃)₂, —C(NH₂)═N(OH), —C(NH₂)—NH, —CN, —CH₂OH, —SO₂NH₂, —CH₂NH₂, —C(O)CH₃, 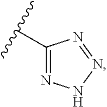 [SMILES: *c1nn[nH]n1]
—CH(OH) CH₃, —C(O)CF₃, or —OCH₃;
and
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide;
c) X and Y are independently selected from CR¹⁵ and N;
Z is selected from CR³ and N;
Ar is a phenyl ring substituted with at least one non-protium (1H) substituent or a substituted or unsubstituted heteroaryl ring;
L₁ is absent or selected from substituted or unsubstituted alkyl and heteroalkyl; and
G, J, K, and M are all absent or, independently for each occurrence, are selected from CR¹⁶ and N;
A, B, and E, independently for each occurrence, are selected from CR¹⁶ and N; provided that no more than three of A, B, E, G, J, K, and M are N, and at least one of E and M is N, and that if G, J, K, and M are absent then the carbon atom adjacent to E and M is optionally substituted with R¹⁶;
R³′ is selected from H, halogen, cyano, and substituted or unsubstituted alkyl, cycloalkyl, acylamino, carbamate, sulfonyl, sulfoxido, sulfamoyl, or sulfonamido;
R⁷ is selected from H, hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido; and
R¹⁶, independently for each occurrence, is absent or is selected from H, D, OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, or sulfonamide; or
d) X and Y are independently selected from CR¹⁵ and N;
Z is selected from CR³′ and N;
Ar is selected from substituted or unsubstituted aryl and heteroaryl;
L₁ is absent or selected from substituted or unsubstituted alkyl and heteroalkyl; and
G, J, K, and M are all absent or, independently for each occurrence, are selected from CR¹⁶ and N;
A, B, and E, independently for each occurrence, are selected from CR¹⁶ and N;
provided that no more than three of A, B, E, G, J, K, and M are N, and at least one of E and M is N, and that if G, J, K, and M are absent then the carbon atom adjacent to E and M is optionally substituted with R¹⁶;
R³ is selected from H, halogen, cyano, and substituted or unsubstituted alkyl, cycloalkyl, acylamino, carbamate, sulfonyl, sulfoxido, sulfamoyl, or sulfonamido;
R⁷ is selected from H, hydroxyl, carboxyl, and substituted or unsubstituted alkenyl, alkynyl, cycloalkyl, heterocyclyl, aryl, heteroaryl, acyl, ester, alkoxyl, alkylthio, acyloxy, amino, acylamino, carbamate, amido, amidino, sulfonyl, sulfoxido, sulfamoyl, or sulfonamido;
R¹⁵, independently for each occurrence, is selected from H, halogen, cyano, and substituted or unsubstituted alkyl, cycloalkyl, heterocyclyl, cycloalkylalkyl, heterocyclylalkyl, acylamino, carbamate, sulfonyl, sulfoxido, sulfamoyl, or sulfonamido; and
R¹⁶, independently for each occurrence, is absent or is selected from H, D, OH, halogen, cyano, carboxyl, and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, acyl, ester, alkoxy, alkylthio, acyloxy, amino, acylamino, carbamate, amido, amidino, sulfonyl, sulfoxido, sulfamoyl, and sulfonamide;
wherein B is C—R²⁵ when E is N or K is C—R²⁵ when M is N or both such that at least one of B and K is C—R²⁵, where
R²⁵ is selected from deuterium, halogen, hydroxyl, lower alkyl, and lower alkoxy, such as deuterium, fluorine, chlorine, methyl, ethyl, hydroxy, or methoxy:
iii) Formula III 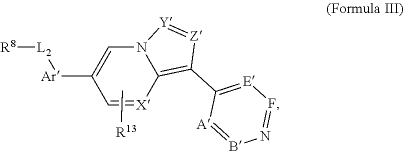
wherein X′ is selected from CR¹⁵ and N;
Y′ is selected from CR¹⁵ and N;
Z′ is selected from CR²⁸ and N;
Ar′ is selected from substituted or unsubstituted aryl and heteroaryl;
L₂ is absent or selected from substituted or unsubstituted alkyl and heteroalkyl;
A and B, independently for each occurrence, are selected from CR¹⁶ and N;
E and F, independently for each occurrence, are selected from CR⁵′ and N;
R²⁸ represents a substituent selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido;
R⁸ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R⁵, independently for each occurrence, represents a substituent selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, or two occurrences of R⁵ taken together with the atoms to which they are attached form a substituted or unsubstituted 5- or 6-membered cycloalkyl, heterocycloalkyl, aryl, or heteroaryl ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido;
R¹⁵′, independently for each occurrence, represents a substituent selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido;
R¹⁶′, independently for each occurrence, represents a substituent selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido; or
iv) any one of compounds 1-7: 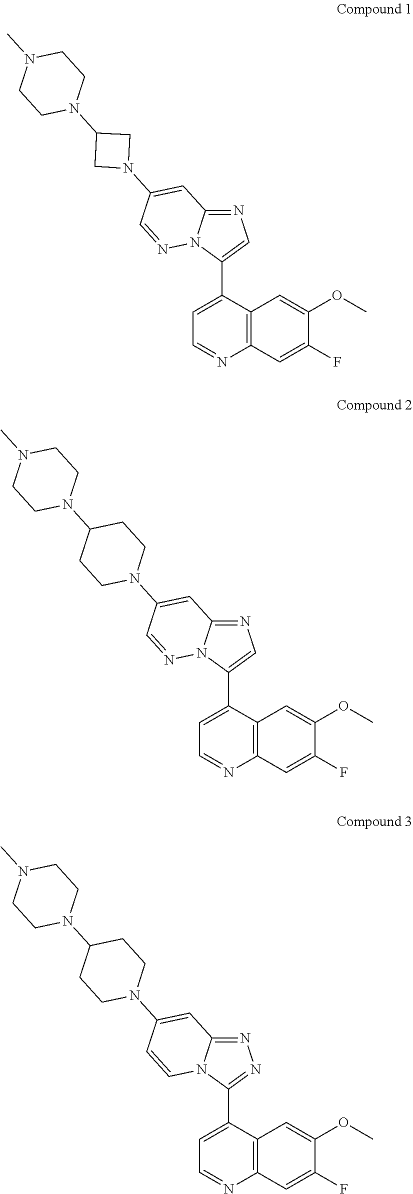 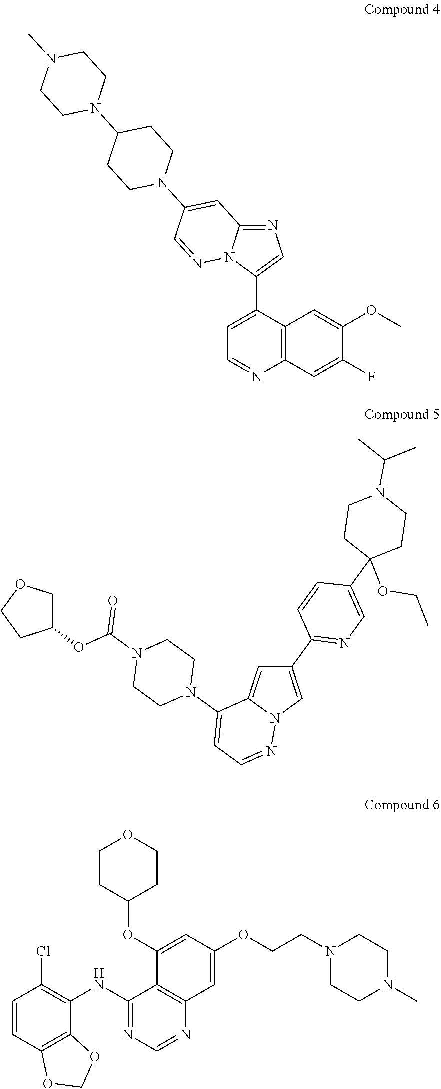 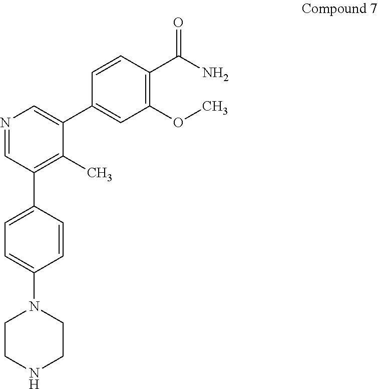 [SMILES: COc1cc(-c2cncc(-c3ccc(N4CCNCC4)cc3)c2C)ccc1C(N)=O]
or a pharmaceutically acceptable salt thereof.

19. The method of claim 18, wherein the small molecule ALK2 inhibitor is a compound of Formula I or a pharmaceutically acceptable salt thereof.

20. The method of claim 19, wherein the compound of Formula I is a compound of any one of Formulas I-1 to 1-200 or a pharmaceutically acceptable salt thereof.

21. The method of claim 20, wherein the compound of Formula I is a compound of Formula I-11 or a pharmaceutically acceptable salt thereof.

22. The method of claim 18, wherein the small molecule ALK2 inhibitor is a compound of Formula II or a pharmaceutically acceptable salt thereof.

23. The method of claim 22, wherein the compound of Formula II is a compound of any one of Formulas II-1 to II-275 or a pharmaceutically acceptable salt thereof.

24. The method of claim 18, wherein the small molecule ALK2 inhibitor is a compound of Formula III or a pharmaceutically acceptable salt thereof.

25. The method of claim 24, wherein the compound of Formula III is a compound of any one of Formulas III-1 to III-35 or a pharmaceutically acceptable salt thereof.

26. The method of claim 18, wherein the small molecule ALK2 inhibitor is Compound 1 or a pharmaceutically acceptable salt thereof.

27. The method of claim 18, wherein the small molecule ALK2 inhibitor is Compound 2 or a pharmaceutically acceptable salt thereof.

28. The method of claim 18, wherein the small molecule ALK2 inhibitor is Compound 3 or a pharmaceutically acceptable salt thereof.

29. The method of claim 18, wherein the small molecule ALK2 inhibitor is Compound 4 or a pharmaceutically acceptable salt thereof.

30. The method of claim 18, wherein the small molecule ALK2 inhibitor is Compound 5 or a pharmaceutically acceptable salt thereof.

31. The method of claim 18, wherein the small molecule ALK2 inhibitor is Compound 6 or a pharmaceutically acceptable salt thereof.

32. The method of claim 18, wherein the small molecule ALK2 inhibitor is Compound 7 or a pharmaceutically acceptable salt thereof.

33. The method of claim 18, wherein the small molecule ALK2 inhibitor is administered in a pharmaceutical composition further comprising one or more pharmaceutically acceptable excipients.
